(19) United States
(12) Patent Application Publication
(10) Pub. No.: US 2023/0072897 A1
(43) Pub. Date: Mar. 9, 2023

(54) ANTI-B7-H4 ANTIBODY-DRUG CONJUGATE AND MEDICINAL USE THEREOF

(71) Applicants: SHANGHAI HANSOH BIOMEDICAL CO., LTD., Shanghai (CN); Jiangsu Hansoh Pharmaceutical Group Co., Ltd., Lianyungang (CN)

(72) Inventors: Haiqing HUA, Lianyungang (CN); Suxia LIU, Lianyungang (CN); Rudi BAO, Lianyungang (CN)

(21) Appl. No.: 17/596,239

(22) PCT Filed: Jun. 8, 2020
(86) PCT No.: PCT/CN2020/094856

§ 371 Date: Dec. 6, 2021

(30) Foreign Application Priority Data
Jun. 6, 2019 (CN) 201910498993.2
Mar. 24, 2020 (CN) 202010215322.3

(51) Int. Cl. A61K 47/68; A61P 35/00

(52) U.S. Cl. CPC A61K 47/6849; A61K 47/6803; A61K 47/6817; A61P 35/00

(57) ABSTRACT

Provided are an anti-B7-H4 antibody-drug conjugate and medicinal use thereof. Specifically, provided is an anti-B7-H4 antibody or antigen-binding fragment thereof, a humanized antibody comprising the CDR region of the anti-B7-H4 antibody, and antibody-drug conjugate thereof or pharmaceutically acceptable salt or solvent compound thereof, and the aforesaid antibody-drug conjugate thereof or pharmaceutical composition of a pharmaceutically acceptable salt or solvent compound thereof, and use thereof as an anti-cancer drug, particularly the use in the preparation of a drug for treating diseases or disorders having high expression of B7-H4.

### FIELD OF THE INVENTION

The present invention belongs to the field of biomedicine. Specifically, the present invention relates to an anti-B7-H4 antibody-drug conjugate and medicinal use thereof.

### BACKGROUND OF THE INVENTION

Tumor immunotherapy is a long-term sustainable hotspot of research and development in the field of tumor therapy, and T cell tumor immunotherapy is at a core position. Tumor evasion is a huge obstacle to tumor immunotherapy. Most tumors express antigens that can be recognized by the host immune system to varying degrees. However, in many cases, the inefficient activation of effector T cells triggers an insufficient immune response, and thus tumor cells promote tumor growth by their inhibitory effects on immune system. Tumor immunotherapy is to make full use of and mobilize the killer T cells and/or other immune cells in patients with tumor to kill the tumor.

Research on the CD28 receptor and ligands thereof has led to the characterization of molecules referred to as B7 superfamily. The members of the B7 family are a class of immunoglobulins with immunoglobulin-like V domains (IgV) and immunoglobulin-like C domains (IgC). Its members include co-stimulatory factors B7.1 (CD80) and B7.2 (CD86), inducible co-stimulatory factor ligand (ICOS-L/B7-H2), programmed death-1 ligand (PD-L1/B7-H1), programmed death-2 ligand (PD-L2/B7-DC), B7-H3 and B7-H4, and the like.

Human B7-H4 is a type I transmembrane protein consisting of 282 amino acids. Its coding gene is located in the p11.1 region of chromosome 1 (Choi I H et al., J Immunol. 2003 Nov. 1; 171(9): 4650-4). B7-H4 has a negative regulatory effect on the immune response of T cells. B7-H4 plays a broad inhibitory role in the differentiation and development, cell cycle progression and cytokine production of CD4+ and CD8+ T cells (Sica G L et al., Immunity. 2003 June; 18(6): 849-61). No immune cell disorders or autoimmune phenomena were found in B7-H4 knockout mice (Zhu G et al., Blood. 2009 Feb. 19; 113(8): 1759-67; Suh W K et al., Blood. Mol Cell Biol. 2006 September; 26(17): 6403-11). At present, the receptor of B7-H4 and signaling transduction pathway thereof are still unclear.

Recent studies have found that B7-H4 protein is abundantly expressed in a variety of tumor tissues, allowing tumor cell evasion from the attack by immune system of the body. Taking the B7-H4 molecule as a target of tumor therapy provides a new method for tumor immunotherapy.

It is currently known that human B7-H4 is expressed on cancer cells such as breast cancer, ovarian cancer, lung cancer, cervical cancer, kidney cancer, bladder cancer, and liver cancer. The expression of B7-H4 mRNA is found in the spleen, lung, thymus, liver, skeletal muscle, kidney, pancreas, testis and ovary. At protein level, low level of B7-H4 expression is found in tissues such as the breast (duct and lobule), fallopian tube epithelium, and endometrial gland. Related studies have also shown that B7-H4 is overexpressed in tumor-associated macrophages (TAM) (Kryczek, I. et al., J. Exp. Med. 2006, 203(4): 871-881), and macrophages constitute an important component of the tumor microenvironment and can represent as much as 50% of the tumor mass.

One strategy for cancer treatment is to use antibodies as carriers to deliver cytotoxic molecules into cancer cells, and then kill the cancer cells by the dissociated small molecules. Drugs used in this strategy are called antibody-drug conjugates. Adcetris and Kadcyla are currently marketed antibody-drug conjugates. At present, many multinational pharmaceutical companies are developing monoclonal antibodies against B7-H4 and/or their drug conjugates to improve the patient's own immune system response to tumors and to achieve the goal of direct killing of tumor cells. Related patents are for example WO2013025779. US20140322129 and the like. The anti-B7-H4 monoclonal antibodies of Medimmune, FivePrime and other companies are currently still under pre-clinical development; the anti-B7-H4 antibody-drug conjugates of Genentech have also been in the pre-clinical development stage.

### SUMMARY OF THE INVENTION

The objective of the present invention is to provide an anti-B7-H4 antibody-drug conjugate, which has high affinity, high selectivity, high endocytic efficiency, high anti-cancer activity, high stability, high safety and low toxic and side effects, achieved through the following technical solutions:

An antibody-drug conjugate of general formula (A) or a pharmaceutically acceptable salt or solvate thereof,

Ab-(L₂-L₁-D)_{y} (A)
wherein:
D is a cytotoxic drug;
L₁ and L₂ are linker units;
y is a number of 1 to 20;
Ab is a B7-H4 antibody or antigen-binding fragment thereof, which comprises antibody light chain variable region and antibody heavy chain variable region;

the antibody heavy chain variable region comprises at least one HCDR as shown in the sequence selected from the group consisting of:

SEQ ID NO:3, SEQ ID NO:4, SEQ ID NO:5,

SEQ ID NO:9, SEQ ID NO:10, SEQ ID NO:11;

the antibody light chain variable region comprises at least one LCDR as shown in the sequence selected from the group consisting of:

SEQ ID NO: 6, SEQ ID NO: 7, SEQ ID NO: 8.

SEQ ID NO: 12, SEQ ID NO: 13, SEQ ID NO: 14.

In one embodiment of the present invention, the antibody heavy chain variable region can also comprise at least one HCDR as shown in the sequence selected from the group consisting of:

SEQ ID NO:3, SEQ ID NO:4, SEQ ID NO:5,

SEQ ID NO:9, SEQ ID NO:10, SEQ ID NO:11,

SEQ ID NO:23, SEQ ID NO:24, SEQ ID NO:25,

SEQ ID NO:29, SEQ ID NO:30, SEQ ID NO:31, or,

the antibody heavy chain variable region can also comprise at least one HCDR as shown in the sequence selected from the group consisting of:

SEQ ID NO:43, SEQ ID NO:44, SEQ ID NO:45:

the antibody light chain variable region comprises at least one LCDR as shown in the sequence selected from the group consisting of:

SEQ ID NO: 6, SEQ ID NO: 7, SEQ ID NO: 8.

SEQ ID NO: 12, SEQ ID NO: 13, SEQ ID NO: 14,

SEQ ID NO:26, SEQ ID NO:27, SEQ ID NO:28,

SEQ ID NO:32, SEQ ID NO:33, SEQ ID NO:34.

In one embodiment of the present invention, the antibody-drug conjugate of general formula (A) or a pharmaceutically acceptable salt or solvate thereof as described above, wherein, the anti-B7-H4 antibody or antigen-binding fragment thereof comprises antibody heavy chain variable region, wherein the antibody heavy chain variable region comprises:

HCDR1 as shown in SEQ ID NO:3, HCDR2 as shown in SEQ ID NO:4 and HCDR3 as shown in SEQ ID NO:5:

or,

HCDR1 as shown in SEQ ID NO:9, HCDR2 as shown in SEQ ID NO:10 and HCDR3 as shown in SEQ ID NO:11.

In one embodiment of the present invention, the antibody-drug conjugate of general formula (A) or a pharmaceutically acceptable salt or solvate thereof as described above, wherein, the anti-B7-H4 antibody or antigen-binding fragment thereof comprises antibody heavy chain variable region, wherein the antibody heavy chain variable region comprises:

HCDR1 as shown in SEQ ID NO:23, HCDR2 as shown in SEQ ID NO:24 and HCDR3 as shown in SEQ ID NO:25:

or,

HCDR1 as shown in SEQ ID NO:29, HCDR2 as shown in SEQ ID NO:30 and HCDR3 as shown in SEQ ID NO:31.

In one embodiment of the present invention, the antibody-drug conjugate of general formula (A) or a pharmaceutically acceptable salt or solvate thereof as described above, wherein, the anti-B7-H4 antibody or antigen-binding fragment thereof comprises antibody heavy chain variable region, wherein the antibody heavy chain variable region comprises:

HCDR1 as shown in SEQ ID NO:43, HCDR2 as shown in SEQ ID NO:44 and HCDR3 as shown in SEQ ID NO:25:

or,

HCDR1 as shown in SEQ ID NO:29, HCDR2 as shown in SEQ ID NO:45 and HCDR3 as shown in SEQ ID NO:31.

In one embodiment of the present invention, the antibody-drug conjugate of general formula (A) or a pharmaceutically acceptable salt or solvate thereof as described above, wherein the antibody heavy chain variable region of the Ab comprises the CDRs of any one selected from the group consisting of the following (1) to (4):

(1) HCDR1 as shown in SEQ ID NO:3, HCDR2 as shown in SEQ ID NO:4 and HCDR3 as shown in SEQ ID NO:5:

(2) HCDR1 as shown in SEQ ID NO:9, HCDR2 as shown in SEQ ID NO:10 and HCDR3 as shown in SEQ ID NO:11;

(3) HCDR1 as shown in SEQ ID NO:23, HCDR2 as shown in SEQ ID NO:24 and HCDR3 as shown in SEQ ID NO:25; or,

(4) HCDR1 as shown in SEQ ID NO:29, HCDR2 as shown in SEQ ID NO:30 and HCDR3 as shown in SEQ ID NO:31.

The antibody heavy chain variable region of the Ab can also comprise CDRs selected from the group consisting of the following (5) to (6):

(5) HCDR1 as shown in SEQ ID NO:43, HCDR2 as shown in SEQ ID NO:44 and HCDR3 as shown in SEQ ID NO:25:

or,

(6) HCDR1 as shown in SEQ ID NO:29, HCDR2 as shown in SEQ ID NO:45 and HCDR3 as shown in SEQ ID NO:31.

In one embodiment of the present invention, the antibody-drug conjugate of general formula (A) or a pharmaceutically acceptable salt or solvate thereof as described above, wherein, the anti-B7-H4 antibody or antigen-binding fragment thereof comprises antibody light chain variable region, wherein the antibody light chain variable region comprises:

LCDR1, LCDR2 and LCDR3 as shown in SEQ ID NO:6, SEQ ID NO: 7 and SEQ ID NO:8, respectively;

or LCDR1, LCDR2 and LCDR3 as shown in SEQ ID NO:12, SEQ ID NO:13 and SEQ ID NO:14, respectively.

In some embodiment of the present invention, the antibody-drug conjugate of general formula (A) or a pharmaceutically acceptable salt or solvate thereof as described above, wherein, the anti-B7-H4 antibody or antigen-binding fragment thereof comprises antibody light chain variable region, wherein the antibody light chain variable region comprises:

LCDR1, LCDR2 and LCDR3 as shown in SEQ ID NO:26, SEQ ID NO:27 and SEQ ID NO:28, respectively;

or LCDR1, LCDR2 and LCDR3 as shown in SEQ ID NO:32, SEQ ID NO:33 and SEQ ID NO:34, respectively.

In one embodiment of the present invention, the antibody-drug conjugate of general formula (A) or a pharmaceutically acceptable salt or solvate thereof as described above, wherein, the antibody heavy chain variable region of the Ab comprises the CDRs of any one selected from the group consisting of the following (1) to (4):

(1) LCDR1 as shown in SEQ ID NO:6, LCDR2 as shown in SEQ ID NO:7 and LCDR3 as shown in SEQ ID NO:8;

(2) LCDR1 as shown in SEQ ID NO:12, LCDR2 as shown in SEQ ID NO:13 and LCDR3 as shown in SEQ ID NO:14;

(3) LCDR1 as shown in SEQ ID NO:26, LCDR2 as shown in SEQ ID NO:27 and LCDR3 as shown in SEQ ID NO:28; or,

(4) LCDR1 as shown in SEQ ID NO:32, LCDR2 as shown in SEQ ID NO:33 and LCDR3 as shown in SEQ ID NO:34.

In one embodiment of the present invention, the antibody-drug conjugate of general formula (A) or a pharmaceutically acceptable salt or solvate thereof as described above, wherein, the heavy chain and light chain variable regions of the anti-B7-H4 antibody or antigen-binding fragment thereof comprises:

(1) HCDR1 as shown in SEQ ID NO:3, HCDR2 as shown in SEQ ID NO:4 and HCDR3 as shown in SEQ ID NO:5, respectively; and

LCDR1 as shown in SEQ ID NO:6, LCDR2 as shown in SEQ ID NO:7 and LCDR3 as shown in SEQ ID NO:8; or,

(2) HCDR1 as shown in SEQ ID NO:9, HCDR2 as shown in SEQ ID NO:10 and HCDR3 as shown in SEQ ID NO:11, respectively; and

LCDR1 as shown in SEQ ID NO:12, LCDR2 as shown in SEQ ID NO:13 and LCDR3 as shown in SEQ ID NO:14.

In one embodiment of the present invention, the antibody-drug conjugate of general formula (A) or a pharmaceutically acceptable salt or solvate thereof as described above, wherein, the heavy chain and light chain variable regions of the anti-B7-H4 antibody or antigen-binding fragment thereof comprises:

(3) HCDR1 as shown in SEQ ID NO:23, HCDR2 as shown in SEQ ID NO:24 and HCDR3 as shown in SEQ ID NO:25; and

LCDR1 as shown in SEQ ID NO:26, LCDR2 as shown in SEQ ID NO:27 and LCDR3 as shown in SEQ ID NO:28; or,

(4) HCDR1 as shown in SEQ ID NO:29, HCDR2 as shown in SEQ ID NO:30 and HCDR3 as shown in SEQ ID NO:31; and

LCDR1 as shown in SEQ ID NO:32, LCDR2 as shown in SEQ ID NO:33 and LCDR3 as shown in SEQ ID NO:34.

In one embodiment of the present invention, the antibody-drug conjugate of general formula (A) or a pharmaceutically acceptable salt or solvate thereof as described above, wherein, the heavy chain and light chain variable regions of the anti-B7-H4 antibody or antigen-binding fragment thereof comprises:

(5) HCDR1 as shown in SEQ ID NO:43, HCDR2 as shown in SEQ ID NO:44 and HCDR3 as shown in SEQ ID NO:25, respectively; and

LCDR1 as shown in SEQ ID NO:26, LCDR2 as shown in SEQ ID NO:27 and LCDR3 as shown in SEQ ID NO:28; or,

(6) HCDR1 as shown in SEQ ID NO:29, HCDR2 as shown in SEQ ID NO:45 and HCDR3 as shown in SEQ ID NO:31, respectively; and

LCDR1 as shown in SEQ ID NO:32, LCDR2 as shown in SEQ ID NO:33 and LCDR3 as shown in SEQ ID NO:34.

In one embodiment of the present invention, the antibody-drug conjugate of general formula (A) or a pharmaceutically acceptable salt or solvate thereof as described above, wherein, the antibody heavy chain variable region and the antibody light chain variable region of the Ab comprises the CDRs of any one selected from the group consisting of the following (1) to (4):

(1) HCDR1 as shown in SEQ ID NO:3, HCDR2 as shown in SEQ ID NO:4 and HCDR3 as shown in SEQ ID NO:5; and

LCDR1 as shown in SEQ ID NO:6, LCDR2 as shown in SEQ ID NO:7 and LCDR3 as shown in SEQ ID NO:8;

(2) HCDR1 as shown in SEQ ID NO:9, HCDR2 as shown in SEQ ID NO:10 and HCDR3 as shown in SEQ ID NO:11; and

LCDR1 as shown in SEQ ID NO:12, LCDR2 as shown in SEQ ID NO:13 and LCDR3 as shown in SEQ ID NO:14;

(3) HCDR1 as shown in SEQ ID NO:23, HCDR2 as shown in SEQ ID NO:24 and HCDR3 as shown in SEQ ID NO:25; and

LCDR1 as shown in SEQ ID NO:26, LCDR2 as shown in SEQ ID NO:27 and LCDR3 as shown in SEQ ID NO:28; or,

(4) HCDR1 as shown in SEQ ID NO:29, HCDR2 as shown in SEQ ID NO:30 and HCDR3 as shown in SEQ ID NO:31; and

LCDR1 as shown in SEQ ID NO:32, LCDR2 as shown in SEQ ID NO:33 and LCDR3 as shown in SEQ ID NO:34.

The antibody heavy chain variable region and antibody light chain variable region of the Ab can also comprise CDRs selected from the group consisting of the following (5) or (6):

(5) HCDR1 as shown in SEQ ID NO:43, HCDR2 as shown in SEQ ID NO:44 and HCDR3 as shown in SEQ ID NO:25; and

LCDR1 as shown in SEQ ID NO:26, LCDR2 as shown in SEQ ID NO:27 and LCDR3 as shown in SEQ ID NO:28;

(6) HCDR1 as shown in SEQ ID NO:29, HCDR2 as shown in SEQ ID NO:45 and HCDR3 as shown in SEQ ID NO:31; and

LCDR1 as shown in SEQ ID NO:32, LCDR2 as shown in SEQ ID NO:33 and LCDR3 as shown in SEQ ID NO:34.

In a preferred embodiment, the antibody-drug conjugate of general formula (A) or a pharmaceutically acceptable salt or solvate thereof as described above, wherein the Ab is a murine antibody or fragment thereof, a chimeric antibody or fragment thereof, a human antibody or fragment thereof, and a humanized antibody or fragment thereof.

In a preferred embodiment, the antibody-drug conjugate of general formula (A) or a pharmaceutically acceptable salt or solvate thereof as described above, wherein, the Ab further comprises light chain framework region and heavy chain framework region sequences, which are respectively derived from human germline light chain and heavy chain sequences or mutant sequence(s) thereof;

In a preferred embodiment, the antibody-drug conjugate of general formula (A) or a pharmaceutically acceptable salt or solvate thereof as described above, wherein, the Ab further comprises heavy chain constant region(s), wherein the heavy chain constant region(s) comprise those derived from human IgG1 or variant thereof, IgG2 or variant thereof, IgG3 or variant thereof or IgG4 or variant thereof, preferably those derived from human IgG1, IgG2 or IgG4, more preferably IgG1 heavy chain constant region with enhanced ADCC toxicity after amino acid mutation, most preferably the heavy chain constant region as shown in SEQ ID NO: 54;

In a preferred embodiment, the antibody-drug conjugate of general formula (A) or a pharmaceutically acceptable salt or solvate thereof as described above, wherein, the Ab further comprises light chain constant region derived from human κ chain, λ chain or variant thereof, preferably that derived from human κ chain, more preferably the light chain constant region as shown in SEQ ID NO:55.

In a preferred embodiment, the antibody-drug conjugate of general formula (A) or a pharmaceutically acceptable salt or solvate thereof as described above, wherein, the Ab comprises light chain variable region with the following sequences: SEQ ID NO:16 or SEQ ID NO:18.

In a preferred embodiment, the antibody-drug conjugate of general formula (A) or a pharmaceutically acceptable salt or solvate thereof as described above, wherein, the Ab comprises light chain variable region with the following sequences: SEQ ID NO:16, SEQ ID NO:18, SEQ ID NO:36, SEQ ID NO:38, or light chain variable regions with at least 70%, 75%, 80%, 85%, 90%, 95% or 99% homology to SEQ ID NO:16, SEQ ID NO:18, SEQ ID NO:36, SEQ ID NO:38.

In a preferred embodiment, the antibody-drug conjugate of general formula (A) or a pharmaceutically acceptable salt or solvate thereof as described above, wherein, the Ab comprises heavy chain variable region with the following sequences: SEQ ID NO:15 or SEQ ID NO:17.

In a preferred embodiment, the antibody-drug conjugate of general formula (A) or a pharmaceutically acceptable salt or solvate thereof as described above, wherein, the Ab comprises heavy chain variable region with the following sequences: SEQ ID NO:15, SEQ ID NO:17, SEQ ID NO:35, SEQ ID NO:37, or heavy chain variable regions with at least 70%, 75%, 80%, 85%, 90%, 95% or 99% homology to SEQ ID NO:15, SEQ ID NO:17, SEQ ID NO:35, SEQ ID NO:37.

In a preferred embodiment, the antibody-drug conjugate of general formula (A) or a pharmaceutically acceptable salt or solvate thereof as described above, wherein, the heavy chain variable region and the light chain variable region of the Ab is any one selected from the group consisting of the following:

(1) the heavy chain variable region as shown in SEQ ID NO: 15 and the light chain variable region as shown in SEQ ID NO: 16;

(2) the heavy chain variable region as shown in SEQ ID NO: 17 and the light chain variable region as shown in SEQ ID NO: 18.

In a preferred embodiment, the antibody-drug conjugate of general formula (A) or a pharmaceutically acceptable salt or solvate thereof as described above, wherein, the heavy chain variable region and the light chain variable region of the Ab is any one selected from the group consisting of the following:

(1) the heavy chain variable region as shown in SEQ ID NO: 15 and the light chain variable region as shown in SEQ ID NO: 16:

(2) the heavy chain variable region as shown in SEQ ID NO: 17 and the light chain variable region as shown in SEQ ID NO: 18;

(3) the heavy chain variable region as shown in SEQ ID NO: 35 and the light chain variable region as shown in SEQ ID NO: 36; or,

(4) the heavy chain variable region as shown in SEQ ID NO: 37 and the light chain variable region as shown in SEQ ID NO: 38.

In a preferred embodiment, the antibody-drug conjugate of general formula (A) or a pharmaceutically acceptable salt or solvate thereof as described above, wherein, the Ab comprises light chain with the following sequences: SEQ ID NO:20 or SEQ ID NO:22.

In a preferred embodiment, the antibody-drug conjugate of general formula (A) or a pharmaceutically acceptable salt or solvate thereof as described above, wherein, the Ab comprises light chain with the following sequences: SEQ ID NO:20, SEQ ID NO:22, SEQ ID NO:40, SEQ ID NO:42, or full-length light chain with at least 80%, 85%, 90%, 95% or 99% homology to SEQ ID NO:20, SEQ ID NO:22, SEQ ID NO:40, SEQ ID NO:42.

In a preferred embodiment, the antibody-drug conjugate of general formula (A) or a pharmaceutically acceptable salt or solvate thereof as described above, wherein, the Ab comprises heavy chain with the following sequences: SEQ ID NO:19 or SEQ ID NO:21.

In a preferred embodiment, the antibody-drug conjugate of general formula (A) or a pharmaceutically acceptable salt or solvate thereof as described above, wherein, the Ab comprises heavy chain with the following sequences: SEQ ID NO:19, SEQ ID NO:21, SEQ ID NO:39, SEQ ID NO:41, or full-length heavy chain with at least 80%, 85%, 90%, 95% or 99% homology to SEQ ID NO:19, SEQ ID NO:21, SEQ ID NO:39, SEQ ID NO:41.

In a preferred embodiment, the antibody-drug conjugate of general formula (A) or a pharmaceutically acceptable salt or solvate thereof as described above, wherein, the Ab comprises:

(1) the light chain of SEQ ID NO: 20 and the heavy chain of SEQ ID NO: 19; or,

(2) the light chain of SEQ ID NO: 22 and the heavy chain of SEQ ID NO: 21.

In a preferred embodiment, the antibody-drug conjugate of general formula (A) or a pharmaceutically acceptable salt or solvate thereof as described above, wherein, the Ab comprises:

(1) the light chain of SEQ ID NO: 20 and the heavy chain of SEQ ID NO: 19; or,

(2) the light chain of SEQ ID NO: 22 and the heavy chain of SEQ ID NO: 21; or,

(3) the light chain of SEQ ID NO: 40 and the heavy chain of SEQ ID NO: 39; or,

(4) the light chain of SEQ ID NO: 42 and the heavy chain of SEQ ID NO: 41.

In a preferred embodiment, the antibody-drug conjugate of general formula (A) or a pharmaceutically acceptable salt or solvate thereof as described above, wherein, the antigen-binding fragment of the anti-B7-H4 antibody is selected from the group consisting of Fab, Fab′, F(ab′)₂, single-chain antibody (scFv), dimerized V region (diabody), disulfide bond stabilized V region (dsFv) and antigen-binding fragments of a peptide comprising CDRs.

In a preferred embodiment, the antibody-drug conjugate of general formula (A) or a pharmaceutically acceptable salt or solvate thereof as described above, wherein, the cytotoxic drug is selected from the group consisting of toxin, chemotherapeutic, antibiotic, radioisotope and nucleolytic enzyme; preferably tubulin inhibitor or DNA topoisomerase inhibitor that inhibits cell division; more preferably DM1, DM3, DM4, SN-38, MMAF or MMAE; further preferably the tubulin inhibitor SN-38, MMAE or MMAF. Wherein, the structure of MMAF and SN-38 is as shown in the following formula:

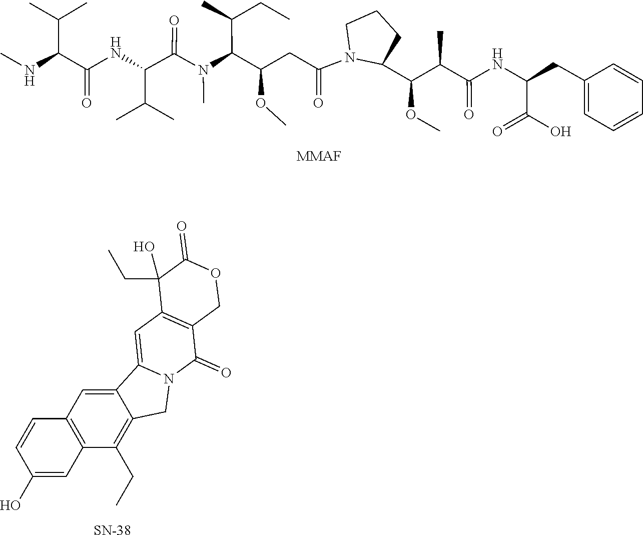

In a preferred embodiment, the antibody-drug conjugate of general formula (A) or a pharmaceutically acceptable salt or solvate thereof as described above, wherein, the cytotoxic drug is selected from camptothecin derivatives, preferably Exatecan,

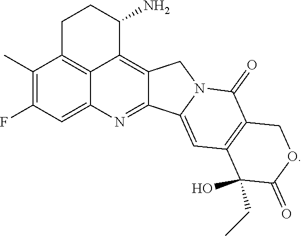

In a preferred embodiment, the antibody-drug conjugate of general formula (A) or a pharmaceutically acceptable salt or solvate thereof as described above, which is a compound of general formula (I) or a pharmaceutically acceptable salt or solvate thereof,

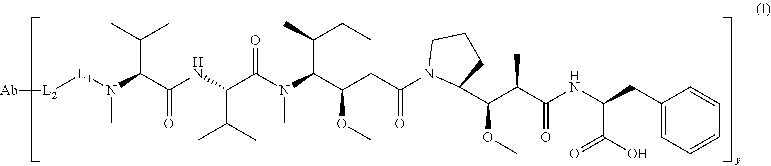

wherein:

L₁ and L₂ are linker units;

y is a number selected from 1 to 8, preferably a number selected from 2 to 4;

Ab is an anti-B7-H4 antibody or antigen-binding fragment thereof as defined above.

In a preferred embodiment, the antibody-drug conjugate of general formula (A) or a pharmaceutically acceptable salt or solvate thereof as described above, which is an antibody-drug conjugate of general formula (II) or a pharmaceutically acceptable salt or solvate thereof:

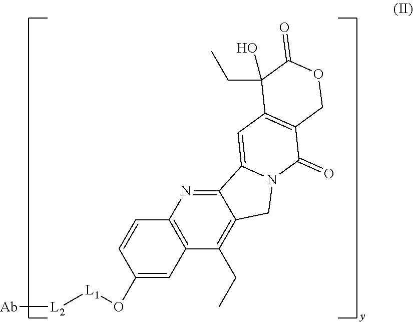

wherein:

L₁ and L₂ are linker units;

y is a number selected from 1 to 8, preferably a number selected from 2 to 4;

Ab is an anti-B7-H4 antibody or antigen-binding fragment thereof as defined above.

In a preferred embodiment, the antibody-drug conjugate of general formula (A) or a pharmaceutically acceptable salt or solvate thereof as described above, which is an antibody-drug conjugate of general formula (III) or a pharmaceutically acceptable salt or solvate thereof,

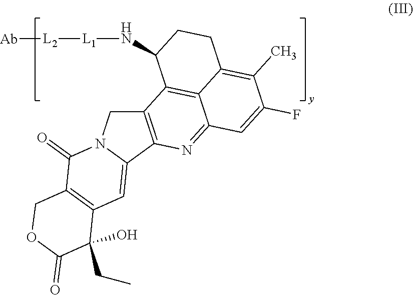

wherein:

L₁ and L₂ are linker units;

y is a number selected from 1 to 10, preferably a number selected from 2 to 8, more preferably a number selected from 4 to 8;

or, y is preferably a number selected from 2 to 10, further preferably a number selected from 6 to 10, more preferably a number of 7 to 9, and most preferably an integer of 7, 8, 9;

Ab is an anti-B7-H4 antibody or antigen-binding fragment thereof as defined above.

In a preferred embodiment, the antibody-drug conjugate or a pharmaceutically acceptable salt or solvate thereof as described above, wherein, the L₁ is as shown in the following general formula (B):

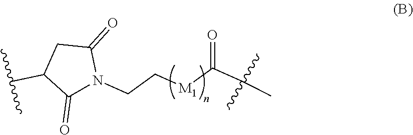

wherein:

M₁ is —CR₁R₂—;

R₁ and R₂ are the same or different, and are independently selected from the group consisting of hydrogen, alkyl, halogen, hydroxyl and amino;

N is an integer of 0 to 5, preferably 1, 2 or 3.

In a preferred embodiment, the antibody-drug conjugate or a pharmaceutically acceptable salt or solvate thereof as described above, wherein, the L₂ is as shown in the following general formula (C):

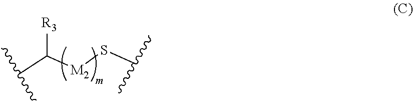

wherein:

M₂ is —CR₄R₅—;

R₃ is selected from the group consisting of hydrogen atom, halogen, hydroxyl, amino, alkyl, alkoxyl and cycloalkyl;

R₄ and R₅ are the same or different, and are independently selected from the group consisting of hydrogen, alkyl, halogen, hydroxyl and amino;

m is an integer of 0 to 5, preferably 1, 2 or 3.

In a preferred embodiment, the antibody-drug conjugate or a pharmaceutically acceptable salt or solvate thereof as described above, wherein, the L₂ is as shown in the following general formula (D):

-K¹-K²-K³-K⁴- (D)

wherein:

K¹ is

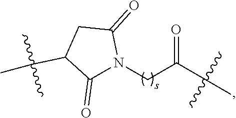

s is an integer of 2 to 8, further preferably an integer of 4 to 8, more preferably an integer of 4 to 6;

K² is —NR¹(CH₂CH₂O)ₚCH₂CH₂C(O)—, —NR¹(CH₂CH₂O)ₚCH₂C(O)—, —S(CH₂)ₚC(O)— or a single bond, p is an integer of 1 to 20, preferably 1 to 6;

R₁ is selected from the group consisting of hydrogen, deuterium, hydroxyl, amino, alkyl, halogen, haloalkyl, deuterated alkyl and hydroxyallyl;

K³ is a tetrapeptide residue, preferably, the tetrapeptide residue is a peptide residue formed by amino acids selected from the group consisting of two or more of phenylalanine, glycine, valine, lysine, citrulline, serine, glutamate and aspartate; more preferably the tetrapeptide residue GGFG:

K⁴ is —NR¹(CR³R⁴)t-, R², R³ or R⁴ are each independently hydrogen, deuterium, hydroxyl, amino, alkyl, halogen, haloalkyl, deuterated alkyl and hydroxyalkyl, and t is 1 or 2, preferably, as for the L₂,

K¹ is

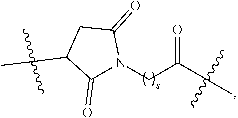

s is 5;

K² is a bond;

K³ is the tetrapeptide residue GGFG:

K⁴ is —NR²(CR³R⁴)t-, R², R³ or R⁴ are each independently hydrogen, deuterium, hydroxyl, amino, C₁₋₆ alkyl, halogen, C₁₋₆ haloalkyl, C₁₋₆ deuterated alkyl and C₁₋₆ hydroxyalkyl, and t is 1 or 2.

In a preferred embodiment, the antibody-drug conjugate or a pharmaceutically acceptable salt or solvate thereof as described above, wherein, the K¹ terminus of the linker unit -L₂- is linked to the Ab, and the K⁴ terminus is linked to L₁.

In a preferred embodiment, the antibody-drug conjugate or a pharmaceutically acceptable salt or solvate thereof as described above, wherein, L₁ is —O—(CR^{a}R^{b})ₘ—CR⁵R⁶—C(O)—, —O—CR⁵R⁶—(CR^{a}R^{b})ₘ—, —O—CR⁵R₆—, —NH—(CR^{a}R^{b})ₘ—CR⁵R⁶—C(O)— or —S—(CR^{a}R^{b})ₘCR⁵R⁶—C(O)—;

R^{a} and R^{b} are each independently selected from the group consisting of hydrogen, deuterium, halogen and alkyl;

R⁵ is haloalkyl or cycloalkyl;

R⁶ is selected from the group consisting of hydrogen, haloalkyl and cycloalkyl;

or, R⁵ and R⁶ and the carbon atom to which they are linked form a cycloalkyl;

m is 0, 1, 2, 3 or 4.

Preferably, the 0 terminus of L₁ is linked to the linker unit L₂.

In a preferred embodiment, the antibody-drug conjugate or a pharmaceutically acceptable salt or solvate thereof as described above, wherein, the L₁ is as shown in the following general formula (E):

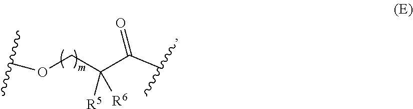

R⁵ is haloalkyl or cycloalkyl,

R⁶ is selected from the group consisting of hydrogen, haloalkyl and cycloalkyl,

or, R⁵ and R⁶ and the carbon atom to which they are linked form a cycloalkyl;

preferably,

R⁵ is selected from the group consisting of C₁₋₆ haloalkyl and C₃₋₆ cycloalkyl,

R⁶ is selected from the group consisting of hydrogen, C₁₋₆ haloalkyl and cycloalkyl,

or, R¹ and R⁶ and the carbon atom to which they are linked form a C₃₋₆ cycloalkyl;

m is an integer of 0 to 4;

more preferably, general formula (E) is selected from the group consisting of the following substituents:

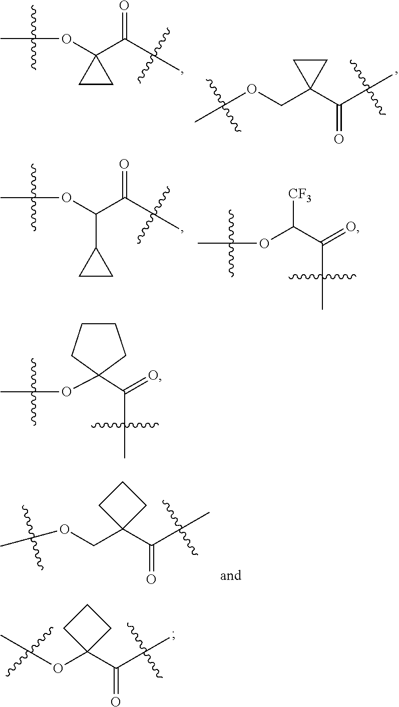

In a more preferred embodiment, the antibody-drug conjugate or a pharmaceutically acceptable salt or solvate thereof as described above, wherein, the -L₂-L₁- is the following structure:

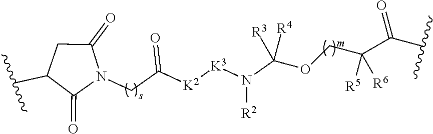

K¹ is a bond;

K³ is the tetrapeptide residue GGFG;

R⁵ is haloalkyl or C₃₋₆ cycloalkyl;

R⁶ is selected from the group consisting of hydrogen, haloalkyl and C₃₋₆ cycloalkyl;

or, R⁵ and R⁶ and the carbon atom to which they are linked form a C₃₋₆ cycloalkyl;

R², R³ or R⁴ are each independently hydrogen or alkyl;

s is an integer of 2 to 8; preferably, s is 4, 5 or 6;

m is an integer of 0 to 4;

preferably, the -L₂-L₁- is selected from the group consisting of the following structures:

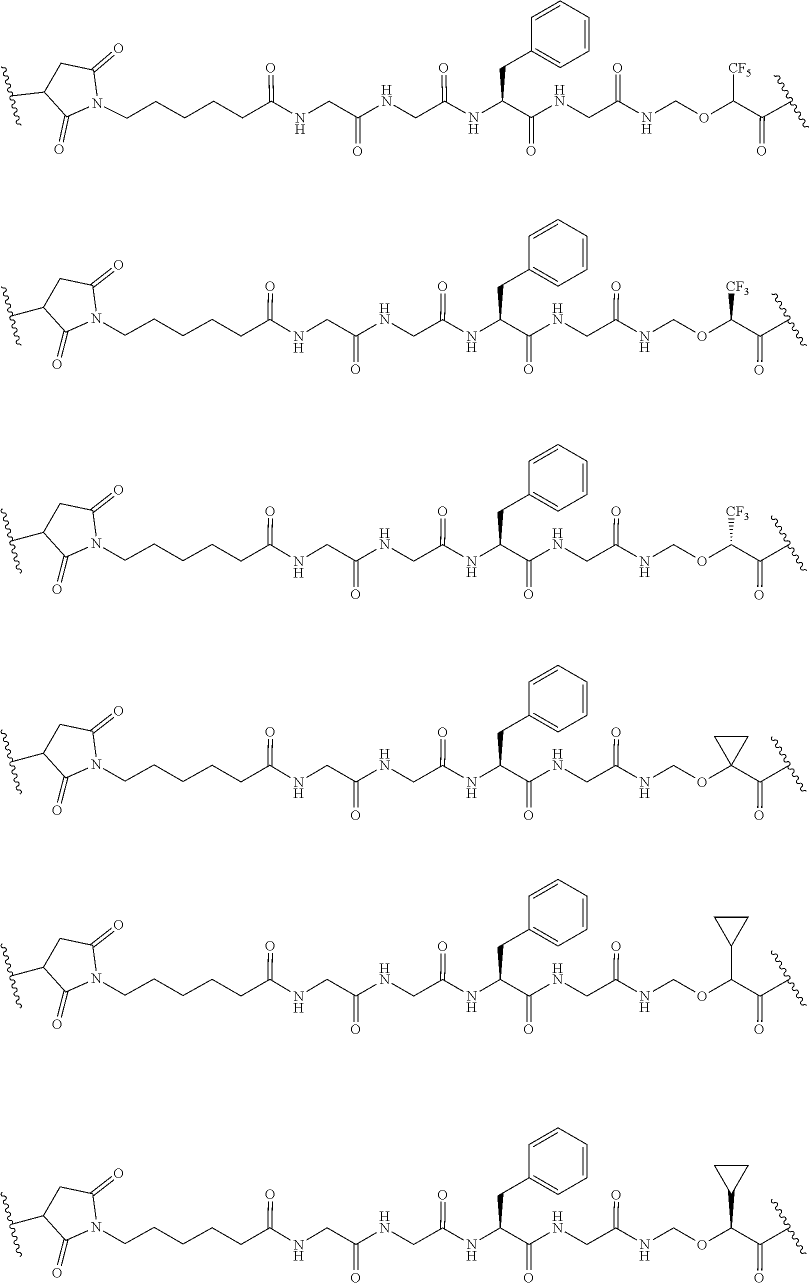

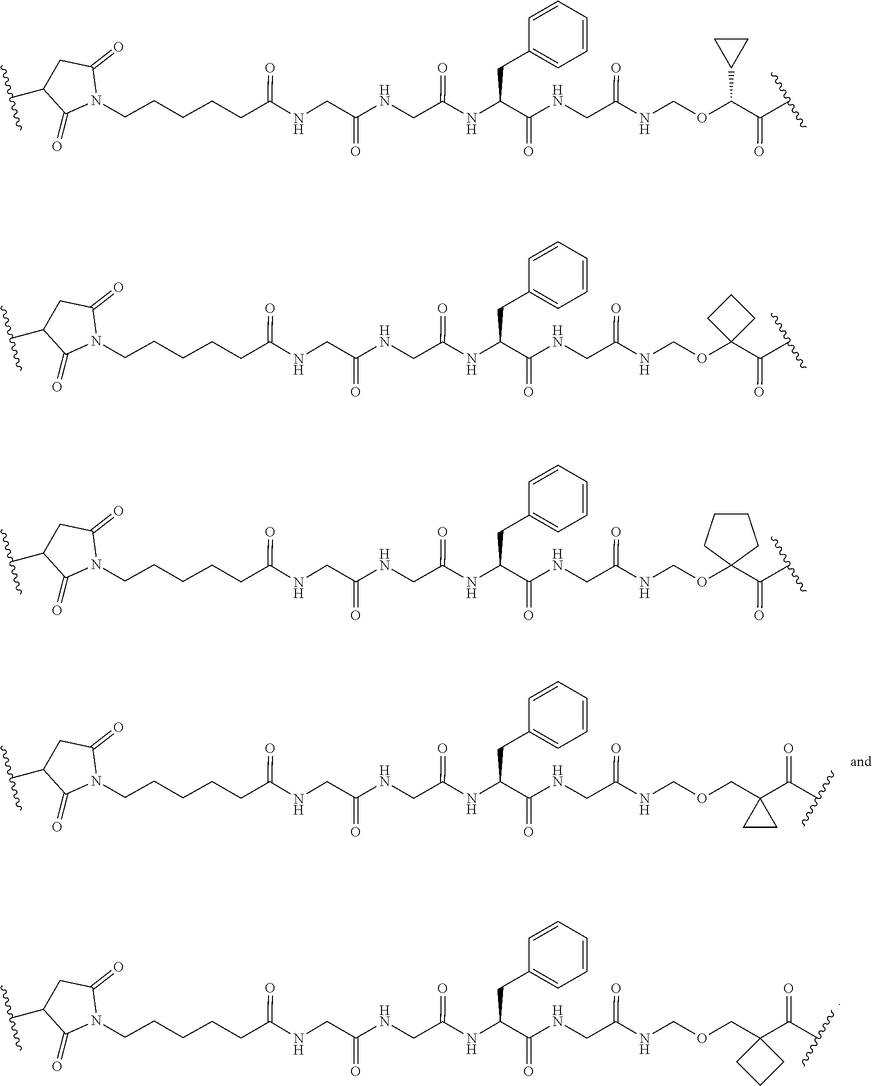 [SMILES: *C(=O)[C@H](OCNC(=O)CNC(=O)[C@H](Cc1ccccc1)NC(=O)CNC(=O)CNC(=O)CCCCCN1C(=O)CC(*)C1=O)C1CC1 ; *C(=O)C1(OCNC(=O)CNC(=O)[C@H](Cc2ccccc2)NC(=O)CNC(=O)CNC(=O)CCCCCN2C(=O)CC(*)C2=O)CCC1 ; *C(=O)C1(OCNC(=O)CNC(=O)[C@H](Cc2ccccc2)NC(=O)CNC(=O)CNC(=O)CCCCCN2C(=O)CC(*)C2=O)CCCC1 ; *C(=O)C1(COCNC(=O)CNC(=O)[C@H](Cc2ccccc2)NC(=O)CNC(=O)CNC(=O)CCCCCN2C(=O)CC(*)C2=O)CC1 ; *C(=O)C1(COCNC(=O)CNC(=O)[C@H](Cc2ccccc2)NC(=O)CNC(=O)CNC(=O)CCCCCN2C(=O)CC(*)C2=O)CCC1]

In a preferred embodiment, the antibody-drug conjugate or a pharmaceutically acceptable salt or solvate thereof as described above, which is the antibody-drug conjugate of general formula (IV) or a pharmaceutically acceptable salt or solvate thereof:

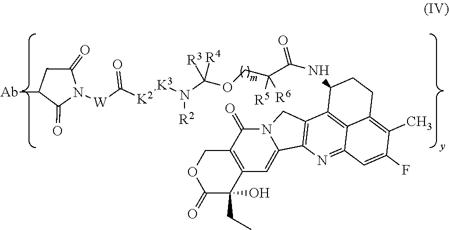

wherein:

W is selected from the group consisting of C₁₋₈ alkyl, C₁₋₈ alkyl-cycloalkyl or linear heteroalkyl of 1 to 8 atoms, the heteroalkyl comprises 1 to 3 heteroatom(s) selected from the group consisting of N, O or S, wherein the C₁₋₈ alkyl, cycloalkyl or linear heteroalkyl is each independently optionally further substituted with one or more substituents selected from the group consisting of halogen, hydroxyl, cyano, amino, alkyl, chloroalkyl, deuterated alkyl, alkoxyl and cycloalkyl;

K² is selected from the group consisting of —NR¹(CH₂CH₂O)ₚ₁CH₂CH₂C(O)—, —NR¹(CH₂CH₂O)ₚ₁CH₂C(O)—, —S(CH₂)ₚ₁C(O)— or bond, R¹ is selected from the group consisting of hydrogen atom, alkyl, haloalkyl, deuterated alkyl and hydroxyalkyl, and p₁ is an integer of 1 to 20;

K³ is a peptide residue consisting of 2 to 7 amino acids, the amino acids can be substituted or unsubstituted. When substituted, the substituents can be substituted at any available attachment point, and the substituents are one or more independently selected from the group consisting of halogen, hydroxyl, cyano, amino, alkyl, chloroalkyl, deuterated alkyl, alkoxyl and cycloalkyl;

R² is independently selected from the group consisting of hydrogen atom, alkyl, haloalkyl, deuterated alkyl and hydroxyalkyl;

R³ and R⁴ are each independently selected from the group consisting of hydrogen atom, halogen, alkyl, haloalkyl, deuterated alkyl and hydroxyalkyl:

R⁵ is selected from the group consisting of halogen, haloalkyl, deuterated alkyl, cycloalkyl, heterocyclyl, aryl and heteroaryl;

R⁶ is selected from the group consisting of hydrogen atom, halogen, haloalkyl, deuterated alkyl, cycloalkyl, heterocyclyl, aryl and heteroaryl;

or, R⁵ and R⁶ and the carbon atom to which they are linked form a cycloalkyl or heterocyclyl:

m is an integer of 0 to 4;

y is 1 to 10 and y is a decimal or an integer, preferably, y is a number of 2 to 10, more preferably y is a number of 4 to 10, further preferably a number of 6 to 9, and most preferably an integer of 7, 8 or 9;

Ab is an anti-B7-H4 antibody or antigen-binding fragment thereof.

In a more preferred embodiment, the antibody-drug conjugate or a pharmaceutically acceptable salt or solvate thereof as described above, which is an antibody-drug conjugate of general formula (I-A) or a pharmaceutically acceptable salt or solvate thereof:

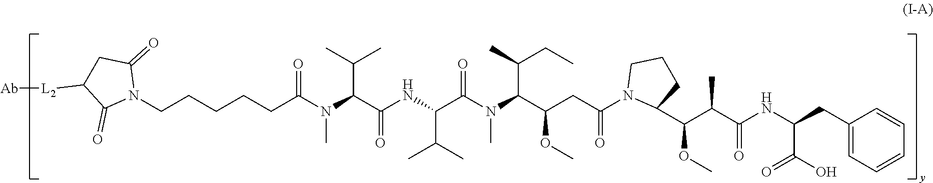

y is 1 to 10, y is a decimal or an integer.

In a more preferred embodiment, the antibody-drug conjugate or a pharmaceutically acceptable salt or solvate thereof as described above, which is an antibody-drug conjugate of general formula (I-B) or a pharmaceutically acceptable salt or solvate thereof:

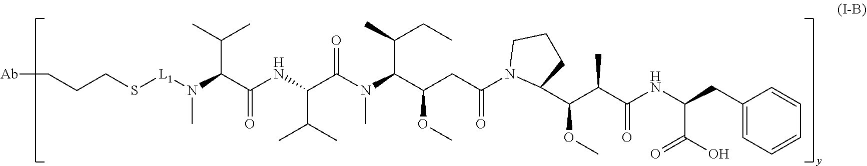

y is 1 to 10, y is a decimal or an integer.

In a more preferred embodiment, the antibody-drug conjugate or a pharmaceutically acceptable salt or solvate thereof as described above, which is an antibody-drug conjugate of general formula (II-A) or a pharmaceutically acceptable salt or solvate thereof:

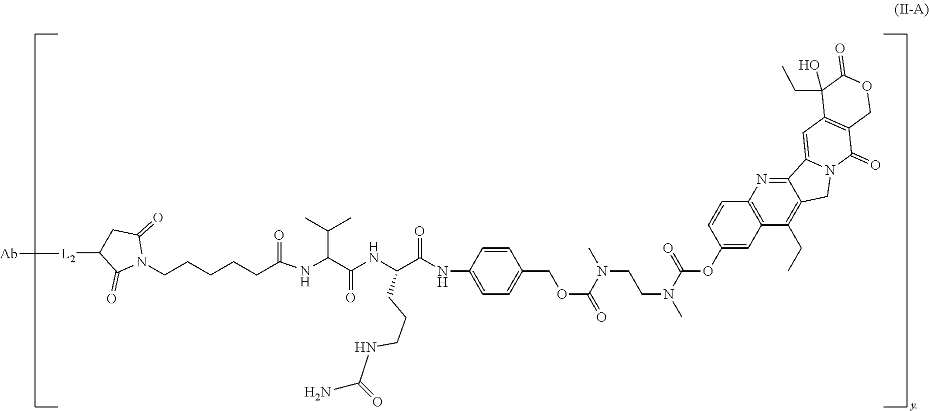

In a more preferred embodiment, the antibody-drug conjugate or a pharmaceutically acceptable salt or solvate thereof as described above, which is an antibody-drug conjugate of general formula (II-B) or a pharmaceutically acceptable salt or solvate thereof:

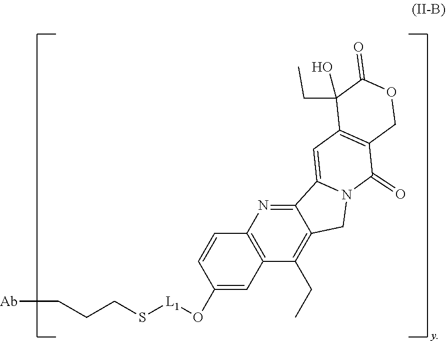

In a more preferred embodiment, the antibody-drug conjugate or a pharmaceutically acceptable salt or solvate thereof as described above, which is an antibody-drug conjugate of general formula (IV-A) or a pharmaceutically acceptable salt or solvate thereof.

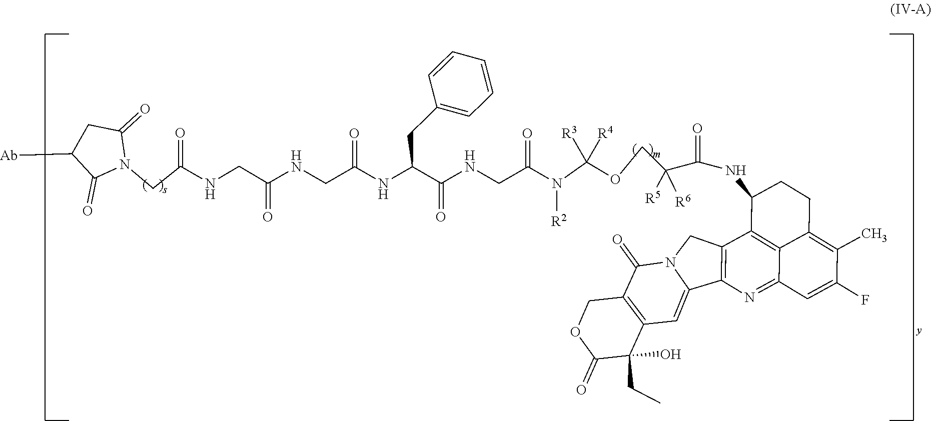

wherein, s is an integer of 2 to 8, R² to R⁶, m and y are as defined in the above general formula (IV);

preferably, s is an integer of 4, 5 or 6, y is a number of 4 to 10, preferably a number of 6 to 9, and more preferably 7 or 8.

In a more preferred embodiment, the antibody-drug conjugate or a pharmaceutically acceptable salt or solvate thereof as described above, selected from the group consisting of the following compounds:

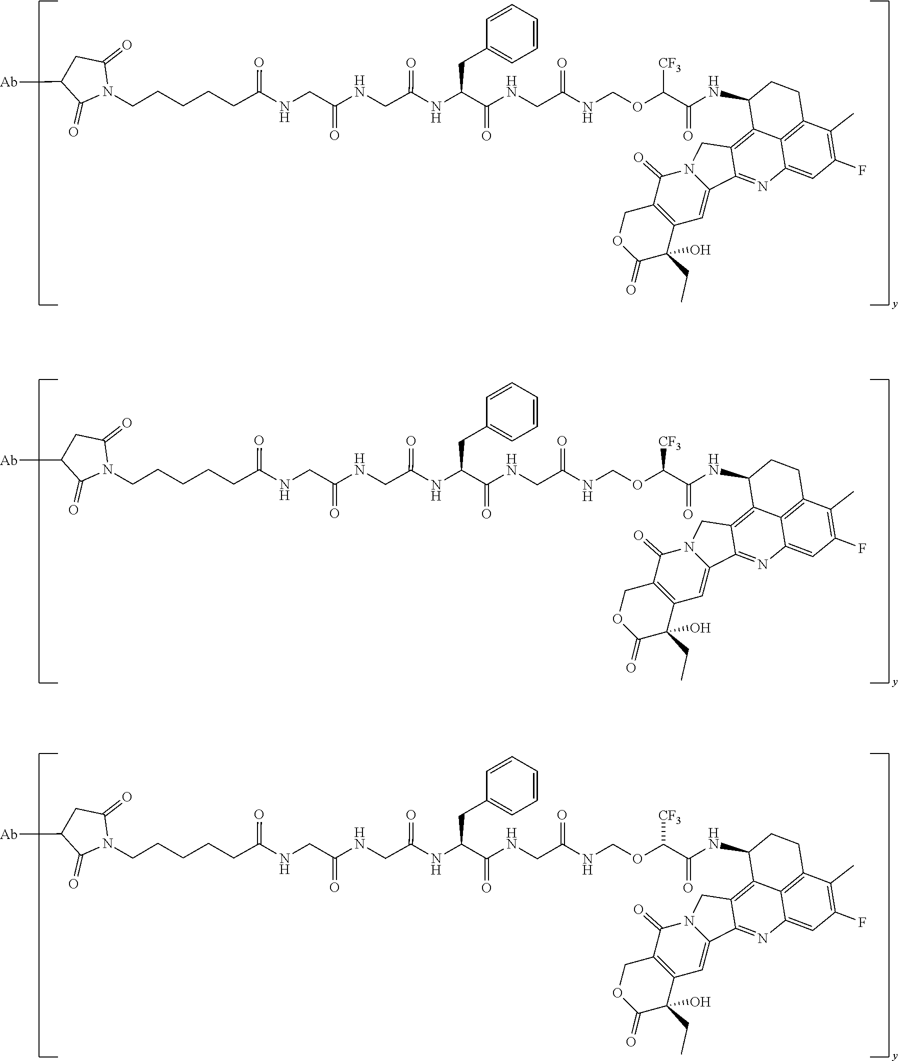

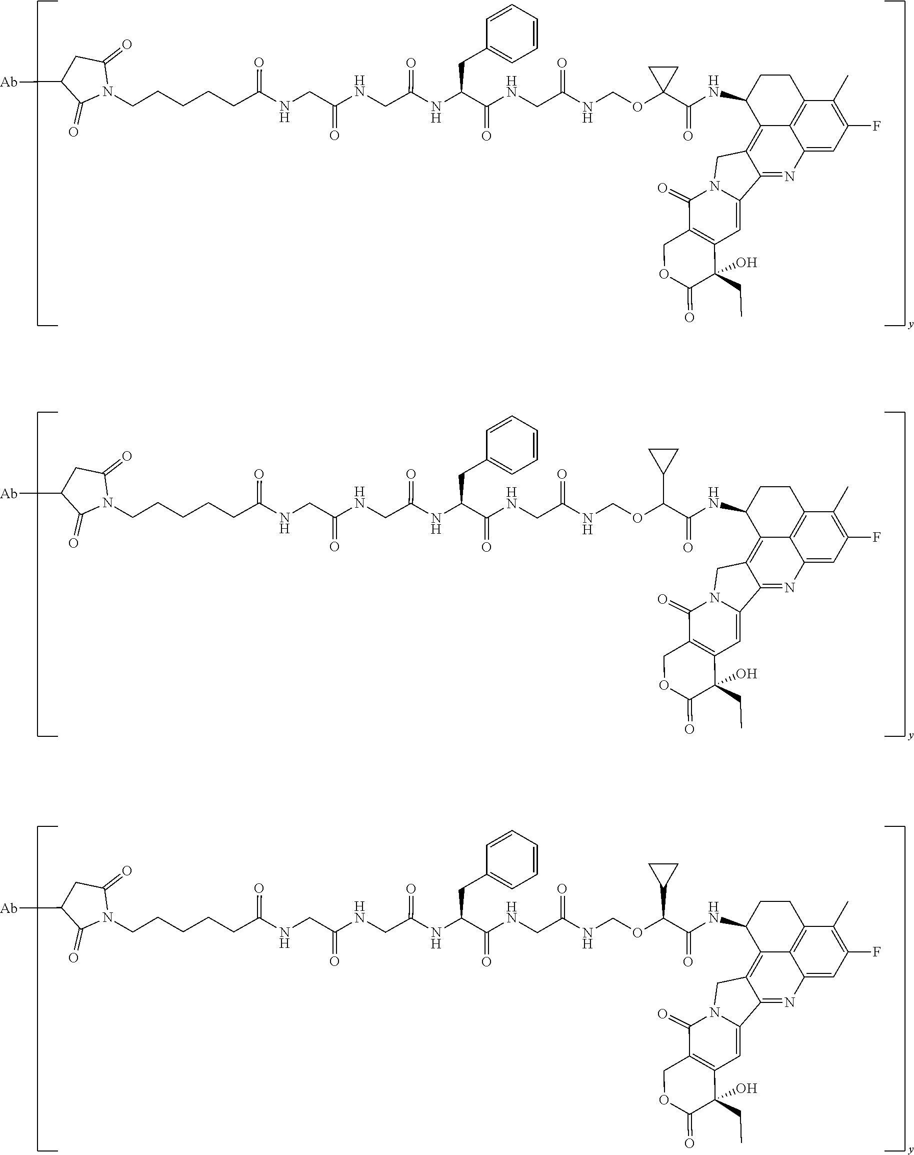

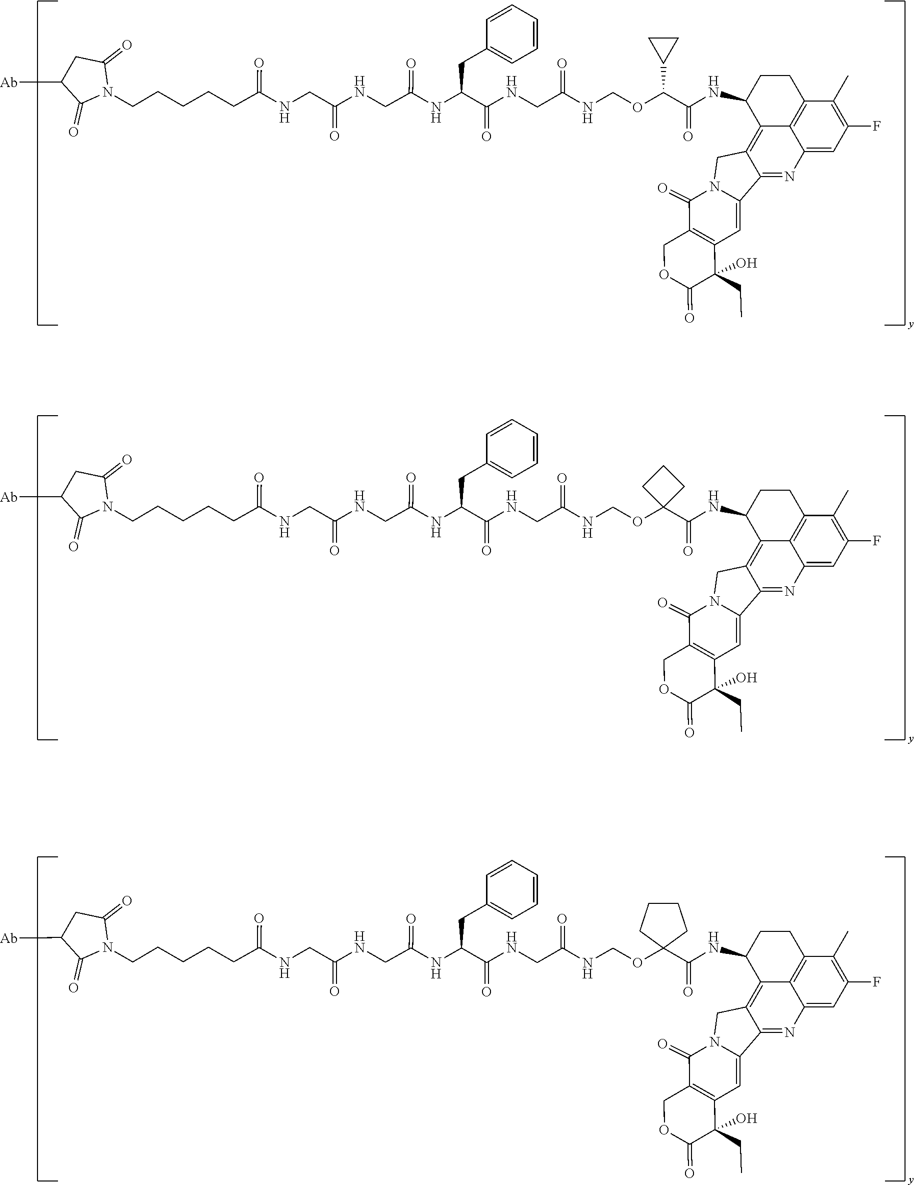

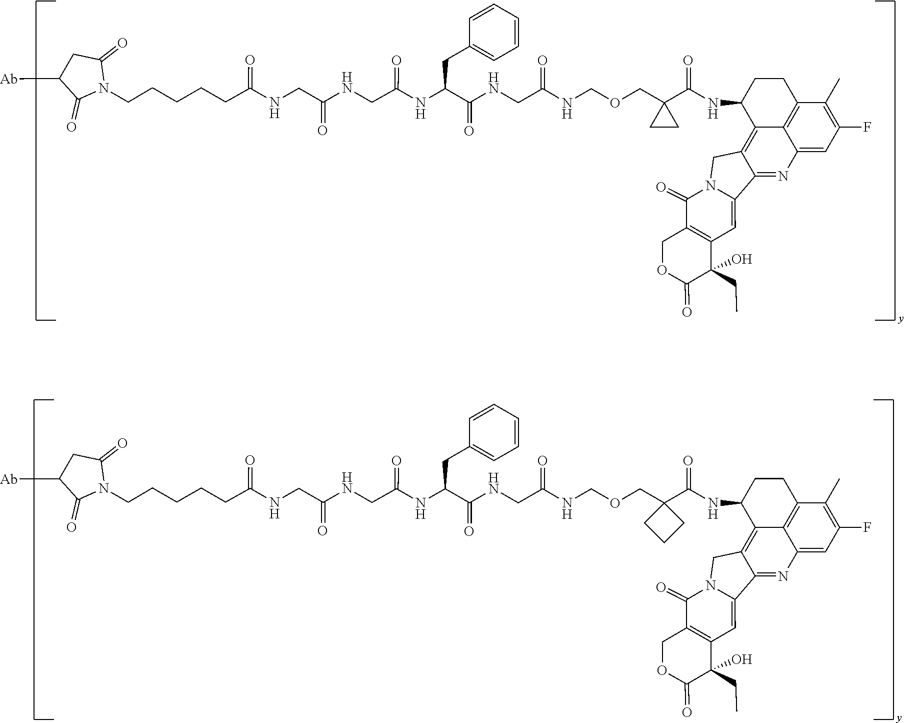

In a most preferred embodiment, the antibody-drug conjugate or a pharmaceutically acceptable salt or solvate thereof as described above, which is selected from the group consisting of the following compounds:

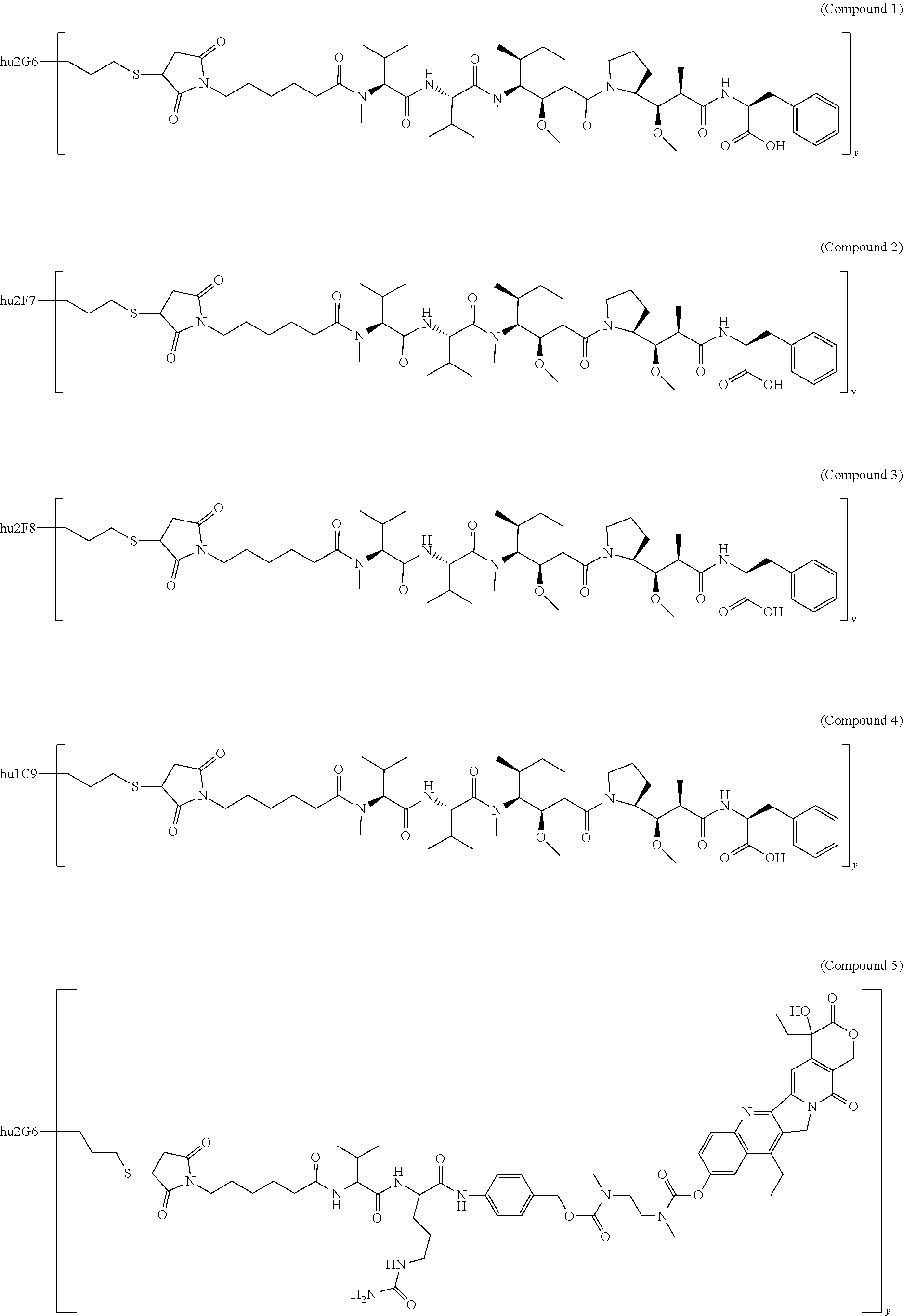

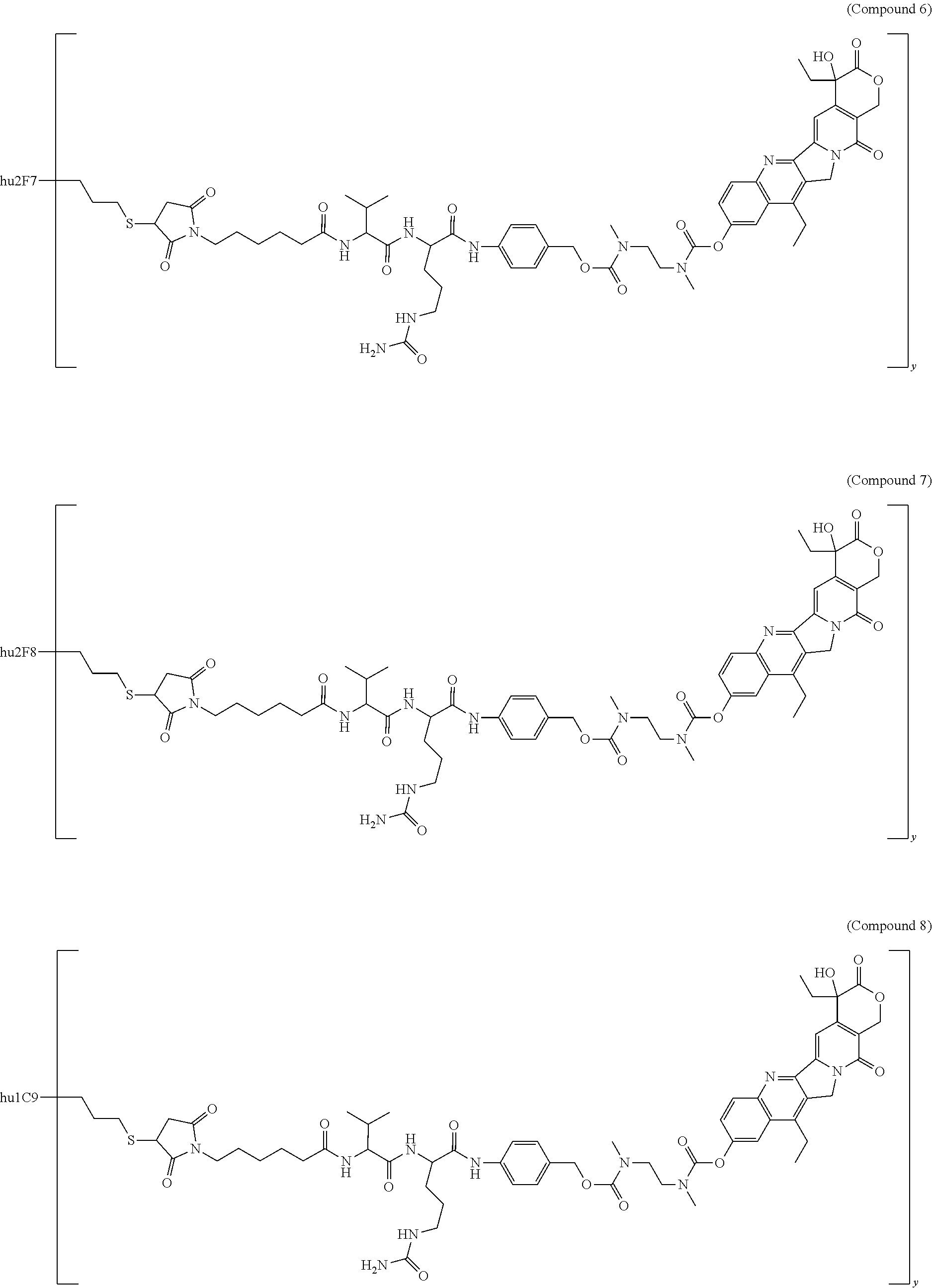

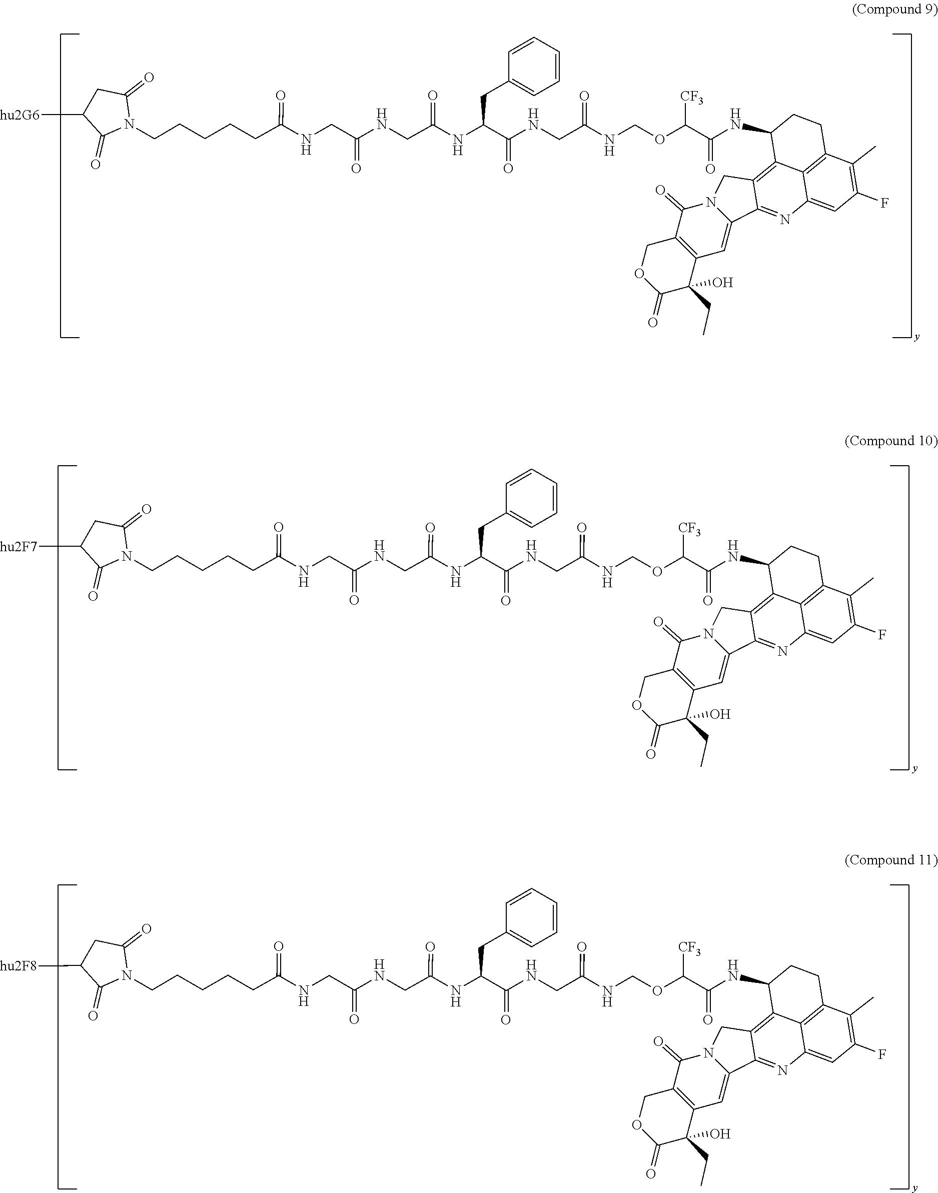

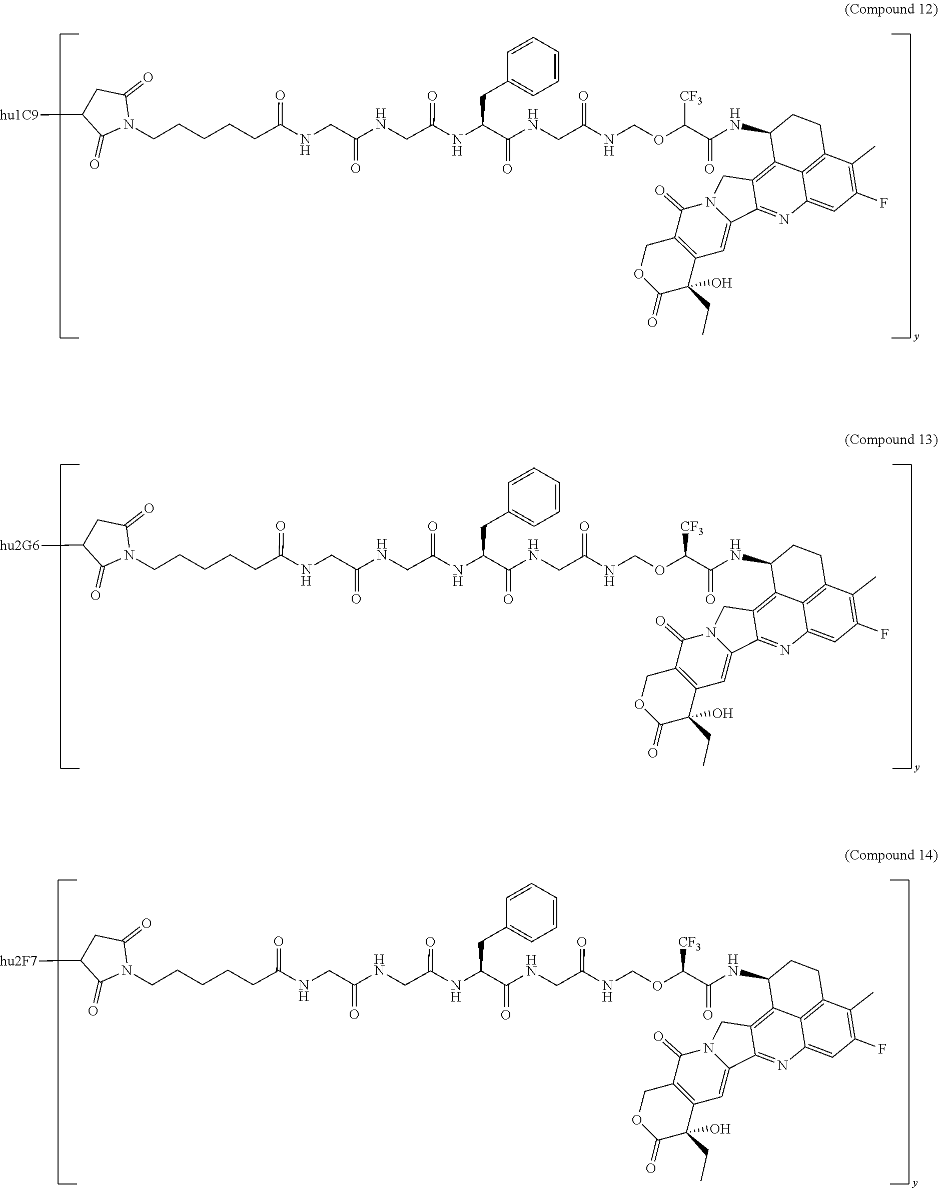

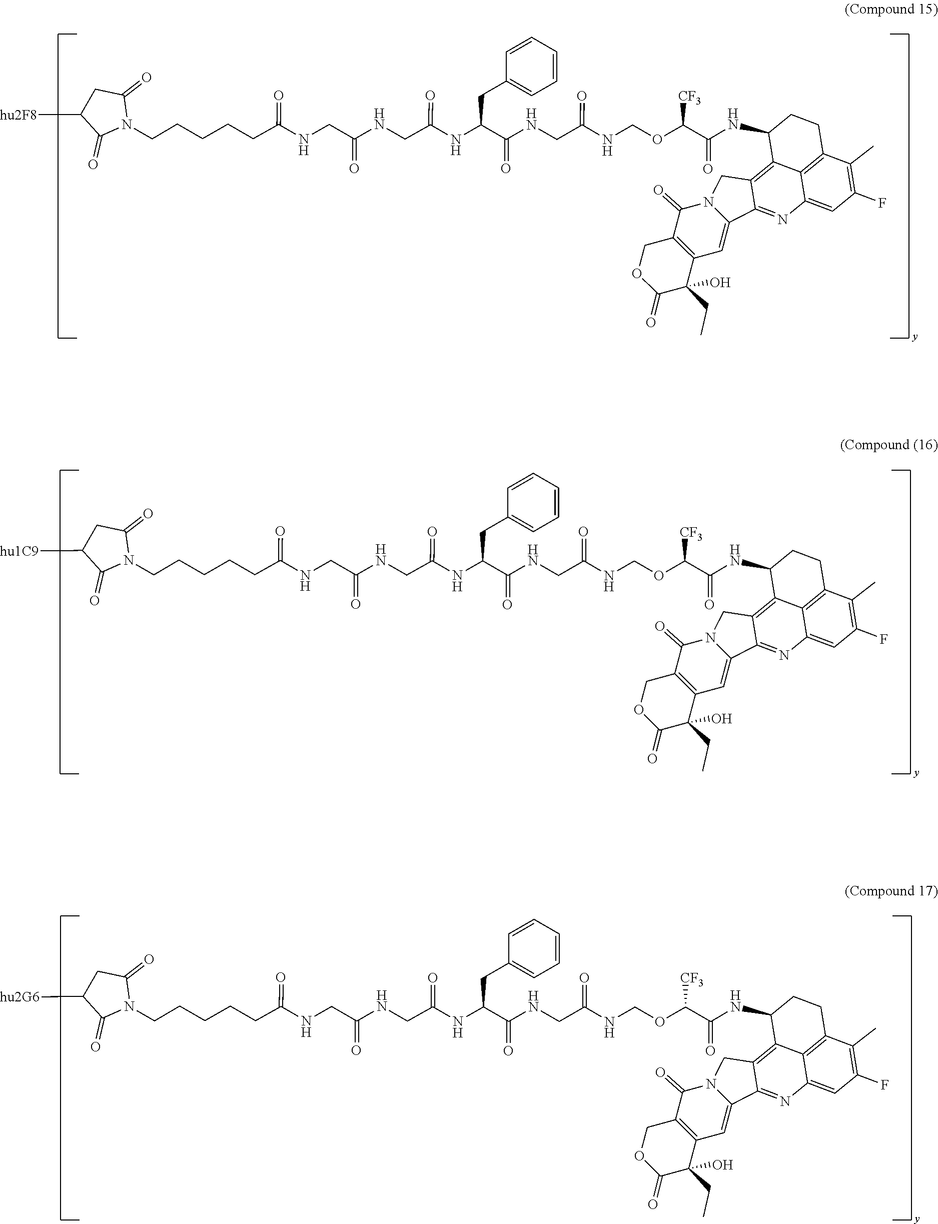

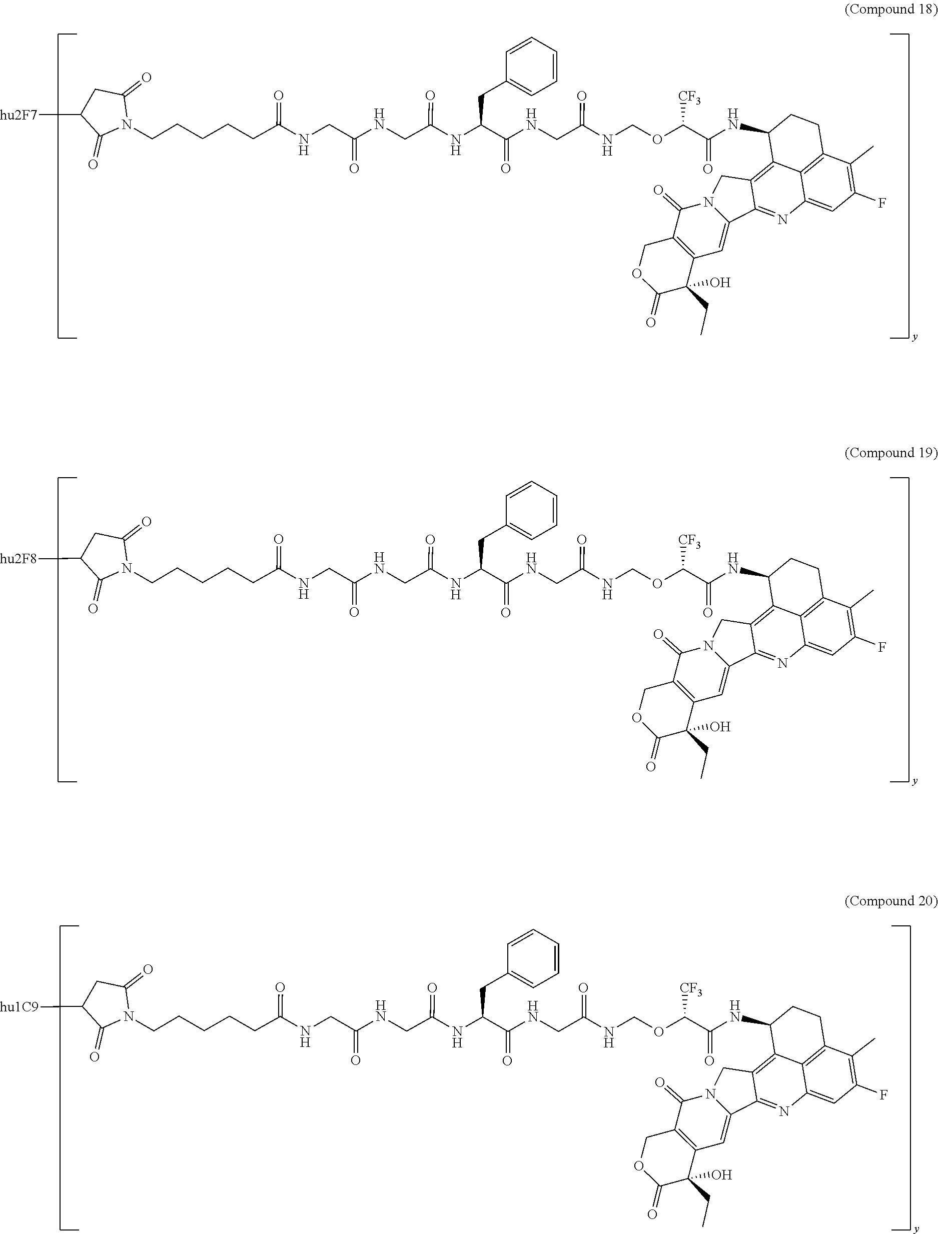

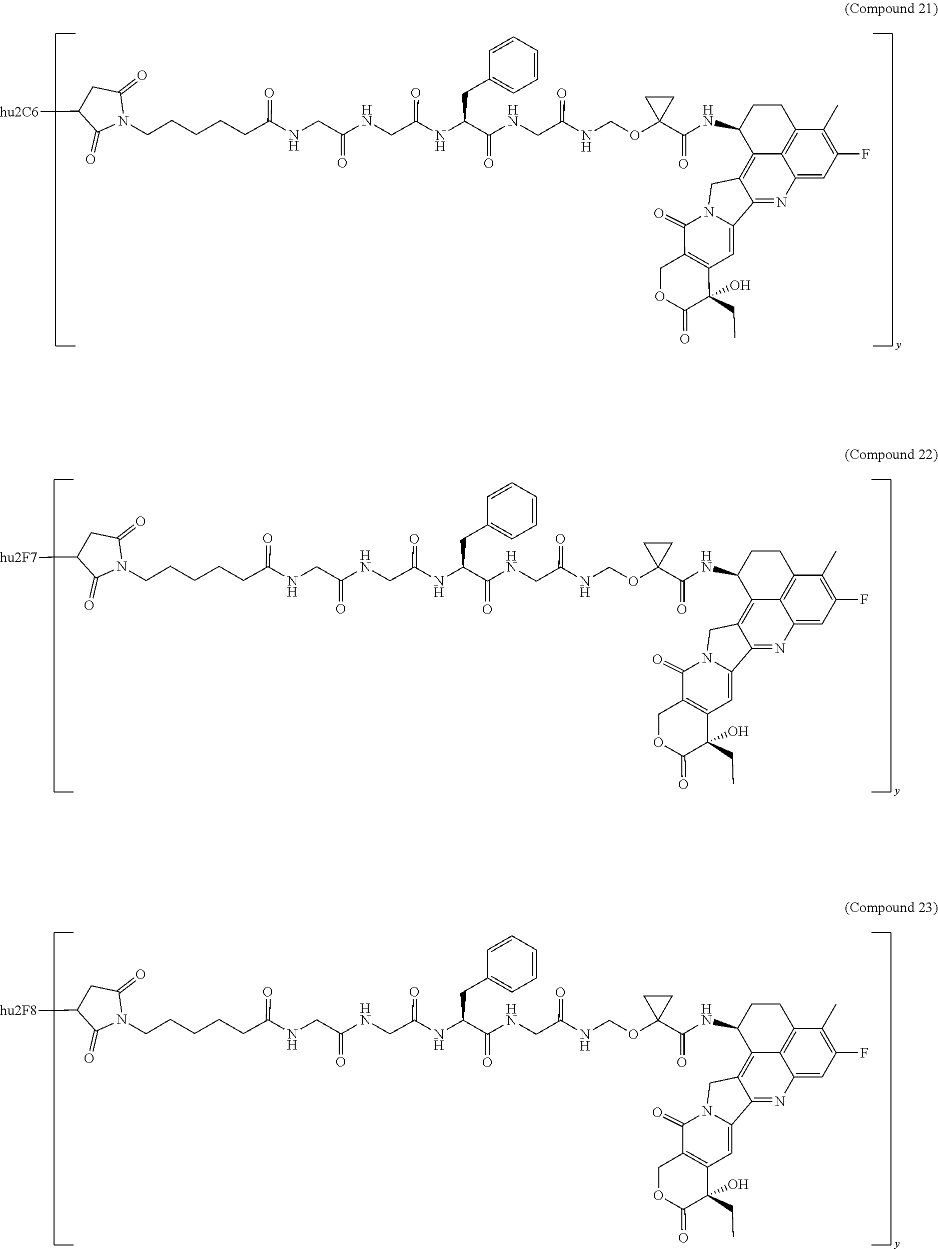

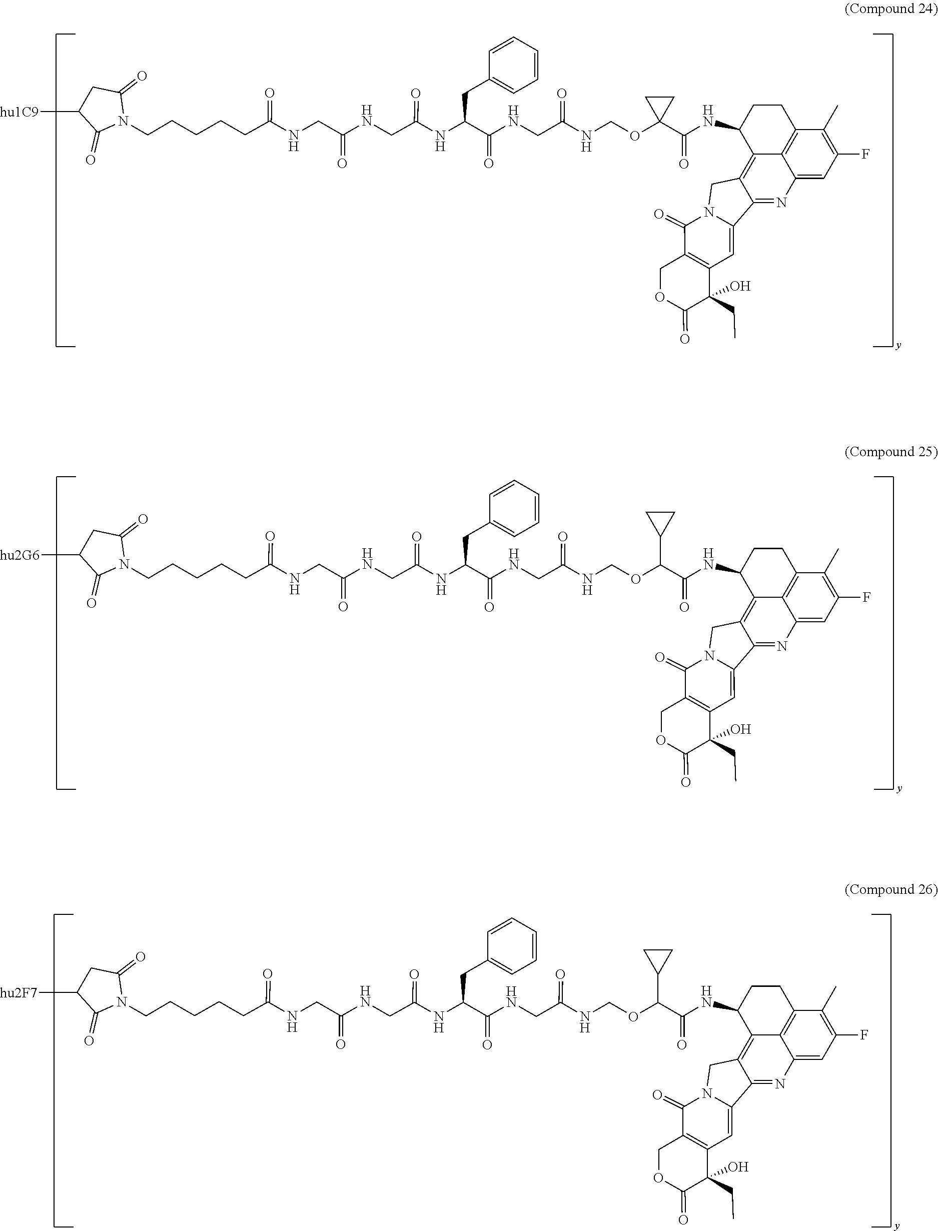

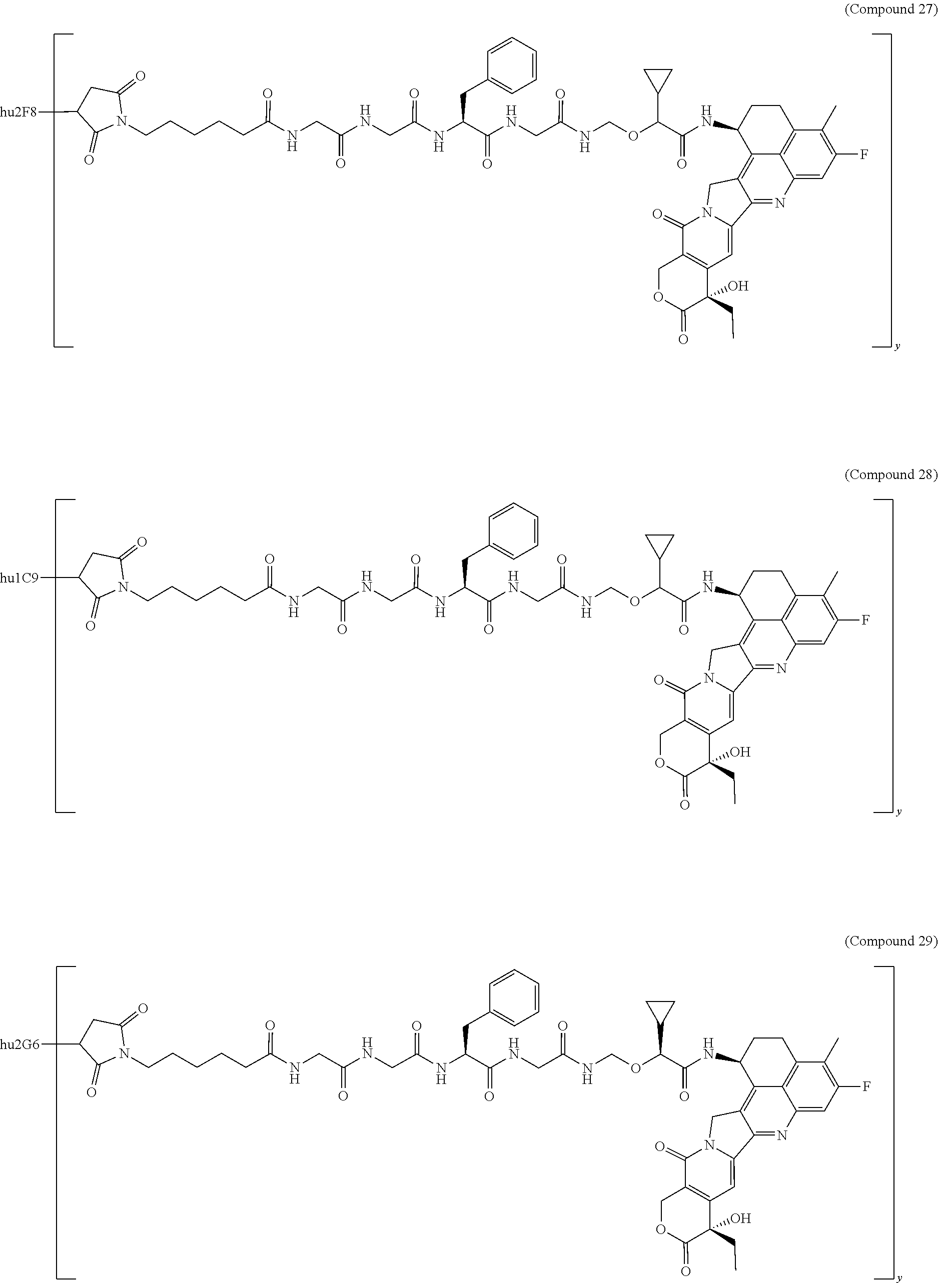

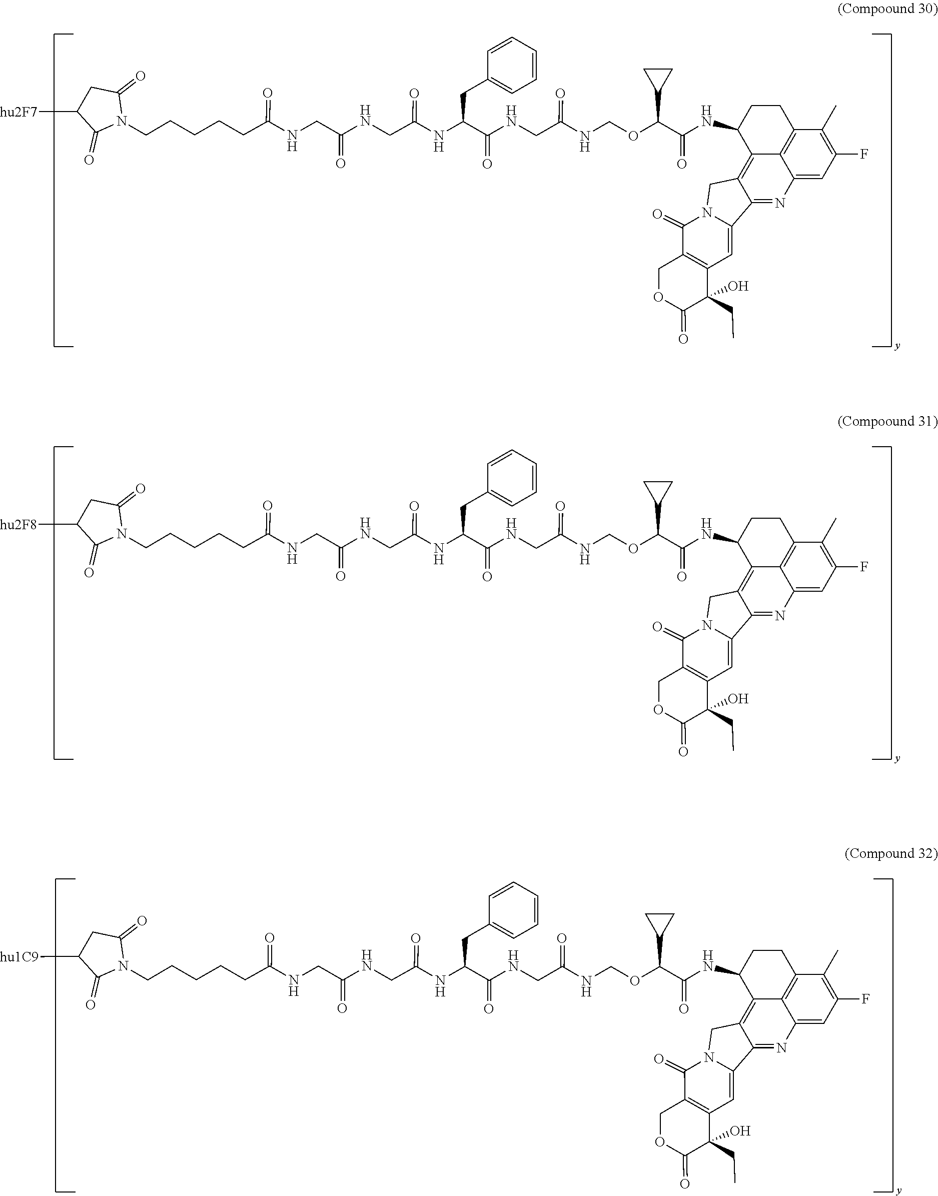

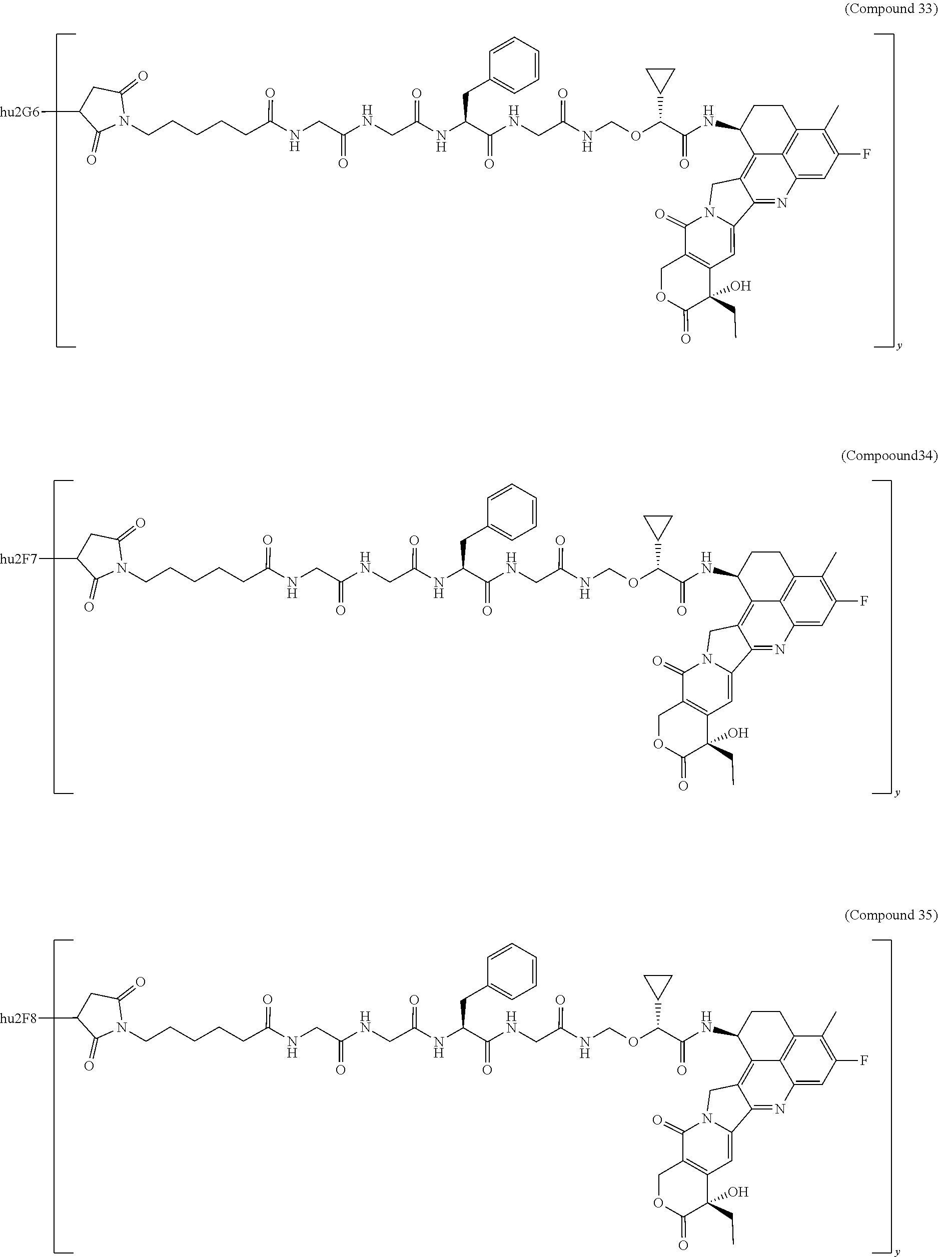

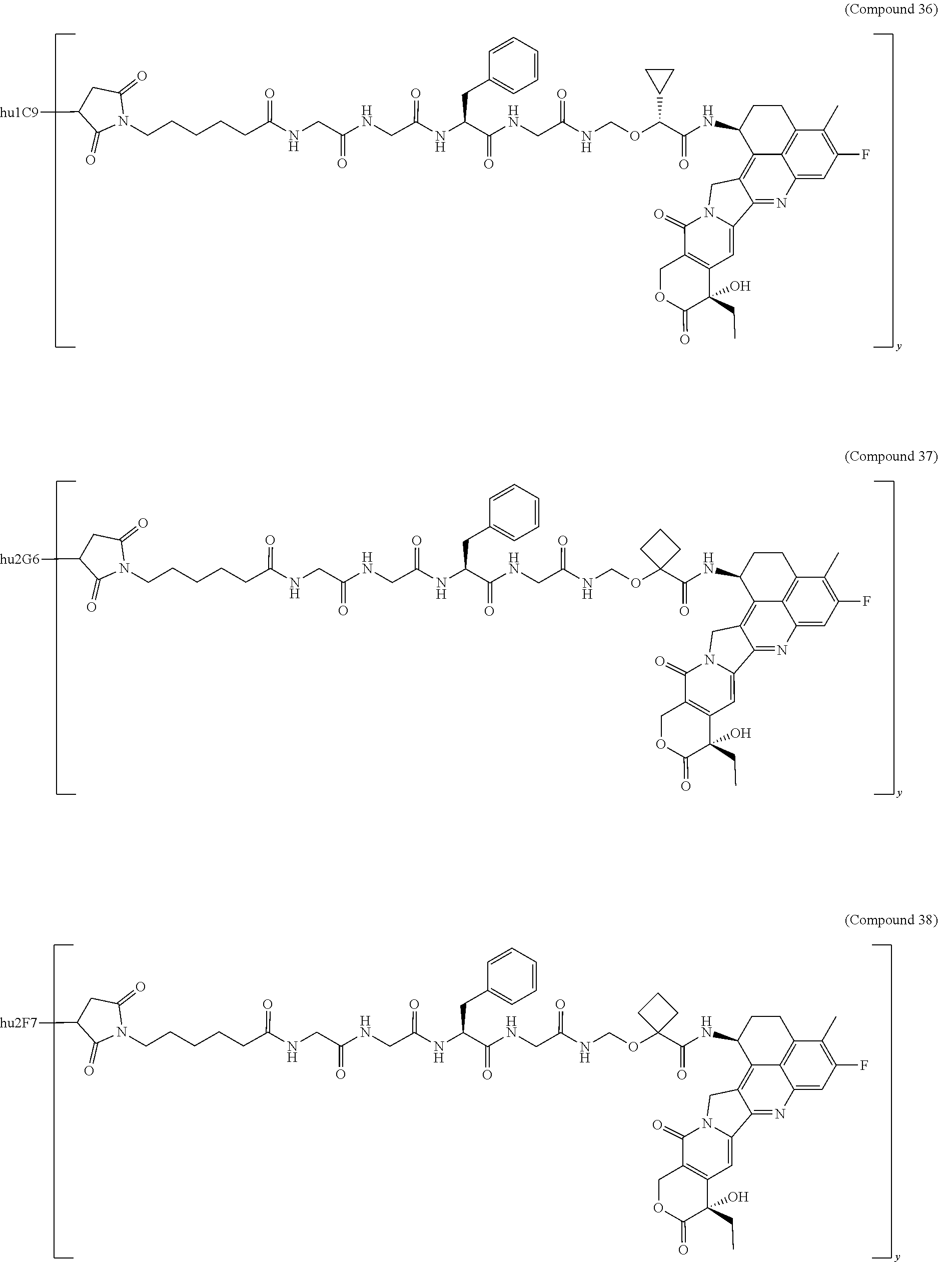

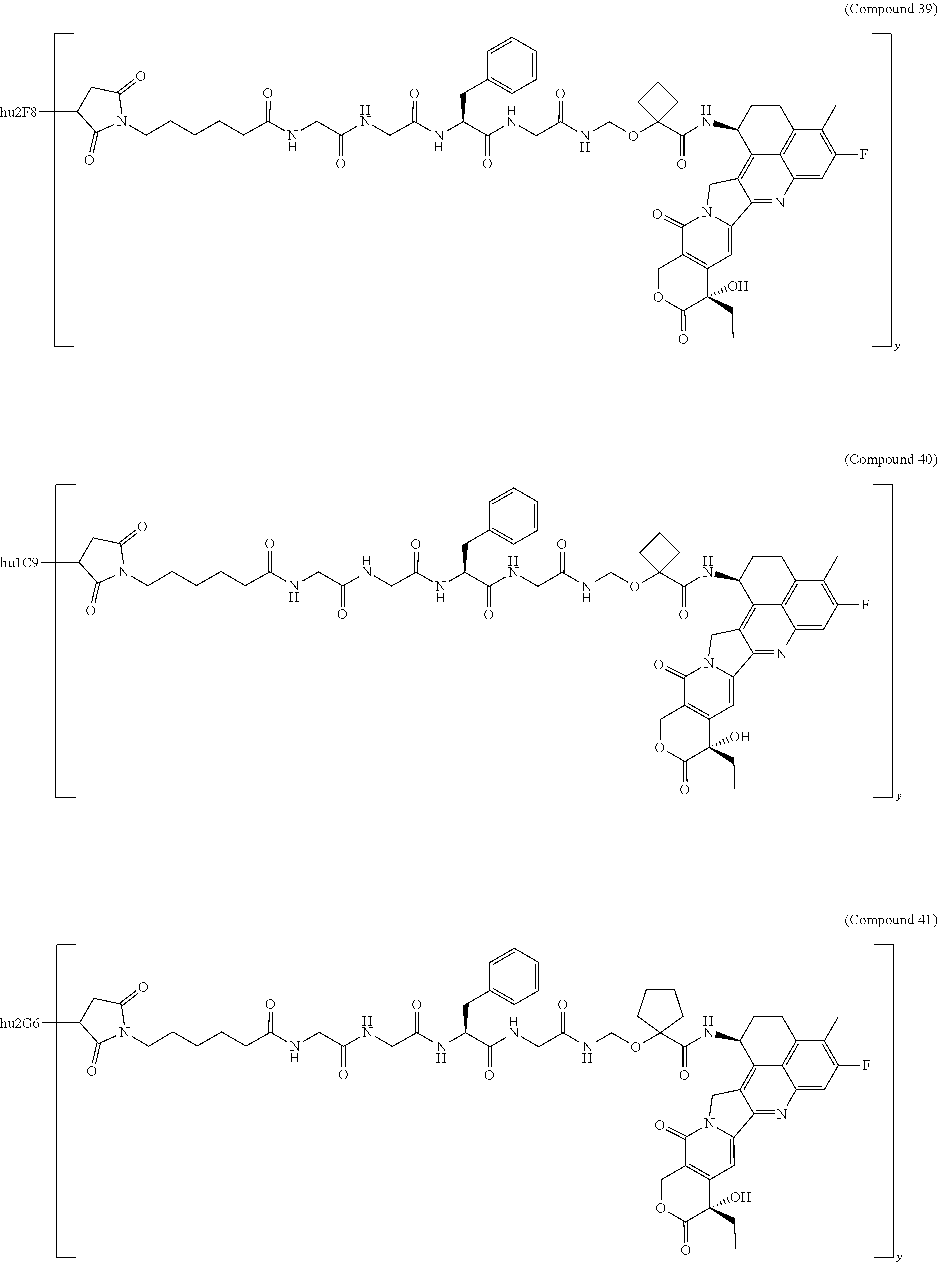

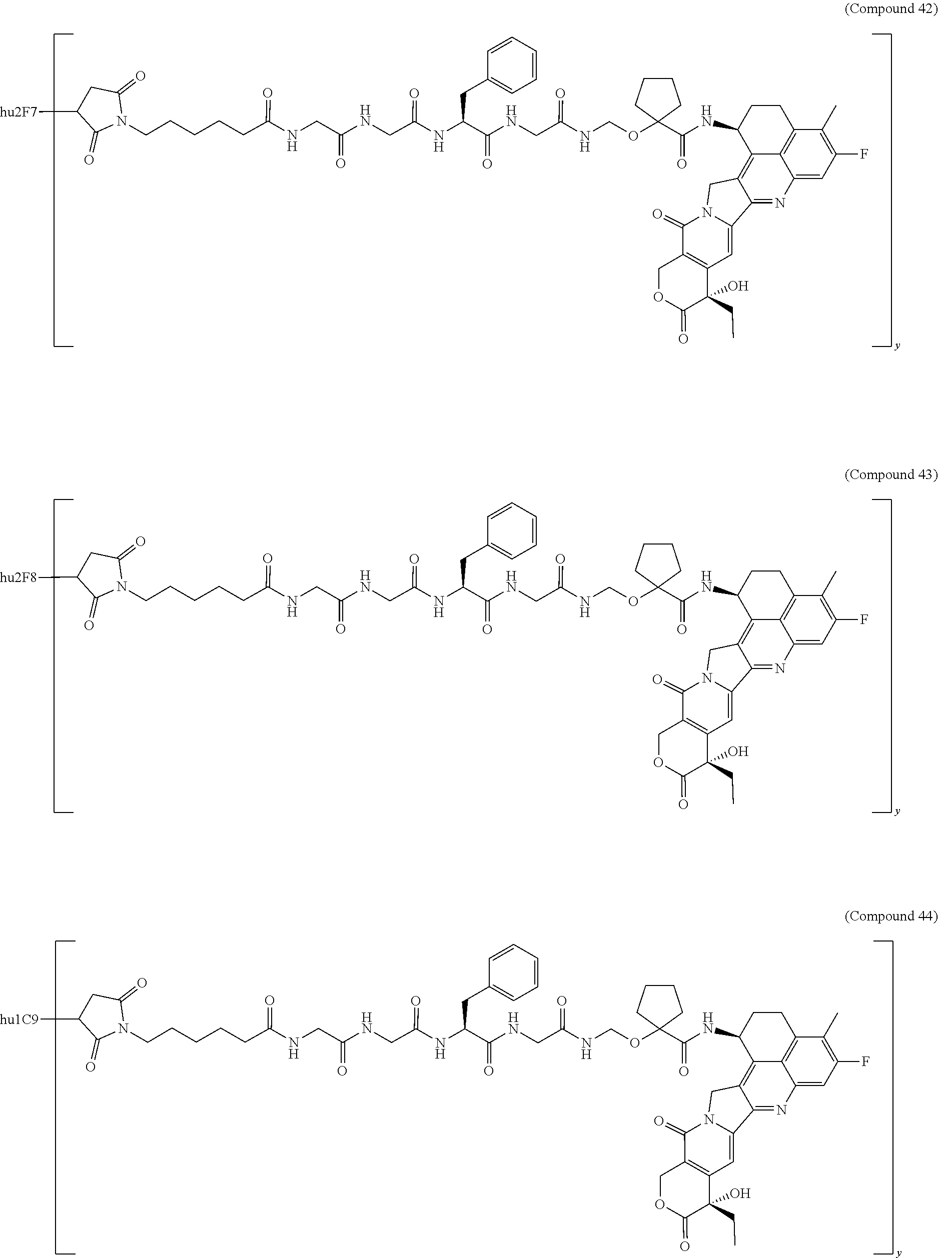

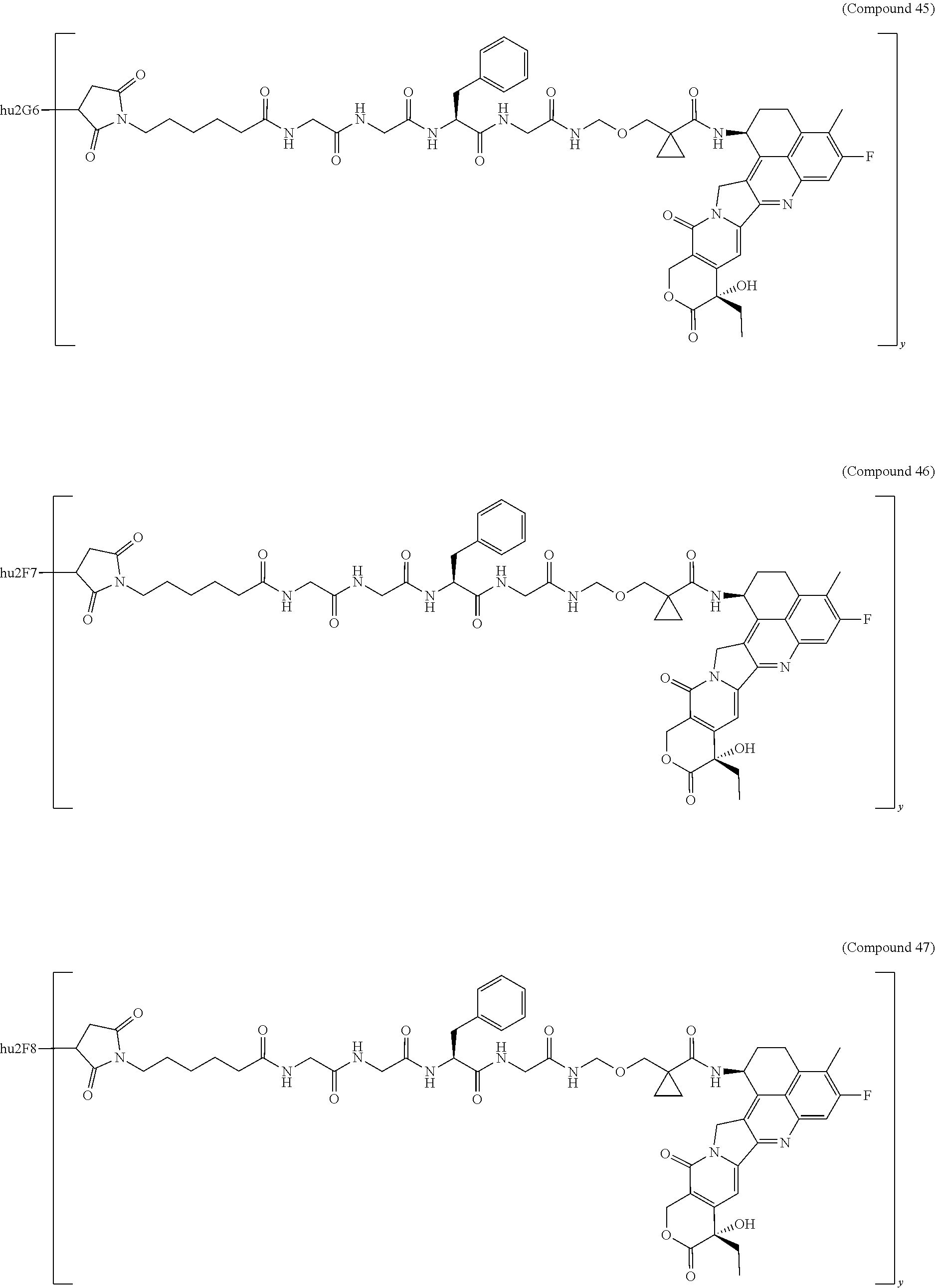

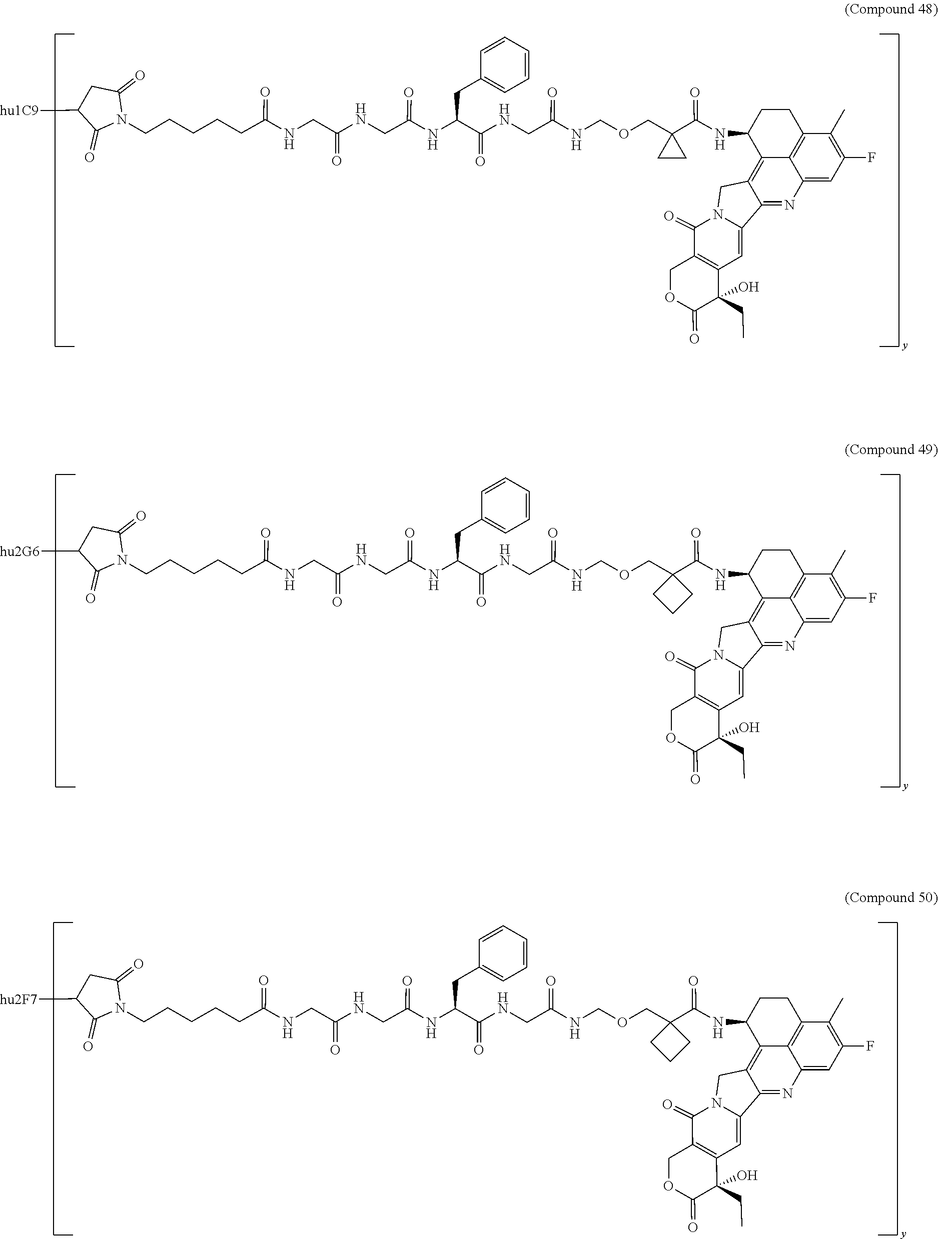

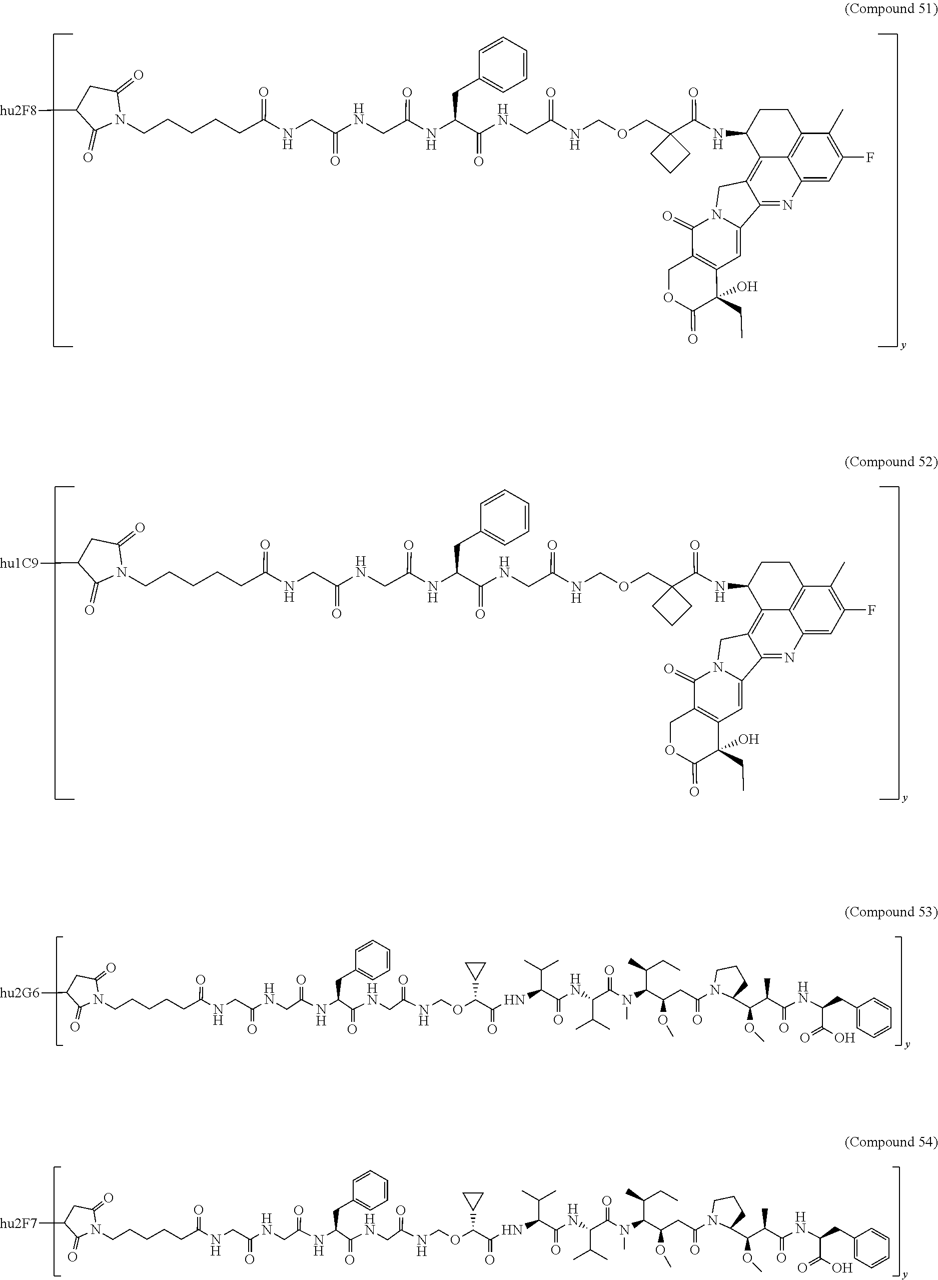

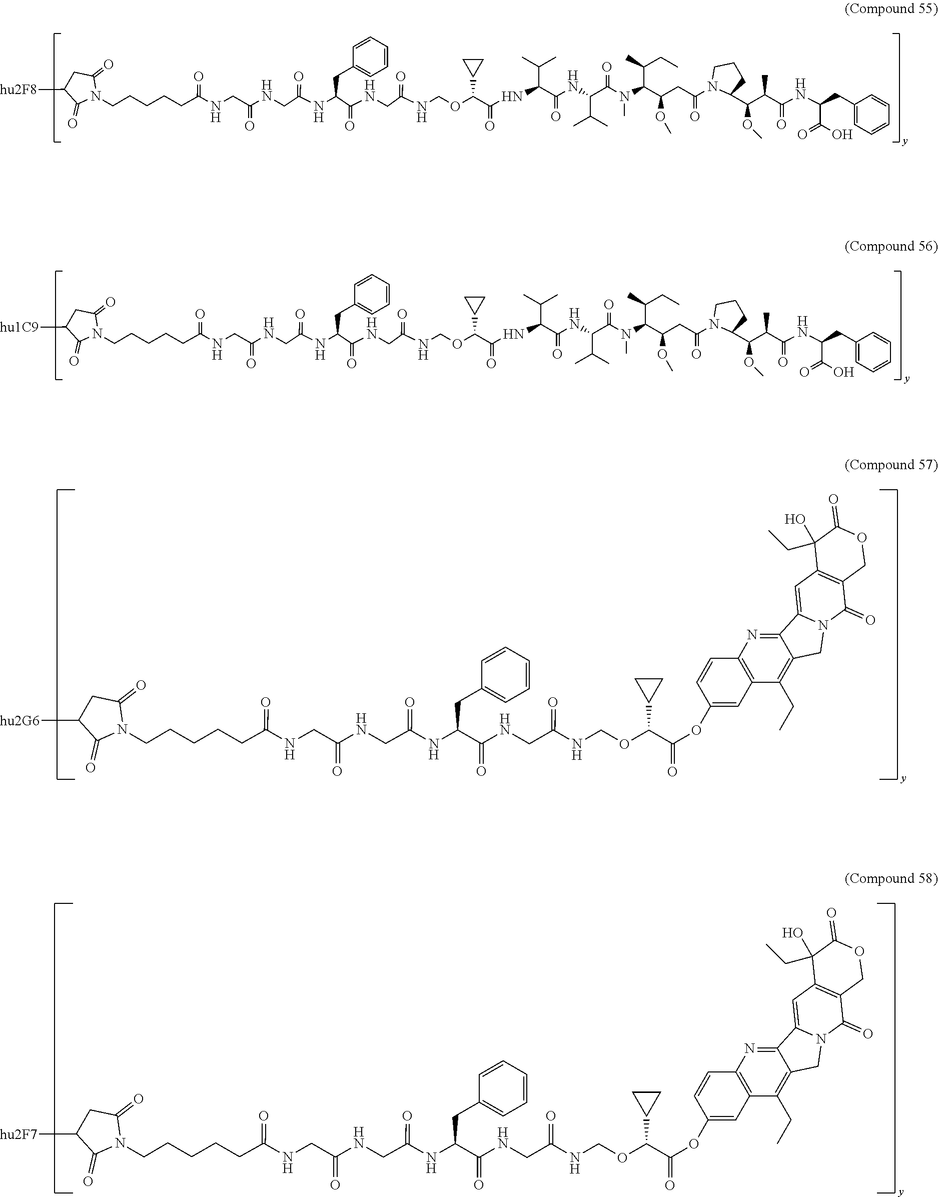

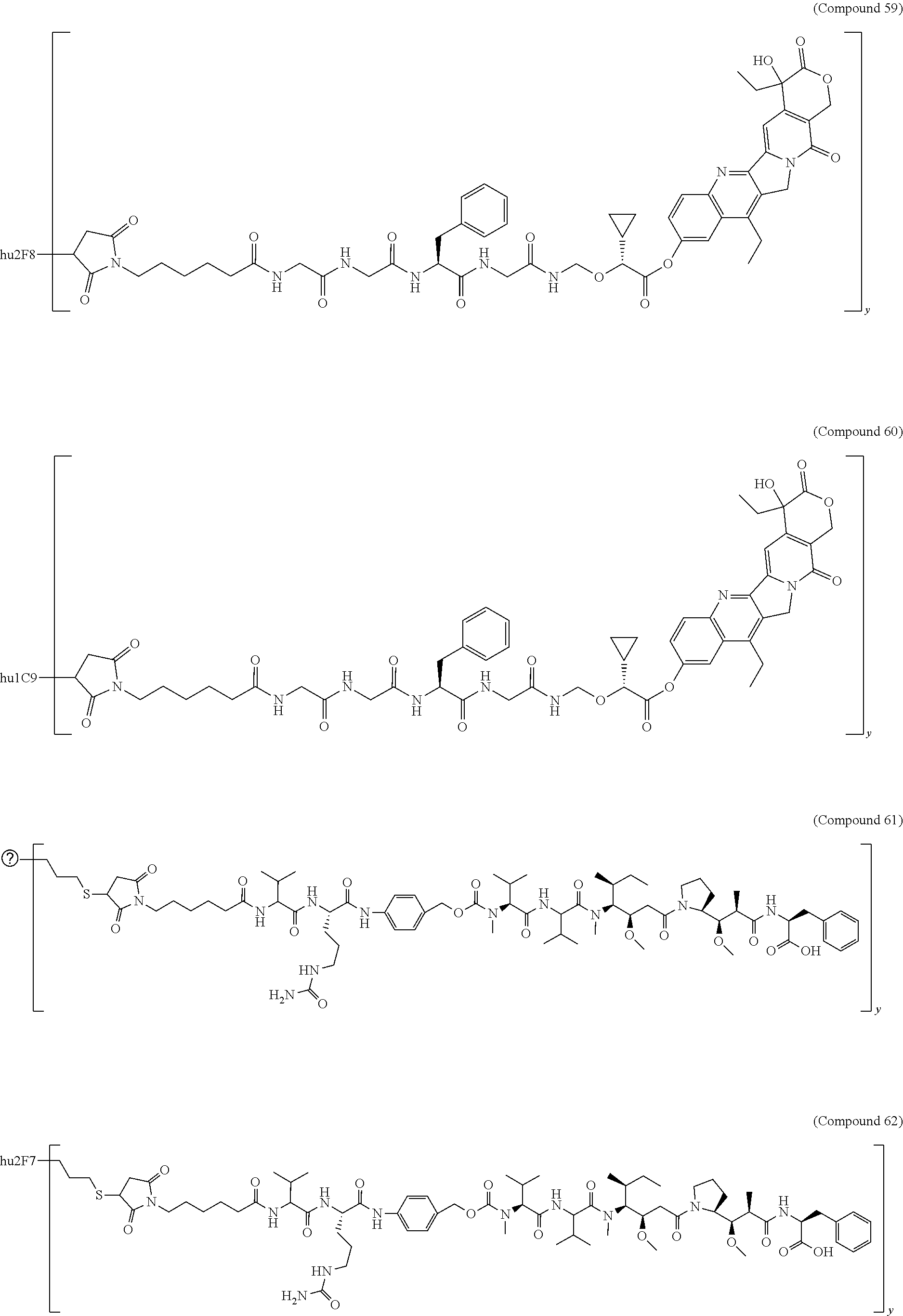

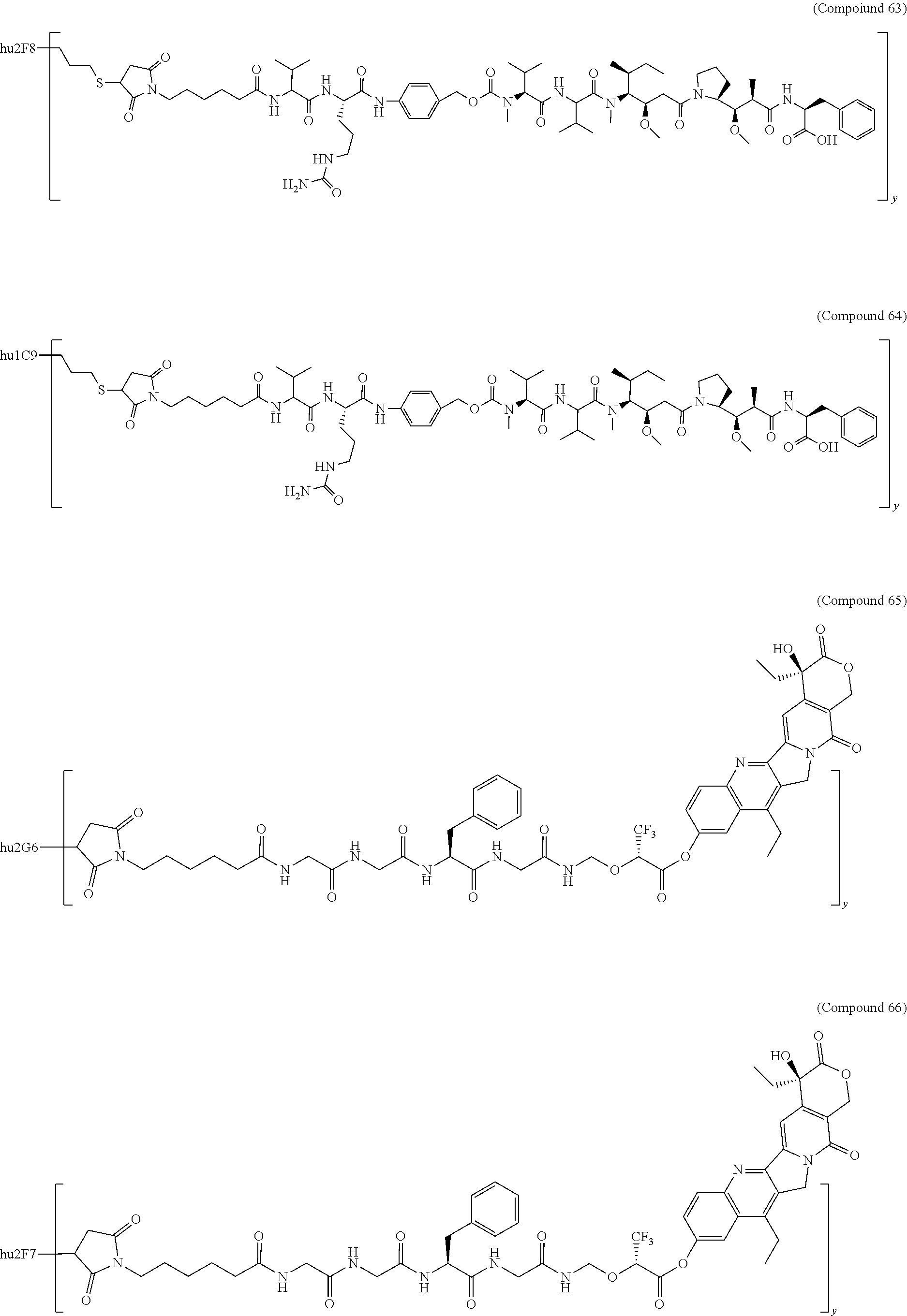

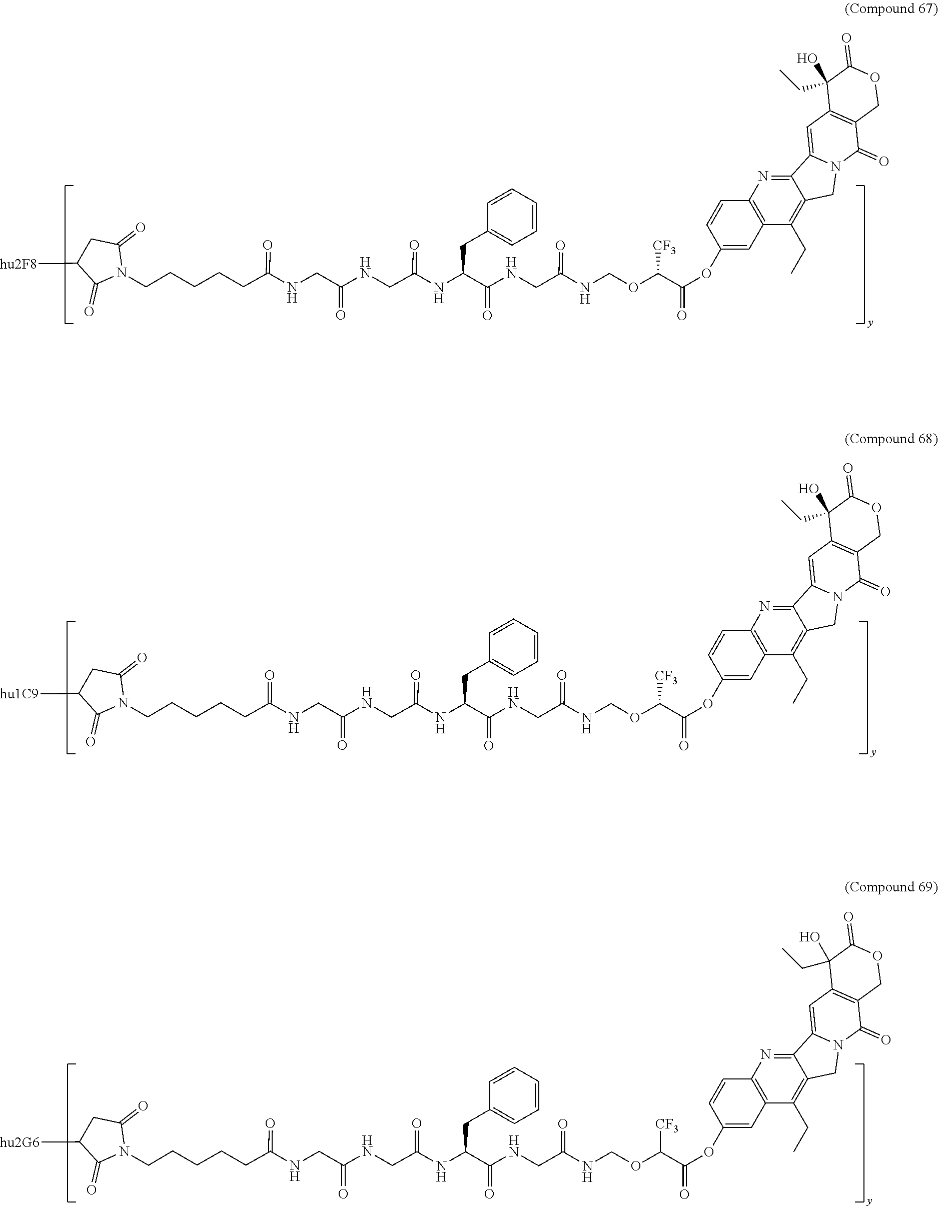

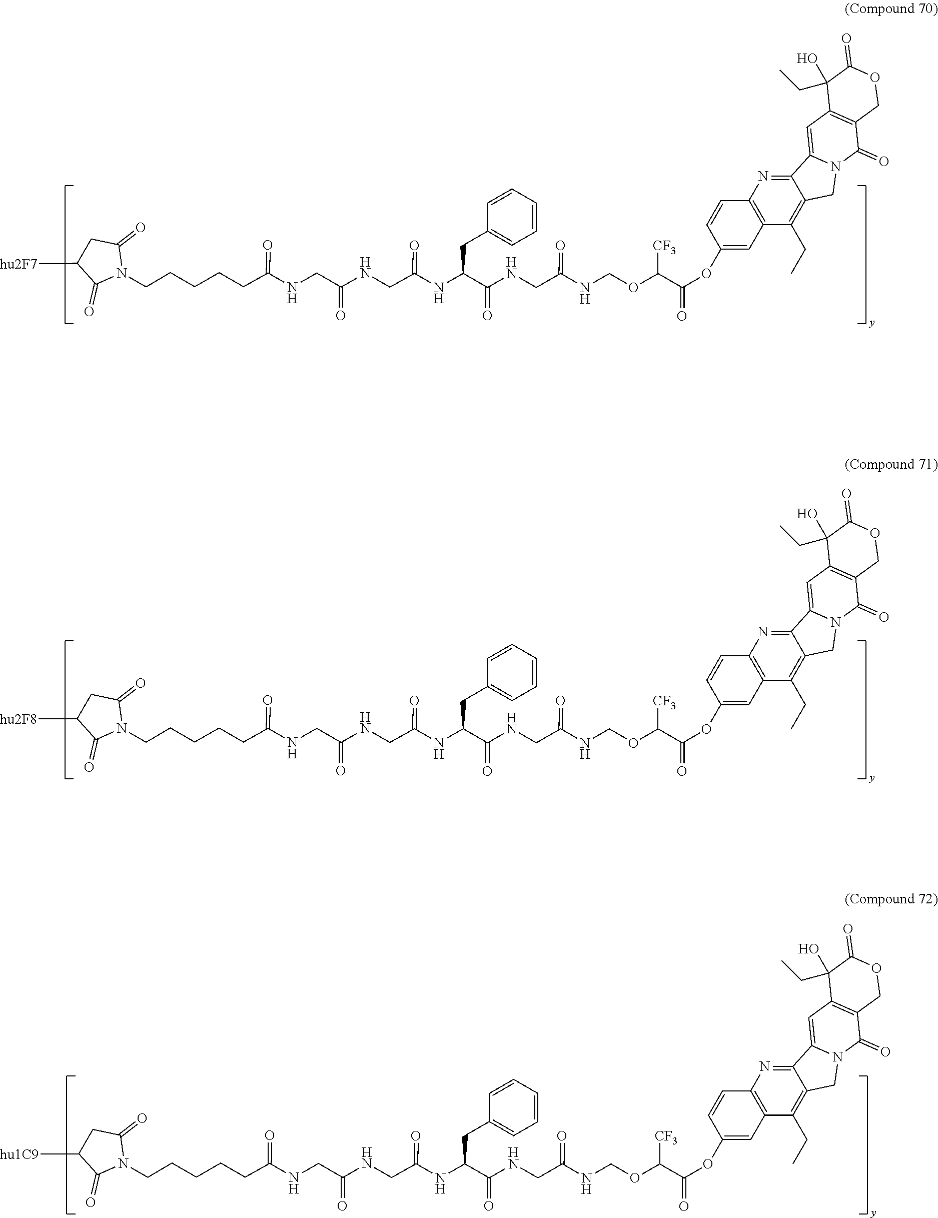

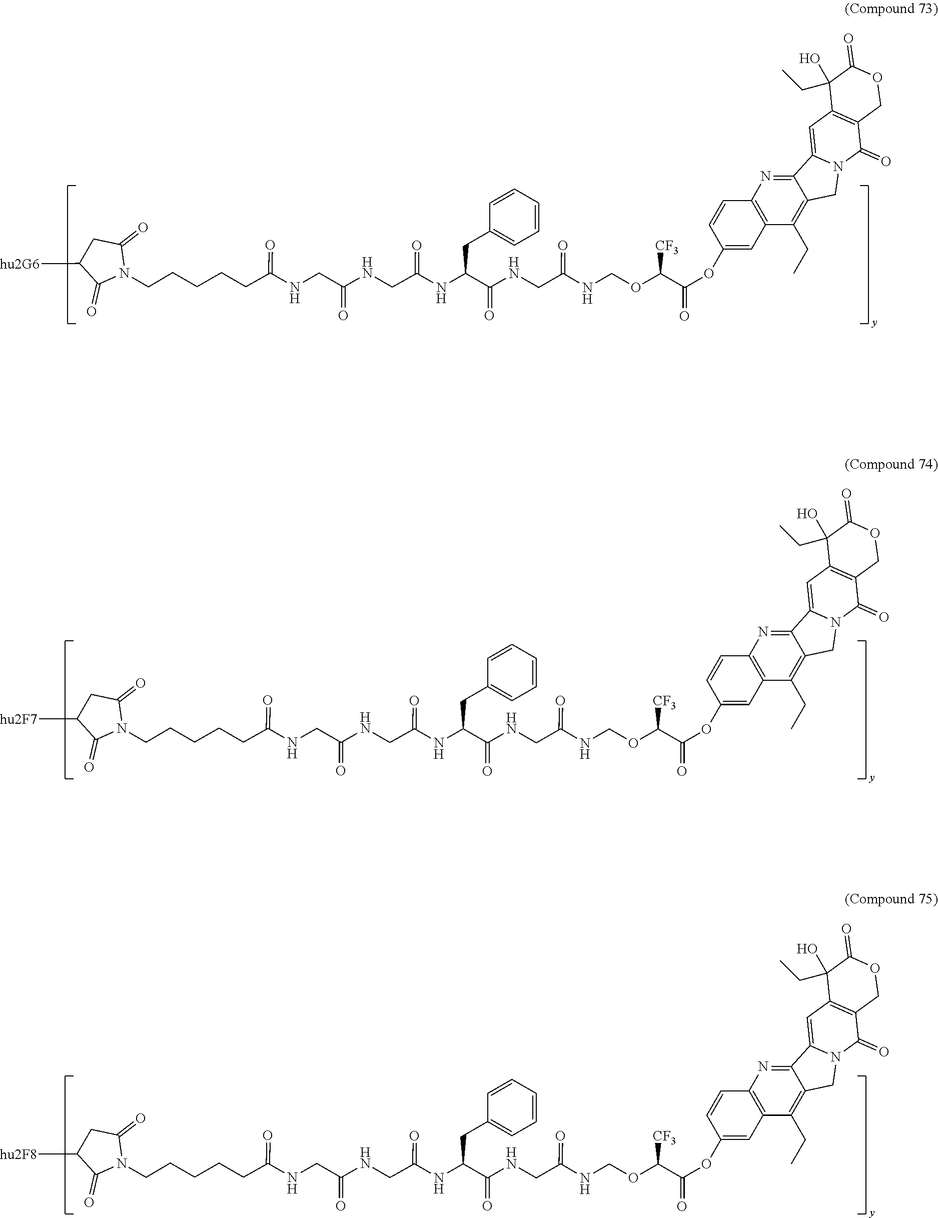

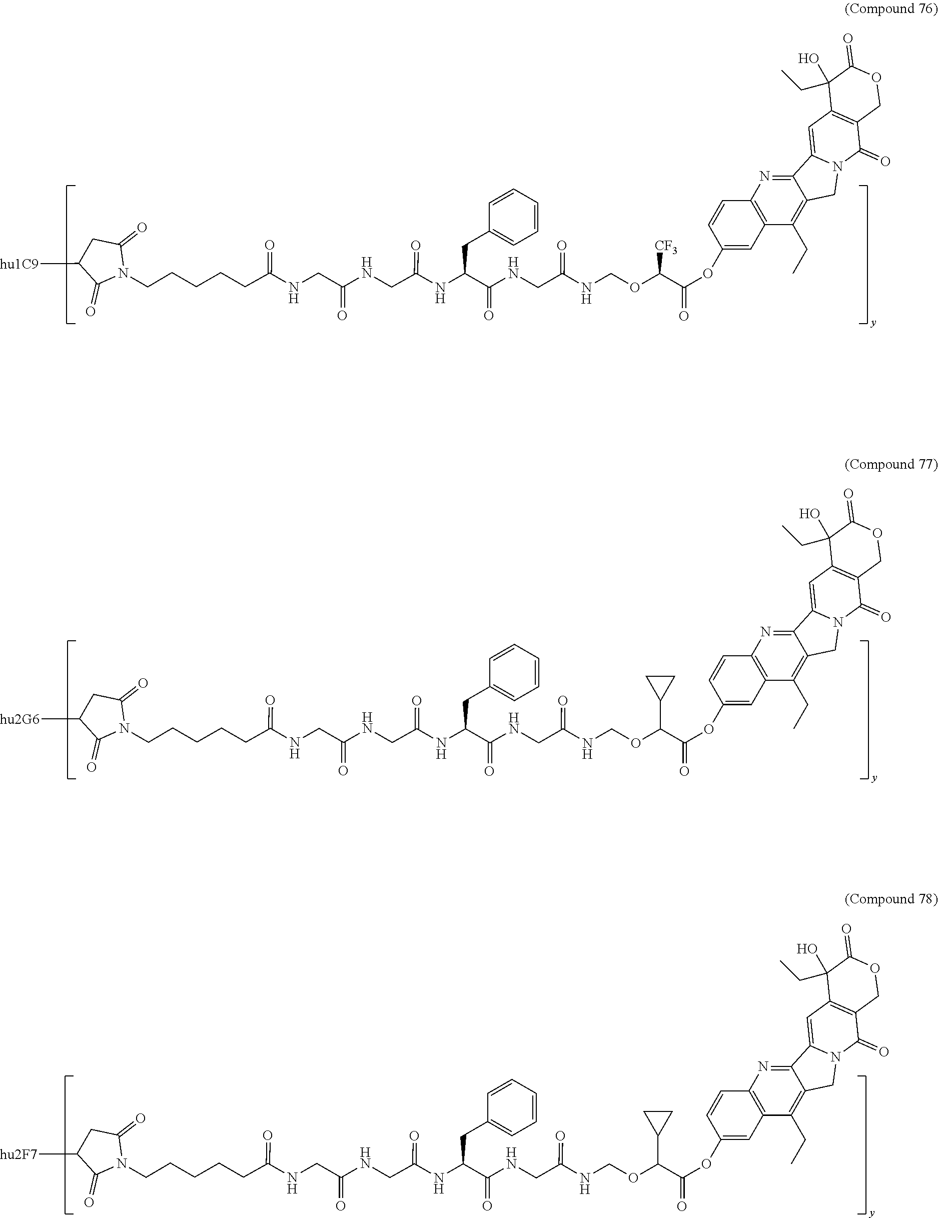

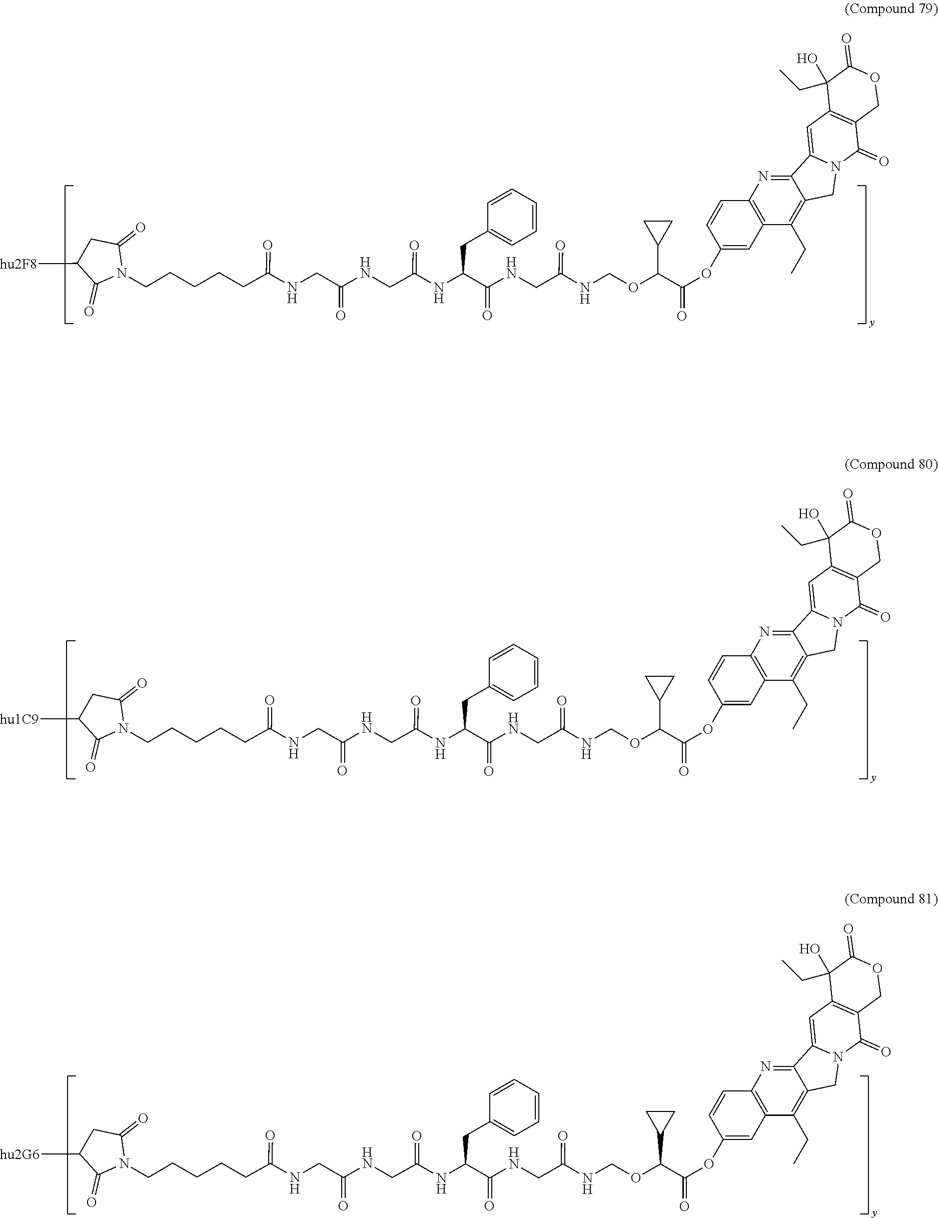

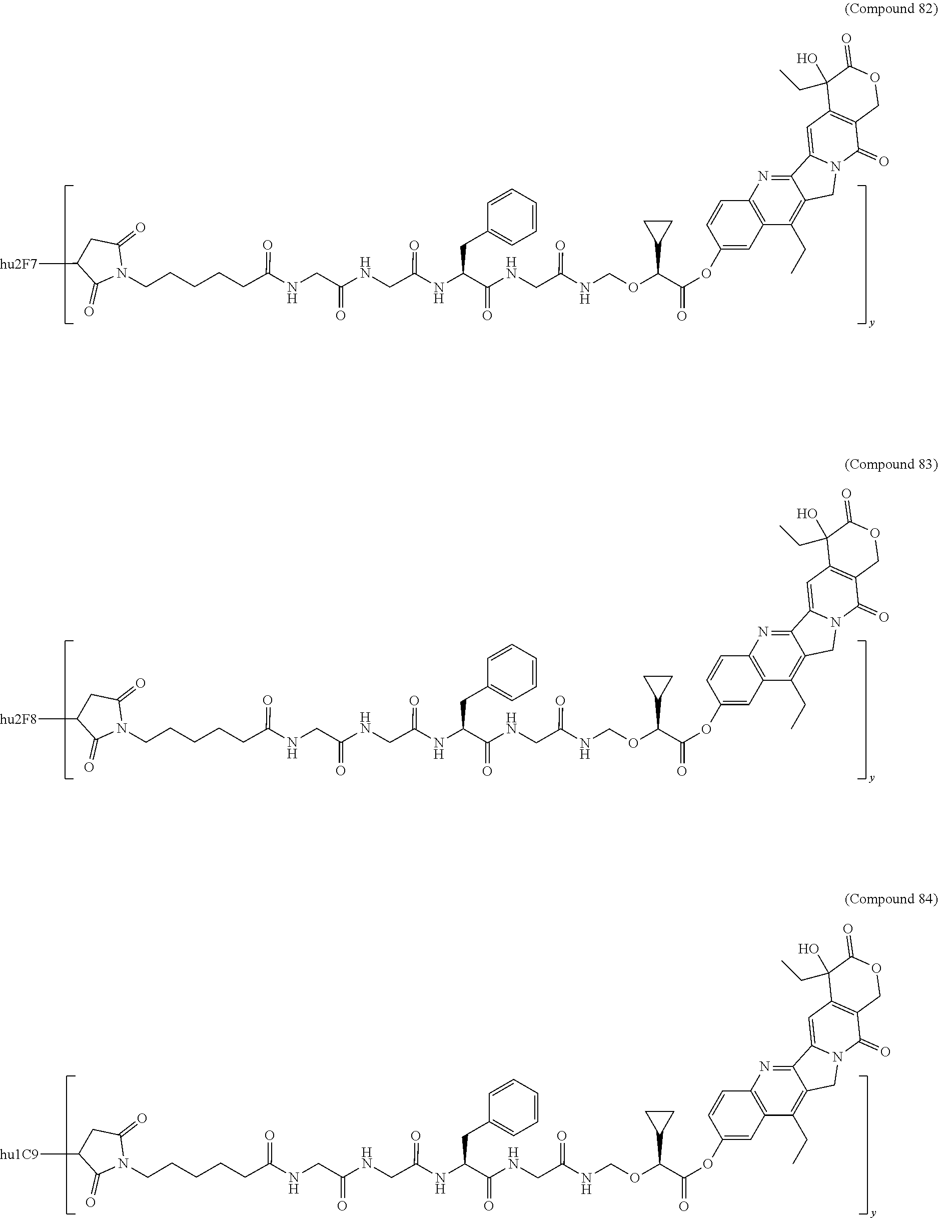

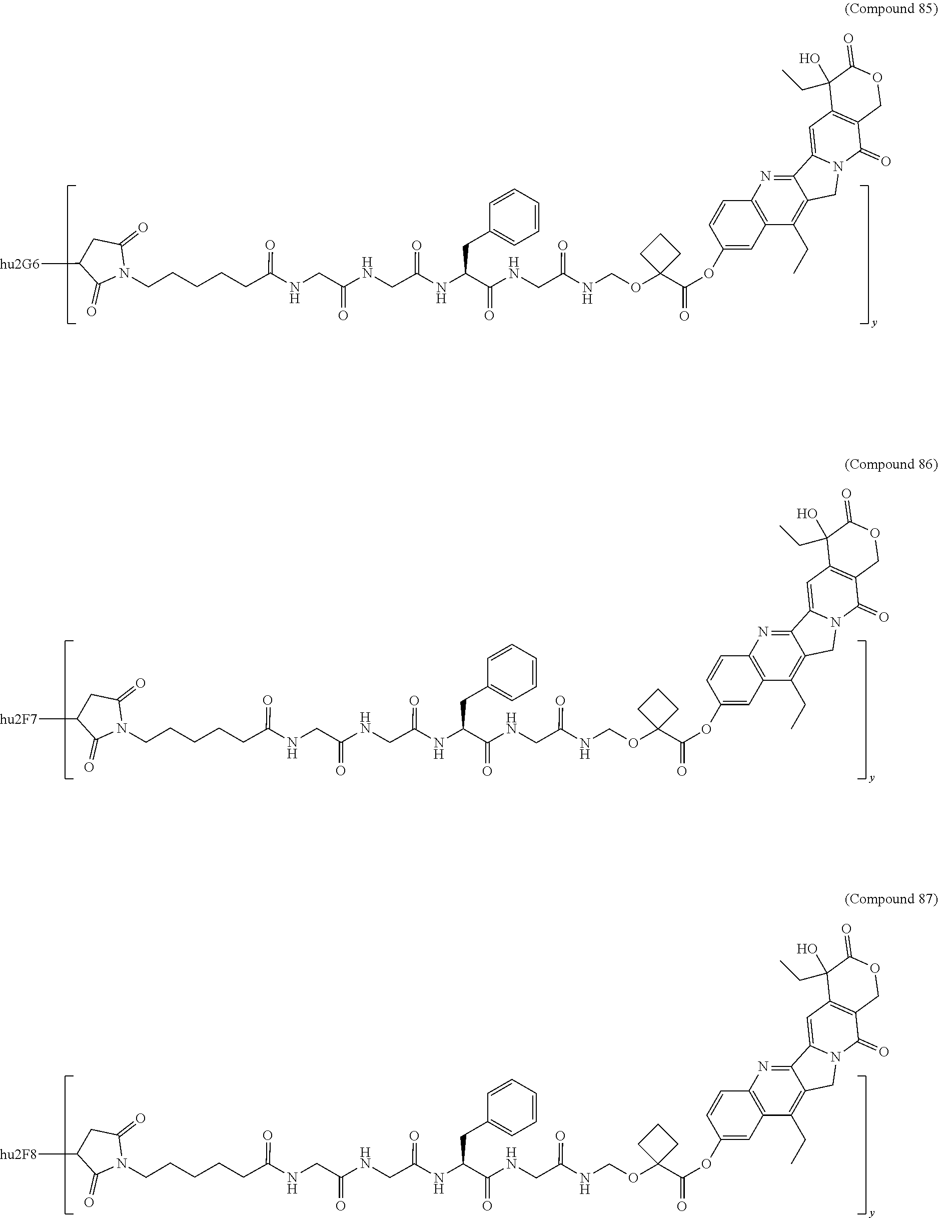

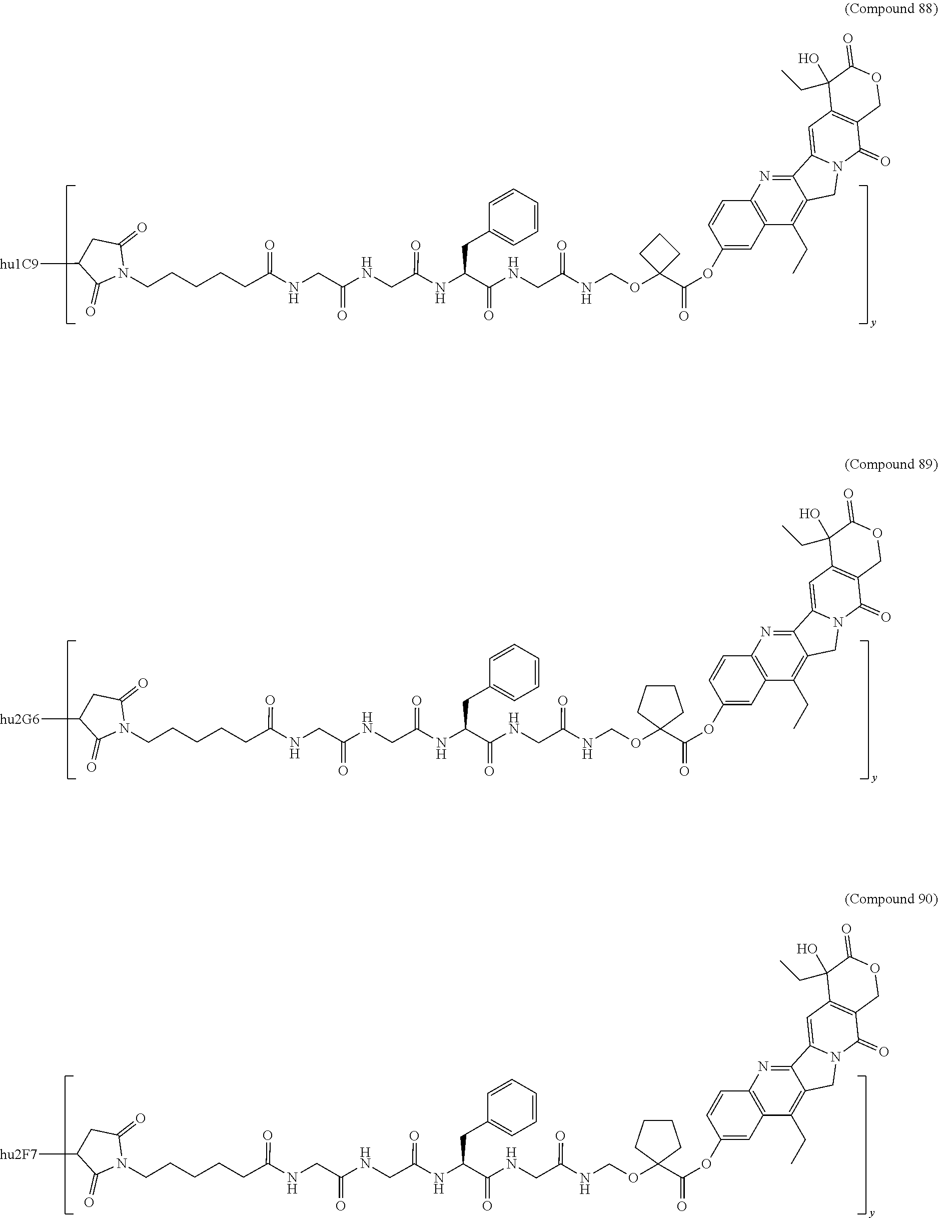

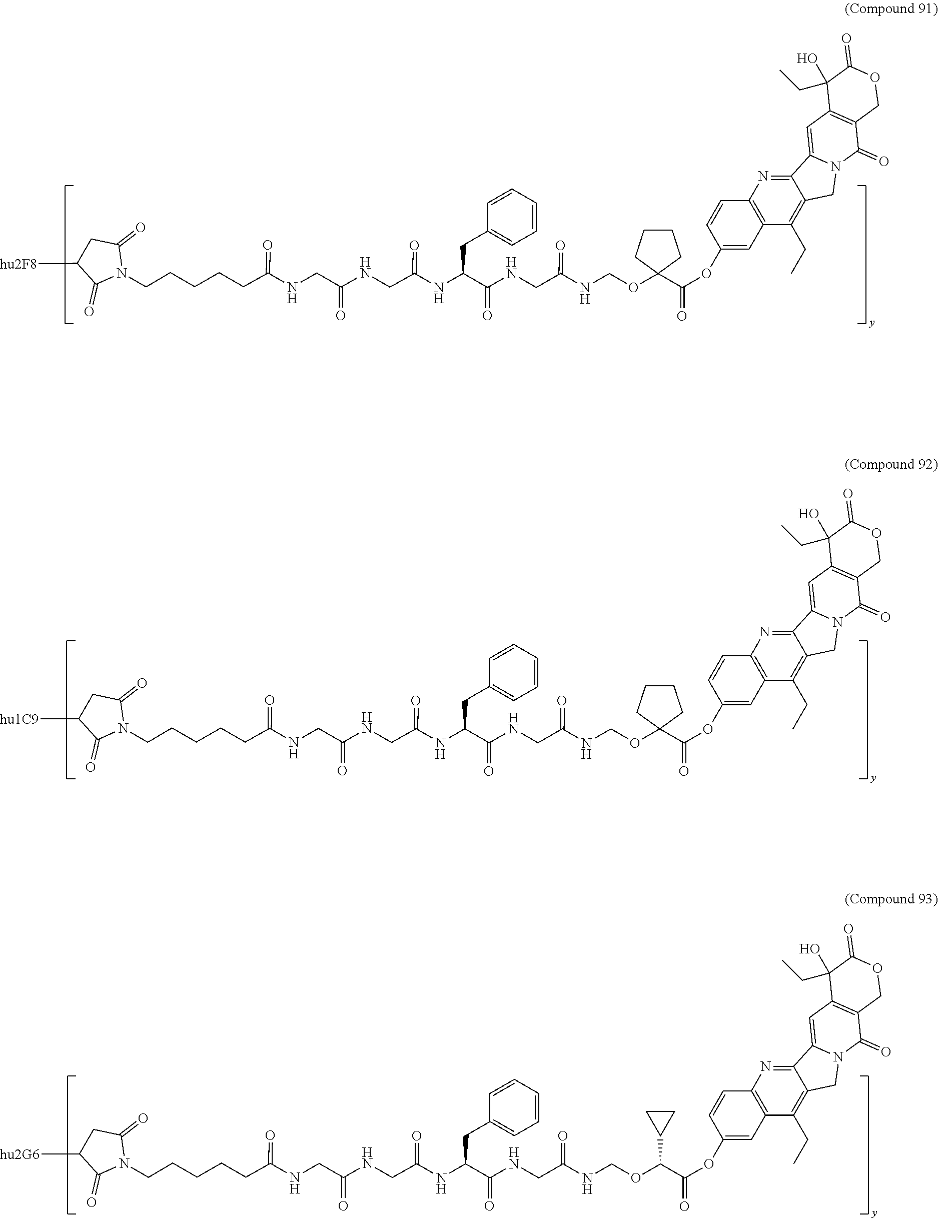

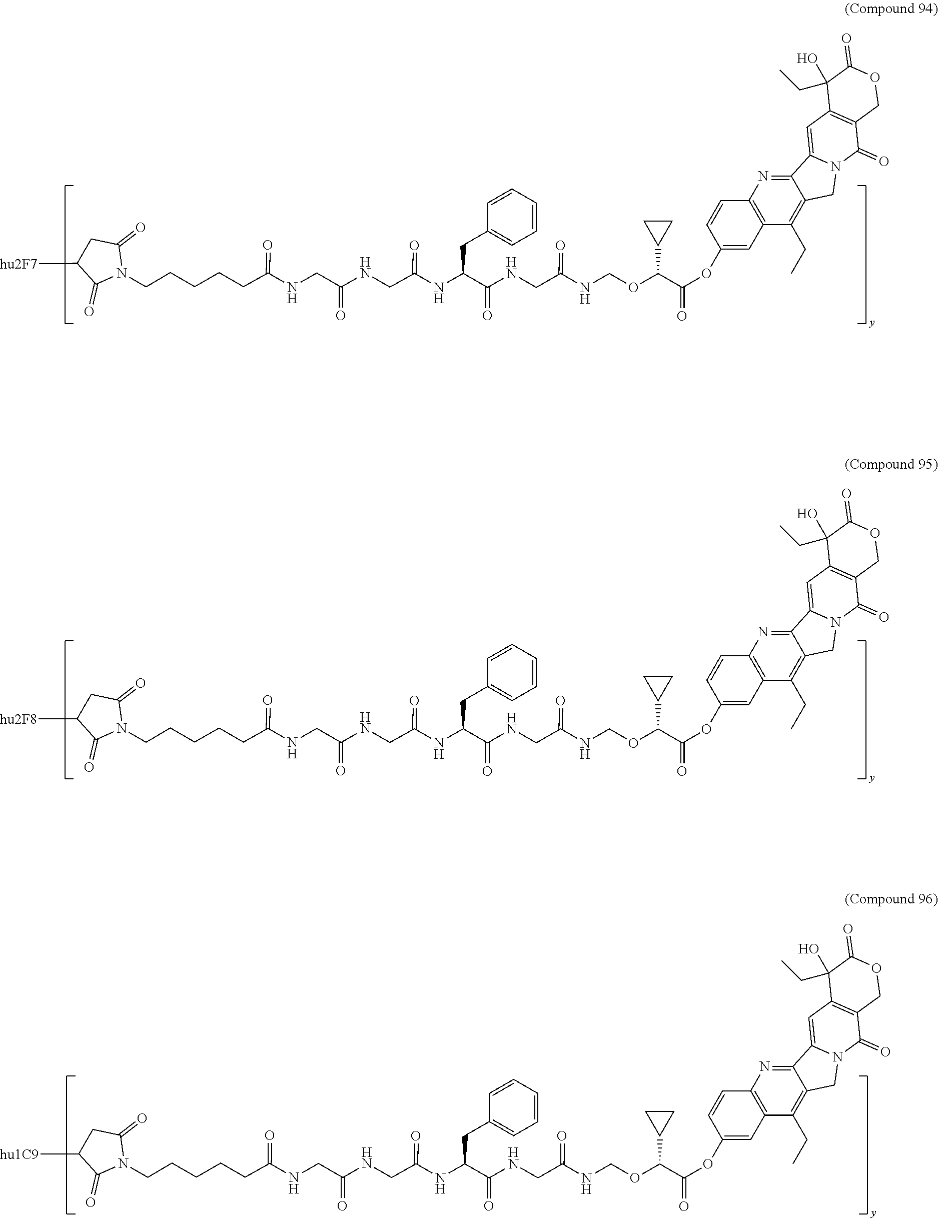

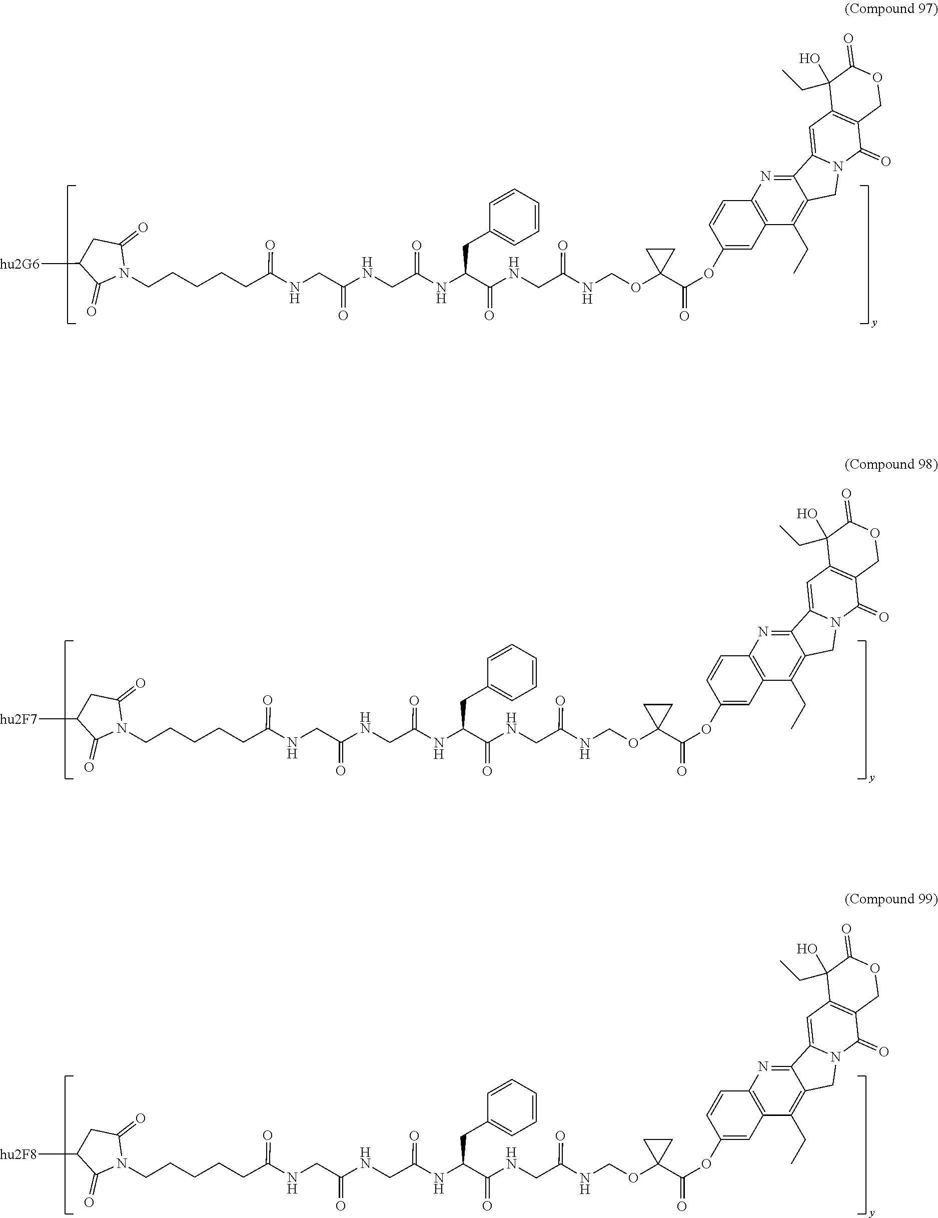

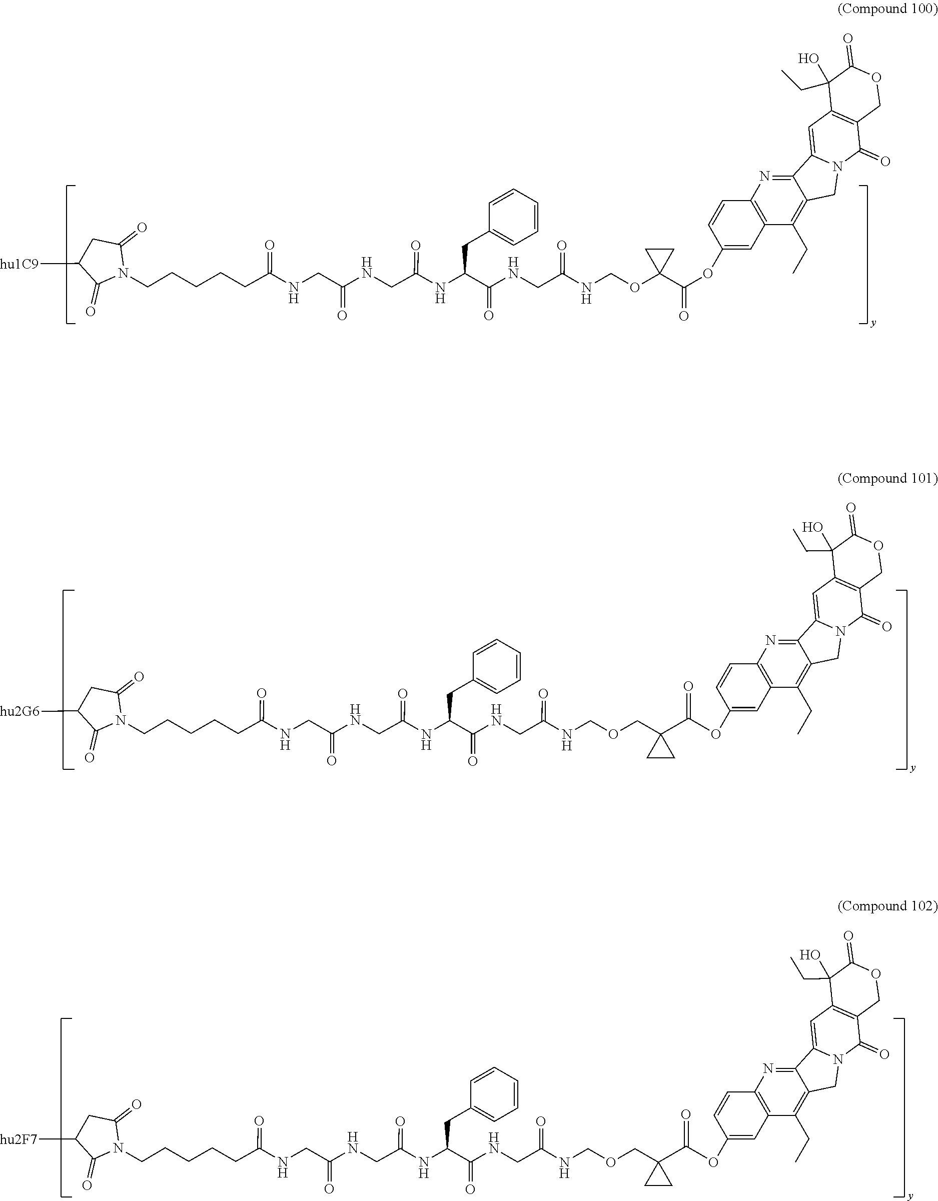

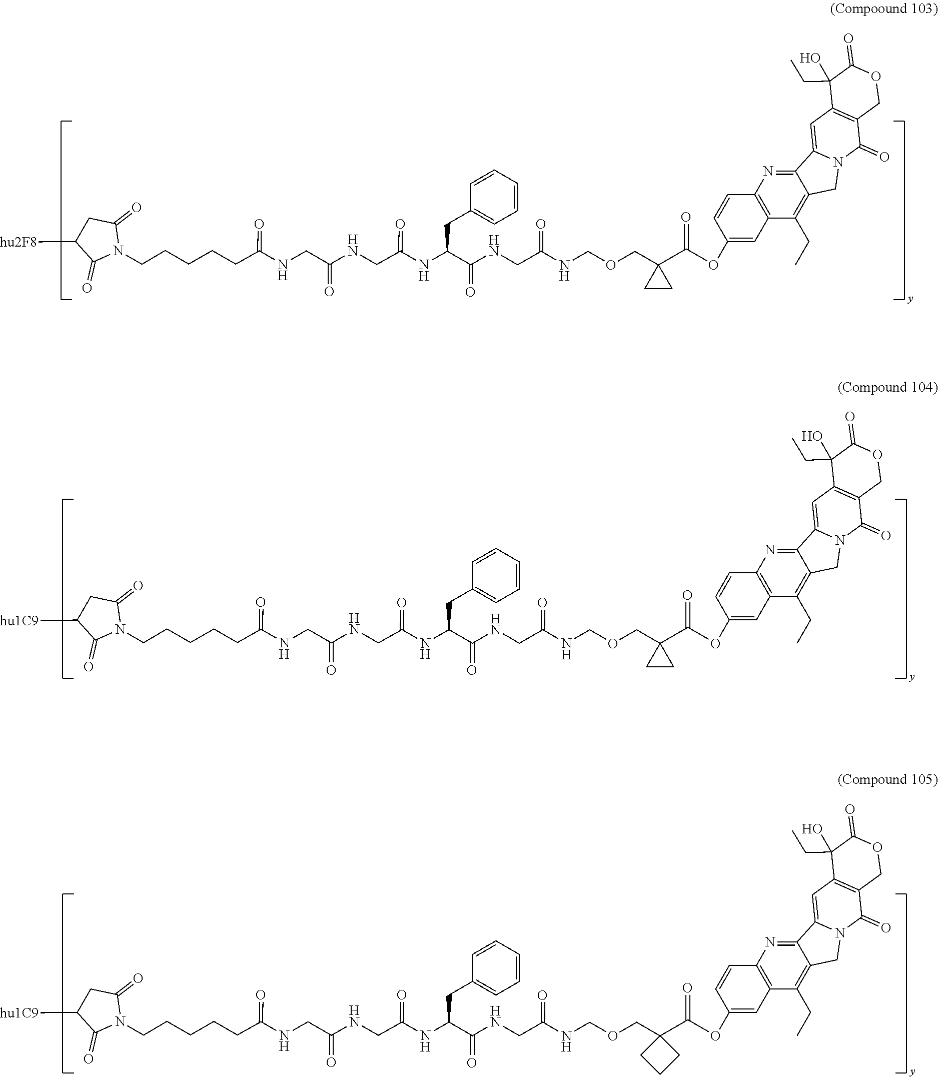

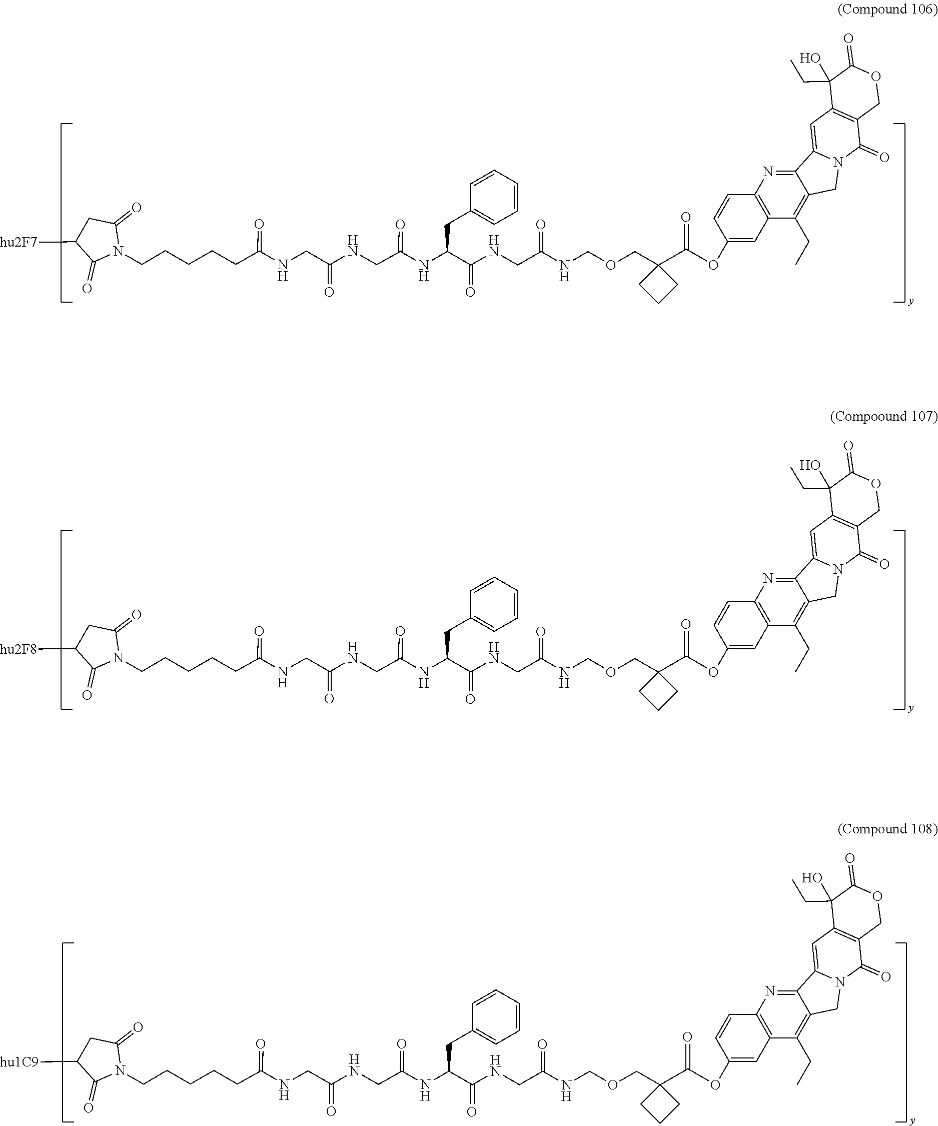

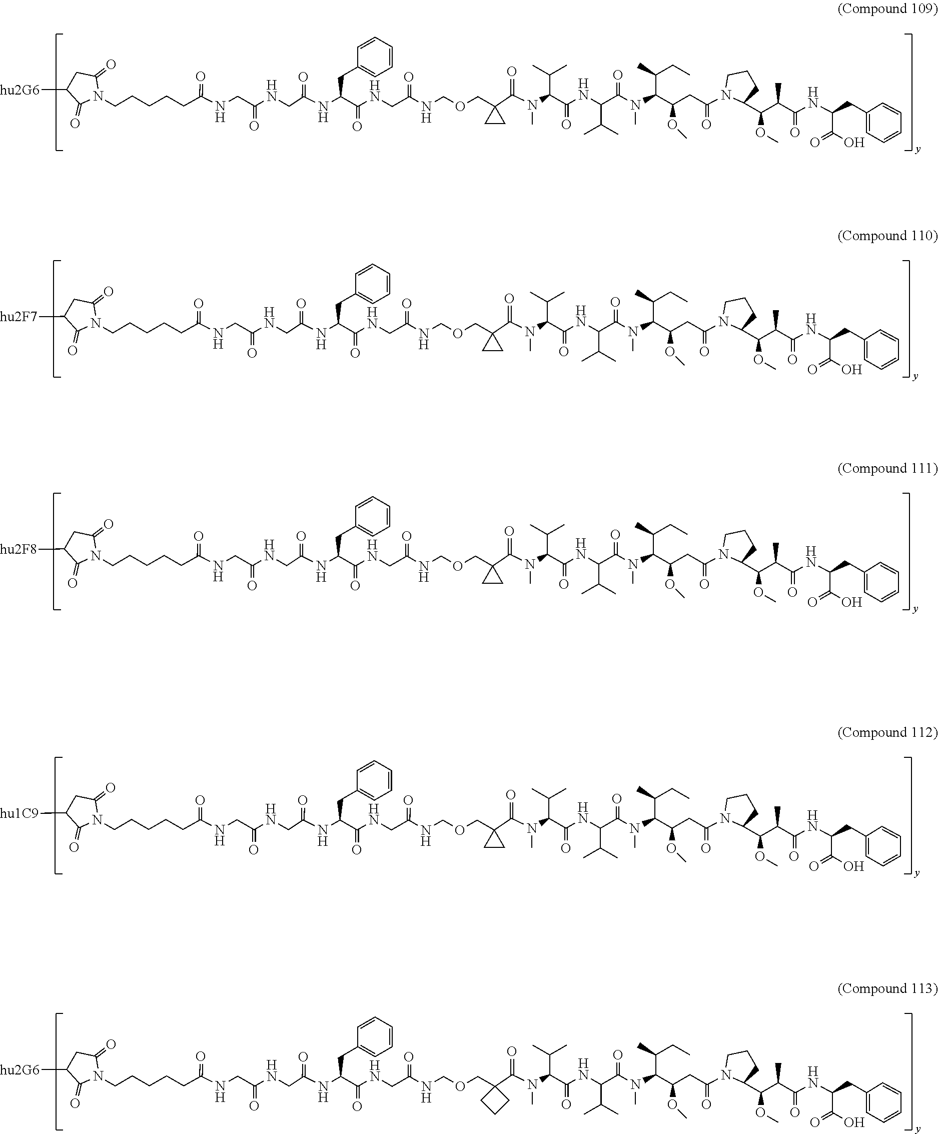

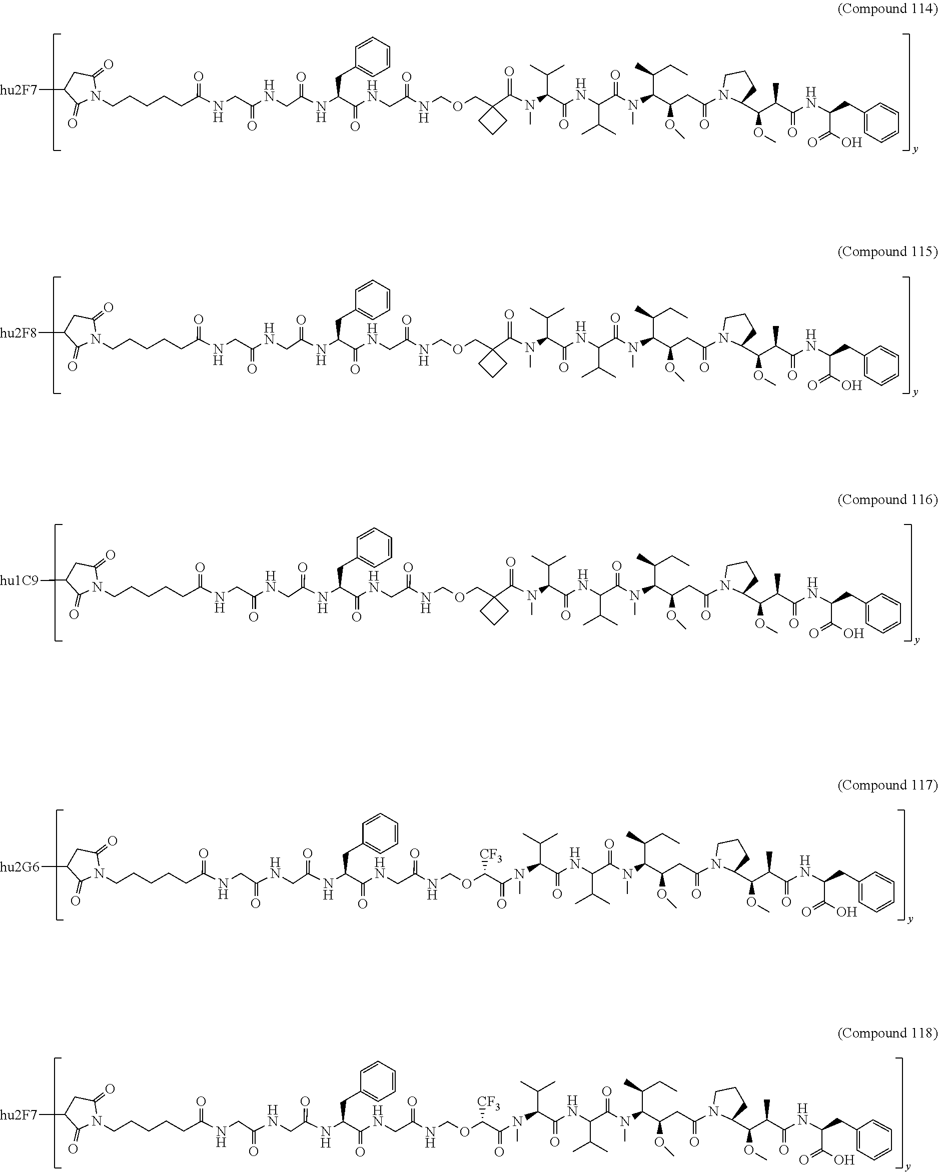

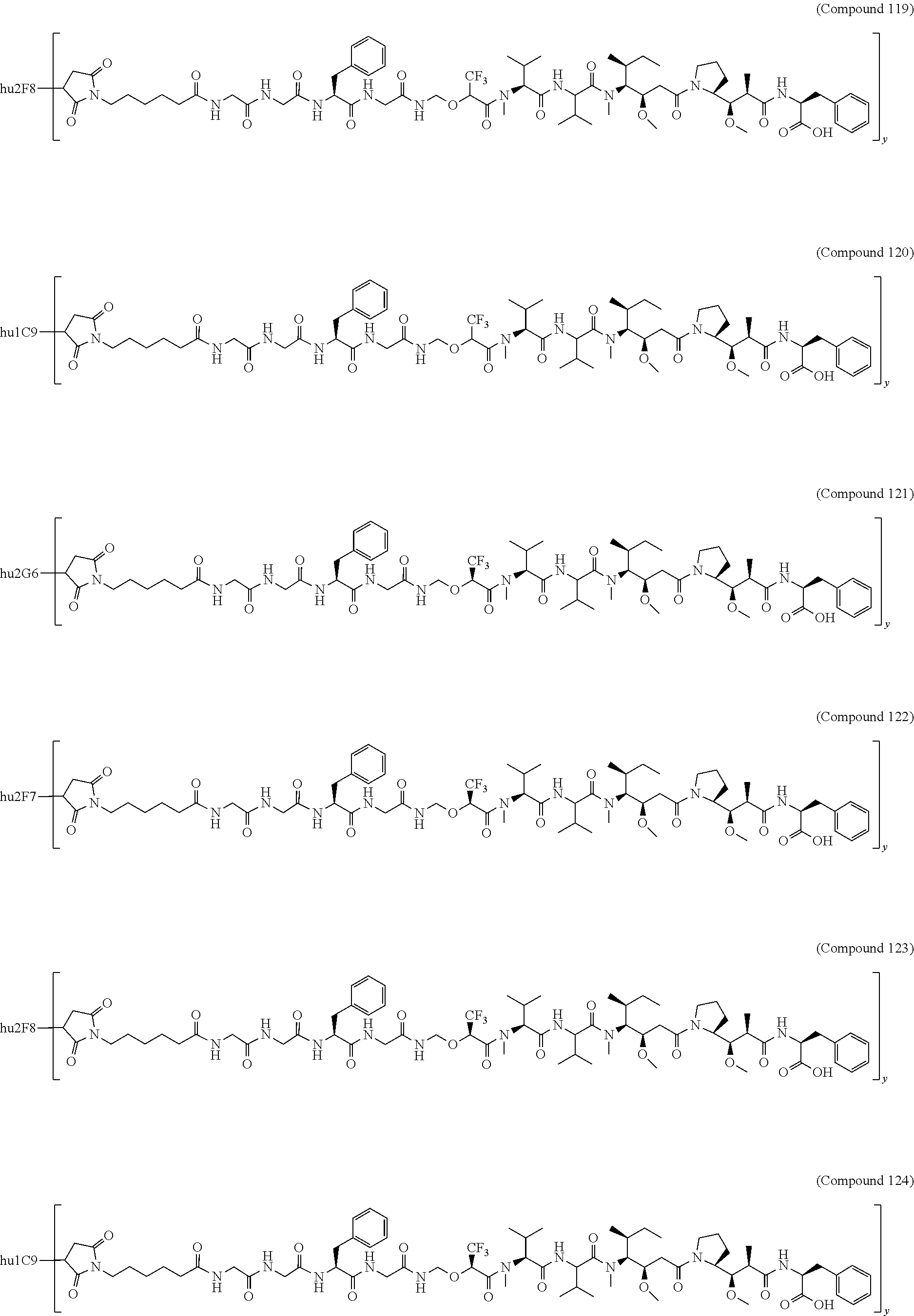

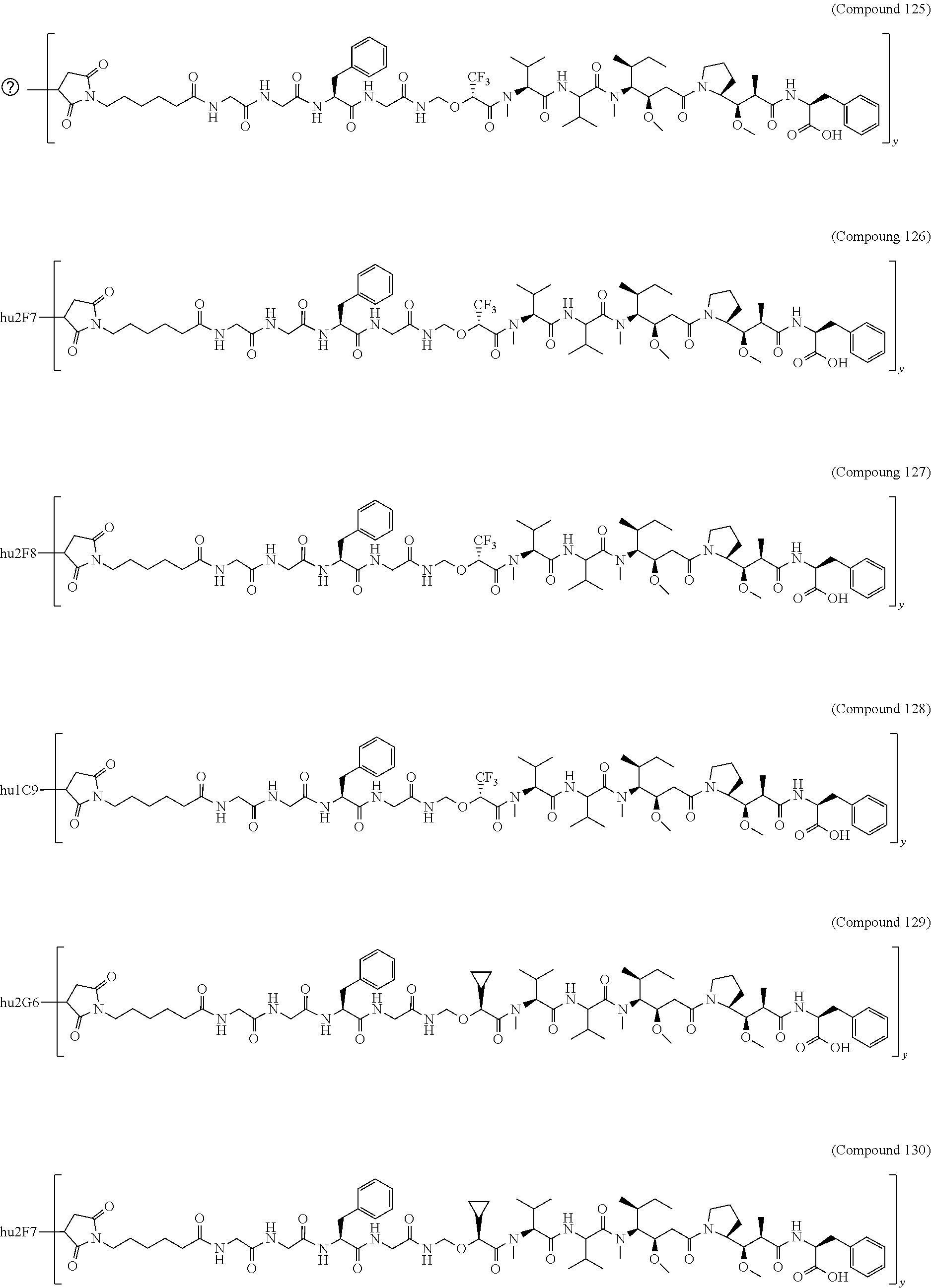

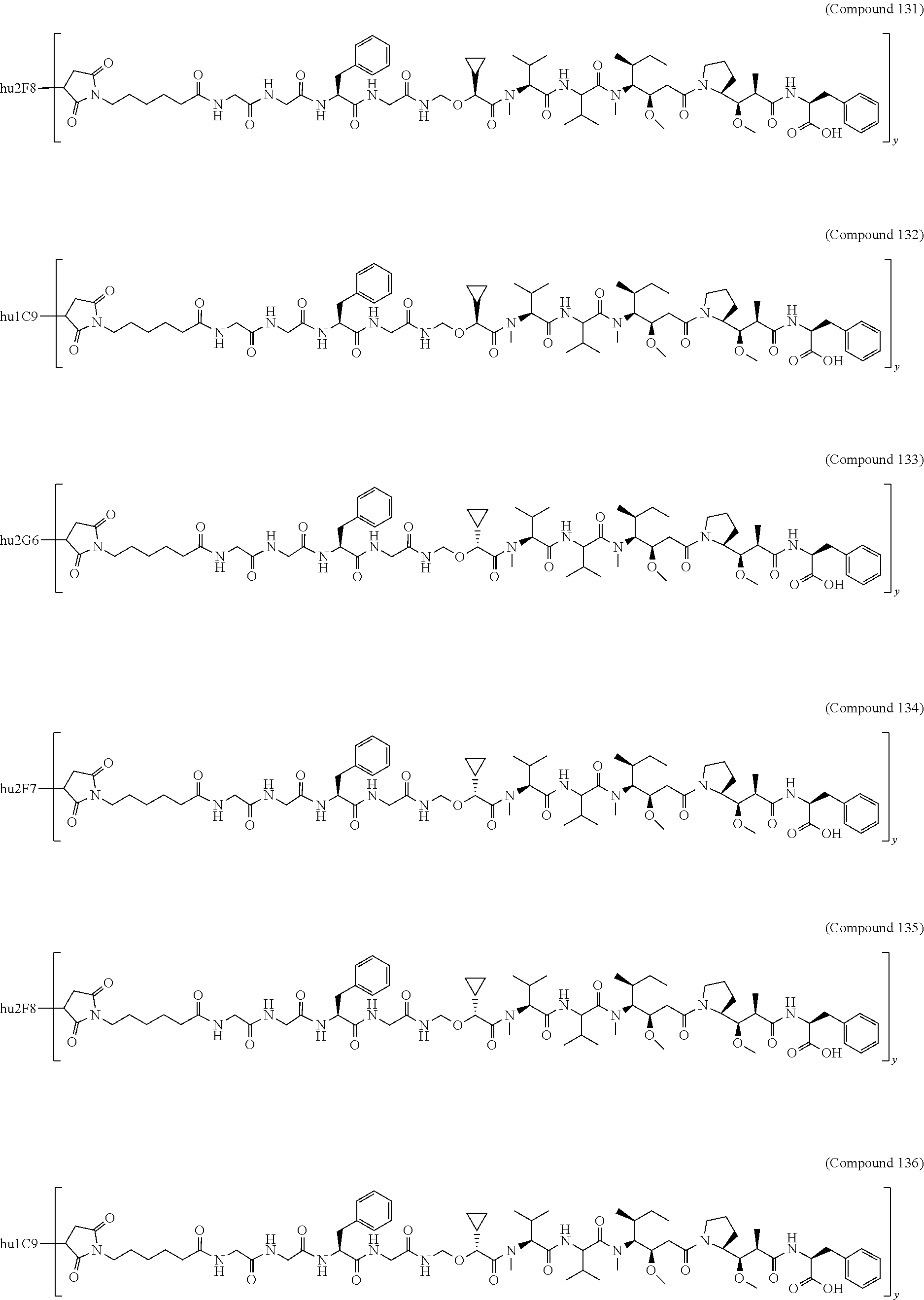

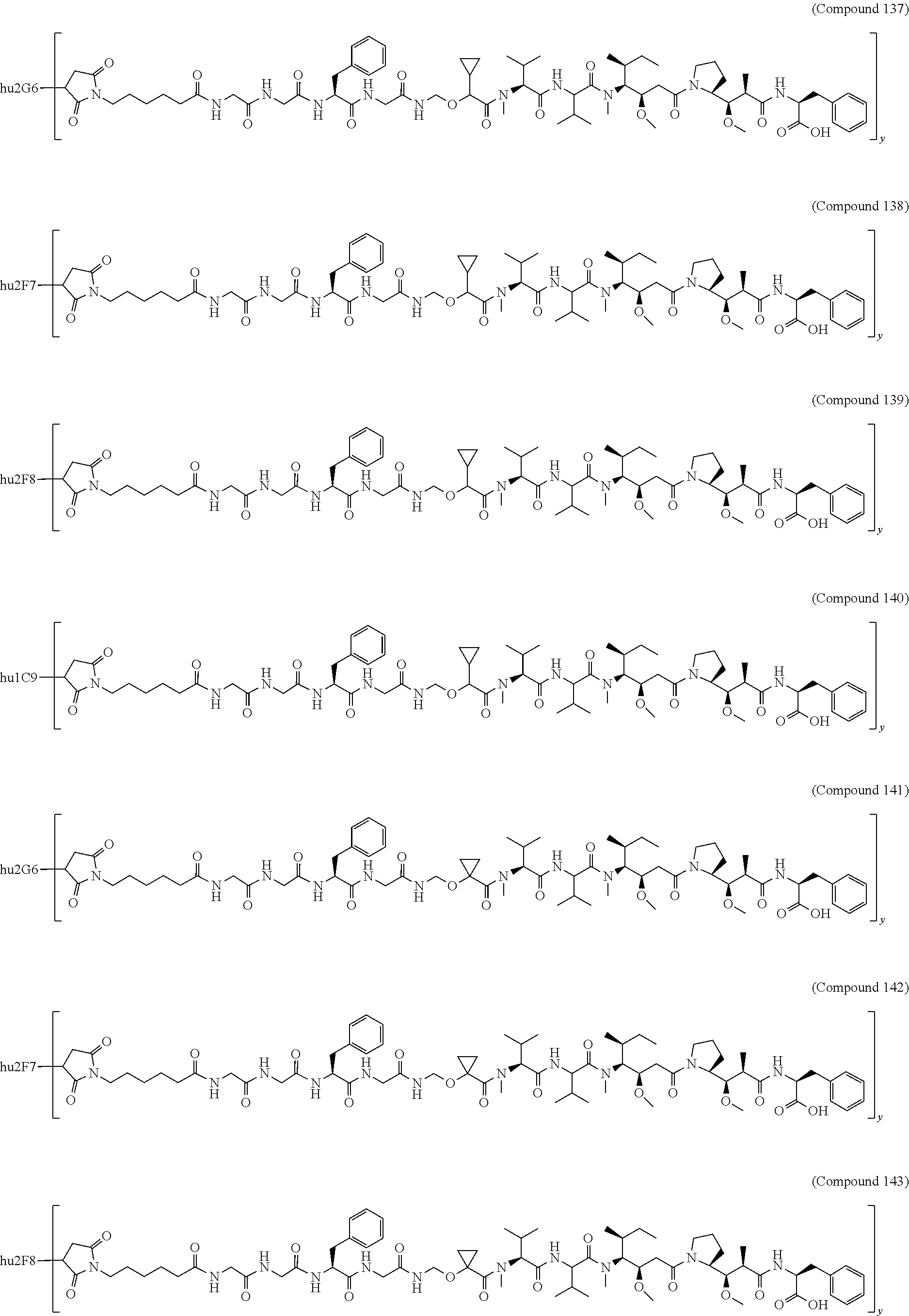

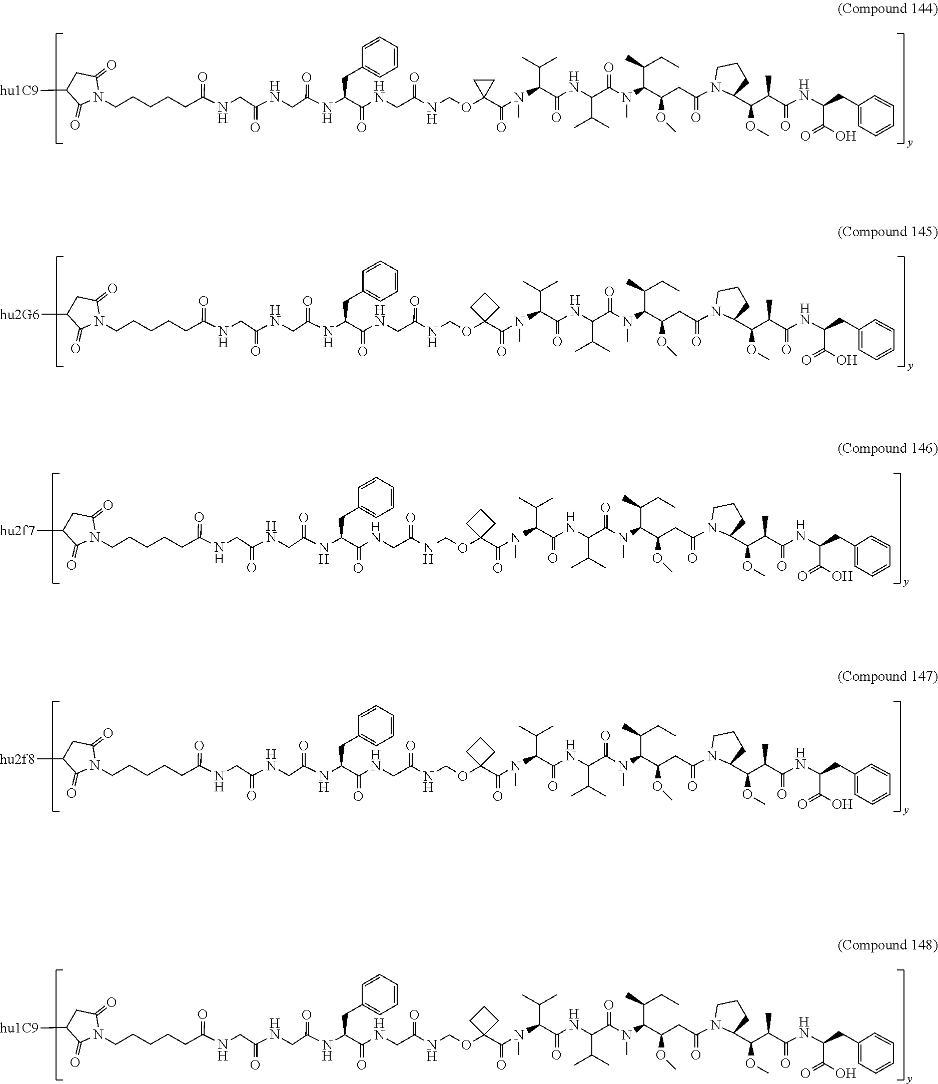

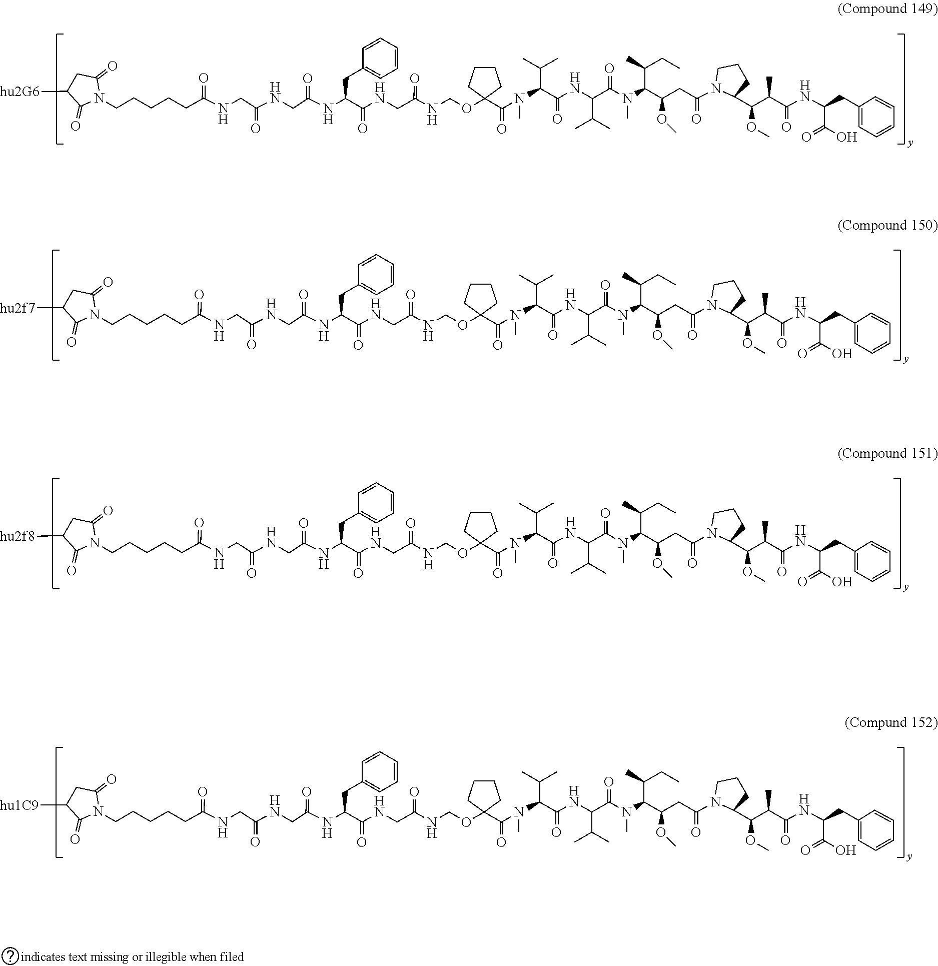

wherein, y is a number selected from 1 to 10, preferably a number selected from 2 to 10, further, a number selected from 6 to 10, more preferably a number of 7 to 9, and most preferably an integer of 7, 8, 9; or, y is a number selected from 2 to 10, preferably a number of 4 to 8, more preferably a number of 6 to 8, further preferably a number of 7 to 8, and most preferably 8.

In a preferred embodiment, the present invention relates to method for preparing the antibody-drug conjugate of general formula (IV) or a pharmaceutically acceptable salt or solvate thereof, which comprises the following steps:

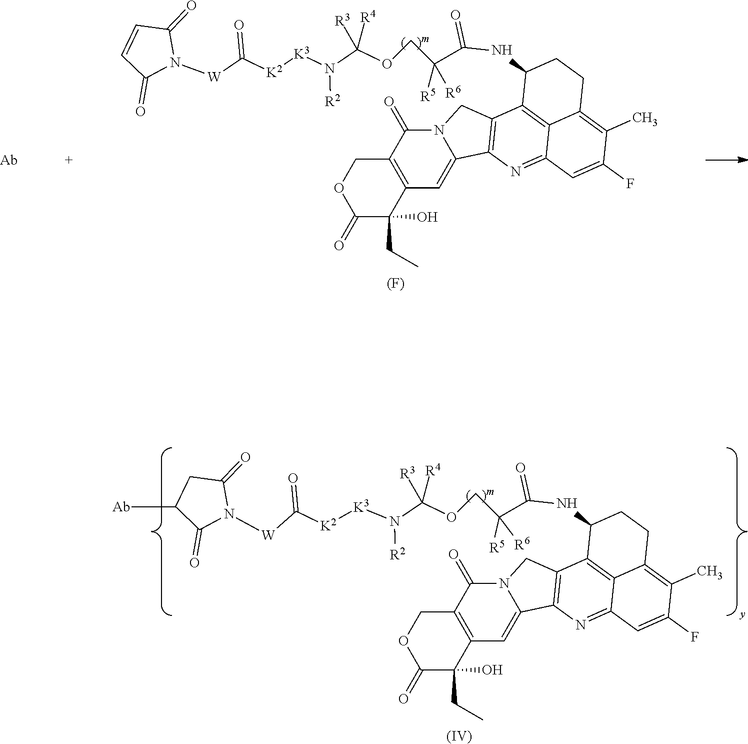

after Ab is reduced, it is subjected to coupling reaction with general formula (F) to obtain the compound of general formula (IV);

wherein:

Ab is an anti-B7H4 antibody or antigen-binding fragment thereof;

W, K², K³, R² to R⁶, m and y are as defined in general formula (IV).

In a preferred embodiment, the general formula (F) is a compound of general formula (F-1):

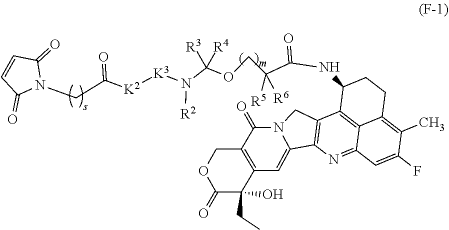

or a tautomer, mesomer, racemate, enantiomer, diastereomer or mixture form thereof, or a pharmaceutically acceptable salt thereof,

wherein

K², K³, R² to R⁶, s and m are as defined in the above -L₂-L₁-

In a preferred embodiment, the compound of general formula (F) or general formula (F-1) is selected from the group consisting of:

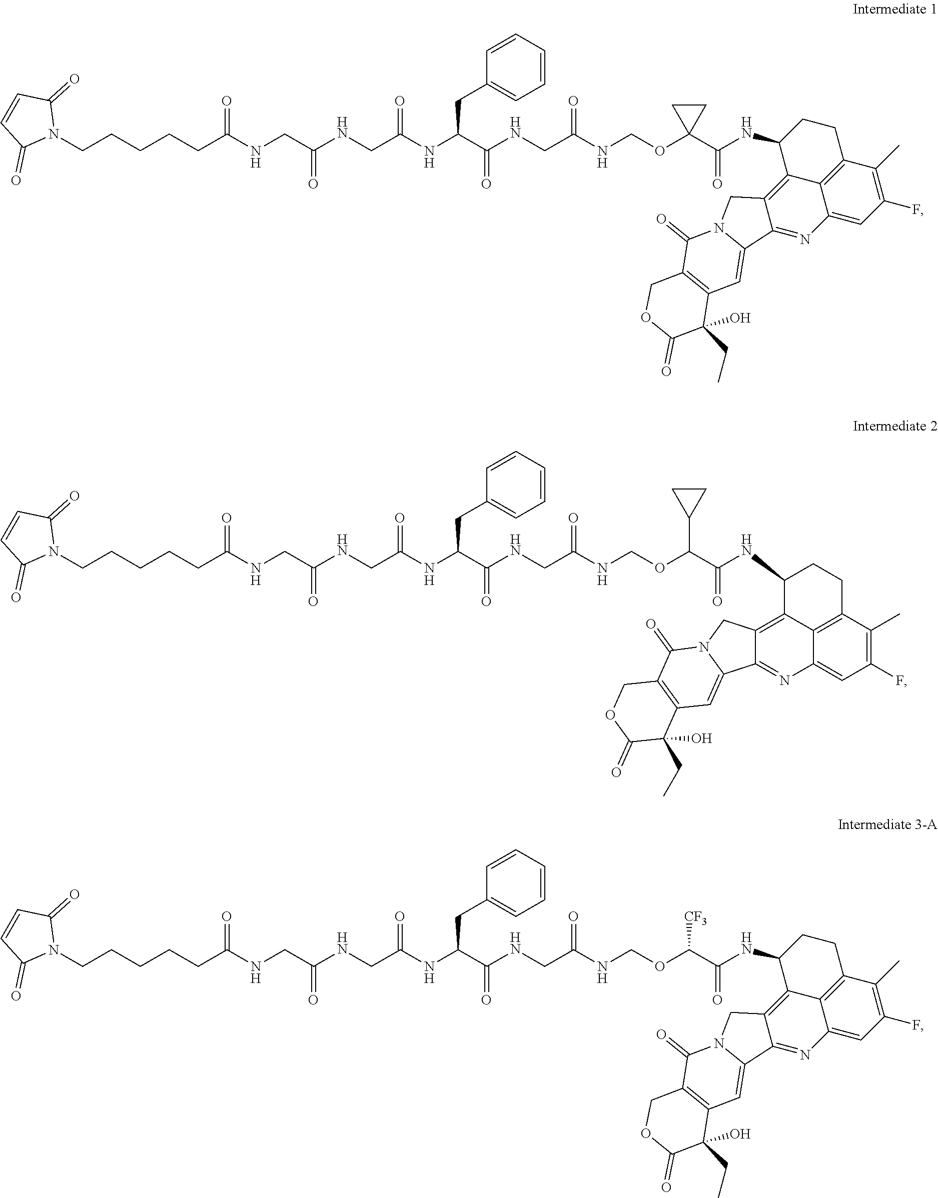

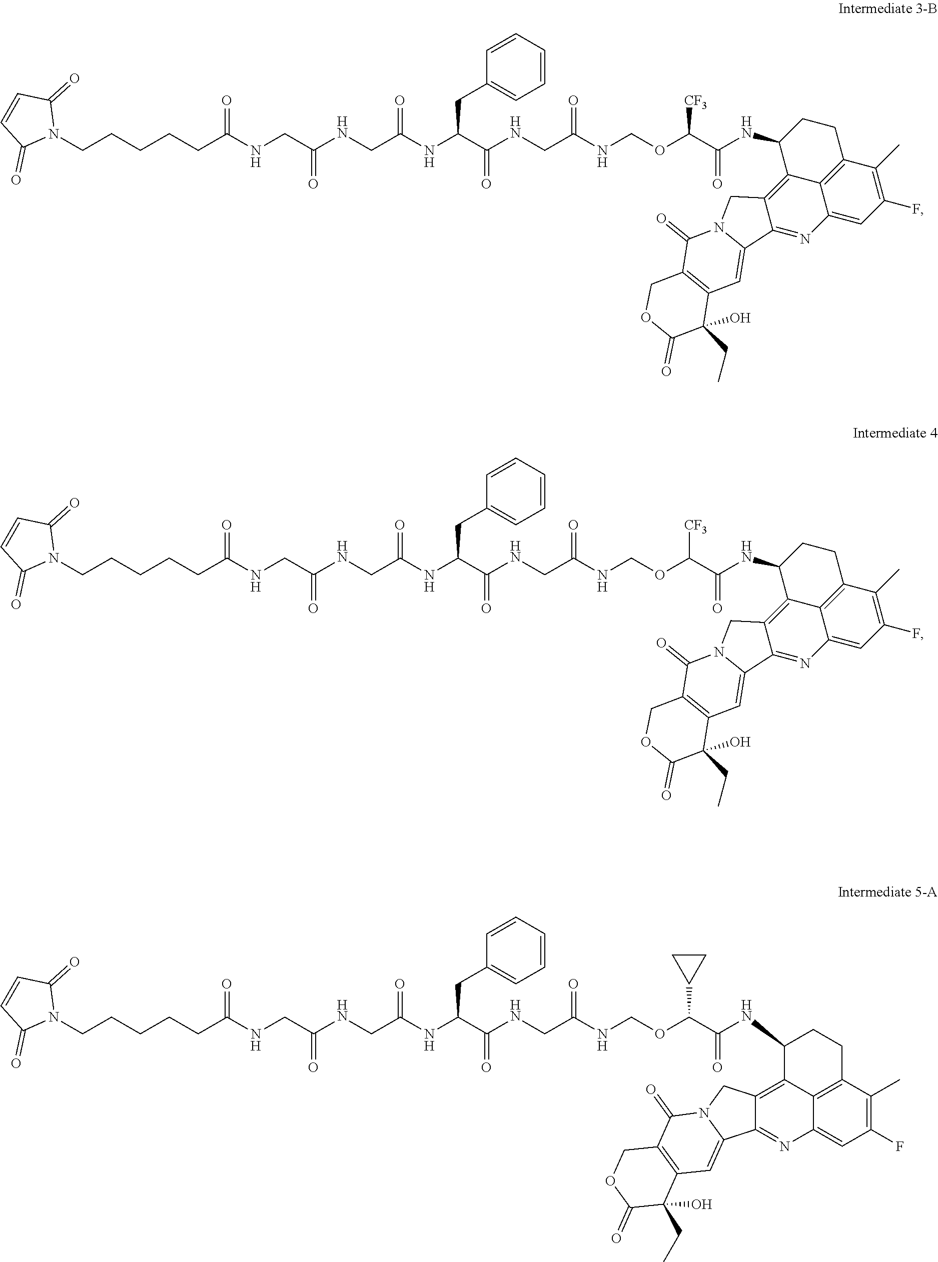

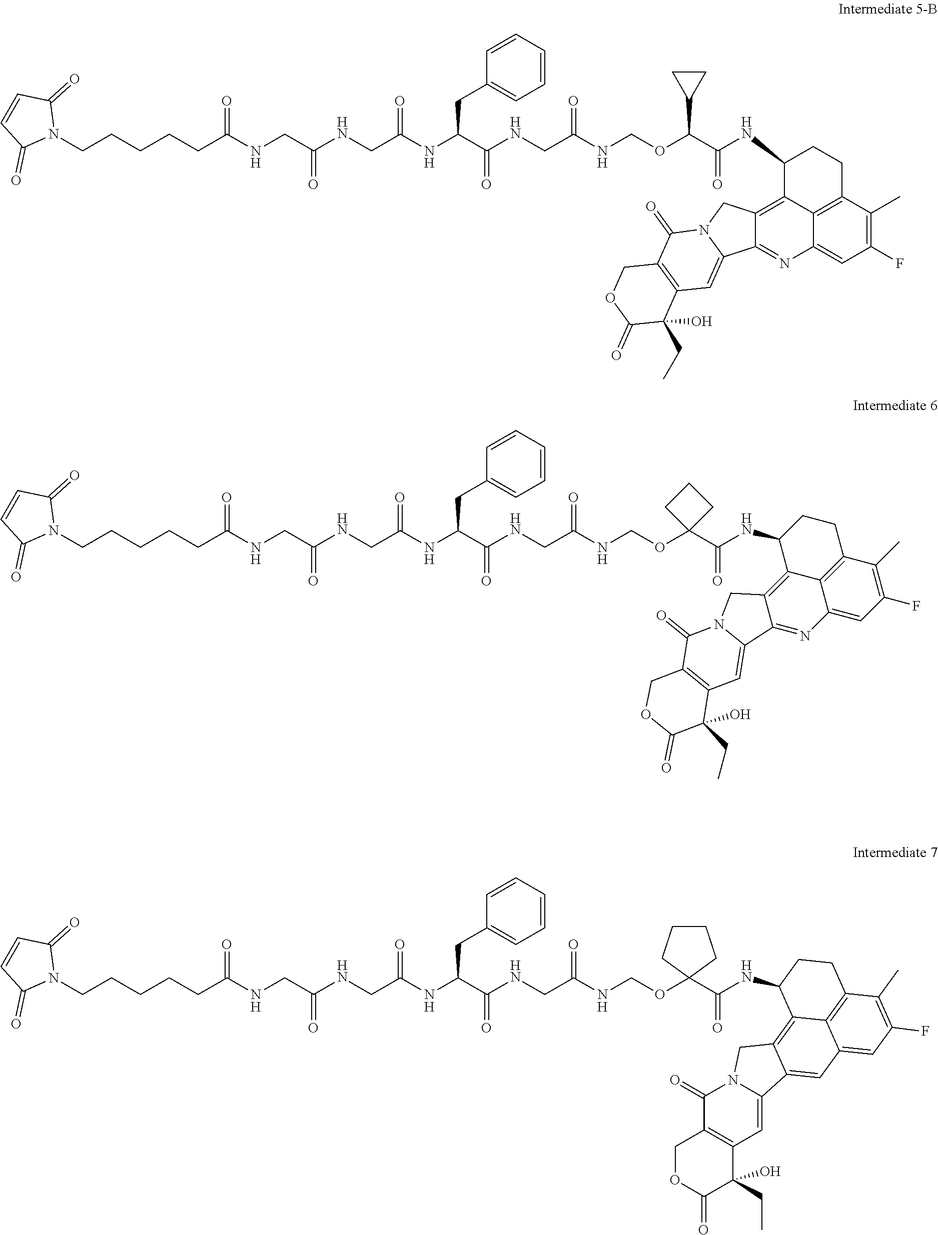

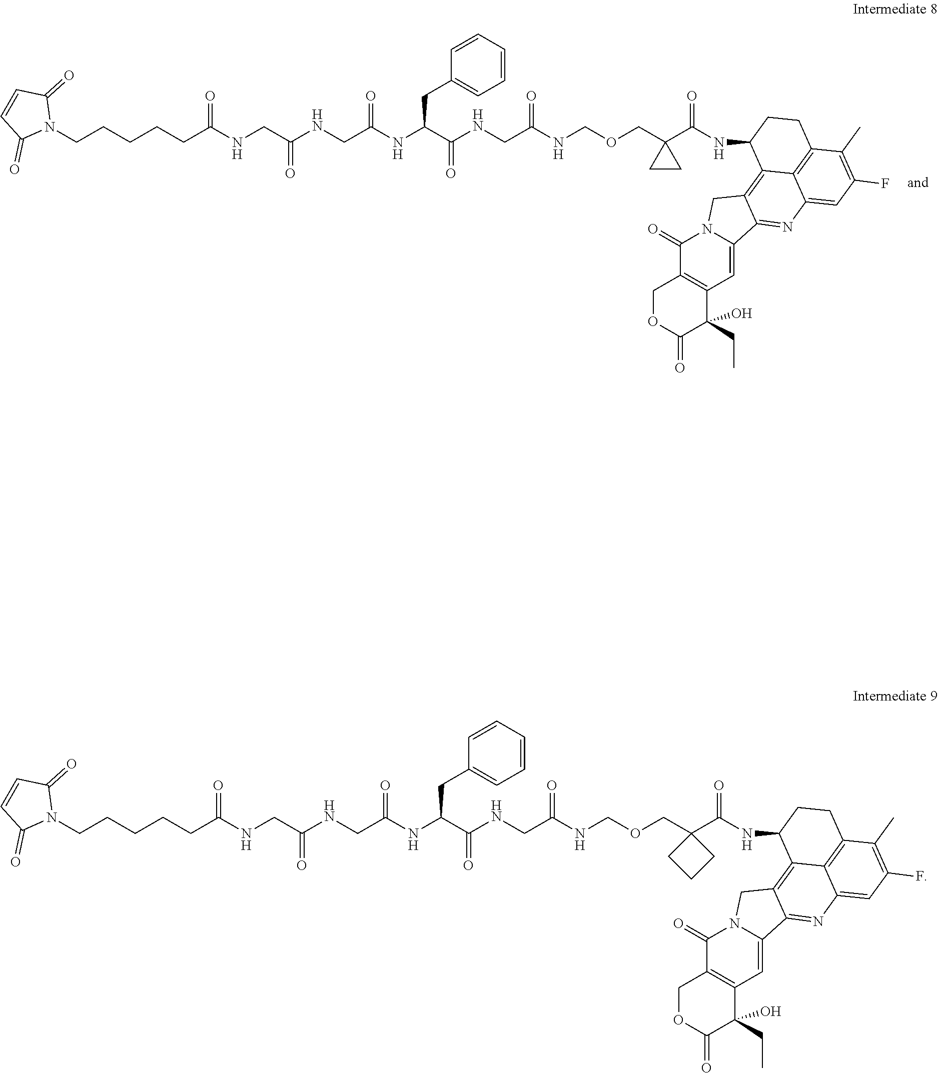

In a preferred embodiment, the present invention relates to a pharmaceutical composition, which comprises the antibody-drug conjugate according to any one of the present invention, or a pharmaceutically acceptable salt or solvate thereof, and one or more pharmaceutically acceptable excipients, diluents or carriers.

In a preferred embodiment, the present invention relates to use of the antibody-drug conjugate of general formula (A), or a pharmaceutically acceptable salt or solvate thereof, or a pharmaceutical composition thereof, in preparing a medicament for treating a disease related to human B7-H4, preferably a medicament for treating a cancer with high B7-H4 expression.

In a more preferred embodiment, the cancer is selected from the group consisting of astroblastoma of human brain, human pharyngeal cancer, adrenal tumor, AIDS-related cancer, alveolar soft-part sarcoma, astrocytoma, bladder cancer, bone cancer, brain and spinal cord cancer, metastatic brain tumor, breast cancer, carotid body tumor, cervical cancer, chondrosarcoma, chordoma, chromophobe cell carcinoma of kidney, clear cell carcinoma, colon cancer, colorectal cancer, connective tissue proliferative small round cell tumor, ependymoma, Ewing's sarcoma, extraosseous mucoid chondrosarcoma, fibrogenesis imperfecta ossium of bone, fibrous dysplasia of bone, gallbladder or cholangiocarcinoma, gastric cancer, gestational trophoblastic disease, germ cell tumor, head and neck cancer, hepatocellular carcinoma, islet cell tumor, Kaposi's sarcoma, kidney cancer; leukemia, liposarcoma/malignant lipomatous tumor, liver cancer, lymphoma, lung cancer, medulloblastoma, melanoma, meningioma, multiple endocrine neoplasia, multiple myeloma, myelodysplastic syndrome, neuroblastoma, neuroendocrine tumor, ovarian cancer, pancreatic cancer, papillary thyroid cancer, parathyroid adenoma, pediatric cancer, peripheral schwannoma, pheocytoma, pituitary tumor, prostate cancer, posterior uveal melanoma, renal metastatic cancer, rhabdoid tumor, rhabdomyosarcoma, sarcoma, skin cancer, soft tissue sarcoma, squamous cell carcinoma, synovial sarcoma, testicular cancer, thymic cancer, thyroid metastatic cancer and uterine cancer.

### DESCRIPTION OF THE DRAWINGS

FIG. 1: In vivo efficacy of antibody-drug conjugates: hu2G6-MC-MMAF and hu2F7-MC-MMAF showed inhibitory and killing effects on MX-1 xenograft tumors at the doses of 1.5 mg/kg and 3 mg/kg.

FIG. 2: HPLC spectra of hu2F7-MC-MMAF with drug loads of 2 and 4, respectively.

FIG. 3: The in vivo anti-tumor efficacy of hu2F7-MC-MMAF with drug loads of 2 and 4, respectively.

FIG. 4: The in vivo anti-tumor efficacy of hu2F7-MC-MMAF with drug loads of 2 and 4, respectively. The figure shows the tumors of mice in each group on day 21.

### DETAILED DESCRIPTION OF THE INVENTION

### 1 Terms

For easier understanding of the present invention, certain technical and scientific terms are specifically defined below. Unless otherwise clearly defined elsewhere in this document, all other technical and scientific terms used herein have the meanings commonly understood by those of ordinary skill in the art to which the present invention belongs.

The three-letter codes and one-letter codes of amino acids used in the present invention are as described in J. Biol. Chem, 243, p3558 (1968).

The term “antibody” described in the present invention refers to an immunoglobulin, which is a tetrapeptide chain structure consisting of two identical heavy chains and two identical light chains linked by interchain disulfide bonds. The composition and order of amino acids in the immunoglobulin heavy chain constant region(s) arc different, so their antigenicity is also different. According to this, immunoglobulins can be classified into five types, also known as isotypes of immunoglobulins, namely IgM, IgD, IgG, IgA and IgE, and their corresponding heavy chains are μ chain, δ chain, γ chain, α chain and ε chain, respectively. The same type of Ig can be classified into different subclasses according to the difference in their amino acid composition of the hinge region and the number and position of heavy chain disulfide bonds. For example, IgG can be classified into IgG1, IgG2, IgG3 and IgG4. The light chain is classified into κ chain or λ chain according to the difference in the constant region. Each of the five types of Ig can have a κ chain or a λ chain.

In the present invention, the antibody light chain variable region of the present invention can further comprise light chain constant region, which comprises human or murine κ, λ chains or variant thereof.

In the present invention, the antibody heavy chain variable region of the present invention can further comprise heavy chain constant region, which comprises human or murine IgG1, 2, 3, 4 or variant thereof.

The sequence of about 110 amino acids near the N-terminus of the antibody heavy and light chains varies greatly and is the variable region (V region); the remaining amino acid sequence near the C-terminus is relatively stable and is the constant region (C region). The variable region comprises 3 hypervariable regions (HVR) and 4 framework region(s) (FR) with relatively conservative sequences. The 3 hypervariable regions determine the specificity of the antibody, and are also known as complementarity determining regions (CDR). Each light chain variable region (VL) and heavy chain variable region (VH) consists of 3 CDR regions and 4 FR regions, arranged from the amino terminus to the carboxyl terminus in the order of FR1, CDR1, FR2, CDR2, FR3, CDR3, FR4. The 3 CDR regions of the light chain refer to LCDR1, LCDR2 and LCDR3; the 3 CDR regions of the heavy chain refer to HCDR1, HCDR2 and HCDR3. The number and position of the CDR amino acid residues of the VL region and VH region of the antibody or antigen-binding fragment of the present invention comply with the known Kabat or Chothia numbering criteria and Kabat or AbM definition criteria (http://bioinforg.uk/abs/).

The term “antigen presenting cell” or “APC” is a cell that presents foreign antigen complexed with MHC on its surface. T cells utilize T cell receptors (TCRs) to recognize such complexes. Examples of APCs include, but are not limited to, dendritic cells (DCs), peripheral blood mononuclear cells (PBMCs), monocytes. B lymphoblasts and monocyte-derived dendritic cells (DCs). The term “antigen presentation” refers to the process by which APCs capture antigens and enables them to be recognized by T cells, for example as a component of MHC-I/MHC-II conjugate.

The term “B7-H4” refers to a member of the human B7 protein family, also known as CD276, which is a type I transmembrane protein with four Ig-like extracellular domains. B7-H4 is one of the immune checkpoint proteins expressed on the surface of antigen-presenting cells or cancer cells, and has inhibitory effect on the functional activation of T cells. The term “B7-H4” includes any variant or isoform of B7-H4 that is naturally expressed by cells. The antibodies of the present invention can cross-react with B7-H4 derived from non-human species. As another option, the antibodies can also be specific for human B7-H4 and may not show cross-reactivity with other species. B7-H4 or any variant or isoform thereof can be isolated from cells or tissues naturally expressing the same, or produced by recombinant techniques using techniques commonly used in the art and those described herein. Preferably, the anti-B7-H4 antibody targets human B7-H4 with normal glycosylation pattern.

The term “recombinant human antibody” includes human antibodies prepared, expressed, created or isolated by recombinant methods, and the techniques and methods involved are well known in the art, for example (1) antibodies isolated from transgenic or transchromosomal animals (for example mice) expressing human immunoglobulin genes, or hybridomas prepared therefrom; (2) antibodies isolated from host cells transformed to express the antibodies, such as transfectionomas; (3) antibodies isolated from recombinant combinatorial human antibody libraries; as well as (4) antibodies prepared, expressed, created or isolated by splicing human immunoglobulin gene sequences to other DNA sequences and by other methods. Such recombinant human antibodies comprise variable regions and constant region(s), which utilize specific human germline immunoglobulin sequences encoded by germline genes, but also comprise subsequent rearrangements and mutations such as those occur during antibody maturation.

The term “murine antibody” in the present invention is a monoclonal antibody against human B7-H4 prepared according to the knowledge and skills in the art. During preparation, the test subject is injected with B7-H4 antigen, and then hybridomas expressing antibodies with the desired sequences or functional properties are isolated. In a preferred embodiment of the present invention, the murine B7-H4 antibody or antigen-binding fragment thereof can further comprise the light chain constant region of murine κ, λ chain or variant thereof, or further comprise the heavy chain constant region of murine IgG1, IgG2, IgG3 or IgG4 or variant thereof.

The term “human antibody” includes antibodies with variable and constant region(s) of human germline immunoglobulin sequences. The human antibodies of the present invention can comprise amino acid residues that are not encoded by human germline immunoglobulin sequences (such as mutations introduced by random or site-specific mutagenesis in vitro or by somatic mutations in vivo). However, the term “human antibody” does not include antibodies in which CDR sequences derived from the germline of another mammalian species (such as mouse) have been grafted onto human framework sequences (namely “humanized antibodies”).

The term “humanized antibody”, also known as CDR-grafted antibody, refers to the antibody produced by grafting murine CDR sequences into the framework of human antibody variable regions. It can overcome the strong immune response reactions induced by chimeric antibodies carrying a large amount of murine protein components. In order to avoid the decrease in activity caused by the decrease in immunogenicity, the human antibody variable regions can be subjected to minimal reverse mutation to maintain the activity.

The term “chimeric antibody” is an antibody formed by fusing the variable region of a murine antibody with the constant region of a human antibody, which can alleviate the immune response induced by murine antibody. Establishing a chimeric antibody requires first establishing a hybridoma that secrets murine specific monoclonal antibody, then cloning the variable region gene from the murine hybridoma cells, and then cloning the constant region gene of the human antibody as necessary, linking the murine variable region gene with the human constant region gene to form a chimeric gene, which is inserted into a human expression vector, and finally expressing the chimeric antibody molecule in a eukaryotic industrial system or a prokaryotic industrial system. The human antibody constant region(s) can be selected from the heavy chain constant region(s) of human IgG1, IgG2, IgG3 or IgG4 or variant thereof, preferably comprising human IgG2 or IgG4 heavy chain constant region(s), or using IgG1 with enhanced ADCC (antibody-dependent cell-mediated cytotoxicity) toxicity after amino acid mutations.

The term “antigen-binding fragment” refers to antigen-binding fragments and antibody analogs of an antibody, which usually comprise at least part of the antigen-binding region or variable regions (for example, one or more CDRs) of the parental antibody. The antibody fragment retains at least some of the binding specificity of the parental antibody. Generally, when the activity is represented on a mole basis, the antibody fragment retains at least 10% of the parental binding activity. Preferably, the antibody fragment retains at least 20%, 50%, 70%, 80%, 90%, 95% or 100% or more of the binding affinity of the parental antibody to the target. Examples of antigen-binding fragments include, but are not limited to: Fab, Fab′, F(ab′)₂, Fv fragment, linear antibody, single-chain antibody, nanobody; domain antibody and multispecific antibody. Engineered antibody variants are reviewed in Holliger and Hudson (2005) Nat. Biotechnol. 23: 1126-1136.

The “Fab fragment” consists of the CH1 and variable regions of one light chain and one heavy chain. The heavy chain of a Fab molecule cannot form disulfide bonds with another heavy chain molecule.

The “Fe” region comprises two heavy chain fragments comprising the CH1 and CH2 domains of the antibody. The two heavy chain fragments are held together by two or more disulfide bonds and through the hydrophobic interaction of the CH3 domain.

The “Fab′ fragment” comprises a light chain and a part of a heavy chain that comprises the VH domain, the CH1 domain and the region between the CH1 and CH2 domains, so that interchain disulfide bonds can be formed between the two heavy chains of two Fab′ fragments, so as to form the F(ab′)₂ molecule.

The “F(ab′)₂ fragment” comprises two light chains and two heavy chains comprising a part of the constant region between the CH1 and CH2 domains, thereby forming interchain disulfide bonds between the two heavy chains. Therefore, the F(ab′)₂ fragment consists of two Fab′ fragments held together by disulfide bonds between the two heavy chains.

The “Fv region” comprises variable regions from both the heavy chain and the light chain, but lacks the constant region(s).

The term “multispecific antibody” is used in its broadest sense, which encompasses antibodies with polyepitope specificity. These multispecific antibodies include, but are not limited to: antibodies comprising heavy chain variable region (VH) and light chain variable region (VL), wherein the VH-VL unit has multi-epitope specificity; antibodies with two or more VL and VH regions, each VH-VL unit binds to different targets or different epitopes of the same target; antibodies with two or more single variable regions, each single variable region binds to different targets or different epitopes of the same target; full-length antibodies, antibody fragments, diabodies, bispccific diabodies and triabodies, antibody fragments that are covalently or non-covalently linked together, and the like.

The term “single-chain antibody” is a single-chain recombinant protein formed by linking the heavy chain variable region (VH) and light chain variable region (VL) of an antibody through a linker peptide. It is the smallest antibody fragment with complete antigen binding site.

The term “domain antibody fragment” is an immunoglobulin fragment with immunological functions that comprises only the heavy chain variable region or the light chain variable region. In some cases, two or more VH regions are covalently linked to a peptide linker to form a bivalent domain antibody fragment. The two VH regions of the bivalent domain antibody fragment can target the same or different antigens.

The term “binding to B7-H4” in the present invention refers to the ability to interact with human B7-H4. The term “antigen-binding site” of the present invention refers to a three-dimensional site that is scattered on the antigen and is recognized by the antibody or antigen-binding fragment of the present invention.

The terms “specifically binds” and “selectively binds” used in the present invention refer to the binding of an antibody to an epitope on a predetermined antigen. Generally, when recombinant human B7-H4 is used as the analyte and an antibody is used as the ligand, when measured by surface plasmon resonance (SPR) technology in an instrument, the antibody binds to the predetermined antigen at an equilibrium dissociation constant (KD) of about less than 10⁻⁷ M or even less, and its binding affinity to the predetermined antigen is at least twice of its binding affinity to non-specific antigens (other than the predetermined antigen or closely related antigens, such as BSA and the like). The term “antibody that recognizes . . . antigen” can be used interchangeably with the term “antibody that specifically binds to . . . ” herein.

The term “cross-reactivity” refers to the ability of the antibodies of the present invention to bind to B7-H4 from different species. For example, the antibody of the present invention that binds to human B7-H4 can also bind to B7-H4 of another species. Cross-reactivity is measured by detecting specific reactivity with purified antigens in binding assays (for example SPR and ELISA), or by binding or functional interaction with cells that physiologically express B7-H4. Methods of determining cross-reactivity include standard binding assays as described herein, for example surface plasmon resonance (SPR) analysis or flow cytometry.

The terms “inhibition” or “blocking” can be used interchangeably and encompass both partial and complete inhibition/blocking. The inhibition/blocking of a ligand preferably reduces or alters the normal level or type of activity that present when ligand binding occurs without inhibition or blocking. Inhibition and blocking are also intended to include any measurable reduction in binding affinity of the ligand when being in contact with anti-B7-H4 antibody, compared to the binding affinity of the ligand not being in contact with anti-B7-H4 antibody.

The term “inhibition of growth” (for example when referring to cells) is intended to include any measurable reduction in cell growth.

The terms “induced immune response” and “enhanced immune response” can be used interchangeably and refer to immune response to the stimulation by a specific antigen (i.e. passive or adaptive). The term “induce” used in expression “inducing CDC or ADCC” refers to stimulating a specific direct cell killing mechanism.

The “ADCC” in the present invention, i.e. antibody-dependent cell-mediated cytotoxicity, means that cells expressing Fc receptors directly kill the target cells coated with antibodies by recognizing the Fc segment of the antibodies. The ADCC effect function of antibodies can be enhanced, reduced or eliminated by modifying the Fc segment of IgG. The modification refers to mutations in the heavy chain constant region of an antibody.

The methods for producing and purifying antibodies and antigen-binding fragments are well-known and can be found in the prior art, such as Antibodies: A Laboratory Manual, Cold Spring Harbor, chapters 5-8 and 15. For example, mice can be immunized with human B7-H4 or fragment thereof, and the obtained antibodies can be renatured and purified, and amino acid sequencing can be performed by using conventional methods. Antigen-binding fragments can also be prepared by using conventional methods. The antibody or antigen-binding fragment of the present invention is genetically engineered to introduce one or more human FR regions onto the non-human CDR regions. The human FR germline sequences can be obtained from the ImmunoGeneTics (IMGT) website http://imgt.cines.fr, or from The Immunoglobulin FactsBook, 2001ISBN012441351.

The engineered antibodies or antigen-binding fragments of the present invention can be prepared and purified by conventional methods. The eDNA sequences of the corresponding antibodies can be cloned and recombined into GS expression vectors. The recombinant immunoglobulin expression vectors can stably transfect CHO cells. As a more recommended prior art, mammalian expression systems can lead to glycosylation of antibodies, especially at the highly conserved N-terminus of the Fc region. Stable clones are obtained by expressing antibodies that specifically bind to human antigens. Positive clones are expanded in serum-free medium of bioreactors to produce antibodies. The culture medium into which the antibodies are secreted can be purified and collected by conventional techniques. The antibodies can be filtered and concentrated by conventional methods. Soluble mixtures and multimers can also be removed by conventional methods, for example molecular sieves and ion exchange. The resulting product needs to be frozen immediately, such as at −70° C., or lyophilized.

The antibody of the present invention refers to monoclonal antibody. The monoclonal antibody (mAb) described in the present invention refers to an antibody obtained from a single cloned cell strain, the cell strain is not limited to a eukaryotic, prokaryotic or phage cloned cell strain. Monoclonal antibodies or antigen-binding fragments can be obtained by recombination using, for example, hybridoma technology, recombination technology, phage display technology, synthesis technology (such as CDR-grafting) or other existing technologies.

“administering” and “treating”, when applied to animals, humans, experimental subjects, cells, tissues, organs or biological fluids, refer to contacting the exogenous medicament, therapeutic agent, diagnostic agent or composition with the animals, humans, subjects, cells, tissues, organs or biological fluids. “administering” and “treating” can refer to for example treatment, pharmacokinetics, diagnosis, research and experimental methods. Treating cells includes contacting reagents with the cells, and contacting reagents with fluids, wherein the fluids are in contact with the cells. “administering” and “treating” also mean treating for example cells with reagents, diagnostics, binding compositions or with another type of cells in vitro and ex vivo. “Treating”, when applied to human, veterinary or research subjects, refers to therapeutic treatment, preventive or prophylactic measures, research and diagnostic applications.

“Treatment” refers to administering an internal or external therapeutic agent, for example a composition comprising any one of the binding compounds of the present invention, to a patient with one or more disease symptoms on which the therapeutic agent is known to have therapeutic effect. Generally, the therapeutic agent is given in an amount effective to alleviate one or more disease symptoms in the treated subject or population, either to induce the regression of such symptoms or to inhibit the development of such symptoms to any clinically measurable extent. The amount of therapeutic agent that is effective to alleviate any specific disease symptom (also referred to as a “therapeutically effective amount”) can vary according to a variety of factors, for example the patient's disease state, age and body weight, and the ability of the drug to produce the desired therapeutic effect in the patient. Whether the disease symptoms have been alleviated can be evaluated by any clinical testing methods commonly used by doctors or other health care professionals for evaluating the severity or progression of the symptoms. Although the embodiments of the present invention (for example treatment methods or products) may be ineffective in alleviating each disease symptom of interest, but they should reduce the disease symptom of interest in a statistically significant number of patients, as determined by any statistical testing methods known in the art, such as Student t-test, chi-square test, Mann and Whitney's U test, Kruskal-Wallis test (H test), Jonckheere-Terpstra test and Wilcoxon test.

The term “essentially consisting of” or variant thereof used throughout the specification and claims means to comprise all the elements or element groups described, and optionally comprise other elements similar or different in nature to the elements described, which does not significantly change the basic or new properties of the given dosing regimen, method, or composition. As a non-limiting example, the binding compound essentially consisting of the mentioned amino acid sequence can also comprise one or more amino acids, which do not significantly affect the properties of the binding compound.

The term “naturally occurring” applied to a certain object in the present invention refers to the fact that the object can be found in nature. For example, a polypeptide sequence or pol nucleotide sequence is deemed as naturally occurring, if it exists in organisms (including virus) and can be isolated from natural sources, and has not been intentionally modified artificially in the laboratory.

The “effective amount” includes an amount sufficient to ameliorate or prevent a symptom or condition of a medical condition. The effective amount also refers to an amount sufficient to allow or facilitate diagnosis. The effective amount for a particular patient or veterinary subject can vary depending on the following factors: such as the condition to be treated, the general health condition of the patient, the method, route and dose of drug administration, and the severity of side effects. The effective amount can be the maximum dose or dosing regimen that avoids significant side effects or toxic effects.

“Exogenous” refers to substances produced outside organisms, cells or human bodies according to backgrounds. “Endogenous” refers to substances produced inside cells, organisms or human bodies according to backgrounds.

“Homology” refers to the sequence similarity between two polynucleotide sequences or between two polypeptides. When the positions in the two sequences aligned arc occupied by the same base or amino acid monomer subunit, for example if each position of two DNA molecules is occupied by adenine, then the molecules are deemed as homologous at that position. The homology percentage between two sequences is a function of the number of matched or homologous positions shared by the two sequences divided by the number of positions aligned×100%. For example, in the optimal sequence alignment, if 6 out of 10 positions in the two sequences are matched or homologous, then the two sequences are 60% homologous. Generally, the alignment is made when two sequences are aligned to obtain the maximum homology percentage.

The expressions “cell”. “cell line” and “cell culture” used herein can be used interchangeably, and all such names include progeny thereof. Therefore, the words “transformant” and “transformed cell” include primary test cells and cultures derived therefrom, regardless of the number of passages. It should also be understood that due to intentional or unintentional mutations, all offspring cannot be exactly the same in terms of DNA content. Mutant progeny with the same function or biological activity as those screened in the original transformed cells is included. It can be clearly understood from the context, when different names are referred to.

“Optional” or “optionally” means that: an event or circumstance that follows the wording “optional” or “optionally” could occur, but does not have to occur, and the description includes occasions when the event or circumstance occurs or does not occur. For example. “optionally comprising 1 to 3 antibody heavy chain variable regions” means that the antibody heavy chain variable regions of particular sequences can but does not have to be present.

“Pharmaceutical composition” means a mixture containing one or more of the compounds described herein, or a physiologically/pharmaceutically acceptable salt or a prodrug thereof, and other chemical components, as well as other components such as physiological/pharmaceutically acceptable carriers and excipients. The object of the pharmaceutical composition is to promote drug administration to organisms, to facilitate the absorption of the active ingredient and thereby exerting the biological activity. The preparation of conventional pharmaceutical compositions is described in the Chinese Pharmacopoeia.

“Pharmaceutically acceptable salt” refers to a salt of the antibody-drug conjugate of the present invention, which is safe and effective for use in mammals and has the desired biological activity. The antibody-drug conjugate of the present invention comprises at least one amino group, thus it can form a salt with an acid. Non-limiting examples of pharmaceutically acceptable salts include: hydrochloride, hydrobromide, hydroiodide, sulfate, hydrogen sulfate, citrate, acetate, succinate, ascorbate, oxalate, nitrate, sorbate, hydrogen phosphate, dihydrogen phosphate, salicylate, hydrogen citrate, tartrate, maleate, fumarate, formate, benzoate, methanesulfonate, ethanesulfonate, benzenesulfonate and p-toluenesulfonate.

The “solvate” refers to a pharmaceutically acceptable solvate formed by the antibody-drug conjugate compound of the present invention and one or more solvent molecules. Non-limiting examples of solvent molecules include: water, ethanol, acetonitrile, isopropanol and ethyl acetate.

The “cytotoxic drug”, when used in the present invention, refers to a substance that inhibits the function of cells and/or causes cell death or destruction.

The “tubulin inhibitor” refers to a class of compounds that interfere with the process of cell mitosis by inhibiting the polymerization of tubulin or promoting the aggregation of tubulin, thereby exerting an anti-tumor effect. Non-limiting examples thereof include: maytansinoids, calicheamicin, taxanes, vincristine, colchicine, dolastatin/auristatin/monomethyl auristatin E (MMAE)/monomethyl auristatin F (MMAF).

“Linker” refers to a chemical moiety by which an antibody is covalently attached to a covalent bond or atomic chain of a drug. Non-limiting examples of linkers include: arylene, heteroarylene, PEG, polymethyleneoxy, succinate, succinamide, diglycolate, malonate and caproamide.

“Drug load” (DAR) is represented by y, which is the average number of cytotoxic drugs per antibody in formula (A). The range of drug load in the present invention can be 1-20 cytotoxic drugs (D) per antibody. The antibody-drug conjugate of general formula (A) is a collection of antibodies conjugated to a certain range (1-20) of cytotoxic drugs. The drug load (DAR) in an antibody-drug conjugate from coupling reaction can be characterized by conventional means, for example mass spectrometry, HPLC and ELISA. By these means, the quantitative distribution of the antibody-drug conjugate on the y value can be determined.

### 2. Abbreviations

MC=6-maleimidohexanoyl

VC=valine-citrulline

PAB=p-aminobenzyloxycarbonyl

MMAE=monomethyl auristatin E (MW 718)

MMAF=a variant of monomethyl auristatin E, which has phenylalanine at the C-terminus of the molecule (MW 731.5)

The examples are incorporated below for further description of the present invention, but these examples do not limit the scope of the present invention. The experimental methods with unspecified conditions in the examples of the present invention generally follow conventional conditions, such as Antibodies: A Laboratory Manual and Molecular Cloning: A Laboratory Manual. Cold Spring Harbor; or according to the conditions recommended by the raw material or commodity manufacturer. The reagents with unspecified sources are conventional reagents purchased on the market.

### Example 1: Antigen Preparation and Stable Cell Line Construction

The sequence encoding his-tagged human B7-H4 (huB7-H4-His) and the sequence encoding Fe-tagged human B7-H4 (huB7-H4-Fc) were synthesized by Integrated DNA Technology (IDT) of CRO (the template sequences of the above B7-H4 recombinant proteins were all designed by the present invention) and respectively cloned into pTT5 vectors (Biovector). After the recombinant B7-H4 proteins were expressed in 293T cells, they were purified by the following experimental methods, and the purified proteins could be used in the following experiments of the examples.

Sequence of huB7-H4-His:
SEQ ID NO: 1
MASLGQILFWSIISIIIILAGAIALIIGFGISGRHSITVTTVASAGNIG
EDGILSCTFEPDIKLSDIVIQWLKEGVLGLVHEFKEGKDELSEQDEMFR
GRTAVFADQVIVGNASLRLKNVQLTDAGTYKCYIITSKGKGNANLEYKT
GAFSMPEVNVDYNASSETLRCEAPRWFPQPTVVWASQVDQGANFSEVSN
TSFELNSENVTMKVVSVLYNVTINNTYSCMIENDIAKATGDIKVTESEI
KRRSHLQLLNSKADYKDDDDKGSHHHHHHHH
Sequence of huB7-H4-Fc:
SEQ ID NO: 2
FGISGRHSITVTTVASAGNIGEDGILSCTFEPDIKLSDIVIQWLKEGVL
GLVHEFKEGKDELSEQDEMFRGRTAVFADQVIVGNASLRLKNVQLTDAG
TYKCYIITSKGKGNANLEYKTGAFSMPEVNVDYNASSETLRCEAPRWFP
QPTVVWASQVDQGANFSEVSNTSFELNSENVTMKVVSVLYNVTINNTYS
CMIENDIAKATGDIKVTESEIKRRSHLQLLNSKAGSGGGGDKTHTCPPC
PAPELLGGPSVFLFPPKPKDTLMISRTPEVTCVVVDVSHEDPEVKFNWY
VDGVEVHNAKTKPREEQYNSTYRVVSVLIVLHQDWLNGKEYKCKVSNKA
LPAPIEKTISKAKGQPREPQVYTLPPSRDELTKNQVSLTCLVKGFYPSD
IAVEWESNGQPENNYKTTPPVLDSDGSFFLYSKLTVDKSRWQQGNVFSC
SVMHEALHNHYTQKSLSLSPGK

Purification Steps of huB7-H4-his:

The HEK293 cell expression supernatant sample was centrifuged at high speed to remove impurities. The buffer was replaced against PBS and added with imidazole to a final concentration of 5 mM. The nickel column was equilibrated with PBS solution containing 5 mM imidazole and washed with 2-5 times the column volume. The supernatant sample obtained after replacement was loaded onto the column. The column was rinsed with PBS solution containing 5 mM imidazole until the A280 reading dropped to baseline. Then the chromatography column was rinsed with PBS+10 mM imidazole to remove non-specifically bound protein impurities, and the effluent was collected. The target protein was eluted with PBS solution containing 300 mM imidazole, and the elution peak was collected. The collected eluate was further purified by ion exchange (SP column). Preparing A solution: 0.01 M PB, pH 8.0. Preparing B solution: A solution+1 M NaCl. First, the PBS solution of imidazole with eluted target protein was replaced against solution A, the SP column was equilibrated by using solution A. and the sample was loaded. The concentration gradient of solution B was 0-1(10%. The sample was eluted with 10 times the column volume, and each elution peak was collected. The obtained protein was identified as correct by electrophoresis, peptide map and liquid chromatography-mass spectrometry (LC-MS), and then aliquoted for use.

Purification Steps of huB7-H4-Fc:

The HEK293 cell expression supernatant sample was centrifuged at high speed to remove impurities. The buffer was replaced against PBS. The Protein A affinity column was equilibrated with 10 mM PBS buffer and was washed 2-5 times the column volume. The supernatant sample obtained after replacement was loaded onto the column. The column was washed with buffer 25 times the column volume until the A280 reading dropped to baseline. The target protein was eluted with 0.8% acetate buffer of pH 3.5, and the elution peaks were collected. After aliquoting, 1 M Tris-Cl pH 8.0 buffer was immediately added for neutralization. Then the solution was replaced against PBS pH 6.9 by using Millipore's Amico-15 filter column. The obtained protein was identified by electrophoresis, peptide map and liquid chromatography-mass spectrometry (LC-MS), and then aliquoted for use.

Construction of Stable CHO—S Cell Pool:

The full-length sequences encoding human or cynomolgus monkey B7-H4 protein (huB7-H4 or cyB7-H4) were synthesized by Integrated DNA Technology (IDT) (the template sequences of the above B7-H4 recombinant proteins were all designed by the present invention), and respectively cloned into the modified pcDNA3.1 vector, pcDNA3.1/puro (Invitrogen #V79020). CHO—S(ATCC) cells were cultured in CD-CHO medium (Life Technologies, #10743029) to 0.5×10⁶ cells/ml. 10 μg of vector encoding huB7-H4 or cyB7-H4 gene was mixed with 50 μl LF-LTX (Life Technologies, #A 12621) in 1 ml Opti-MEM medium (Life Technologies, #31985088). The mixture was incubated at room temperature for 20 minutes and added into the culture medium of CHO cells. The cells were placed in a carbon dioxide incubator for culture. After 24 hours, the culture medium was replaced with new medium and added with 10 μg/ml puromycin. After that, the culture medium was replaced with new medium every 2-3 days, and a stable CHO—S cell pool was obtained after 10-12 days of selection.

### Example 2: Obtaining Mouse Hybridoma and Antibody Sequences

A total of 5 Balb/c and 5 A/J female 10-weeks-old mice were immunized with the human antigen huB7-H4-Fc. Sigma Complete Freund's Adjuvant (CFA) and Sigma Incomplete Freund's Adjuvant (WA) were used. The immunogen and the immune adjuvant were fully mixed and emulsified at a ratio of 1:1 to make a stable “water-in-oil” liquid. The injection dose was 25 μg/200 μL/mouse.

Day 01: First immunization, complete Freund's adjuvant.

Day 21: Second immunization, incomplete Freund's adjuvant.

Day 35: Third immunization, incomplete Freund's adjuvant.

Day 42: Blood sampling and serum titer test (blood after 3 immunizations).

Day 49: Fourth immunization, incomplete Freund's adjuvant.

Day 56: Blood sampling and serum titer test (blood after 4 immunizations).

Indirect ELISA method was used to detect the affinity of antibody or serum: huB7-H4-His protein was diluted to a concentration of 1 μg/ml with PBS pH7.4, and added at 100 μl/well into a 96-well high-affinity ELISA plate, and refrigerated at 4° C. for incubation overnight (16-20 hours). The plate was washed 4 times with PBST (pH 7.4 PBS containing 0.05% Tween-20). 3% bovine serum albumin (BSA) blocking solution diluted with PBST was added at 150 μl/well and incubated at room temperature for 1 hour for blocking. After completion of the blocking, the blocking solution was discarded, and the plate was washed 4 times with PBST buffer. The antibody or serum to be tested was diluted with PBST containing 3% BSA, to obtain a gradient of 10 doses (10-fold dilution) starting from 1 μM, and added into the microtiter plate at 100 μl/well and incubated at room temperature for 1 hour. After completion of the incubation, the plate was washed 4 times with PBST, HRP-labeled goat anti-human secondary antibody (Abeam, cat #ab97225) diluted with PBST containing 3% BSA was added at 100 μl/well, and incubated for 1 hour at room temperature. The plate was washed 4 times with PBST, then TMB chromogenic substrate (Cell Signaling Technology, cat #70045) was added at 100 μl/well and incubated at room temperature in dark for 1 minute. The stop solution (Cell Signaling Technology, cat #70025) was added at 100 μl/well to terminate the reaction. The absorbance value was read at 450 nm with a microplate reader (BioTek, model Synergy H1). The data were analyzed to obtain the binding affinity of the antibody or serum to the human B7-H4 antigen.

The scrum titer and the ability of the immunized mouse serum to bind to cell surface antigens were evaluated by using indirect ELISA method. The cell fusion was performed according to the detection results of titer (greater than 100,000 times of dilution). The immunized mice with high serum titer, affinity and FACS binding were selected for one final immunization and then sacrificed. The spleen cells and SP2/0 myeloma cells were fused and plated to obtain hybridomas. The target hybridomas were screened by indirect ELISA and monoclonal cell strains were established by limiting-dilution method. Among the obtained positive antibody strains, the hybridoma strains expressing non-specific binding antibodies were further excluded by comparing CHO—S cells stably expressing huB7-H4 and cyB7-H4, with blank CHO—S cells. The target hybridomas were screened by using methods similar to that of the in vitro cell binding experiment in Example 5, and established as monoclonal cell strains by limiting-dilution method. Hybridoma cells at logarithmic growth phase were collected. RNA was extracted with Trizol (Invitrogen, 15596-018) and subjected to reverse transcription (PrimcScript™ Reverse Transcriptase, Takara #2680A). The eDNA obtained by reverse transcription was amplified by PCR with a mouse Ig-primer set (Novagen, TB326 Rev.B 0503) and sequenced. Finally, the sequences of 4 strains of murine antibodies were obtained for humanization and construction of antibody-drug conjugates.

The heavy chain and light chain variable region sequences of murine monoclonal antibody 2G6 are as follows:

2G6 HCVR
SEQ ID NO: 46
EVQLVESGGGLVKPGGSLKLSCAASGFTFSRYGMSWVRQTPEKRLEWVA
GINGGGSYTYYLDTVKGRFTISRDNSRNTLYLQMSSLRSEDTAMYYCVS
QGSNYYFDYWGQGTTLTVSS
2G6 LCVR
SEQ ID NO: 47
DIRMTQSPSSMSVSLGDTVSITCHASQGISSNIGWLQQKPGKSFKALIY
HGTNLEDGVPSRFSGSGSGADYSLTISSLESEDFADYYCVQYAQFPYTF
GGGTKLEIK

The heavy chain and light chain variable regions of murine monoclonal antibody 2G6 comprise the following CDR sequences:

| Name | Sequence | No. |
|---|---|---|
| HCDR1 | GFTFSRYGMS | SEQ ID NO: 3 |
| HCDR2 | GINGGGSYTYYLDTVKG | SEQ ID NO: 4 |
| HCDR3 | QGSNYYFDY | SEQ ID NO: 5 |
| LCDR1 | HASQGISSNIG | SEQ ID NO: 6 |
| LCDR2 | HGTNLED | SEQ ID NO: 7 |
| LCDR3 | VQYAQFPYT | SEQ ID NO: 8 |

The sequences of the heavy chain and light chain variable regions of murine monoclonal antibody 2F7 are as follows:

2F7 HCVR
SEQ ID NO: 48
EVQLVESGGGLVQPGGSLKLSCAASGFTFSNYYMSWVRQTPEKRLEWVA
YVSSGGGSTYYSDSVKGRFTISRDNAKNTLYLQMSSLKPEDTAMYYCTR
ESYSQGNYFDYWGQGTTLTVSS
2F7 LCVR
SEQ ID NO: 49
DIVMTQSPATLSVTPGDRVSLSCRASQSISDYLHWYQQKSHESPRLLIK
FASQSISGIPSRFSGSGSGSDFTLSINSVEPEDVGVYYCQNGHSFSLTF
GAGTKLELK

The heavy chain and light chain variable regions of murine monoclonal antibody 2F7 comprise the following CDR sequences:

| Name | Sequence | No. |
|---|---|---|
| HCDR1 | GFTFSNYYMS | SEQ ID NO: 9 |
| HCDR2 | YVSSGGGSTYYSDSVKG | SEQ ID NO: 10 |
| HCDR3 | ESYSQGNYFDY | SEQ ID NO: 11 |
| LCDR1 | RASQSISDYLH | SEQ ID NO: 12 |
| LCDR2 | FASQSIS | SEQ ID NO: 13 |
| LCDR3 | QNGHSFSLT | SEQ ID NO: 14 |

The sequences of the heavy chain and light chain variable regions of murine monoclonal antibody 2F8 arc as follows:

2F8 HCVR
SEQ ID NO: 50
QVQLQQPGSVLVRPGASVKLSCKASGYTFTNSWMNWAKLRPGQGLEWIG
GIYPNSGNIEYNEKFKGKATLTVDTSSSTAYMDLTSLTSEDSAVYYCAR
DSRFSYWGQGTLVTVSA
2F8 LCVR
SEQ ID NO: 51
DIVMTQSHKFMSTSVGDRVSITCKASQDVRTAVAWYQQKPGQSPKLLIS
STSYRYTGVPDRFTGSGSGTDFTFIISSVQAEDLAVYYCQQHYSTPLTF
GAGTKLELK

The heavy chain and light chain variable regions of murine monoclonal antibody 2F8 comprise the following CDR sequences:

| Name | Sequence | No. |
|---|---|---|
| HCDR1 | GYTFTNSWMN | SEQ ID NO: 23 |
| HCDR2 | GIYPNSGNIEYNEKFKG | SEQ ID NO: 24 |
| HCDR3 | DSRFSY | SEQ ID NO: 25 |
| LCDR1 | KASQDVRTAVA | SEQ ID NO: 26 |
| LCDR2 | STSYRYT | SEQ ID NO: 27 |
| LCDR3 | QQHYSTPLT | SEQ ID NO: 28 |

The sequences of the heavy chain and light chain variable regions of murine monoclonal antibody 1C9 are as follows:

1C9 HCVR
SEQ ID NO: 52
QVQLQQPGSVLVRPGASVKLSCKASGDTFTTYWMNWVKQRPGQGLEWIG
GIYLNSGSSEYNEKFKGKATLSVDTSSSTAYMDLSSLTSEDSAVYYCAR
DSRFSYWGQGTLVTVSA
1C9 LCVR
SEQ ID NO: 53
DIVMTQSHKFLSTSVGDRVSITCKASQDVSTAVAWYQQKPGQSPELLIS
SASYRYTGVPDRFTGSGSGTDFTFTISSVQAEDLAVYYCQQHYNTPLTF
GAGTQLELK

The heavy chain and light chain variable regions of murine monoclonal antibody 1C9 comprises the following CDR sequences:

| Name | Sequence | No. |
|---|---|---|
| HCDR1 | GDTFTTY | SEQ ID NO: 29 |
| HCDR2 | YLNSGS | SEQ ID NO: 30 |
| HCDR3 | DSRFSY | SEQ ID NO: 31 |
| LCDR1 | KASQDVSTAVA | SEQ ID NO: 32 |
| LCDR2 | SASYRYT | SEQ ID NO: 33 |
| LCDR3 | QQHYNTPLT | SEQ ID NO: 34 |

### Example 3: Humanization Experiment of Murine Antibodies

Humanization of the murine anti-human B7-H4 monoclonal antibodies was performed by using methods as published in many documents in the art. Briefly, parental (murine antibody) constant domains were replaced with human constant domains. In the present invention, human germline antibody sequences were selected according to the homology between the murine antibodies and human antibodies, and the murine antibodies 2G6, 2F7, 2F8 and 1C9 were humanized. The CDR regions of the murine antibodies 2G6 and 2F7 were grafted onto the selected corresponding humanized templates to replace the humanized variable regions, and then recombined with the IgG constant region(s) (preferably. IgG1 for the heavy chain, and κ for the light chain). Then, based on the three-dimensional structure of the murine antibodies, the embedded residues, the residues directly interacting with the CDR regions and the residues with significant influence on the conformation of VL and VH were subjected to back mutation, and chemically unstable amino acid residues of the CDR regions were optimized, wherein the HCDR1 of murine monoclonal antibody 2F8 was optimized to GYTFTSSWMN (SEQ ID NO: 43), HCDR2 was optimized to GIYPNRGNIEY NEKFKG (SEQ ID NO: 44), the HCDR2 of murine monoclonal antibody 1C9 was optimized to YLNRGS (SEQ ID NO: 45), and the designed humanized antibodies comprising the following combination of light and heavy chain variable region sequences were obtained.

The heavy and light chain variable regions of humanized antibody hu2G6 are as follows:

hu2G6 HCVR
SEQ ID NO: 15
EVQLLESGGGLVQPGGSLRLSCAASGFTFSRYGMSWVRQAPGKGLEWVS
GINGGGSYTYYLDTVKGRFTISRDNARNTLYLQMSSLRAEDTAVYYCVS
QQSNYYFDYWGQGTLVTVSS
hu2G6 LCVR
SEQ ID NO: 16
DIRMTQSPSSLSASVGDRVTITCHASQGISSNIGWLQQKPGKAPKALIY
HGTNLEDGVPSRFSGSGSGADYTLTISSLQPEDFATYYCVQYAQFPYTF
GGGTKVEIK

The heavy and light chain variable regions of humanized antibody hu2F7 are as follows:

hu2F7 HCVR
SEQ ID NO: 17
EVQLVESGGGLVQPGGSLRLSCAASGFTFSNYYMSWVRQAPGKGLEWVA
YVSSGGGSTYYSDSVKGRFTISRDNAKNTLYLQMSSLRAEDTAVYYCTR
ESYSQGNYFDYWGQGTTVTVSS
hu2F7 LCVR
SEQ ID NO: 18
EIVMTQSPATLSLSPGERATLSCRASQSISDYLHWYQQKPGQSPRLLIK
FASQSISGIPARFSGSGSGTDFTLTISSLEPEDFAVYYCQNGHSFSLTF
GQGTKLEIK

The heavy and light chain variable regions of humanized antibody hu2F8 are as follows:

hu2F8 HCVR
SEQ ID NO: 35
EVQLVQSGAEVKKPGASVKVSCKASGYTFTSSWMNWVRQAPGQRLEWMG
GIYPNRGNIEYNEKFKGRVTLTVDTSASTAYMELSSLRSEDTAVYYCAR
DSRFSYWGQGTLVTVSS
hu2F8 LCVR
SEQ ID NO 36
DIQMTQSPSSLSASVGDRVTITCKASQDVRTAVAWYQQKPGKAPKLLIS
STSYRYTGVPSRFSGSGSGTDFTFTISSLQPEDIATYYCQQHYSTPLTF
GGGTKVEIK

The heavy and light chain variable regions of humanized antibody hu1C9 are as follows:

hu1C9 HCVR
SEQ ID NO: 37
EVQLVQSGAEVKKPGASVKVSCKASGDTFTTYWMNWVRQAPGQRLEWMG
GIYLNRGSSEYNEKFKGRVTLTVDTSASTAYMELSSLRSEDTAVYYCAR
DSRFSYWGQGTLVTVSS
hu1C9 LCVR
SEQ ID NO 38
DIQMTQSPSSLSASVGDRVTITCKASQDVSTAVAWYQQKPGKAPKLLIS
SASYRYTGVPSRFSGSGSGTDFTFTISSLQPEDIATYYCQQHYNTPLTF
GGGTKVEIK;

The variable regions were recombined with the IgG constant region(s) (preferably, IgG1 for the heavy chain, and κ for the light chain). Exemplary heavy and light chain

constant region sequences are shown as follows,

IgG1 C:
SEQ ID NO: 54
ASTKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDKKVEPKS
CDKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYKC
KVSNKALPAPIEKTISKAKGQPREPQVYTLPPSREEMTKNQVSLTCLVKGF
YPSDIAVEWESNGQPENNYKTTPPVLDSDGSFFLYSKLTVDKSRWQQGNVF
SCSVMHEALHNHYTQKSLSISPGK
Ig kappa C:
SEQ ID NO: 55
RTVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKSF
NRGEC;

Humanized antibodies consisting of the following exemplary light and heavy chain sequences were obtained after linking:

hu2G6 HC
SEQ ID NO: 19
EVQLLESGGGLVQPGGSLRLSCAASGFTFSRYGMSWVRQAPGKGLEWVSGI
NGGGSYTYYLDTVKGRFTISRDNARNTLYLQMSSLRAEDTAVYYCVSQGSN
YYFDYWGQGTLVTVSSASTKGPSVFPLAPSSKSTSGGTAALGCLVKDYFPE
PVTVSWNSGALTSGVHTFPAVLQSSGLYSLSSVVTVPSSSLGTQTYICNVN
HKPSNTKVDKKVEPKSCDKTHTCPPCPAPELLGGPSVFLFPPKPKDTLMIS
RTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSRE
EMTKNQVSLTCLVKGFYPSDIAVEWESNGQPENNYKTTPPVLDSDGSFFLY
SKLTVDKSRWQQGNVFSCSVMHEALHNHYTQKSLSLSPGK
hu2G6 LC
SEQ ID NO: 20
DIRMTQSPSSLSASVGDRVTITCHASQGISSNIGWLQQKPGKAPKALIYHG
TNLEDGVPSRFSGSGSGADYTLTISSLQPEDFATYYCVQYAQFPYTFGGGT
KVEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSP
VTKSFNRGEC
hu2F7 HC
SEQ ID NO: 21
EVQLVESGGGLVQPGGSLRLSCAASGFTFSNYYMSWVRQAPGKGLEWVAYV
SSGGGSTYYSDSVKGRFTISRDNAKNTLYLQMSSLRAEDTAVYYCTRESYS
QGNYFDYWGQGTTVTVSSASTKGPSVFPLAPSSKSTSGGTAALGCLVKDYF
PEPVTVSWNSGALTSGVHTFPAVLQSSGLYSLSSVVTVPSSSLGTQTYICN
VNHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQWTLPPSR
EEMTKNQVSLTCLVKGFYPSDIAVEWESNGQPENNYKTTPPVLDSDGSFFL
YSKLTVDKSRWQQGNVFSCSVMHEALHNHYTQKSLSLSPGK
hu2F7 LC
SEQ ID NO: 22
EIVMTQSPATLSLSPGERATLSCRASQSISDYLHWYQQKPGQSPRLLIKFA
SQSISGIPARFSGSGSGTDFTLTISSLEPEDFAVYYCQNGHSFSLTFGQGT
KLEIKRTVAAPSVFIFPPSDEQLKSGTASWCLLNNFYPREAKVQWKVDNAL
QSGNSQESVTEQDSKDSTYSLSSTLTLSKADYEKHKVYACEVTHQGLSSPV
TKSFNRGEC
hu2F8 HC
SEQ ID NO: 39
EVQLVQSGAEVKKPGASVKVSCKASGYTFTSSWMNWVRQAPGQRLEWMGGI
YPNRGNIEYNEKFKGRVTLTVDTSASTAYMELSSLRSEDTAVYYCARDSRF
SYWGQGTLVTVSSASTKGPSVFPLAPSSKSTSGGTAALGCLVKDYFPEPVT
VSWNSGALTSGVHTFPAVLQSSGLYSLSSWTVPSSSLGTQTYICNVNHKPS
NTKVDKKVEPKSCDKTHTCPPCPAPELLGGPSVFLFPPKPKDTLMISRTPE
VTCVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSREEMTK
NQVSLTCLVKGFYPSDIAVEWESNGQPENNYKTTPPVLDSDGSFFLYSKLT
VDKSRWQQGNVFSCSVMHEALHNHYTQKSLSLSPGK
hu2F8 LC
SEQ ID NO: 40
DIQMTQSPSSLSASVGDRVTITCKASQDVRTAVAWYQQKPGKAPKLLISST
SYRYTGVPSRFSGSGSGTDFTFTISSLQPEDIATYYCQQHYSTPLTFGGGT
KVEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSP
VTKSFNRGEC
hu1C9 HC
SEQ ID NO: 41
EVQLVQSGAEVKKPGASVKVSCKASGDTFTTYWMNWVRQAPGQRLEWMGGI
YLNRGSSEYNEKFKGRVTLTVDTSASTAYMELSSLRSEDTAVYYCARDSRF
SYWGQGTLVTVSSASTKGPSVFPLAPSSKSTSGGTAALGCLVKDYFPEPVT
VSWNSGALTSGVHTFPAVLQSSGLYSLSSVVTVPSSSLGTQTYICNVNHKP
SNTKVDKKVEPKSCDKTHTCPPCPAPELLGGPSVFLFPPKPKDTLMISRTP
EVTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSREEMT
KNQVSLTCLVKGFYPSDIAVEWESNGQPENNYKTTPPVLDSDGSFFLYSKL
TVDKSRWQQGNVFSCSVMHEALHNHYTQKSLSLSPGK
hu1C9 LC
SEQ ID NO: 42
DIQMTQSPSSLSASVGDRVTITCKASQDVSTAVAWYQQKPGKAPKLLISSA
SYRYTGVPSRFSGSGSGTDFTFTISSLQPEDIATYYCQQHYNTPLTFGGGT
KVEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSP
VTKSFNRGEC.

### Example 4: Expression of Anti-B7-H4 Humanized Antibodies

The recombinant vector was designed according to the sequences of the humanized antibodies. The heavy chain vector was designed as follows: signal peptide+humanized heavy chain. The light chain vector was designed as follows: signal peptide+humanized light chain.

The above sequences were inserted into the pCEP4 vector (ThermoFisher #V04450). The expression vector was synthesized according to the above design. The vector plasmid was obtained and extracted in large scale and sent for sequencing for verification. The qualified plasmid was transfected into human 293F cells (ThermoFisher #R79007) with PEI (ThemioFisher #BMS1003-A). The 293F cells were continuously cultured with serum-free medium (Shanghai OPM Biosciences, OPM-293 CD03) to the logarithmic growth phase and used for cell transfection. 21.4 μg of humanized antibody light chain plasmid and 23.6 μg of humanized antibody heavy chain plasmid were dissolved in 10 ml Opti-MEM® I Reduced Serum Medium (GIBCO, 31985-070) and mixed well, then 200 μg PEI was added and mixed well, incubated at RT for 15 min. 50 mL of cells were then added. Cell culture conditions: 5% CO₂, 37° C., 125 rpm/min. During the culture, supplements were added on the day 1 and day 3 until the cell viability was less than 70%. The cell supernatant was collected, centrifuged and filtered. The centrifuged and filtered cell culture medium was loaded onto the antibody purification affinity column. The column was washed with phosphate buffer. The sample was eluted with glycine hydrochloride buffer (pH 2.7 0.1 M Gly-HCl), neutralized with 1 M Tris hydrochloride pH 9.0, and dialyzed against phosphate buffer to finally obtain the purified humanized antibody, which could be used in experiments of each Example.

### Examples 5: Detection of In Vitro Binding Ability of Humanized Antibodies

The designed humanized antibodies were tested in experiments in vitro as follows:

1. In Vitro Cell Binding Experiment:

The cultured MX-1 cells expressing human B7-H4 (CLS Cell Lines Service GmbH #300296) were collected, the cell density was adjusted with PBS at pH 7.4, and the cells were plated on a 96-well plate with V-shaped bottom at 1×10⁵ cells per well. The plate was centrifuged at 2000 rpm for 5 minutes and the supernatant was removed. 100 μl of the gradient diluted humanized antibody solution (diluted with PBS containing 0.5% BSA, starting from 1 μM, a 3-fold gradient of 10 doses) was added to each well, mixed well and incubated at 4° C. on a shaker for 1 hour. The plate was centrifuged at 2000 rpm for 5 minutes and the supernatant was removed. The cells were washed twice with PBS. 100 μl of FITC-labeled goat anti-human secondary antibody (Abeam, cat #ab97224) diluted with 0.5% BSA in PBS was added to each well, mixed well and incubated for 30 minutes at 4° C. on a shaker. The plate was centrifuged at 2000 rpm for 5 minutes and the supernatant was removed. The cells were washed twice with PBS and then resuspended in PBS. The signal was detected by using a flow cytometer (BECKMAN COULTER, model DxFLEX), and a concentration curve was plotted for result analysis. The results are as shown in Table 1. The humanized antibodies hu2G6 and hu2F7 both bind positively to MX-1 cells with high expression of B7-H4.

TABLE 1
Affinity (EC₅₀) of each humanized antibody to MX-1 cells

| Antibody name | FACS Binding EC₅₀ (nM) with MX-1 cells |
|---|---|
| hu2G6 | 14.3 |
| hu2F8 | 7.26 |
| hu1C9 | 7.25 |
| hu2F7 | 7.32 |

2. Affinity Kinetics Experiment:

The Biacore method is a generally acknowledged method for objectively detecting the affinity and kinetics between proteins. The affinity and binding kinetics of the antibodies to be tested of the present invention were analyzed by Biacore T200 (GE). The anti-B7-H4 antibodies to be tested of the present invention were covalently linked to CM5 (GE) chips by using the kit provided by Biacore and the NHS standard amino coupling method. Then 50 nM of human huB7-H4-His protein diluted in the same buffer was injected at a flow rate of 10 uL/min. After the injection, the samples were all regenerated with the regeneration reagent in the kit. The antigen-antibody binding kinetics was tracked for 3 minutes and the dissociation kinetics was tracked for 10 minutes. The obtained data were analyzed with a 1:1 (Langmuir) binding model by using BIAevaluation software of GE. The affinity kinetics data of the humanized antibodies calculated by this method are as shown in Table 2. The humanized antibodies hu2G6, hu2F7, hu2F8 and hu1C9 all show strong affinity to the human B7-H4 antigen protein.

TABLE 2
Affinity and kinetics characterization of each humanized antibody

| | Binding rate | Dissociation rate | Affinity |
|---|---|---|---|
| Antibody | kₐ(1/M*s) | k_{d} (1/s) | K_{D} |
| hu2G6 | 7.41e+05 | 1.00e−05 | 1.35e−11 |
| hu2F8 | 4.47e+05 | 1.12e−04 | 2.50e−10 |
| hu1C9 | 3.35e+05 | 1.00e−05 | 2.98e−11 |
| hu2F7 | 3.29e+05 | 2.49e−04 | 7.57e−10 |

### Example 6: Endocytosis of Anti-B7-H4 Antibodies

In order to test whether the antibodies of the present invention could be endocytosed along with human B7-H4 after binding to B7-H4, MX-1 cells were used for evaluation. MX-1 cells were trypsinized (first washed with PBS once at 37° C. for about 2 min), collected and resuspended in pre-chilled FACS buffer. The cell concentration was adjusted to 1×10⁶ cells/mL. 1 mL of the cell suspension was added to an EP tube, centrifuged at 1500 rpm for 5 minutes, and the supernatant was removed. 1 mL of the prepared antibody to be tested was added to resuspend the cells, and the final concentration of the antibody was 20 μg/ml. The cells were incubated on a 4° C. shaker for 1 hour, centrifuged, and the supernatant was discarded (4° C., 1500 rpmx5 min). The cells were washed twice with FACS buffer and the supernatant was removed. 100 μL of fluorescent secondary antibody working solution was added to each tube to resuspend the cells. The cells were incubated on a 4° C. shaker for 30 min, centrifuged, and the supernatant was discarded (4° C., 1500 rpmx5 min). The cells were washed twice with FACS buffer and the supernatant was removed. 1.0 mL of pre-warmed MX-1 cell complete medium was added to each tube to resuspend the cells and mixed. The cell suspension was aliquoted into 4 tubes at 200 μL per tube, which were respectively the 0 min group, blank group, 30 min group and 2 h group. The 0 min and blank groups were placed on ice, while the other groups were placed in a 37° C. incubator for endocytosis for 30 min and 2 h respectively. At the corresponding time point, the EP tube was taken out and placed on ice to pre-chill for 5 min. All treatment groups were centrifuged and the supernatant was discarded (4° C., 1500 rpmx5 min). The cells were washed once with FACS buffer and the supernatant was removed. 250 μL strip buffer was added to EP tubes of all treatment groups except the 0 min group and incubated for 8 min at room temperature. The cells were centrifuged and the supernatant was discraded (4° C., 1500 rpm×5 min). The cells were washed twice with FACS buffer and the supernatant was removed. All treatment groups were added with 100 μL immunostaining fixative, placed at 4° C. for more than 30 min, and detected by the flow cytometer DxFlex. Endocytosis percentage of B7-H4 antibody (%)=(average fluorescence intensity value at each time point−average fluorescence intensity value of the blank group)/(average fluorescence intensity value at zero point−average fluorescence intensity value of the blank group)×100%. The data are shown in Table 3:

TABLE 3
Humanized antibody-mediated endocytosis
efficiency of B7-H4 protein

| | 0 hour | 0.5 hour | 2 hours |
|---|---|---|---|
| Antibody | (%) | (%) | (%) |
| IgG control | 0 | 0.1 | 0.9 |
| hu2G6 | 0 | 18.6 | 28.7 |
| hu2F7 | 0 | 13.9 | 25.9 |

### Example 7: Conjugation of Antibodies to MC-MMAF

The antibodies of the present invention have cell affinity activity and endocytosis activity, making them suitable for coupling with drugs to form antibody-drug conjugates for treating B7-H4-mediated diseases. The coupling process is shown in the following equation, wherein Ab represents hu2G6 or hu2F7:

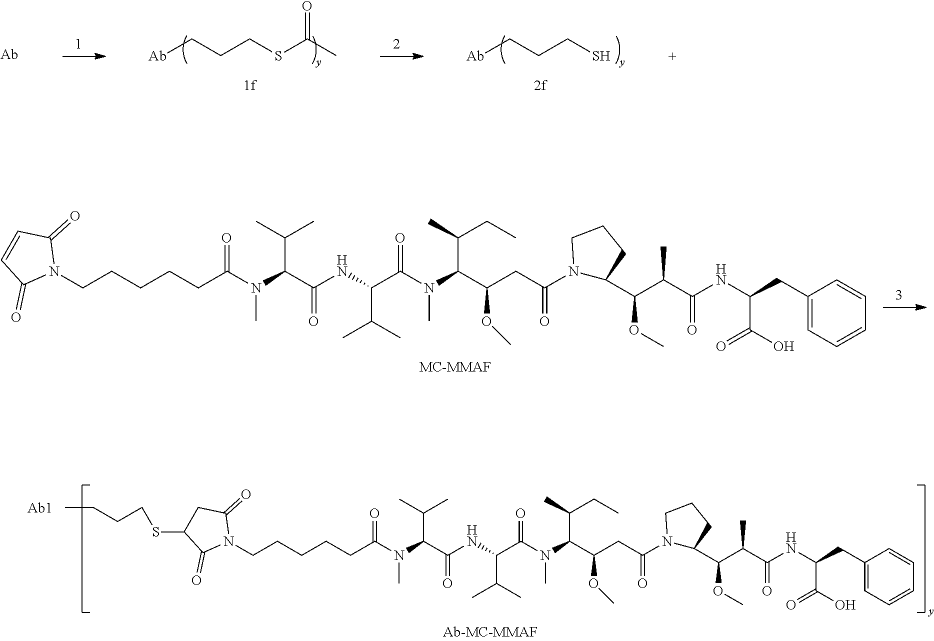

In the first step, S-(3-hydroxypropyl) thioacetate (0.7 mg, 5.3 mol) was dissolved in 0.9 mL acetonitrile to form a solution for later use. The above pre-prepared acetonitrile solution of S-(3-hydroxypropyl) thioacetate was added to the antibody in pH=4.3 acetate/sodium acetate buffer (10.35 mg/mL. 9.0 mL, 0.97 mol). Then 1.0 mL aqueous solution of sodium cyanoborohydride (14.1 mg, 224 mol) was added dropwise into the reaction mixture and reacted under shaking at 25° C. for 2 hours. After completion of the reaction, the reaction mixture was desalted and purified by using Sephadex G25 gel column (elution phase: pH 6.5 0.05 M PBS solution) to obtain a solution of product 1f. The solution was concentrated to 10 mg/mL and directly used in the next reaction.

In the second step, 1f solution (11.0 mL) was added with 0.35 mL of 2.0 M carboxyamine hydrochloride solution and reacted under shaking at 25° C. for 30 minutes. Then the reaction solution was desalted and purified by using Sephadex G25 gel column (elution phase: pH 6.5 0.05 M PBS solution) to obtain a solution of product 2f (concentration 6.17 mg/mL, 14.7 mL).

In the third step, the compound MC-MMAF (1.1 mg, 1.2 mol, prepared by the method disclosed in PCT patent WO2005081711) was dissolved in 0.3 mL acetonitrile, added into 2f solution (concentration 6.17 mg/mL, 3.0 mL) and reacted under shaking at 25° C. for 4 hours. Then the reaction solution was desalted and purified by Sephadex G25 gel column (elution phase: pH 6.5 0.05 M PBS solution), and filtered under sterile conditions with a filter to obtain the product Ab-MC-MMAF antibody-drug conjugate in PBS buffer (3.7 mg/mL, 4.7 mL), which was refrigerated at 4° C. The average value y of the product hu2G6-MC-MMAF determined by HIC-HPLC was 3.8, and samples of hu2G6-MC-MMAF (y=4) were obtained by HIC-HPLC purification. The average value y of the product hu2F7-MC-MMAF determined by HIC-HPLC was 3.2, and samples of hu2F7-MC-MMAF (y=2) and hu2F7-MC-MMAF (y=4) were obtained by HIC-HPLC purification.

### Example 8: Conjugation of Antibodies to SN-38

Antibody-conjugated drugs were prepared through the following coupling process, wherein Ab represents hu2F7:

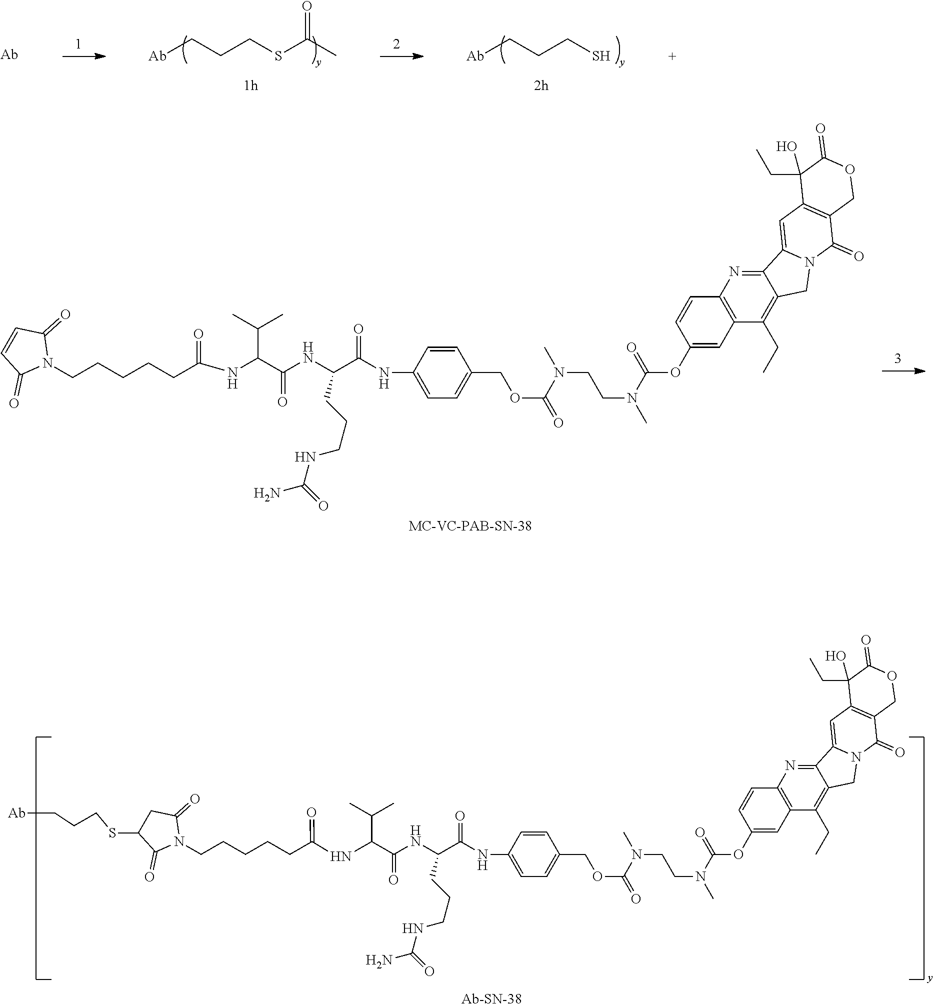

In the first step, S-(3-hydroxypropyl) thioacetate (0.7 mg. 5.3 mol) was dissolved in 0.9 mL acetonitrile to form a solution for later use. The above pre-prepared acetonitrile solution of S-(3-hydroxypropyl) thioacetate was added to the antibody in pH=4.3 acetate/sodium acetate buffer (10.35 mg/mL. 9.0 mL, 0.97 mol). Then 1.0 mL aqueous solution of sodium cyanoborohydride (14.1 mg, 224 mol) was added dropwise into the reaction mixture and reacted under shaking at 25° C. for 2 hours. After completion of the reaction, the reaction mixture was desalted and purified by using Sephadex G25 gel column (elution phase: pH 6.5 0.05 M PBS solution) to obtain a solution of product 1 h. The solution was concentrated to 10 mg/mL and directly used in the next reaction.

In the second step, 1 h solution (11.0 mL) was added with 0.35 mL of 2.0 M carboxyamine hydrochloride solution and reacted under shaking at 25° C. for 30 minutes. Then the reaction solution was desalted and purified by using Sephadex G25 gel column (elution phase: pH 6.5 0.05 M PBS solution) to obtain a solution of product 2 h (concentration 6.2 mg/mL. 15.0 mL). The 2 h solution was concentrated to about 10 mg/ml and used in the next reaction.

In the third step, the compound MC-VC-PAB-SN-38 (1.3 mg, 1.2 mol) was dissolved in 0.3 mL acetonitrile, added into 2 h solution (concentration 6.2 mg/mL, 3.0 mL) and reacted under shaking at 25° C. for 4 hours. Then the reaction solution was desalted and purified by Sephadex G25 gel column (elution phase: pH 6.5 0.05 M PBS solution), and filtered under sterile conditions with a filter to obtain the product hu2F7-SN-38 antibody-drug conjugate in PBS buffer (3.7 mg/mL, 4.7 mL), which was refrigerated at 4° C. The average value y was determined by the ultraviolet method. Cuvettes filled with sodium succinate buffer were placed in the reference absorption cell and in the sample determination absorption cell respectively, and after deducting the solvent blank, the cuvettes filled with the test solution were placed in the sample determination absorption cell. The absorbance at 280 nm and 370 nm was measured.

Data Processing:

The antibody content C_{mab}, was determined by establishing a standard curve and measuring the absorption at the wavelength of 280 nm. The small molecule content C_{Drug} was determined by measuring the absorption at the wavelength of 370 nm.

Average drug load y=C_{Drug}/C_{mab}

The average value y=3.7 of the product hu2F7-SN-38 was determined by the above method. Samples of hu2F7-SN-38 (y=4) were obtained by UV-HPLC purification.

### Example 9: Conjugation of Antibodies to Exatecan

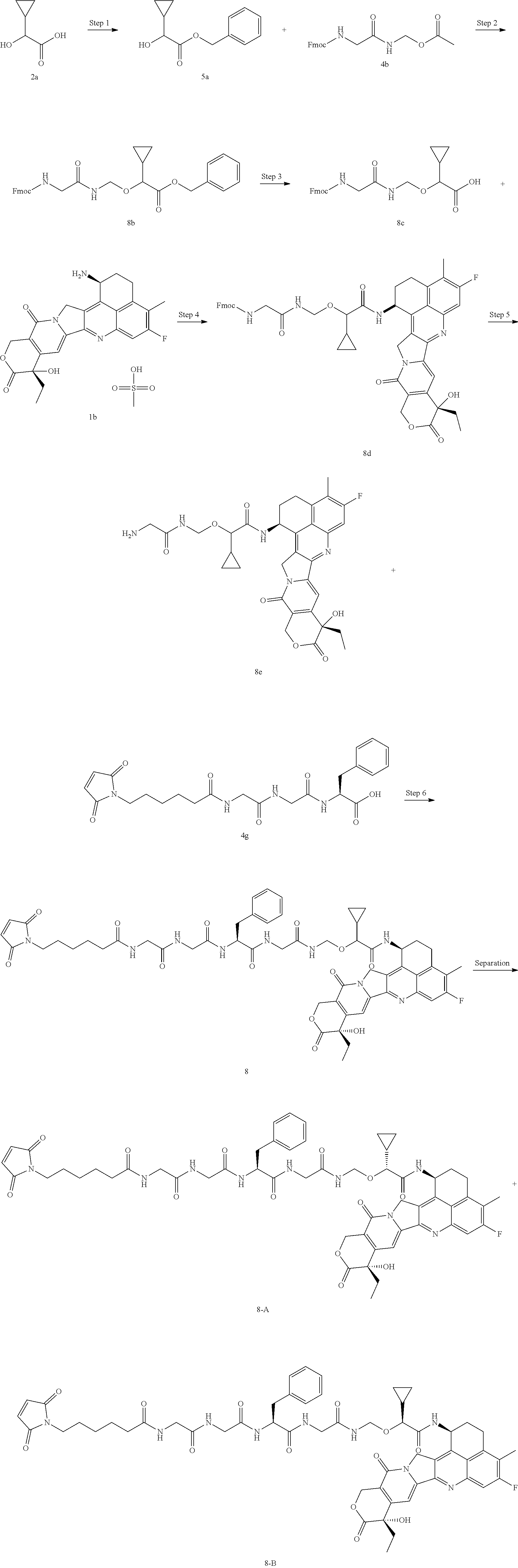

In the first step, 2a (2 g, 17.2 mmol) was dissolved in 75 mL acetonitrile and added successively with potassium carbonate (9.27 g, 67.2 mmol), benzyl bromide (20 mL, 167.2 mmol) and tetrabutylammonium iodide (620 mg, 1.68 mmol). The reaction solution was stirred at room temperature for 48 hours and filtered through diatomaceous earth. The filter cake was rinsed with ethyl acetate (20 ml). The filtrate was pooled and concentrated under reduced pressure. The obtained residues were purified by silica gel column chromatography with developing solvent system C to obtain product 5a (3.2 g, yield: 90.1%).

In the second step, 5a (181.3 mg, 0.879 mmol) and 4b (270 mg, 0.733 mmol) were added into a reaction flask, added with 6 mL tetrahydrofuran and replaced with argon three times. The reaction mixture was cooled to 0-5° C. in an ice-water bath and added with potassium tert-butoxide (164 mg, 1.46 mmol), then warmed to room temperature by removing the ice bath and stirred for 40 minutes. The reaction mixture was added with 15 mL ice water and extracted with ethyl acetate (40 mL/2) and chloroform (20 mL×5). The organic phases were pooled and concentrated. The obtained residues were dissolved in 6 mL dioxane, added with 3 mL water, sodium bicarbonate (73.8 mg, 0.879 mmol) and 9-fluorene methyl chlorofommate (190 mg, 0.734 mmol) and stirred at room temperature for 2 hours. The reaction solution was added with 30 mL water and extracted with ethyl acetate (20 mL×3). The organic phases were washed with saturated sodium chloride solution (30 mL), dried by using anhydrous sodium sulfate and filtered. The filtrate was concentrated under reduced pressure. The obtained residues were purified by silica gel column chromatography with the developing solvent system C to obtain product 5b Benzyl 10-cyclopropyl-1-(9H-fluoren-9-yl)-3,6-dioxo-2,9-dioxa-4,7-diazaundec-11-ate (73 mg, yield: 19.4%).

MS m/z (ESI): 515.0 [M+1].

In the third step, 5b (30 mg, 0.058 mmol) was dissolved in 6.75 mL of a mixed solvent of tetrahydrofuran and ethyl acetate (V:V=2:1), added with palladium on carbon (18 mg, content 10%, dry basis), replaced with hydrogen three times, and reacted under stirring at room temperature for 1 hour. The reaction solution was filtered with diatomaceous earth. The filter cake was rinsed with ethyl acetate. The filtrate was concentrated to obtain the crude product 5c 10-cyclopropyl-1-(9H-fluoren-9-yl)-3,6-dioxo-2,9-dioxa-4,7-diazaundec-1l-acid (20 mg), which was directly used in the next reactopm without purification.

MS m/z (ESI): 424.9 [M+1].

In the fourth step, 1b (15 mg, 28.2 μmol) was added into a reaction flask, added with 1.5 mL N. N-dimethylfomiamide and replaced with argon three times. The reaction mixture was cooled to 0-5° C. in an ice-water bath, added with a drop of triethylamine, added with the crude product 5c (20 mg, 47.1 μmol), added with 4-(4,6-dimethoxy-1,3,5-triazin-2-yl)-4-methylchloromorpholine (25.4 mg, 86.2 μmol) and reacted under stirring in an ice bath for 40 minutes. The reaction mixture was added with 15 mL water and extracted with ethyl acetate (20 mL*3). The organic phases were pooled. The organic phases were washed with saturated sodium chloride solution (20 mL*2), dried by using anhydrous sodium sulfate and filtered. The filtrate was concentrated under reduced pressure. The obtained residues were purified by thin layer chromatography with the developing solvent system B to obtain the title product 5d (9H-fluoren-9-yl)methyl(2-(((1-cyclopropyl-2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-2-oxoethyl)carbamate (23.7 mg, yield: 78.9%).

MS m/z (ES): 842.1[M+1].

In the fifth step, 5d (30 mg, 35.7 μmol) was dissolved in 3 mL dichloromethane, added with 1.5 mL diethylamine and stirred at room temperature for 2 hours. The reaction solution was concentrated under reduced pressure, added with 1.5 mL toluene and concentrated under reduced pressure, repeating twice. The residues were added with 4.5 mL n-hexane and pulped. The supernatant was poured out after letting standing and the solid was kept. The solid residues were concentrated under reduced pressure and pumped dry to obtain the crude product 5e 2-((2-aminoacetamido)methoxy)-2-cyclopropyl-N-((1S,9S)-9-ethyl-5-fluoro-9-hydroxy-4-methyl-10,13-dioxo-2,3,9,10,13, 15-hexahydro-1N,12H-benzo[de]pyrano[3′,4′:6,7]indolozino[1,2-b]quinolin-1-yl) acetamide (23 mg), which was directly used in the next reaction without purification.

MS m/z (ES): 638.0[M+18].

In the sixth step, the crude product 5e (20 mg, 32.3 μmol) was dissolved in 1 mL N. N-dimethylformamide and replaced with argon three times. The reaction mixture was cooled to 0-5° C. in an ice-water bath, added with 0.5 mL of N, N-dimethylformamide solution of 4 g (31.8 mg, 67.3 μmol), added with 4-(4,6-dimethoxy-1,3,5-triazin-2-yl)-4-methylchloromoipholine (27.8 mg, 94.3 μmol) and reacted under stirring in an ice bath for 10 minutes. The reaction mixture was warmed to room temperature by removing the ice bath and reacted under stirring for 1 hour to produce compound 5. The reaction solution was purified by high performance liquid chromatography (separation conditions: column: XBridge Prep C18 OBD 5 μm 19*250 mm; mobile phase: A-water (10 mmol NH₄OAc): B-acetonitrile, gradient elution, flow rate: 18 mL/min). The corresponding components were collected and concentrated under reduced pressure to obtain products 5-A and 5-B (3.6 mg, 2.6 mg).

MS m/z (EST): 1074.4 [M+1].

Single configuration compound 5-A (shorter retention time) UPLC analysis: retention time 1.14 minutes, purity: 85% (column: ACQUITY UPLC BEHC18 1.7 μm 2.1*50 mm, mobile phase: A-water (5 mmol NH₄OAc), B-acetonitrile).

¹H NMR (400 MHz, DMSO-d₆): δ 8.60 (t, 1H), 8.51-8.49 (d, 1H), 8.32-8.24 (m, 1H), 8.13-8.02 (m, 2H), 8.02-7.96 (m, 1H), 7.82-7.75 (m, 1H), 7.31 (s, 1H), 7.26-7.15 (m, 4H), 6.99 (s, 1H), 6.55-6.48 (m, 1H), 5.65-5.54 (m, 1H), 5.41 (s, 2H), 5.35-5.15 (m, 3H), 4.74-4.62 (m, 2H), 4.54-4.40 (m, 2H), 3.76-3.64 (m, 4H), 3.62-3.48 (m, 2H), 3.20-3.07 (m, 2H), 3.04-2.94 (m, 2H), 2.80-2.62 (m, 214), 2.45-2.30 (m, 314), 2.25-2.15 (m, 2H), 2.15-2.04 (m, 2H), 1.93-1.78 (m, 2H), 1.52-1.39 (m, 3H), 1.34-1.12 (m, 5H), 0.87 (t, 31-1), 0.64-0.38 (m,

Single configuration compound 5-B (longer retention time):

UPLC analysis: retention time 1.16 minutes, purity: 89% (column: ACQUITY UPLC BEHC18 1.7 inn 2.1*50 mm, mobile phase: A-water (5 mmol NH₄OAc), B-acetonitrile).

¹H NMR (400 MHz, DMSO-d₆): δ 8.68-8.60 (m, 1H), 8.58-8.50 (m, 1H), 8.32-8.24 (m, 1H), 8.13-8.02 (m, 2H), 8.02-7.94 (m, 1H), 7.82-7.75 (m, 1H), 7.31 (s, 1H), 7.26-7.13 (m, 4H), 6.99 (s, 1H), 6.55-6.48 (m, 1H), 5.60-5.50 (m, 1H), 5.41 (s, 2H), 5.35-5.15 (m, 3H), 4.78-4.68 (in, 1H), 4.60-4.40 (m, 2H), 3.76-3.58 (m, 4H), 3.58-3.48 (m, 1H), 3.20-3.10 (m, 2H), 3.08-2.97 (m, 2H), 2.80-2.72 (m, 2H), 2.45-2.30 (in, 3H), 2.25-2.13 (m, 2H), 2.13-2.04 (m, 2H), 2.03-1.94 (m, 2H), 1.91-1.78 (m, 2H), 1.52-1.39 (m, 3H), 1.34-1.12 (m, 5H), 0.91-0.79 (m, 3H), 0.53-0.34 (m, 4H).

The preparation methods of other intermediates were with reference to that of intermediate 5.

The PBS buffered aqueous solution of antibody hu2F7 (pH=6.5 0.05 M PBS buffered aqueous solution; 7.3 ml, 13.8 mg/ml, 0.681 μmol) was added with a prepared aqueous solution of tris(2-carboxyethyl) phosphine (10 mM. 0.347 mL, 3.47 μmol) at 37° C. The reaction mixture was placed in a water bath shaker and reacted under shaking at 37° C. for 3 hours. The reaction was terminated, and the reaction solution was cooled to 25° C. in a water bath, diluted to 14.0 ml, and 3.3 ml of the solution was taken out for the next reaction.

Compound 5-A (3.0 mg, 3.72 μmol) was dissolved in 0.15 mL DMSO and added into the above 3.3 ml solution. The reaction mixture was placed in a water bath shaker and reacted under shaking at 25° C. for 3 hours. The reaction was terminated. The reaction solution was desalted and purified by using a Sephadex G25 gel column (elution phase: pH 6.5 0.05 M PBS buffered aqueous solution, containing 0.001 M EDTA) to obtain an exemplary product of Ab-Exatecan, hu2F7-Exatecan (Compound 34) in PBS buffer (1.35 mg/mL, 13 mL), which was stored at 4° C. The average value y was determined by the ultraviolet method. Cuvettes filled with sodium succinate buffer were respectively placed in the reference absorption cell and the sample determination absorption cell, and after deducting the solvent blank, the cuvettes filled with the test solution were placed in the sample determination absorption cell. The absorbance at 280 nm and 370 nm was measured.

Data Processing:

The antibody content C_{mab} was determined by establishing a standard curve and measuring the absorption at the wavelength of 280 nm. The small molecule content C_{Drug} was determined by measuring the absorption at the wavelength of 370 nm.

Average drug load y=C_{Drug}/C_{mab}

As for the exemplary product hu2F7-Exatecan (compound 34), y was determined to be 7.6 by the above method. Samples of hu2F7-Exatecan (y=8) were obtained by UV-HPLC purification.

The preparation methods of other antibody conjugates were with reference to that of compound 34.

### Example 10: The Killing Activity of Antibody-Drug Conjugates on Tumor Cells

In order to test the killing effect of the antibody-drug conjugates of the present invention on tumor cells, MX-1 breast cancer cells were used for evaluation. MX-1 cells were collected, centrifuged and counted. The cell density was adjusted to 0.44×10⁶ cells/mL with complete medium, and the cells were plated in 60 wells of a white 96-well plate at 90 μL per well with a cell number of 40,000. The rest peripheral wells were added with 100 μL PBS. The cell plate was placed in a 37° C., 5% CO₂ incubator and cultured overnight. On the second day of the experiment, the antibody-drug conjugate solution was prepared with PBS in a 96-well V-bottom plate, starting from a concentration of 1000 nM (3-fold dilution and 9 concentrations). After completion of the preparation, the solution was added to a white 96-well plate at 10 μL per well in duplicates. The cell plate was placed in a 37° C., 5% CO₂ incubator and the culture lasted for 72 hours. On the fifth day of the experiment, the plate was detected and read. The cell culture plate was taken out. Two control wells were set up. 100 μL medium containing 40,000 cells was added to each well, and after equilibrating to room temperature, 50 μL CTG solution (Promega G7573) was added to each well. The mixture was shaken and mixed, placed in dark and let stand for 10 minutes, and then detected by using the luminescence program of the microplate reader. Maximum killing rate=(1-fluorescence value of 1000 nM well/fluorescence value of the control well) %. The experimental results are as shown in Table 4:

TABLE 4
Evaluation of the killing activity of
antibody-drug conjugates on tumor cells

| | | Killing effect |
|---|---|---|
| Antibody-drug conjugate | IC₅₀ (nM) | at maximum dose |
| IgG-MC-MMAF (control) | ND | ND |
| hu2G6-MC-MMAF (compound 1, y = 4) | 1.1 | 48.2% |
| hu2F7-MC-MMAF (compound 2, y = 4) | 3.4 | 53.9% |
| hu2F7-SN-38 (compound 6, y = 4) | 78.85 | 56.4% |
| hu2F7-Exatecan (compound 34, y = 8) | 80.28 | 60.39% |

ND: No activity detectable.

### Example 11: The Inhibition Effect of Antibody-Drug Conjugates on Growth of Tumor Cells

In order to test the killing effect of the antibody-drug conjugates of the present invention on tumor cells. SK-BR-3 (ATCC #HTB30) breast cancer cells were used for evaluation. SK-BR-3 cells were collected, centrifuged and counted. The cell density was adjusted to 0.44/10° cells/mL with complete medium, and the cells were plated in 60 wells of a white 96-well plate at 90 μL per well with a cell number of 40,000. The rest peripheral wells were added with 100 μL PBS. The cell plate was placed in a 37° C., 5% CO₂ incubator and cultured overnight. On the second day of the experiment, the antibody-drug conjugate solution was prepared with PBS in a 96-well V-bottom plate, starting from a concentration of 1000 nM (3-fold dilution and 9 concentrations). After completion of the preparation, the solution was added to a white 96-well plate at 10 μL per well in duplicates. Two other control wells were set up and 10 μL PBS was added to each well. The cell plate was placed in a 37° C. 5% CO₂ incubator and the culture lasted for 72 hours. On the fifth day of the experiment, the plate was detected and read. The cell culture plate was taken out. After equilibrating to room temperature. 50 μL CTG solution (Promega G7573) was added to each well. The mixture was shaken and mixed, placed in dark and let stand for 10 minutes, and then detected by using the luminescence program of the microplate reader. Maximum inhibition rate=(1-fluorescence value of 1000 nM well/fluorescence value of the control well) %. The experimental results arc as shown in Table 5:

TABLE 5
Evaluation of the inhibition activity of
antibody-drug conjugates on tumor cells

| | | Maximum |
|---|---|---|
| | | inhibition |
| Antibody-drug conjugate | IC₅₀ (nM) | rate |
| IgG-MC-MMAF (control) | ND | ND |
| hu2G6-MC-MMAF (compound 1, y = 4) | 1.0 | 90.3% |
| hu2F7-MC-MMAF (compound 2, y = 4) | 9.5 | 91.8% |

ND: No activity detectable.

### Example 12: Evaluation of the In Vivo Efficacy of Antibody-Drug Conjugates Conjugated to MMAF

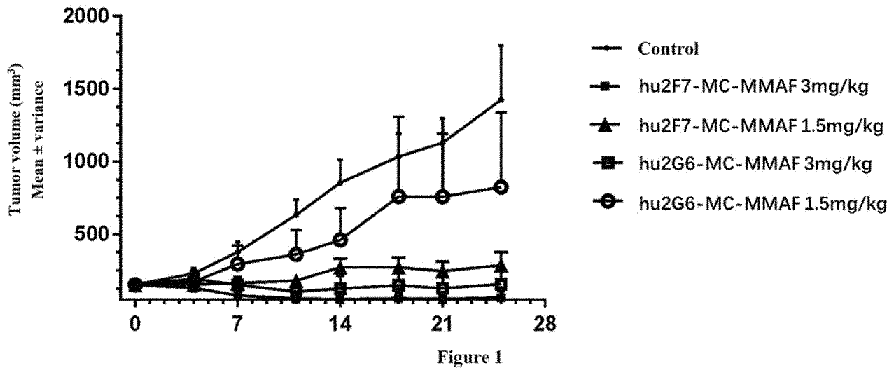

After the formation of transplanted tumors with MX-1 cells in mice, the anti-tumor effect of the antibody-drug conjugates of the present invention was evaluated. 5×10⁶ MX-1 cells were injected subcutaneously into immunodeficient nude mice (BALB/c Nude). After 2 weeks, intravenous injection of the antibody-drug conjugates hu2G6-MC-MMAF and hu2F7-MC-MMAF was performed, at a frequency of once/week and a dose of 1.5 mg/kg or 3 mg/kg. Human IgG1 protein was used as the control at a dose of 3 mg/kg. There were 5 mice in each group of the control group or the administration group. The tumor inhibition rate was calculated by measuring the tumor volume. Tumor inhibition rate=100%−(tumor volume of the administration group on day 21−tumor volume of the administration group on day 0)/(tumor volume of the control group on day 21−tumor volume of the control group on day 0). The experimental results are as shown in FIG. 1 and Table 6. Both hu2G6-MC-MMAF and hu2F7-MC-MMAF show dose-dependent anti-tumor effect. Both hu2G6-MC-MMAF and hu2F7-MC-MMAF show tumor inhibition rate of more than 100% at the dose of 3 mg/kg, which means that the antibody-drug conjugates of the present invention can not only inhibit tumor growth, but also exert killing effect on the tumor already formed.

TABLE 6
Efficacy of administered compounds on MX-1 transplanted
tumor in tumor-bearing nude mice

| | Tumor |
|---|---|
| | inhibition |
| Administration group | rate |
| hu2G6-MC-MMAF (compound 1, y = 4) 1.5 mg/kg | 38.0% |
| hu2G6-MC-MMAF (compound 1, y = 4) 3 mg/kg | 115.6% |
| hu2F7-MC-MMAF (compound 2, y = 4) 1.5 mg/kg | 90.3% |
| hu2F7-MC-MMAF (compound 2, y = 4) 3 mg/kg | 163.8% |

### Example 13: Evaluation of the In Vivo Efficacy of Antibody-Drug Conjugates with Different Drug Loads

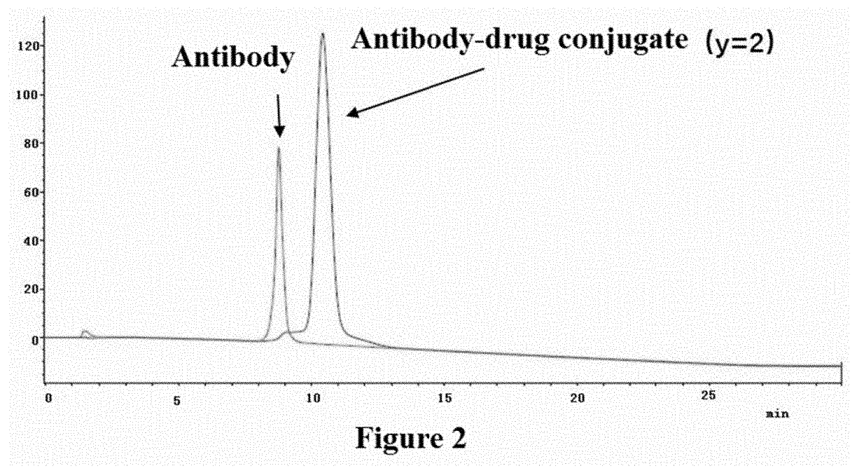

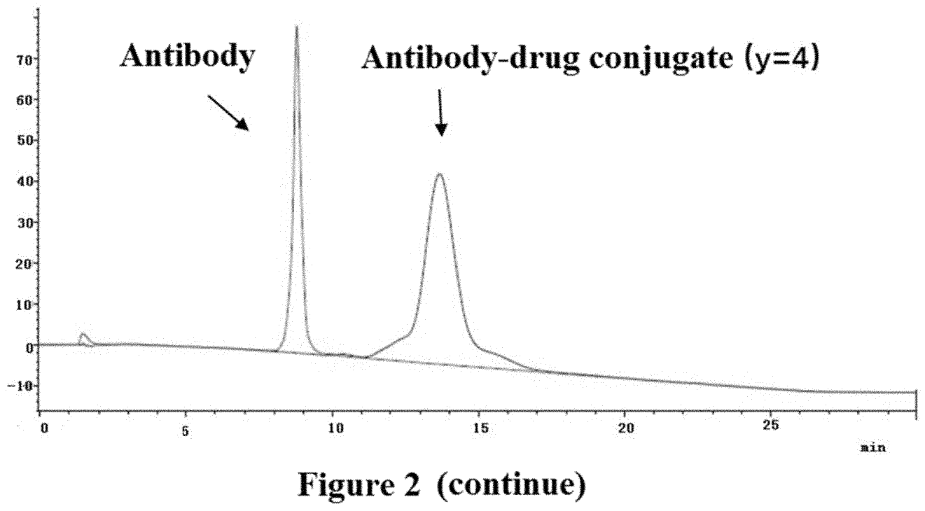

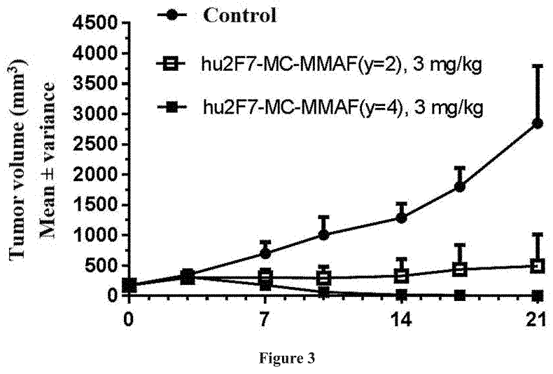

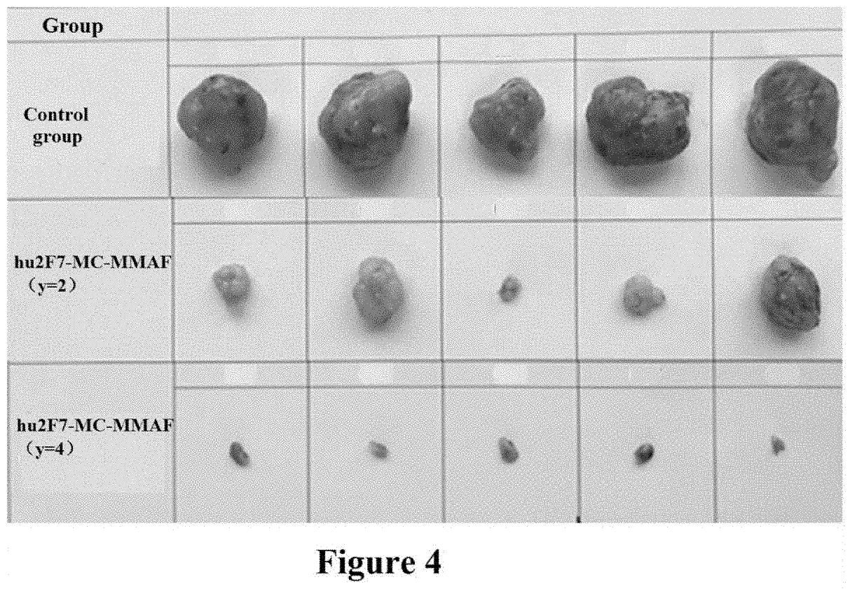

In order to further study the antibody-drug conjugates with different drug loads, the antibody-drug conjugates were prepared by the method of Example 7 and purified by HPLC to obtain hu2F7-MC-MMAF (y=2) and hu2F7-MC-MMAF (y 4) (FIG. 2). After formation of transplanted tumors with MX-1 in mice, the anti-tumor effect of the antibody-drug conjugates of the present invention was evaluated. 5×10⁶ MX-1 cells were injected subcutaneously into immunodeficient nude mice. After 2 weeks, intravenous injection of the antibody-drug conjugates hu2F7-MC-MMAF (y=2) and hu2F7-MC-MMAF (y=4) was performed, at a frequency of once/week and a dose of 3 mg/kg. Human IgG1 protein was used as the control at a dose of 3 mg/kg. There were 5 mice in each group of the control group or the administration group. The tumor inhibition rate was calculated by measuring the tumor volume. Tumor inhibition rate=100%−(tumor volume of the administration group on day 21−tumor volume of the administration group on day 0)/(tumor volume of the control group on day 21−tumor volume of the control group on day 0). The experimental results are as shown in FIG. 3 and Table 7. Both hu2F7-MC-MMAF (y=2) and hu2F7-MC-MMAF (y=4) show anti-tumor effect. hu2F7-MC-MMAF (y=4) has stronger anti-tumor effect than hu2F7-MC-MMAF (y=2) at the dose of 3 mg/kg, showing a tumor inhibition rate of more than 100%, which means that hu2F7-MC-MMAF (y=4) can not only inhibit tumor growth, but also exert killing effect on the tumor already formed (FIG. 4).

TABLE 7
Efficacy of antibody-drag conjugates with different drug loads
on MX-1 transplanted tumor in tumor-bearing nude mice

| | Tumor |
|---|---|
| | inhibition |
| Administration group | rate |
| hu2F7-MC-MMAF (y = 2) 3 mg/kg | 87.99% |
| hu2F7-MC-MMAF (y = 4) 3 mg/kg | 197.87% |

### Example 14: Evaluation of the In Vivo Efficacy of Antibody-Drug Conjugates Conjugated to Exatecan

In order to study the antibody-drug conjugates conjugated to Exatecan, the antibody-drug conjugates were prepared by the method of Example 9 and purified by HPLC to obtain hu2F7-Exatecan (compound 34, y=8). After formation of transplanted tumors with MX-1 in mice, the anti-tumor effect of the antibody-drug conjugates of the present invention was evaluated. 5×10⁶ MX-1 cells were injected subcutaneously into immunodeficient nude mice. After 2 weeks, intravenous injection of the antibody-drug conjugate hu2F7-Exatecan (y=8) was performed, at a frequency of once/week and a dose of 5 mg/kg and 10 mg/kg. Human IgG1 protein was used as the control at a dose of 5 mg/kg. There were 5 mice in each group of the control group or the administration group. The tumor inhibition rate was calculated by measuring the tumor volume. Tumor inhibition rate=100%−(tumor volume of the administration group on day 18−tumor volume of the administration group on day 0)/(tumor volume of the control group on day 18−tumor volume of the control group on day 0). The experimental results are as shown in Table 8. hu2F7-Exatecan shows a tumor inhibition rate of more than 100% at the doses of both 5 mg/kg and 10 mg/kg, which means that hu2F7-Exatecan (y=8) can not only inhibit tumor growth, but also exert killing effect on the tumor already formed.

TABLE 8
Efficacy of antibody-drug conjugates conjugated to Exatecan
on MX-1 transplanted tumor in tumor-bearing nude mice

| | Tumor |
|---|---|
| | inhibition |
| Administration group | rate |
| hu2F7-Exatecan (compound 34, y = 8) 5 mg/kg | 173.55% |
| hu2F7-Exatecan (compound 34, y = 8) 10 mg/kg | 183.48% |

### Example 15: Experiment of the Bystander Killing Activity of Antibody-Drug Conjugates Conjugated to Exatecan

In order to study the effect of the antibody-drug conjugates conjugated to Exatecan of the present invention on killing B7-H4 negative cells through the bystander effect, the killing activity of the antibody-drug conjugates on a myeloma cell line with negative B7-H4 expression was detected by using ONE-Glo™ Luciferase Assay detection kit. MX-1 cells were collected, centrifuged, counted, adjusted to a cell suspension of 7.5×10⁶ cells/mL with complete medium and plated onto a white 96-well plate. 33 μL of MX-1 cell suspension was added to each well in the “mixed cell group”, and 33 μL PBS was added to each well in the “single cell group”. NCI-H929-LUC tumor cells with negative B7-H4 expression (Cobioer catalog number CBP30061L) were collected. The cell density was adjusted to 1.5×10⁶ cells/mL. 33 μL of NCI-H929-LUC cell suspension was added to each well in the “mixed cell group” and “single cell group”. The cell plate was placed in a 37° C., 5% CO₂ incubator for culture overnight. On the second day of the experiment, the highest concentration of the antibody-drug conjugate hu2F7-Exatecan (compound 34, y=8) to be tested was uniformly adjusted to 3000 nM, and diluted to obtained a 5-fold gradient of 9 doses. 33.3 μL of the diluted antibody-drug conjugate working solution was added to each well of the experimental plate and mixed gently. The cell plate was placed in a 37° C., 5% incubator and incubated for 48 hours. The cell culture plate was taken out and equilibrated to room temperature. Then 100 μL ONE-Glo™ Luciferase reagent (Promega E6120) was added to each well. The cells were shaken and lysed at room temperature in dark for 10 minutes. The cell lysate was centrifuged at 1000 rpm/min for 1 minute, and then transferred to a 96-well plate with transparent bottom at 180 μL per well. The plate was read at a wavelength of 490 nm on a microplate reader. The data were analyzed using the Graphpad Prism software, and the experimental results are as shown in Table 9. ONE-Glo™ Luciferase reagent can specifically detect the viability of NCI-H929-LUC cells. hu2F7-Exatecan effectively kills NCI-H929-LUC cells in the “mixed cell group”, indicating that in the presence of B7-H4 positive cells, hu2F7-Exatecan has a bystander killing effect, and it can not only kill B7-H4 positive cells, but can also kill the insensitive B7-H4 negative cells (in “single Cell Group”).

TABLE 9
Evalutaion of bystander killing activity

| | | Maximum |
|---|---|---|
| | | inhibition |
| Experimental group | IC₅₀ (nM) | rate |
| Mixed cell group (MX-1:NCI-LUC = 5:1) | 70.34 | 76.8% |
| Single cell group (NCI-H929-LUC) | ND | ND |

ND: No activity detectable.

1. An antibody-drug conjugate represented by general formula (A) or a pharmaceutically acceptable salt or solvate thereof,
Ab-(L₂-L₁-D)_{y} (A)
wherein:
D is a cytotoxic drug;
L₁ and L₂ are linker units;
y is a number of 1 to 20;
Ab is a B7-H4 antibody or antigen-binding fragment thereof, which comprises antibody light chain variable region and antibody heavy chain variable region,
wherein the antibody heavy chain variable region and antibody light chain variable region of the Ab comprises the CDRS of any one selected from the group consisting of the following (1) to (4):
(1) HCDR1 as shown in SEQ ID NO:9, HCDR2 as shown in SEQ ID NO:10 and HCDR3 as shown in SEQ ID NO:11; and
LCDR1 as shown in SEQ ID NO:12, LCDR2 as shown in SEQ ID NO:13 and LCDR3 as shown in SEQ ID NO:14;
(2) HCDR1 as shown in SEQ ID NO:3, HCDR2 as shown in SEQ ID NO:4 and HCDR3 as shown in SEQ ID NO:5; and
LCDR1 as shown in SEQ ID NO:6, LCDR2 as shown in SEQ ID NO:7 and LCDR3 as shown in SEQ ID NO:8; (3) HCDR1 as shown in SEQ ID NO:23, HCDR2 as shown in SEQ ID NO:24 and HCDR3 as shown in SEQ ID NO:25; and
LCDR1 as shown in SEQ ID NO:26, LCDR2 as shown in SEQ ID NO:27 and LCDR3 as shown in SEQ ID NO:28; or,
(4) HCDR1 as shown in SEQ ID NO:29, HCDR2 as shown in SEQ ID NO:30 and HCDR3 as shown in SEQ ID NO:31; and
LCDR1 as shown in SEQ ID NO:32, LCDR2 as shown in SEQ ID NO:33 and LCDR3 as shown in SEQ ID NO:34.

2.-4. (canceled)

5. The antibody-drug conjugate or the pharmaceutically acceptable salt or solvate thereof according to claim 1, wherein the Ab is a murine antibody or fragment thereof, a chimeric antibody or fragment thereof, a human antibody or fragment thereof, and a humanized antibody or fragment thereof.

6. The antibody-drug conjugate or the pharmaceutically acceptable salt or solvate thereof according to claim 1, wherein the Ab further comprises any one of (a), (b) and (c) or combination thereof:
(a) light chain framework region(s) and heavy chain framework region(s) derived from human germline light chain and heavy chain sequences or mutant sequence(s) thereof;
(b) heavy chain constant region(s) derived from human IgG1 or variant thereof, IgG2 or variant thereof, IgG3 or variant thereof or IgG4 or variant thereof, preferably heavy chain constant region(s) derived from human IgG1, IgG2 or IgG4, more preferably heavy chain constant region(s) of IgG1 with enhanced ADCC toxicity after amino acid mutation, most preferably the heavy chain constant region as shown in SEQ ID NO: 54;
(c) light chain constant region(s) derived from human κ chain, λ chain or variant thereof, preferably light chain constant region(s) derived from human κ chain, more preferably the light chain constant region as shown in SEQ ID NO:55.

7.-10. (canceled)

11. The antibody-drug conjugate or the pharmaceutically acceptable salt or solvate thereof according to claim 1, wherein the heavy chain variable region and light chain variable region of the Ab is any one selected from the group consisting of the following:
(1) the heavy chain variable region as shown in SEQ ID NO: 17 and the light chain variable region as shown in SEQ ID NO: 18;
(2) the heavy chain variable region as shown in SEQ ID NO: 15 and the light chain variable region as shown in SEQ ID NO: 16;
(3) the heavy chain variable region as shown in SEQ ID NO: 35 and the light chain variable region as shown in SEQ ID NO: 36; or,
(4) the heavy chain variable region as shown in SEQ ID NO: 37 and the light chain variable region as shown in SEQ ID NO: 38.

12. The antibody-drug conjugate or the pharmaceutically acceptable salt or solvate thereof according to claim 11, wherein the Ab is any one selected from the group consisting of the following:
(1) the light chain as shown in SEQ ID NO: 22 and the heavy chain as shown in SEQ ID NO: 21;
(2) the light chain as shown in SEQ ID NO: 20 and the heavy chain as shown in SEQ ID NO: 19;
(3) the light chain as shown in SEQ ID NO: 40 and the heavy chain as shown in SEQ ID NO: 39; or,
(4) the light chain as shown in SEQ ID NO: 42 and the heavy chain as shown in SEQ ID NO: 41.

13. The antibody-drug conjugate or the pharmaceutically acceptable salt or solvate thereof according to claim 1, wherein the antigen-binding fragment is selected from the group consisting of Fab, Fab′, F(ab′)₂, single-chain antibody, dimerized V region, disulfide bond stabilized V region and antigen-binding fragments of a peptide comprising CDRs.

14. The antibody-drug conjugate or the pharmaceutically acceptable salt or solvate thereof according to claim 1, wherein the cytotoxic drug is selected from the group consisting of toxin, chemotherapeutic, antibiotic, radioisotope and nucleolytic enzyme.

15. The antibody-drug conjugate or the pharmaceutically acceptable salt or solvate thereof according to claim 14, wherein the cytotoxic drug is selected from the group consisting of tubulin inhibitor, DNA topoisomerase inhibitor that inhibits cell division or camptothecin derivatives.

16. The antibody-drug conjugate or the pharmaceutically acceptable salt or solvate thereof according to claim 15, wherein the cytotoxic drug is selected from DM1, DM3, DM4, SN-38, MMAF, or Exatecan.

17. (canceled)

18. The antibody-drug conjugate or the pharmaceutically acceptable salt or solvate thereof according to claim 1, the antibody-drug conjugate is as shown in general formula; selected from the group consisting of (I), (II) and (III); 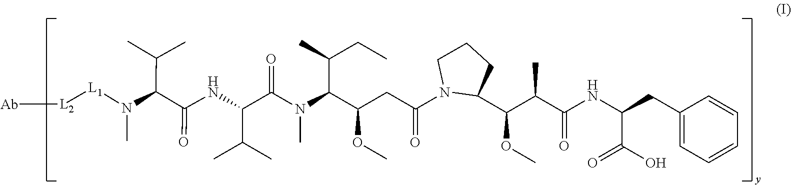
wherein:
L₁ and L₂ are linker units;
y is a number selected from 1 to 8;
Ab is the B7-H4 antibody or antigen-binding fragment thereof according to claim 1, or 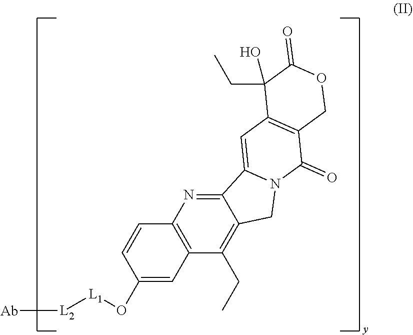
wherein:
L₁ and L₂ are linker units;
y is a number selected from 1 to 8,
Ab is the B7-H4 antibody or antigen-binding fragment thereof according to claim 1; or 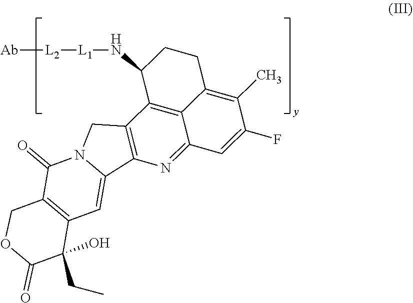
wherein:
L₁ and L₂ are linker units;
y is a number selected from 1 to 10;
Ab is the B7-H4 antibody or antigen-binding fragment thereof according to claim 1.

19.-41. (canceled)

42. The antibody-drug conjugate or the pharmaceutically acceptable salt or solvate thereof according to claim 18, the antibody-drug conjugate is as shown in general formula selected from the group consisting of (I), (II) and (III): 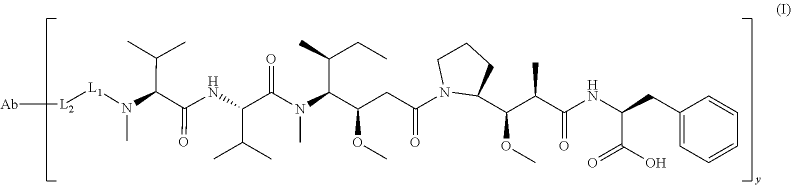
wherein:
y is a number selected from 2 to 4;
or 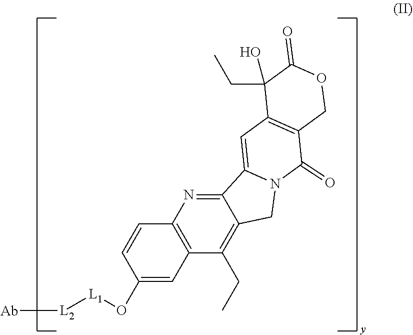
wherein:
y is a number selected from 2 to 4;
or 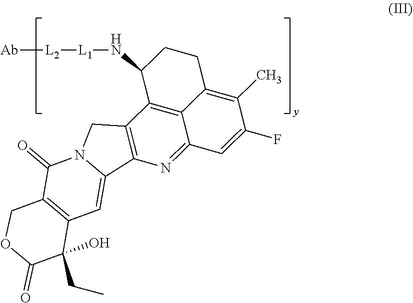
wherein:
y is a number selected from 2 to 8.

43. The antibody-drug conjugate or the pharmaceutically acceptable salt or solvate thereof according to claim 42, the antibody-drug conjugate is as shown in general formula selected from the group consisting of (III): 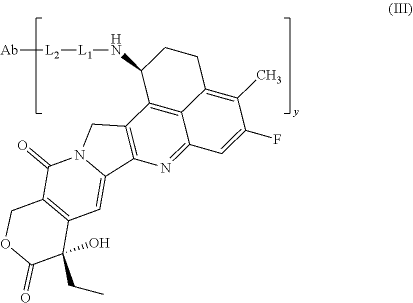
wherein:
y is a number of 4 to 8.

44. The antibody-drug conjugate or the pharmaceutically acceptable salt or solvate thereof according to claim 43, the antibody-drug conjugate is as shown in general formula selected from the group consisting of (III): 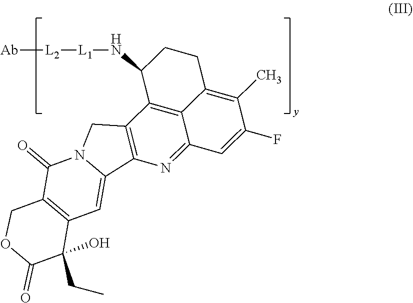
wherein:
y is a number of 6 to 8.

45. The antibody-drug conjugate or the pharmaceutically acceptable salt or solvate thereof according to claim 44, the antibody-drug conjugate is as shown in general formula selected from the group consisting of (III): 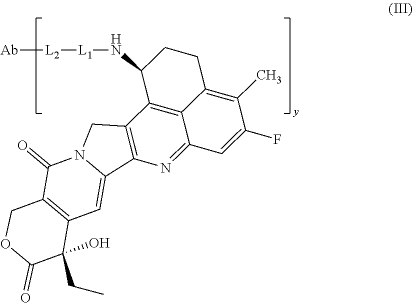
wherein:
y is 8.

46. The antibody-drug conjugate or the pharmaceutically acceptable salt or solvate thereof according to claim 18, wherein L₁ is as shown in general formula (B): 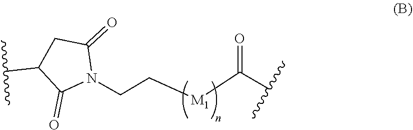
wherein:
M₁ is —CR₁R₂—;
R₁ and R₂ are the same or different, and are independently selected from the group consisting of hydrogen, alkyl, halogen, hydroxyl and amino;
n is an integer of 0 to 5;
or
wherein L₂ is as shown in general formula (C): 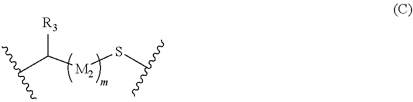
wherein:
M₂ is —CR₄R₅—;
R₃ is selected from the group consisting of hydrogen, halogen, hydroxyl, amino, alkyl, alkoxyl and cycloalkyl:
R₄ and R₅ are the same or different, and are independently selected from the group consisting of hydrogen, alkyl, halogen, hydroxyl and amino;
m is an integer of 0 to 5;
or
wherein L₂ is as shown in general formula (D):
-K¹-K²-K³-K⁴- (D)
wherein:
K¹ is 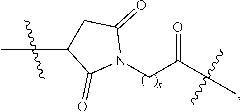
s is an integer of 2 to 8;
K² is selected from the group consisting of —NR¹(CH₂CH₂O)ₚCH₂CH₂C(O)—, —NR¹(CH₂CH₂O)ₚCH₂C(O)—, —S(CH₂)ₚC(O)— and single bond, p is an integer of 1 to 20;
R₁ is selected from the group consisting of hydrogen, deuterium, hydroxyl, amino, alkyl, halogen, haloalkyl, deuterated alkyl and hydroxyalkyl;
K³ is a tetrapeptide residue;
K⁴ is —NR²(CR³R⁴)ₜ—, R², R³ or R⁴ are each independently hydrogen, deuterium, hydroxyl, amino, alkyl, halogen, haloalkyl, deuterated alkyl and hydroxyalkyl, and t is 1 or 2;
or
wherein L₁ is selected from the group consisting of —O—(CR^{a}R^{b})ₘ—CR⁵R⁶—C(O)—, —O—CR⁵R⁶—(CR^{a}R^{b})ₘ—, —O—CR⁵R⁶—, —NH—(CR^{a}R^{b})ₘ—CR⁵R⁶—C(O)— and —S—(CR^{a}R^{b})ₘ—CR⁵ R₆—C(O)—;
R^{a} and R^{b} are each independently selected from the group consisting of hydrogen, deuterium, halogen and alkyl;
R⁵ is haloalkyl or cycloalkyl;
R⁶ is selected from the group consisting of hydrogen, haloalkyl and cycloalkyl;
or, R⁵ and R⁶ and the carbon atom to which they are linked form a cycloalkyl;
m is 0, 1, 2, 3 or 4.

47. The antibody-drug conjugate or the pharmaceutically acceptable salt or solvate thereof according to claim 46, wherein:
L₁ is as shown in general formula (B): 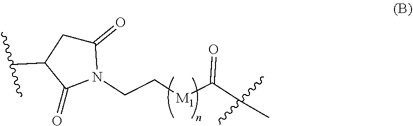
wherein, n is 1, 2 or 3;
or
wherein L₂ is as shown in general formula (C): 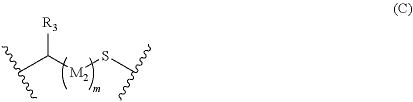
wherein: m is 1, 2 or 3;
or
L₂ is as shown in general formula (D):
-K¹-K²-K³-K⁴- (D)
wherein:
K² is selected from the group consisting of —NR¹(CH₂CH₂O)ₚCH₂CH₂C(O)—, —NR¹(CH₂CH₂O)ₚCH₂C(O)—, —S(CH₂)ₚC(O)— and single bond, p is an integer of 1 to 6;
K³ is a peptide residue formed by amino acids selected from the group consisting of two or more of phenylalanine, glycine, valine, lysine, citrulline, serine, glutamate and aspartate;
the K¹ terminus of the linker unit -L₂- is linked to the Ab, and the K⁴ terminus is linked to L₁;
or
the O terminus of L₁ is linked to the linker unit L₂;
or
the L₁ is as shown in general formula (E): 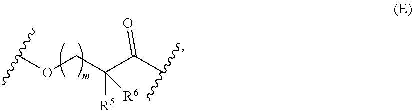
R⁵ is selected from the group consisting of haloalkyl and cycloalkyl,
R⁶ is selected from the group consisting of hydrogen, haloalkyl and cycloalkyl,
or, R⁵ and R⁶ and the carbon atom to which they are linked form a cycloalkyl.

48. The antibody-drug conjugate or the pharmaceutically acceptable salt or solvate thereof according to claim 47, wherein L₂ is as shown in general formula (D):
-K¹-K²-K³-K⁴- (D)
wherein K³ is tetrapeptide residue GGFG;
or
the L₁ is as shown in general formula (E): 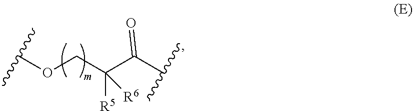
R⁵ is selected from the group consisting of C₁₋₆ haloalkyl and C₃₋₆ cycloalkyl,
R⁶ is selected from the group consisting of hydrogen, C₁₋₆ haloalkyl and C₃₋₆ cycloalkyl,
or, R⁵ and R⁶ and the carbon atom to which they are linked form a C₃₋₆ cycloalkyl;
m is an integer of 0 to 4.

49. The antibody-drug conjugate or the pharmaceutically acceptable salt or solvate thereof according to claim 48, wherein the L₁ is as shown in general formula (E): 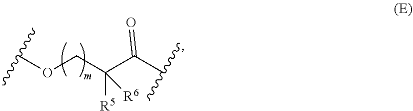
general formula (E) is selected from the group consisting of the following substituents:

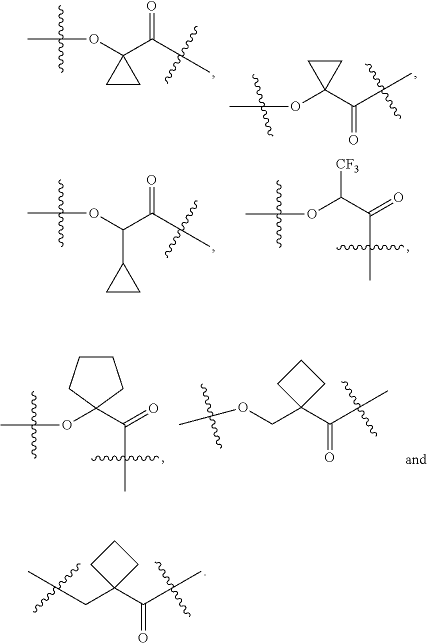

50. The antibody-drug conjugate or the pharmaceutically acceptable salt or solvate thereof according to claim 18, wherein the -L₂-L₁- is as shown in the following structure: 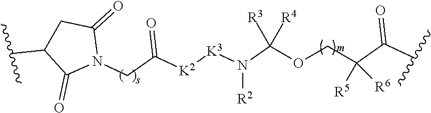
K² is a bond;
K³ is the tetrapeptide residue GGFG;
R⁵ is selected from the group consisting of haloalkyl and C₃₋₆ cycloalkyl;
R⁶ is selected from the group consisting of hydrogen, haloalkyl and C₃₋₆ cycloalkyl;
or, R⁵ and R⁶ and the carbon atom) to which they are linked form a C₃₋₆ cycloalkyl;
R², R³ or R⁴ are each independently hydrogen or alkyl;
s is an integer of 2 to 8;
m is an integer of 0 to 4.

51. The antibody-drug conjugate or the pharmaceutically acceptable salt or solvate thereof according to claim 50, wherein the -L₂-L₁- is selected from the group consisting of the following structures:

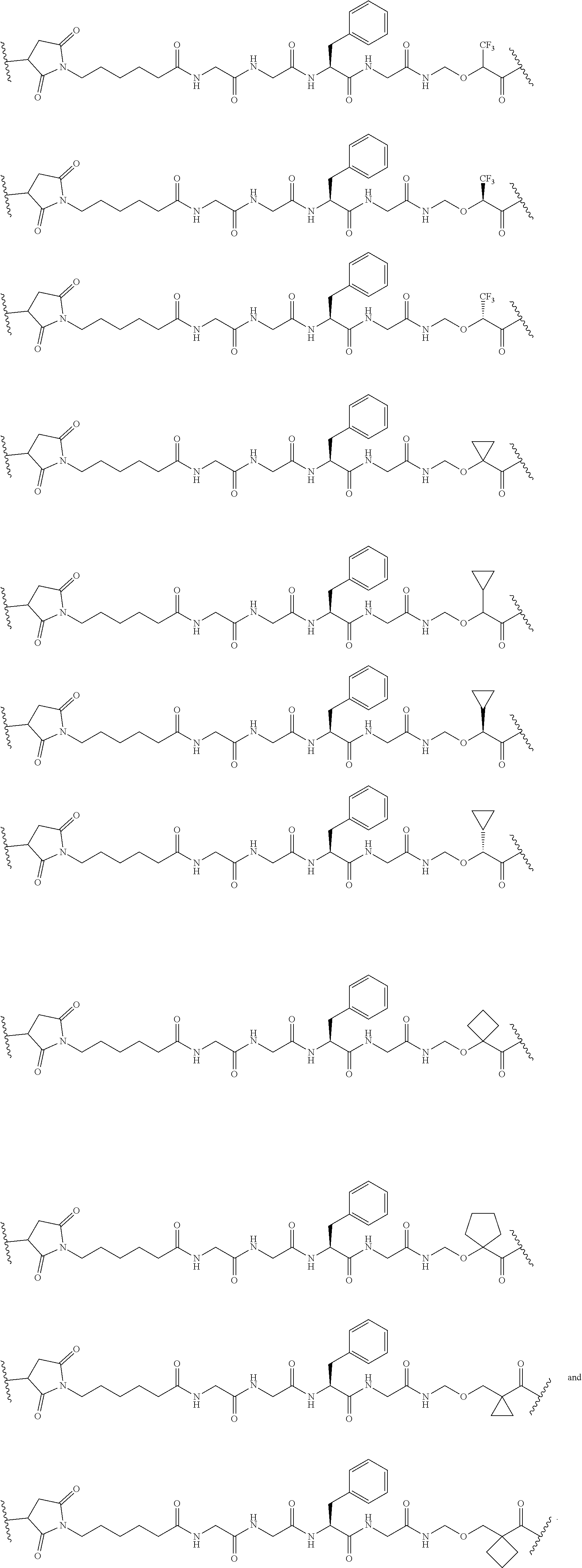

52. The antibody-drug conjugate or the pharmaceutically acceptable salt or solvate thereof according to claim 1, wherein the antibody-drug conjugate is as shown in general formula (IV): 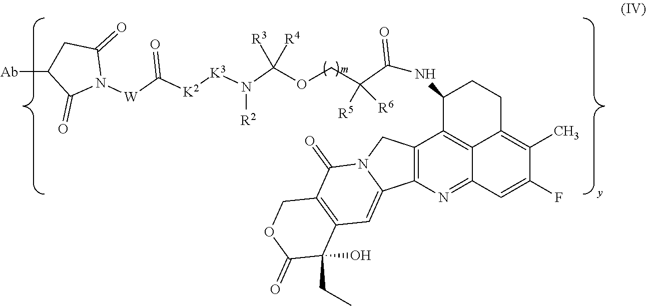
wherein:
W is selected from the group consisting of C₁₋₈ alkyl, C₁₋₈ alkyl-cycloalkyl and linear heteroalkyl of 1 to 8 atoms, the heteroalkyl comprises 1 to 3 heteroatom(s) selected from the group consisting of N, O and S, wherein the C₁₋₈ alkyl, cycloalkyl and linear heteroalkyl are optionally further substituted with one or more substituents selected from the group consisting of halogen, hydroxyl, cyano, amino, alkyl, chloroalkyl, deuterated alkyl, alkoxyl and cycloalkyl;
K₂ is selected from the group consisting of —NR¹(CH₂CH₂O)ₚ₁CH₂CH₂C(O)—, —NR¹(CH₂CH₂O)ₚ₁CH₂C(O)—, —S(CH₂)ₚ₁C(O)— or bond, R¹ is selected from the group consisting of hydrogen atom, alkyl, haloalkyl, deuterated alkyl and hydroxyalkyl, and p₁ is an integer of 1 to 20;
K³ is a peptide residue consisting of 2 to 7 amino acids, the amino acid(s) can be substituted or unsubstituted; if being substituted, the substituent(s) can be substituted at any available attachment point, and the substituent(s) is/are one or more independently selected from the group consisting of halogen, hydroxyl, cyano, amino, alkyl, chloroalkyl, deuterated alkyl, alkoxyl and cycloalkyl;
R² is independently selected from the group consisting of hydrogen atom, alkyl, haloalkyl, deuterated alkyl and hydroxyalkyl;
R³ and R⁴ are each independently selected from the group consisting of hydrogen atom, halogen, alkyl, haloalkyl, deuterated alkyl and hydroxyalkyl;
R⁵ is selected from the group consisting of halogen, haloalkyl, deuterated alkyl, cycloalkyl, heterocyclyl, aryl and heteroaryl;
R⁶ is selected from the group consisting of hydrogen atom, halogen, haloalkyl, deuterated alkyl, cycloalkyl, heterocyclyl, aryl and heteroaryl;
or, R⁵ and R⁶ and the carbon atom to which they are linked form a cycloalkyl or heterocyclyl;
m is an integer of 0 to 4;
y is 1 to 10, y is a decimal or an integer;
Ab is an anti-B7-H4 antibody or antigen-binding fragment thereof.

53. The antibody-drug conjugate or the pharmaceutically acceptable salt or solvate thereof according to claim 52, wherein the antibody-drug conjugate is as shown in general formula (IV-A):

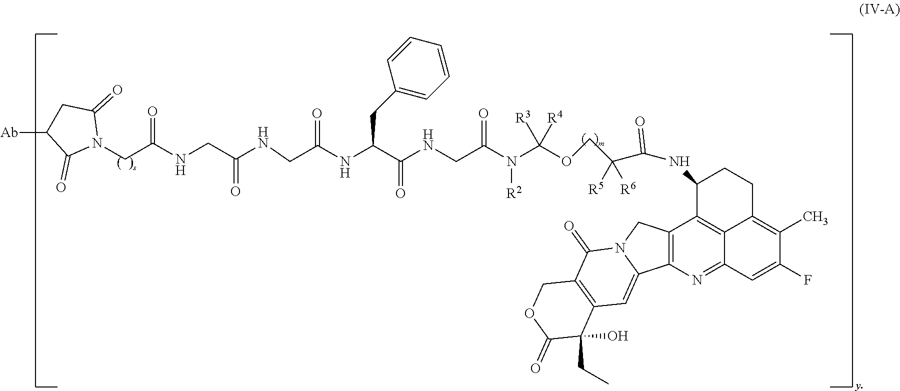

54. The antibody-drug conjugate or the pharmaceutically acceptable salt or solvate thereof according to claim 53, wherein the antibody-drug conjugate is shown as follows:

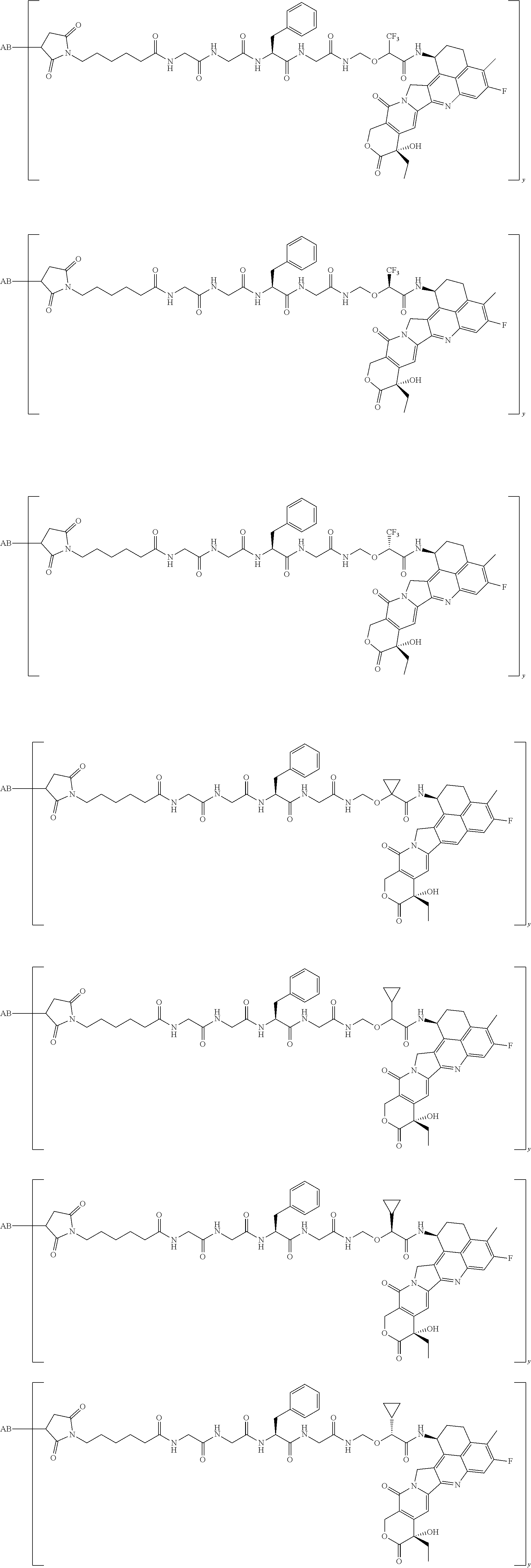

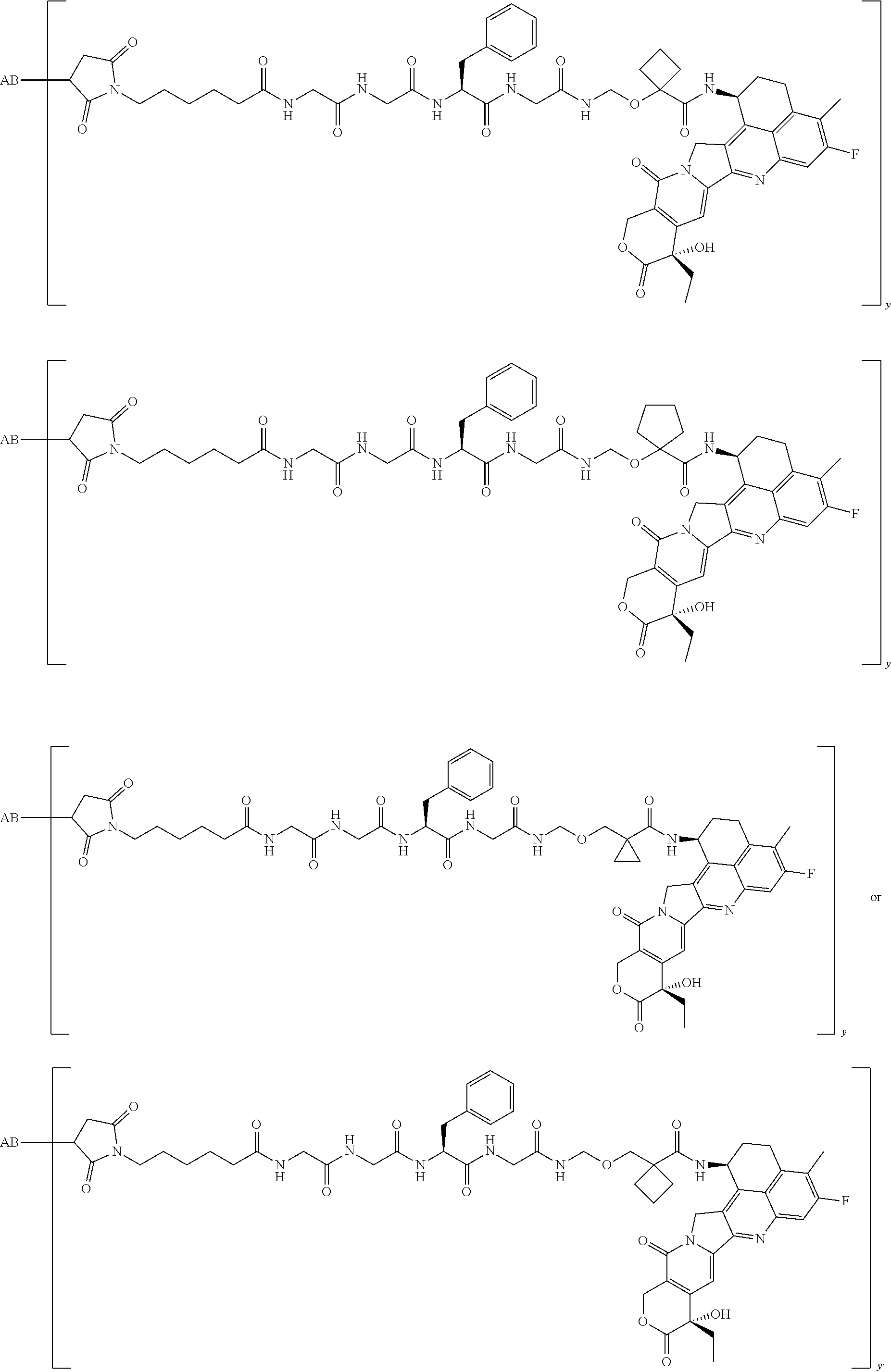

55. The antibody-drug conjugate of general formula (I) or (II) or the pharmaceutically acceptable salt or solvate thereof according to claim 18, the antibody-drug conjugate is as shown in general formula (I-A): 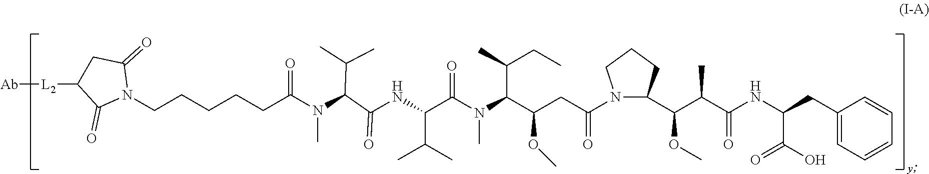
or, the antibody-drug conjugate is as shown in general formula (I-B): 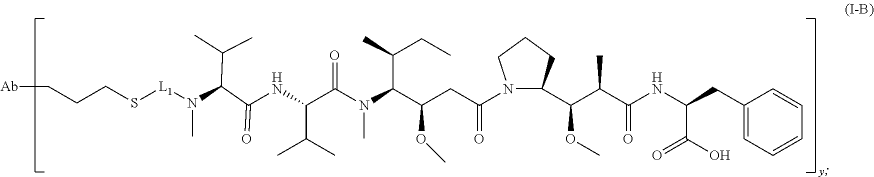
or, the antibody-drug conjugate is as shown in general formula (II-A): 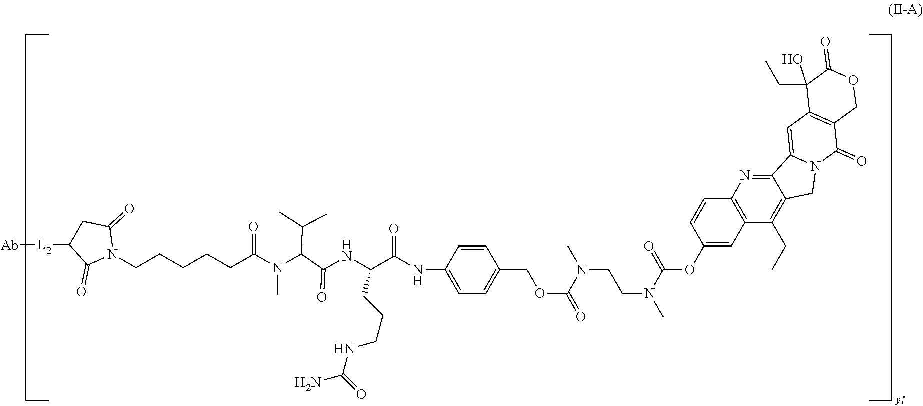
or, the antibody-drug conjugate is as shown in general formula (II-B):

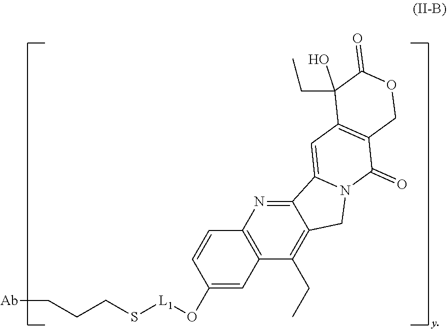

56. The antibody-drug conjugate of general formula (A) or the pharmaceutically acceptable salt or solvate thereof according to claim 18, the antibody-drug conjugate is selected from the group consisting of the following compounds: 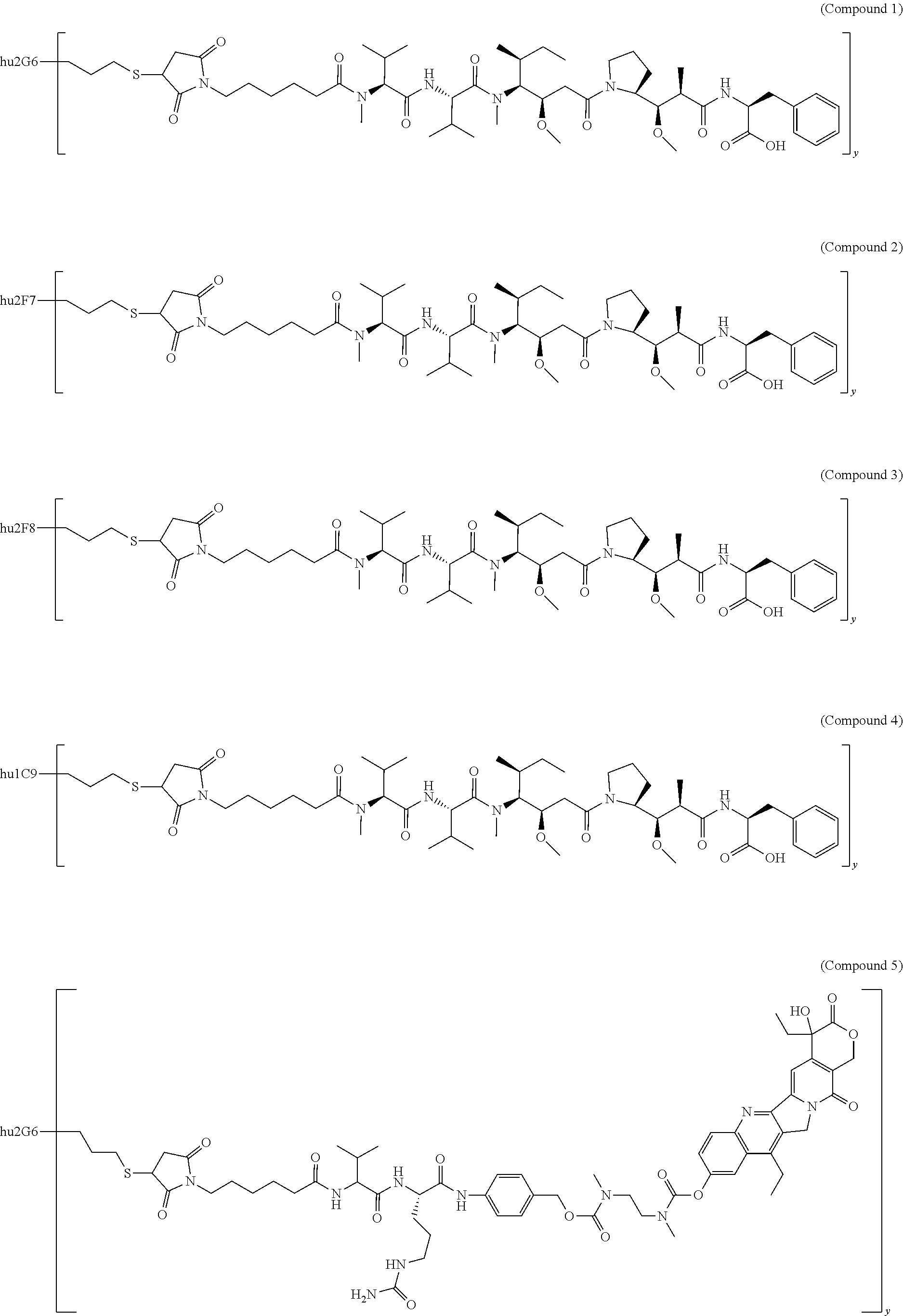 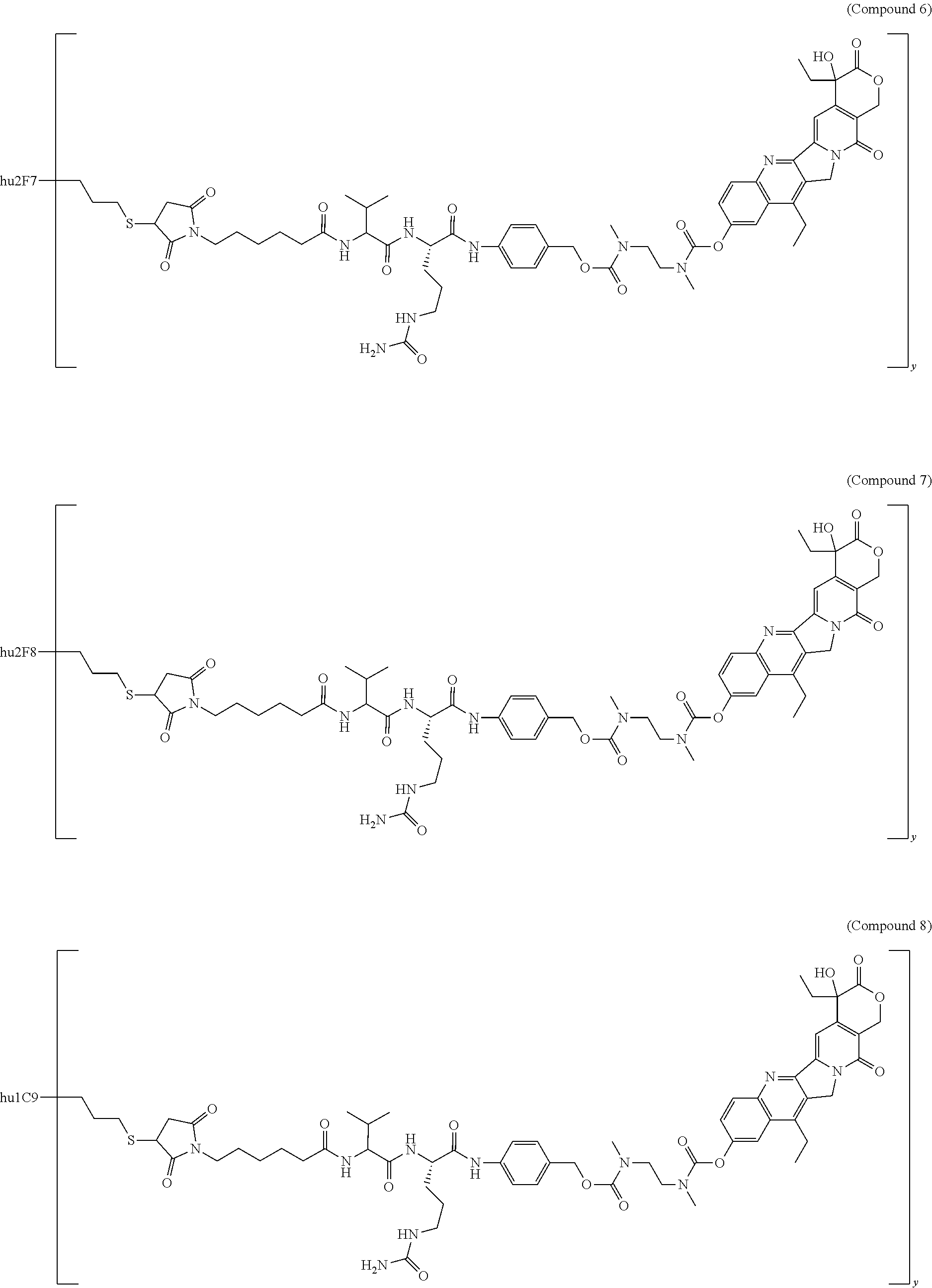 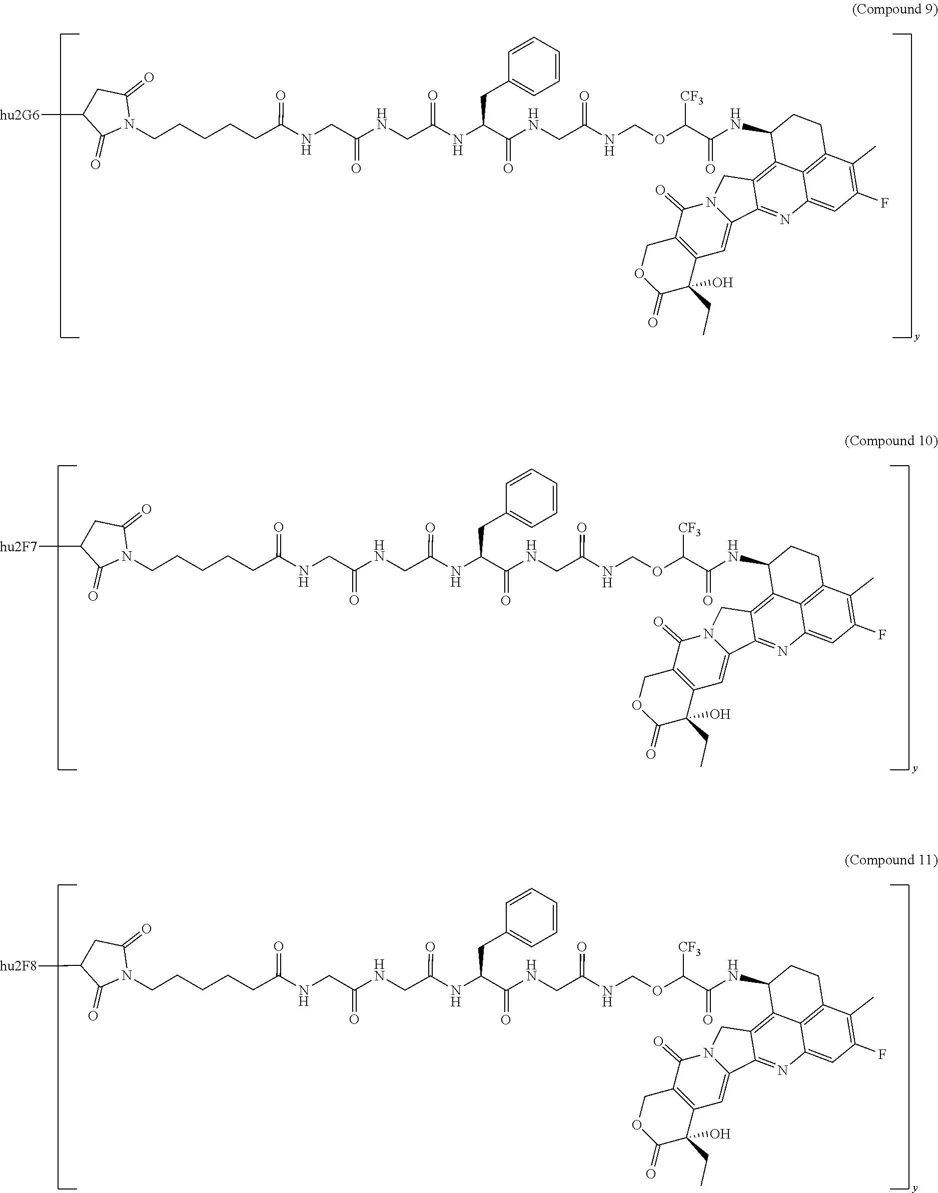 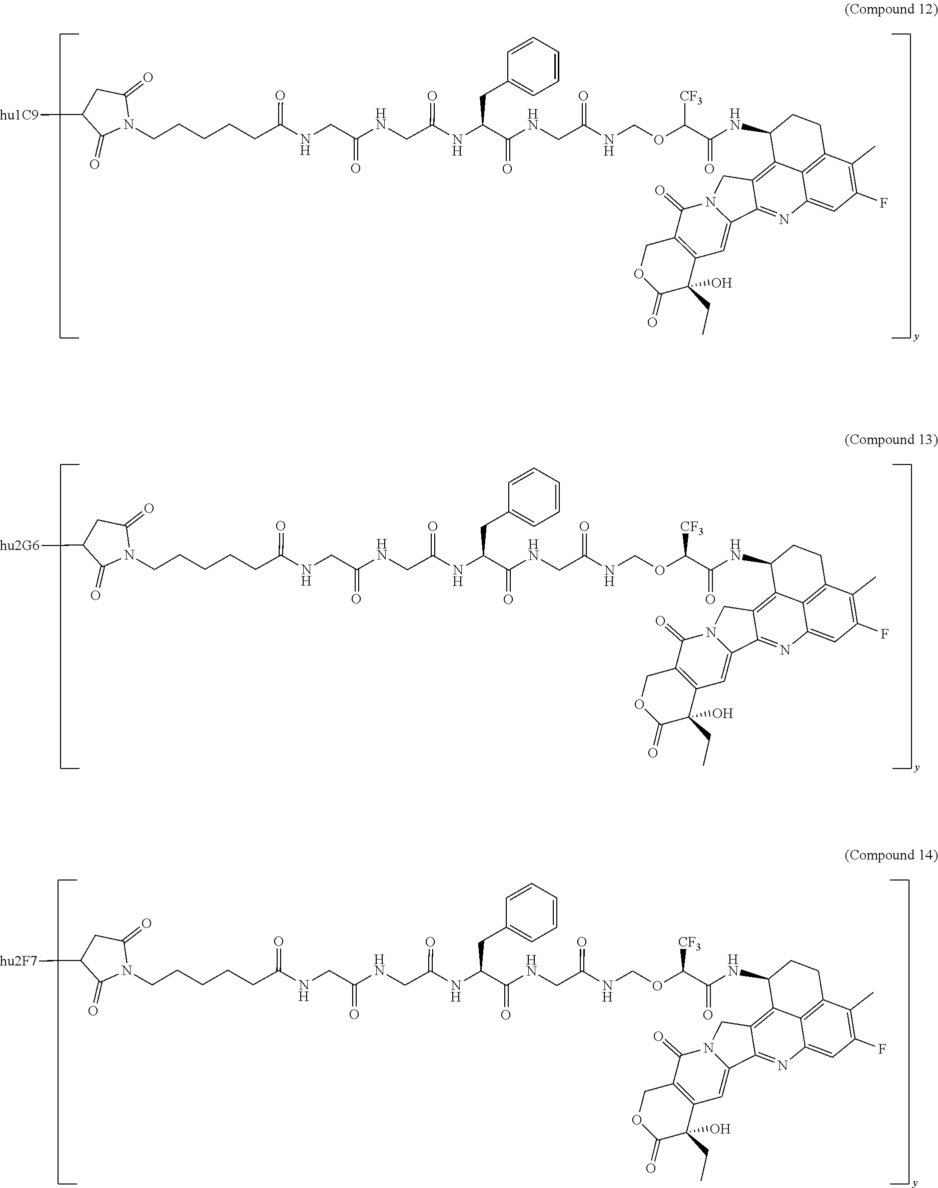 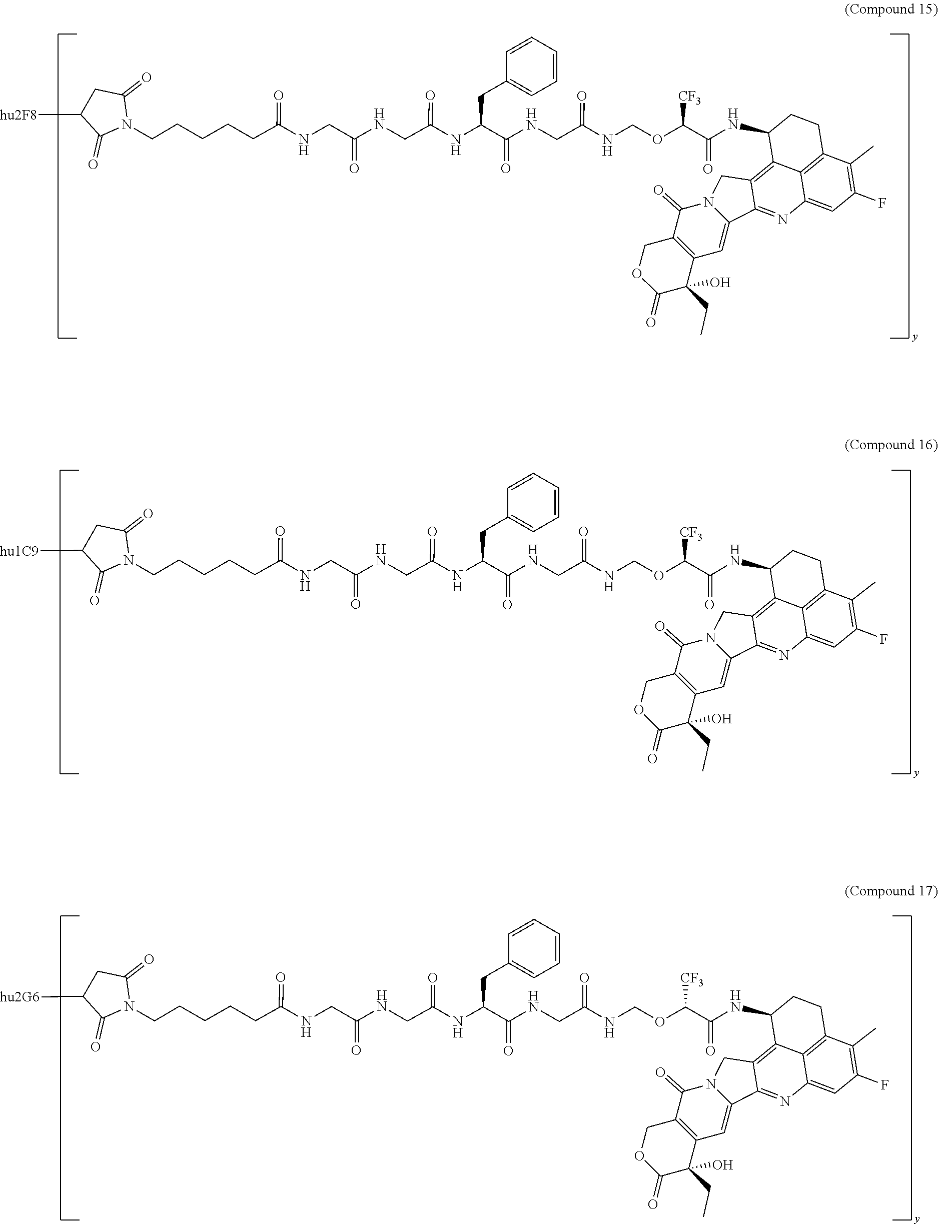 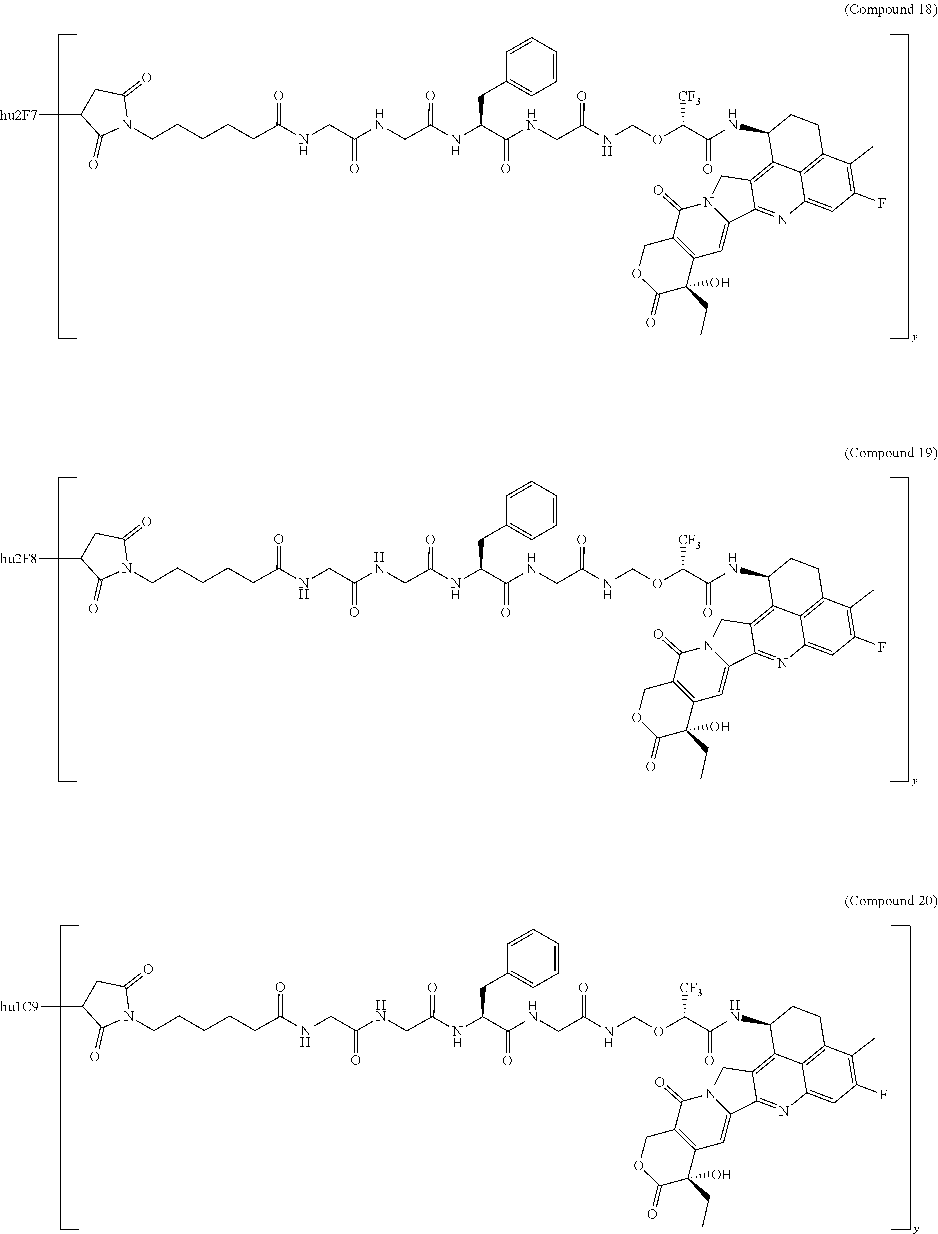 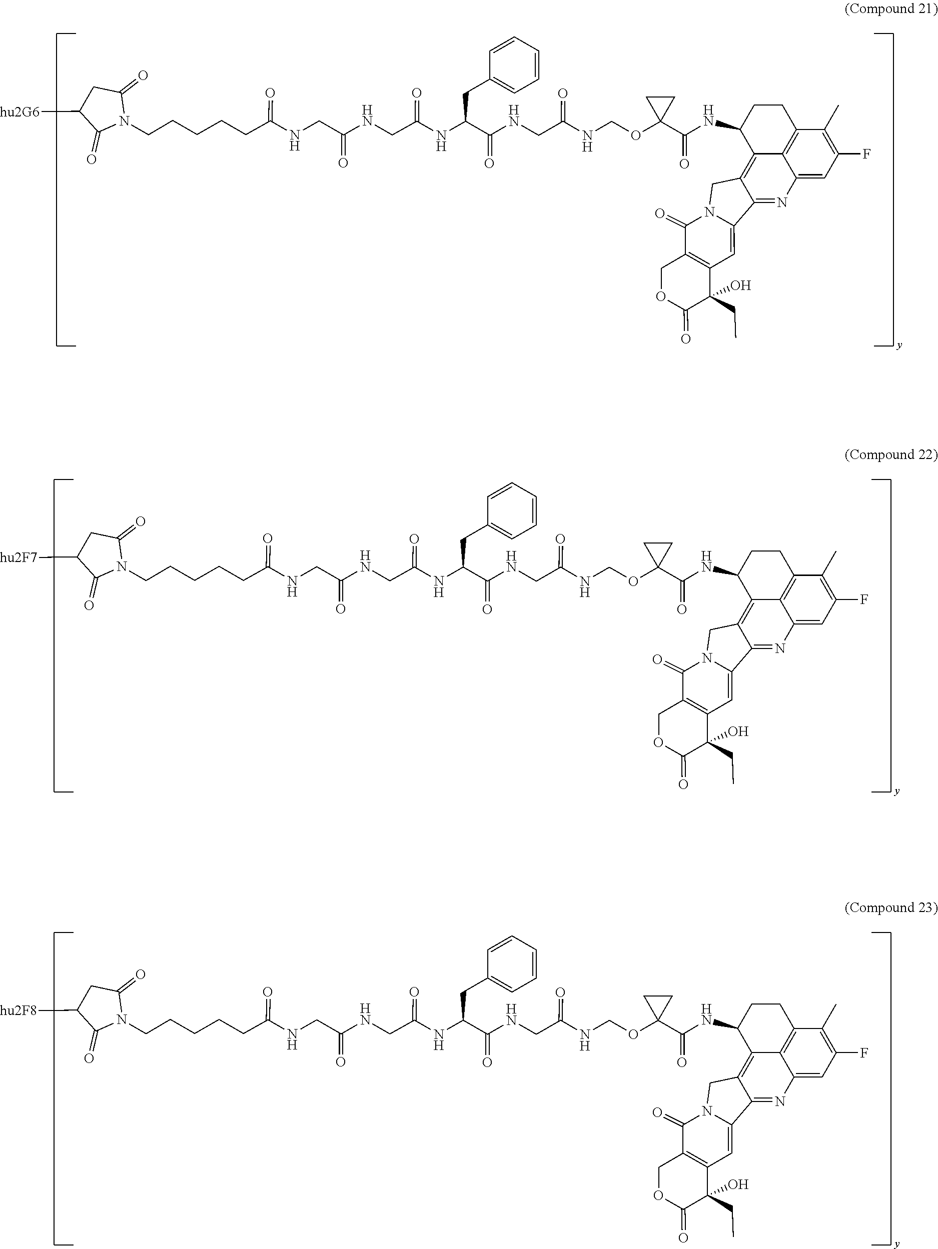 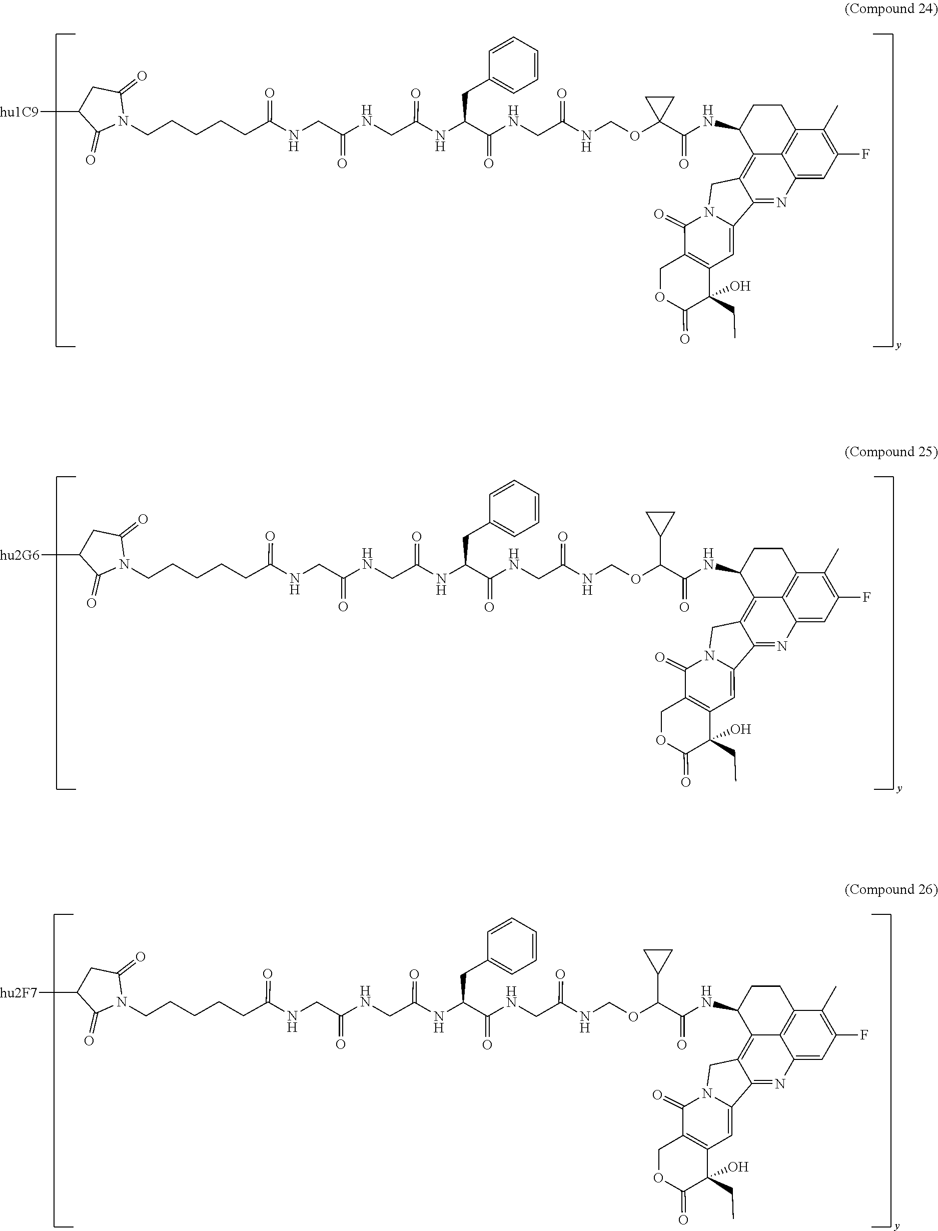 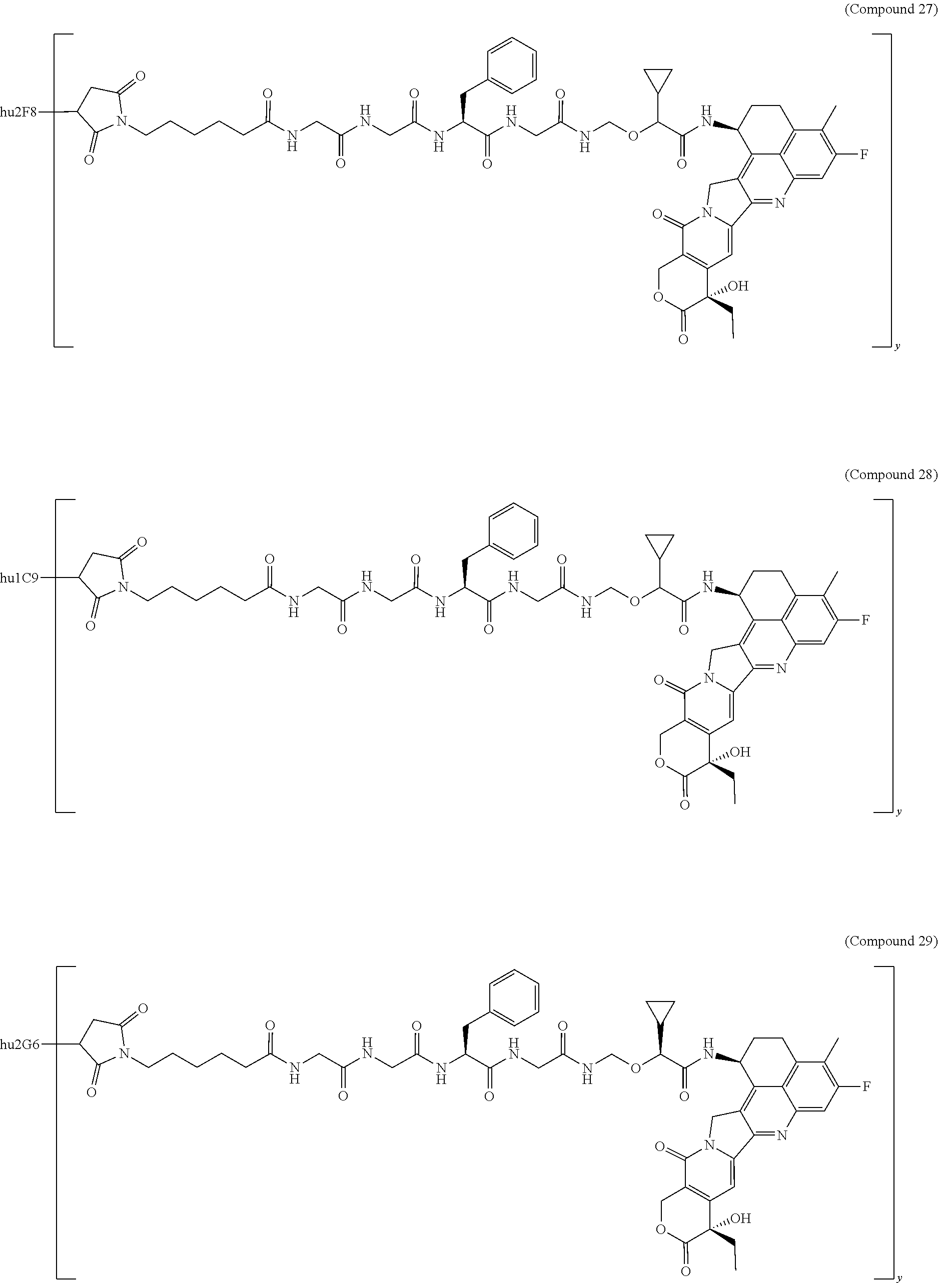 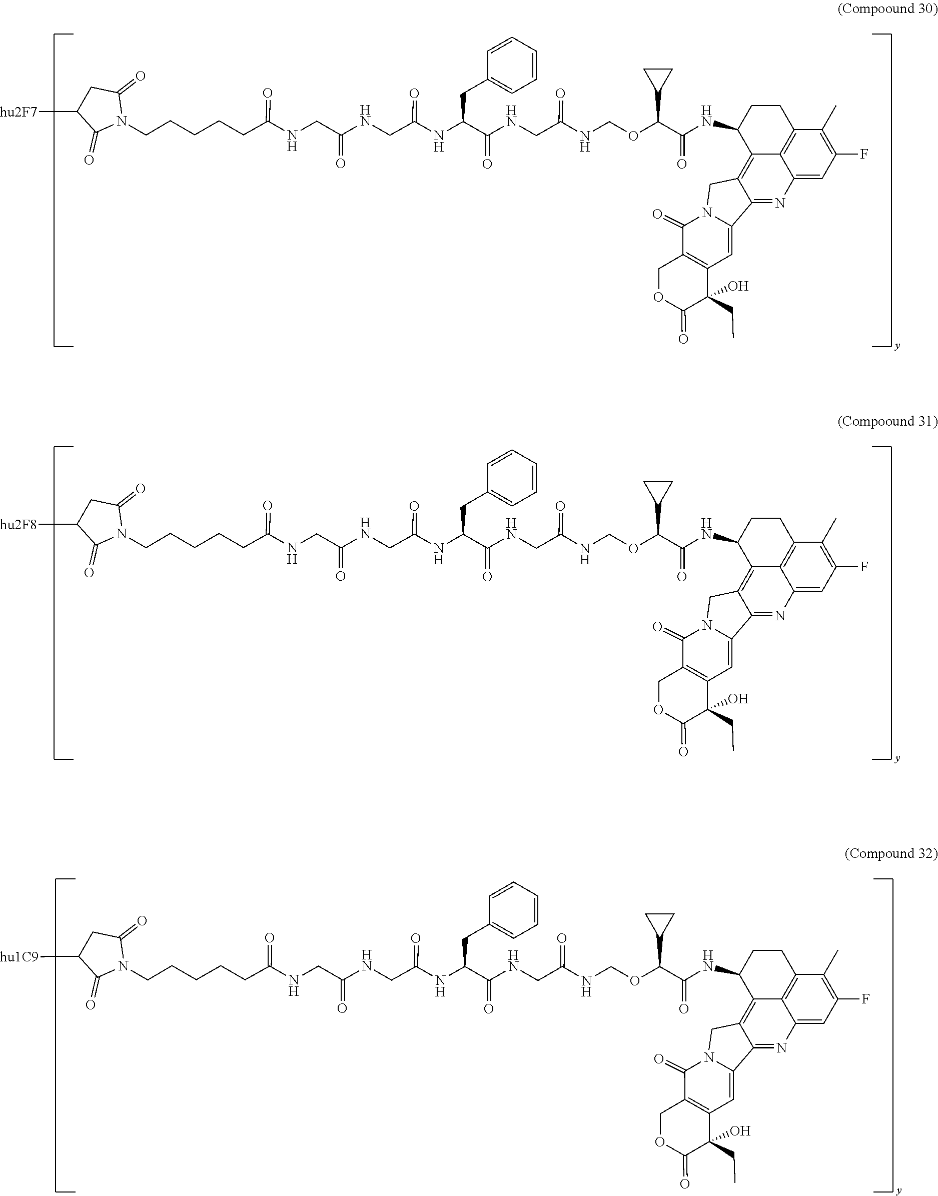 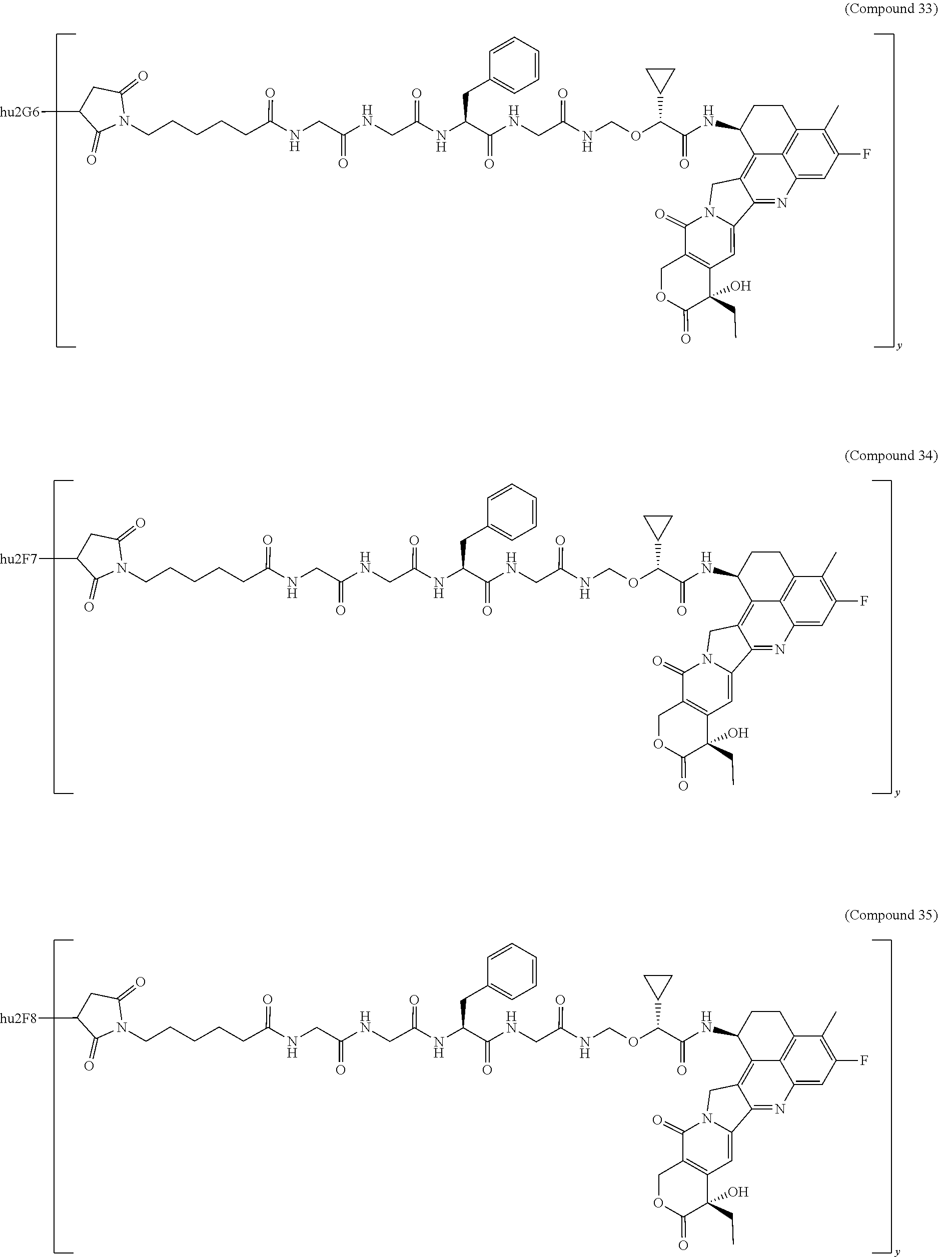 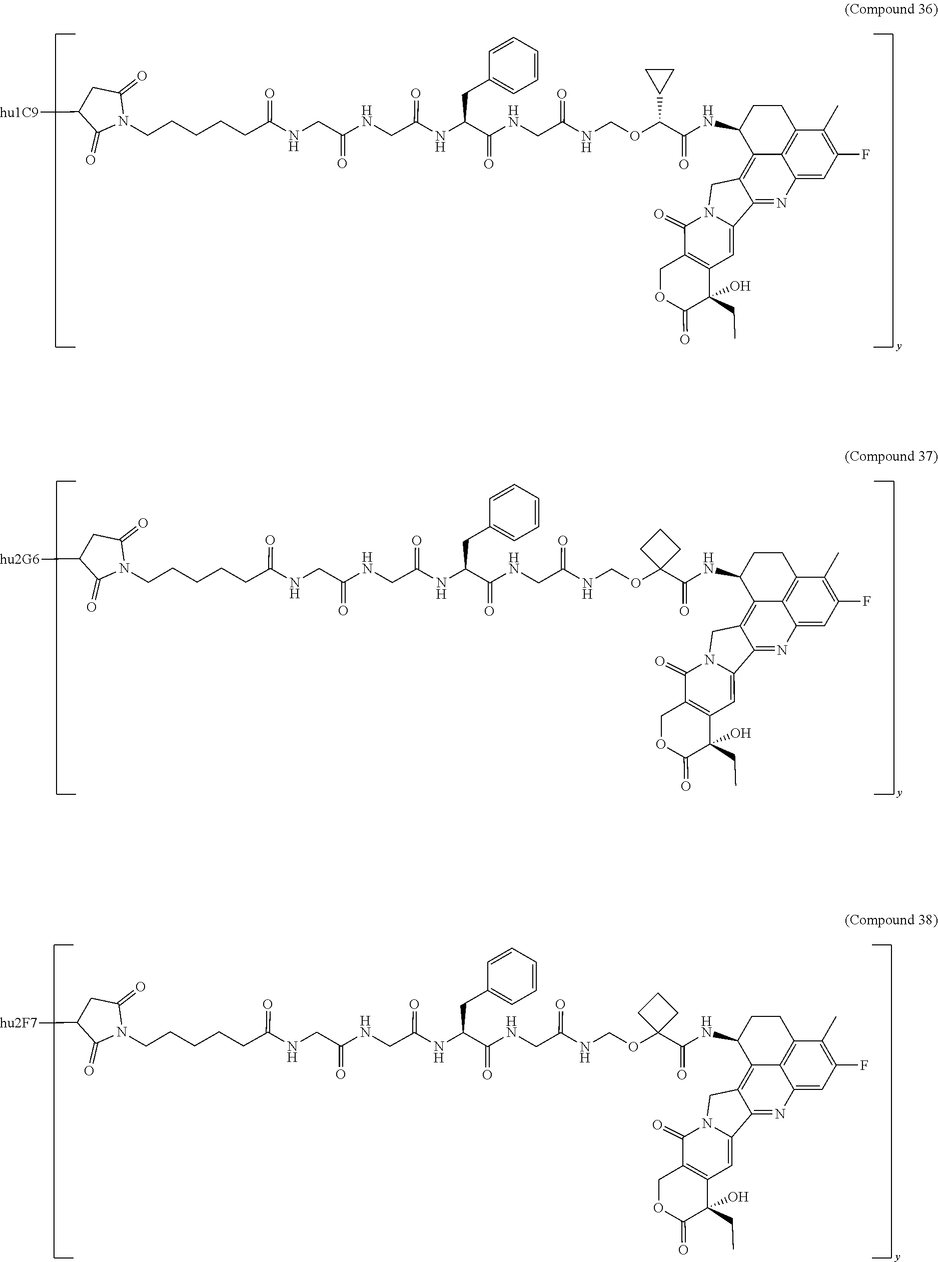 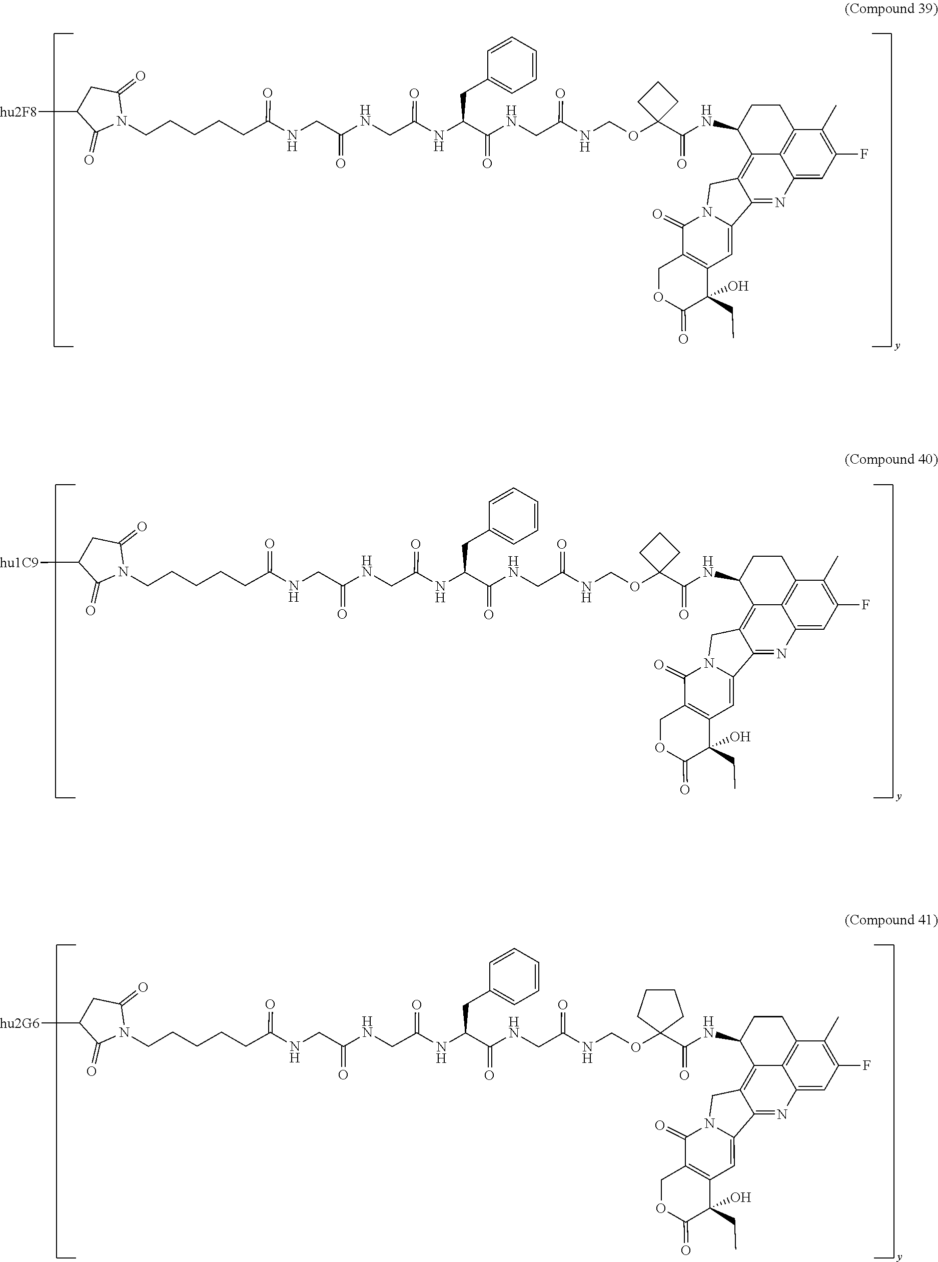 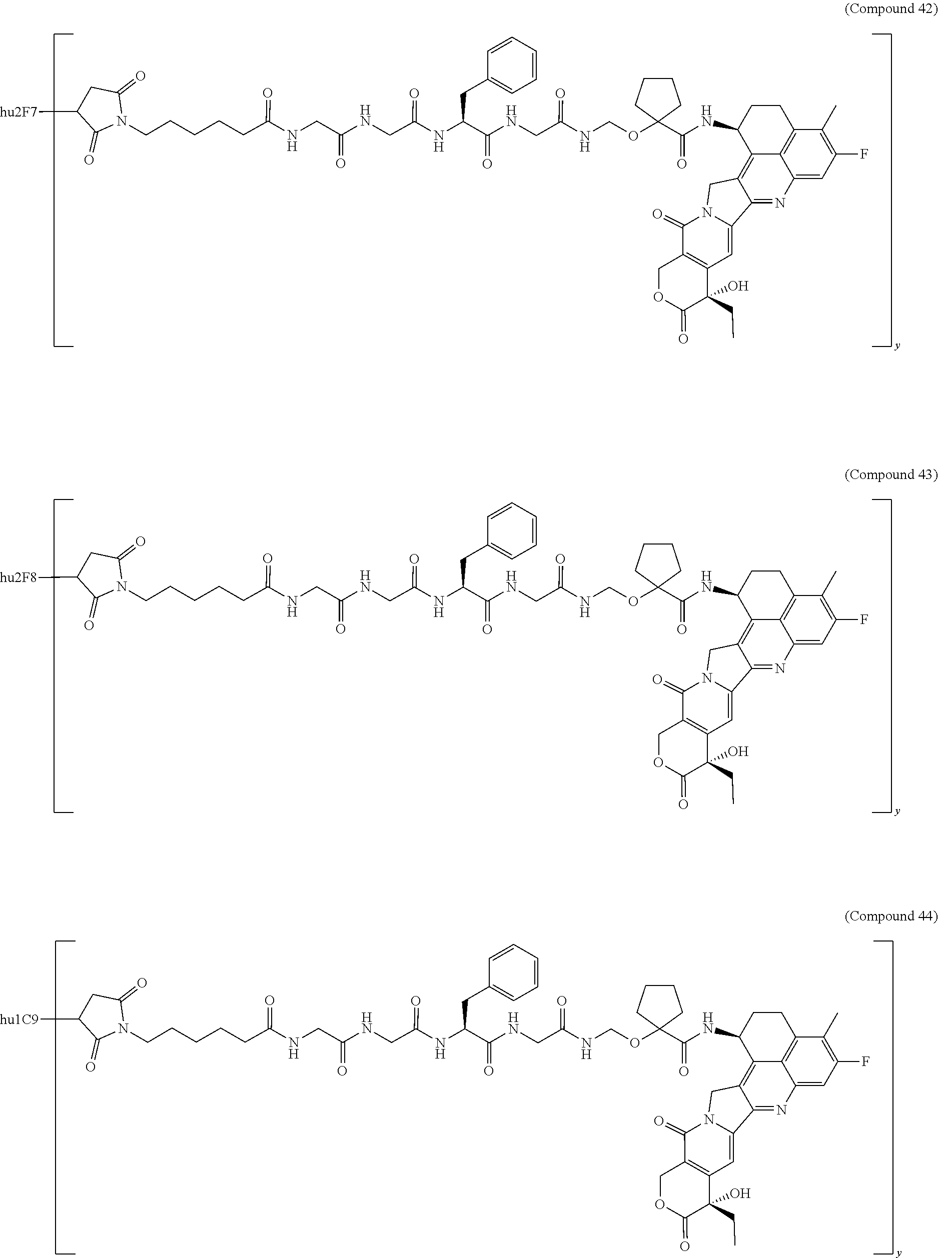 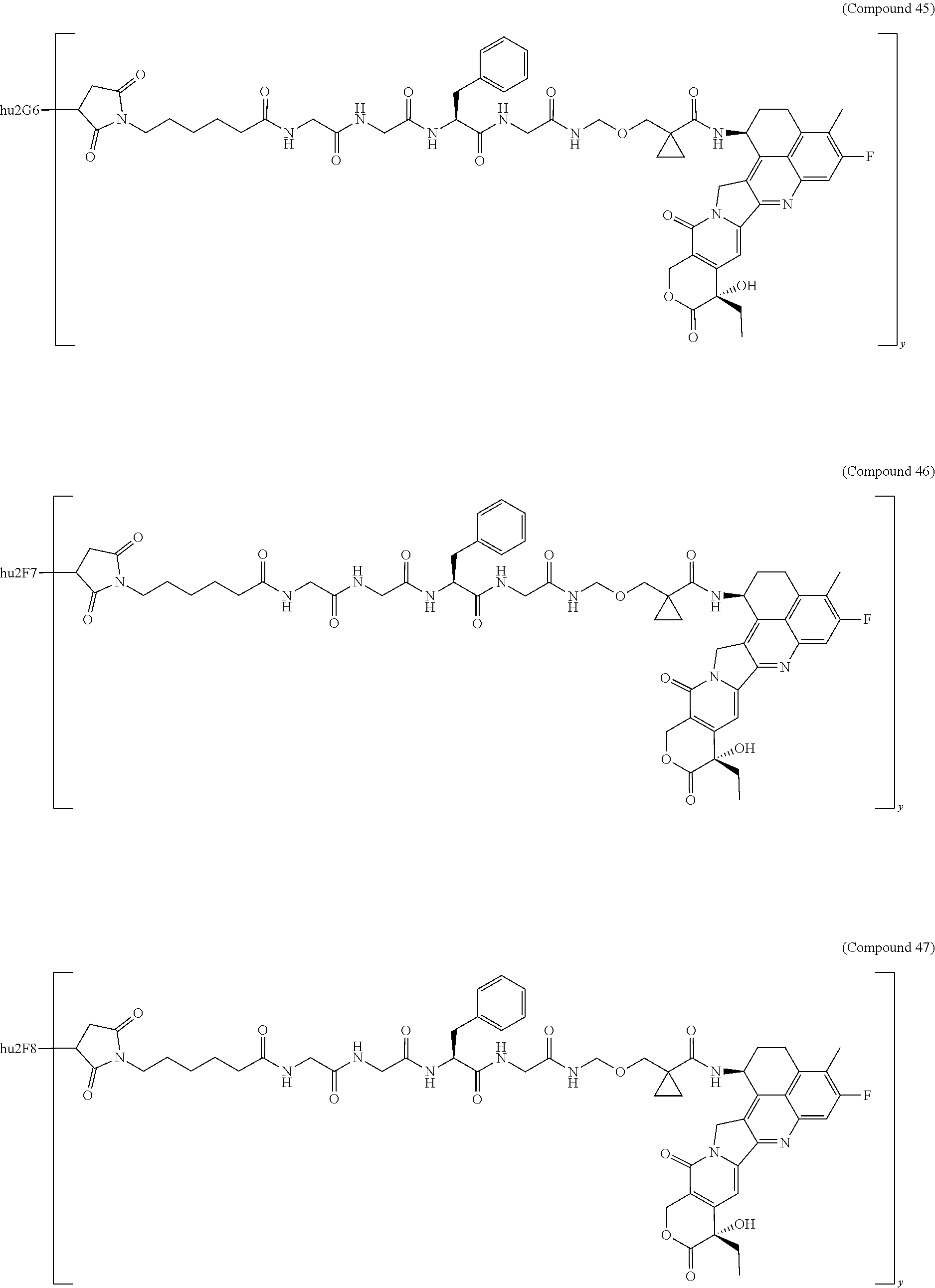 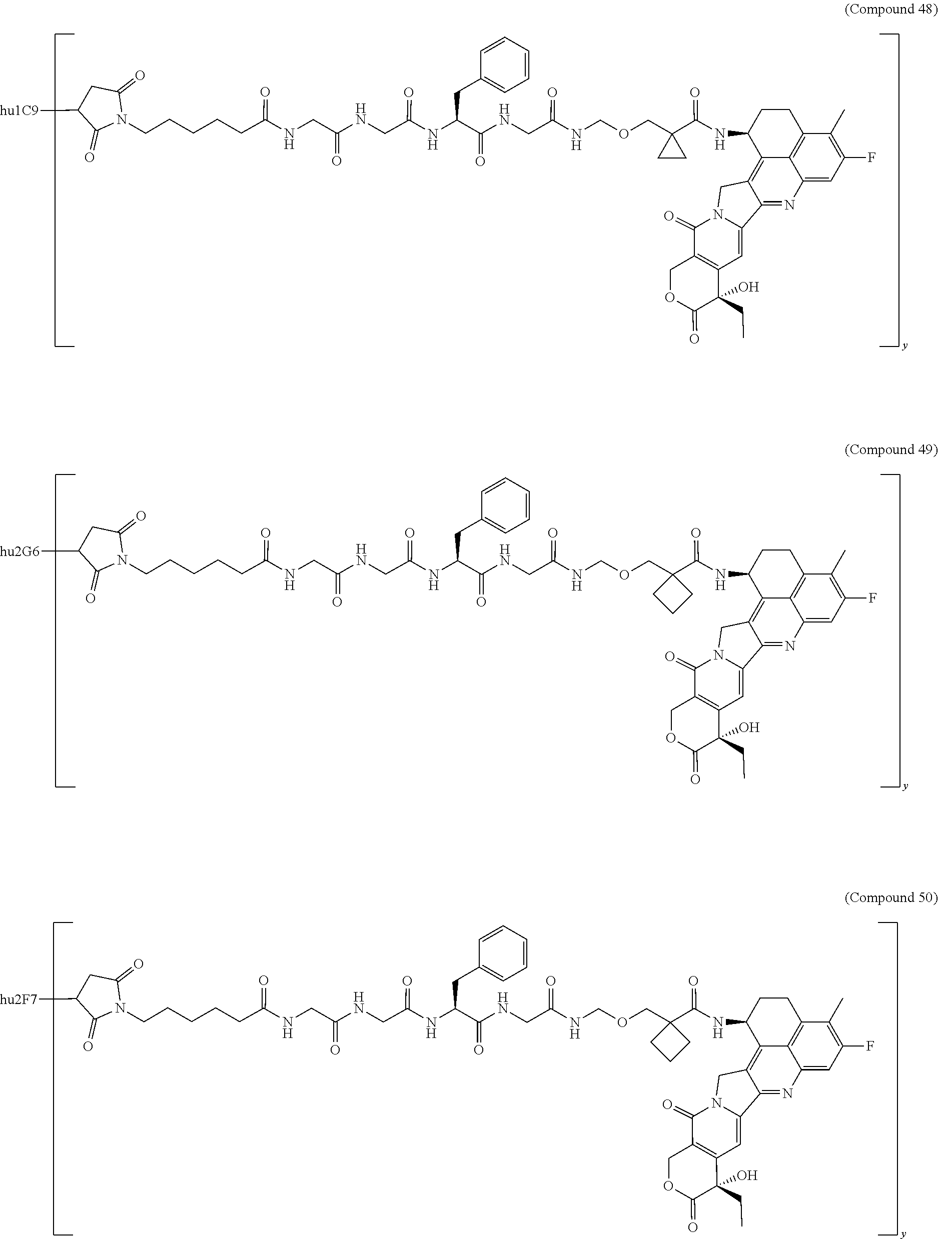 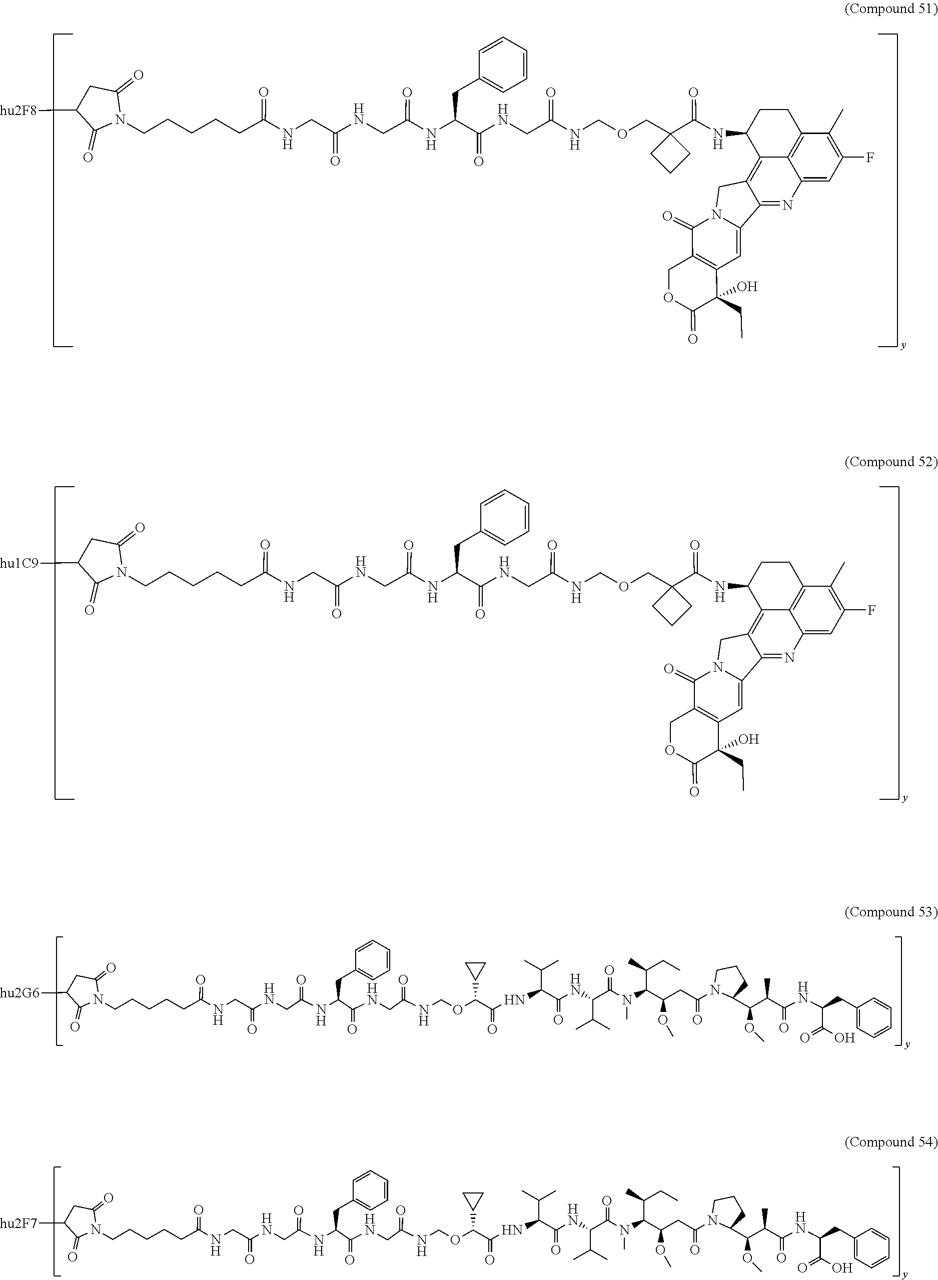 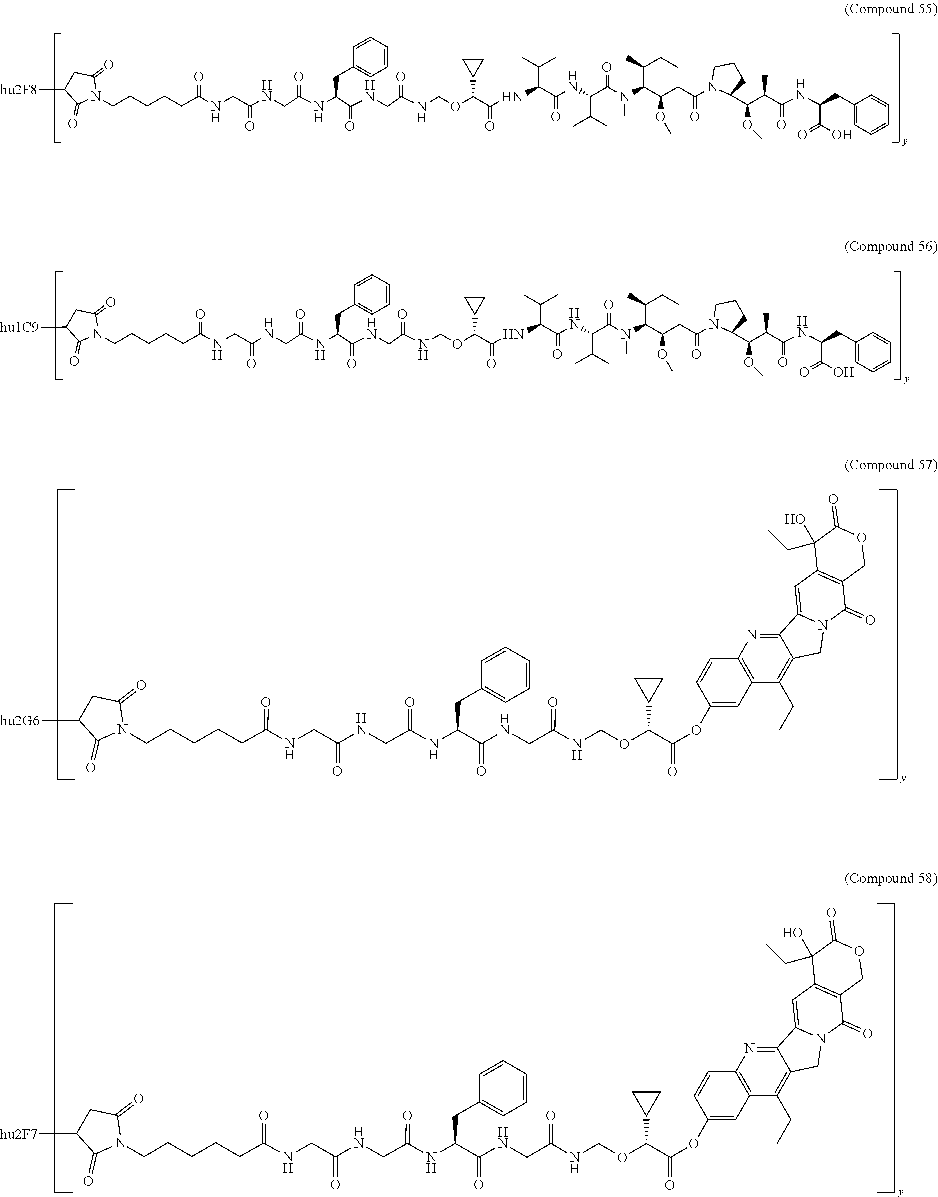 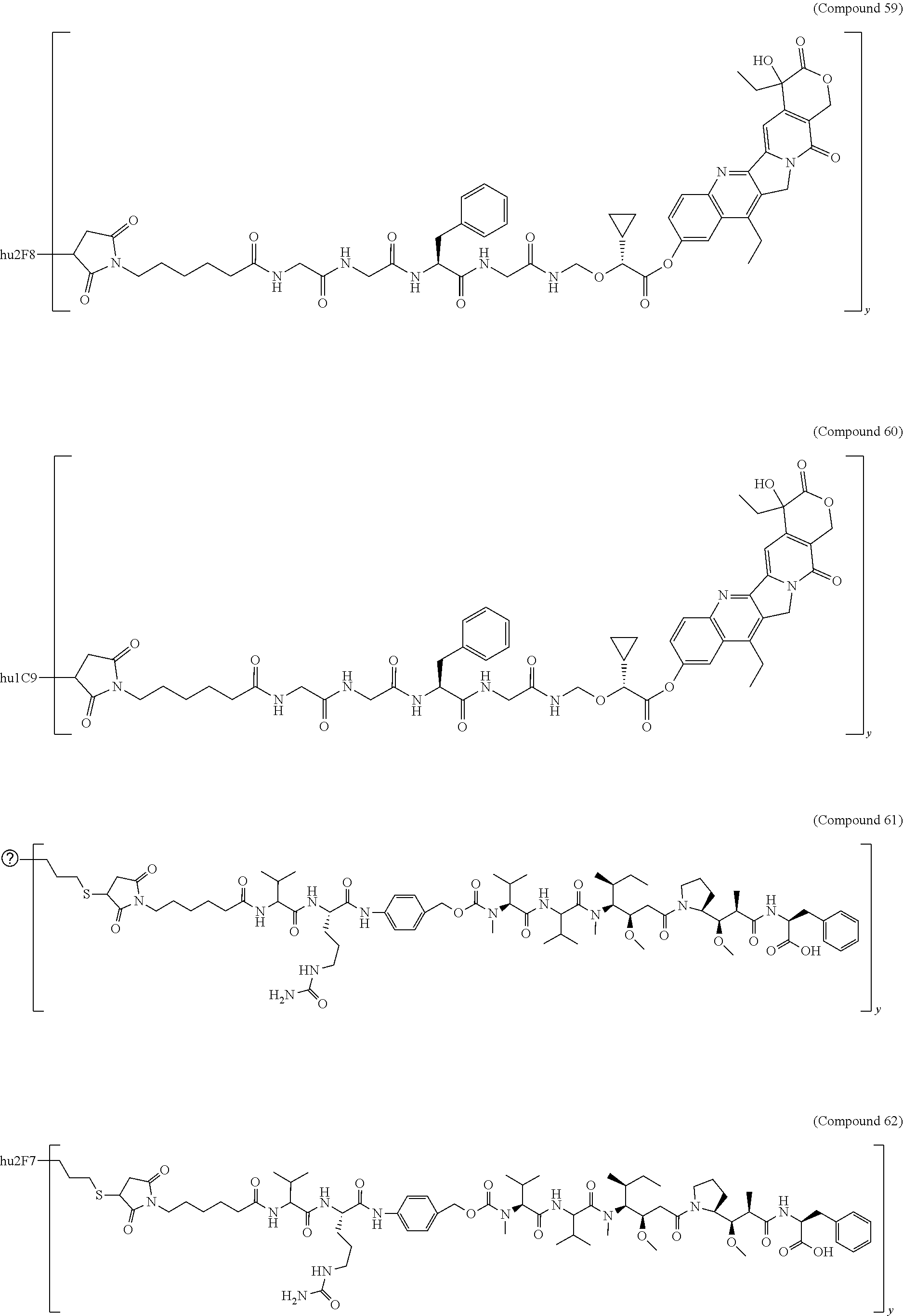 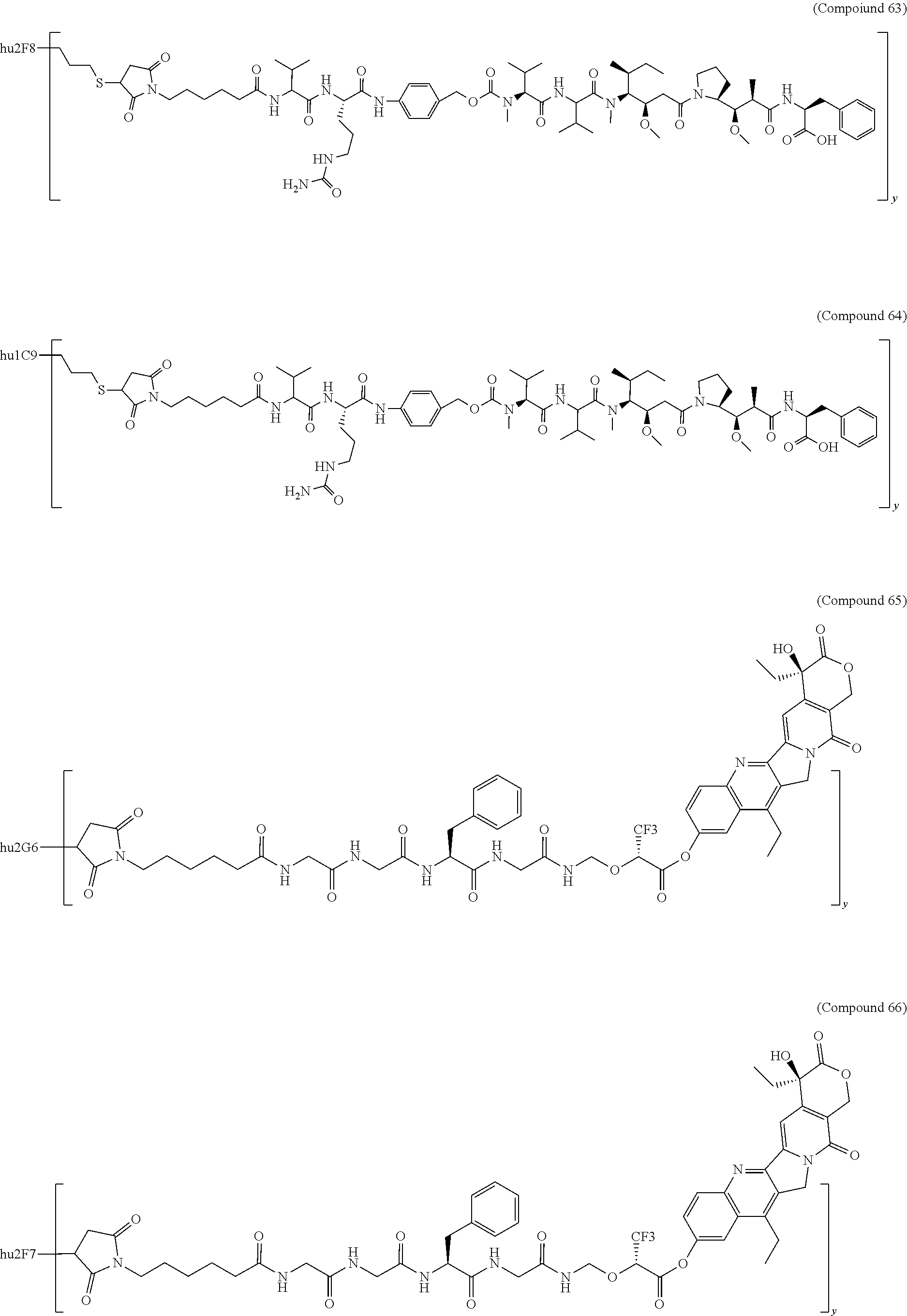 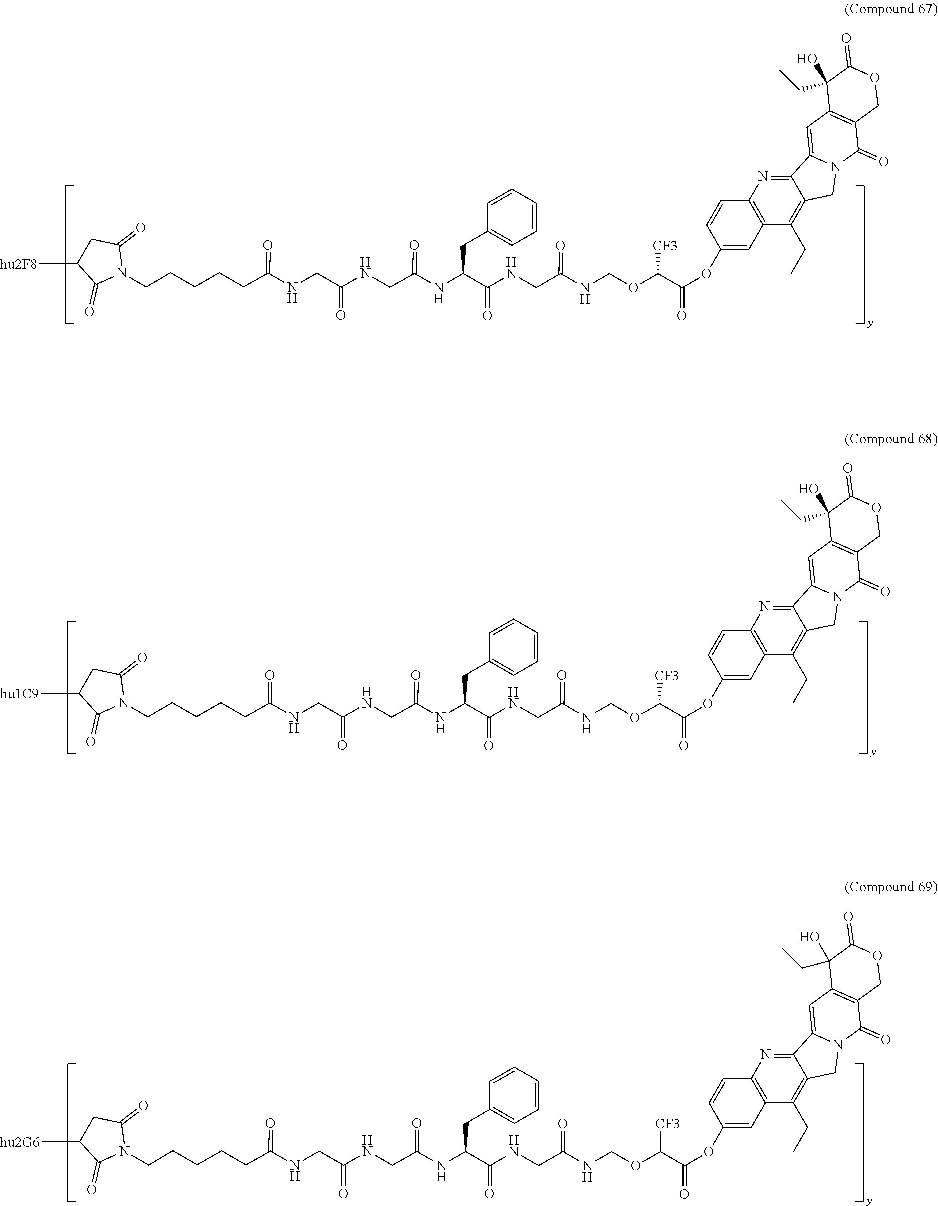 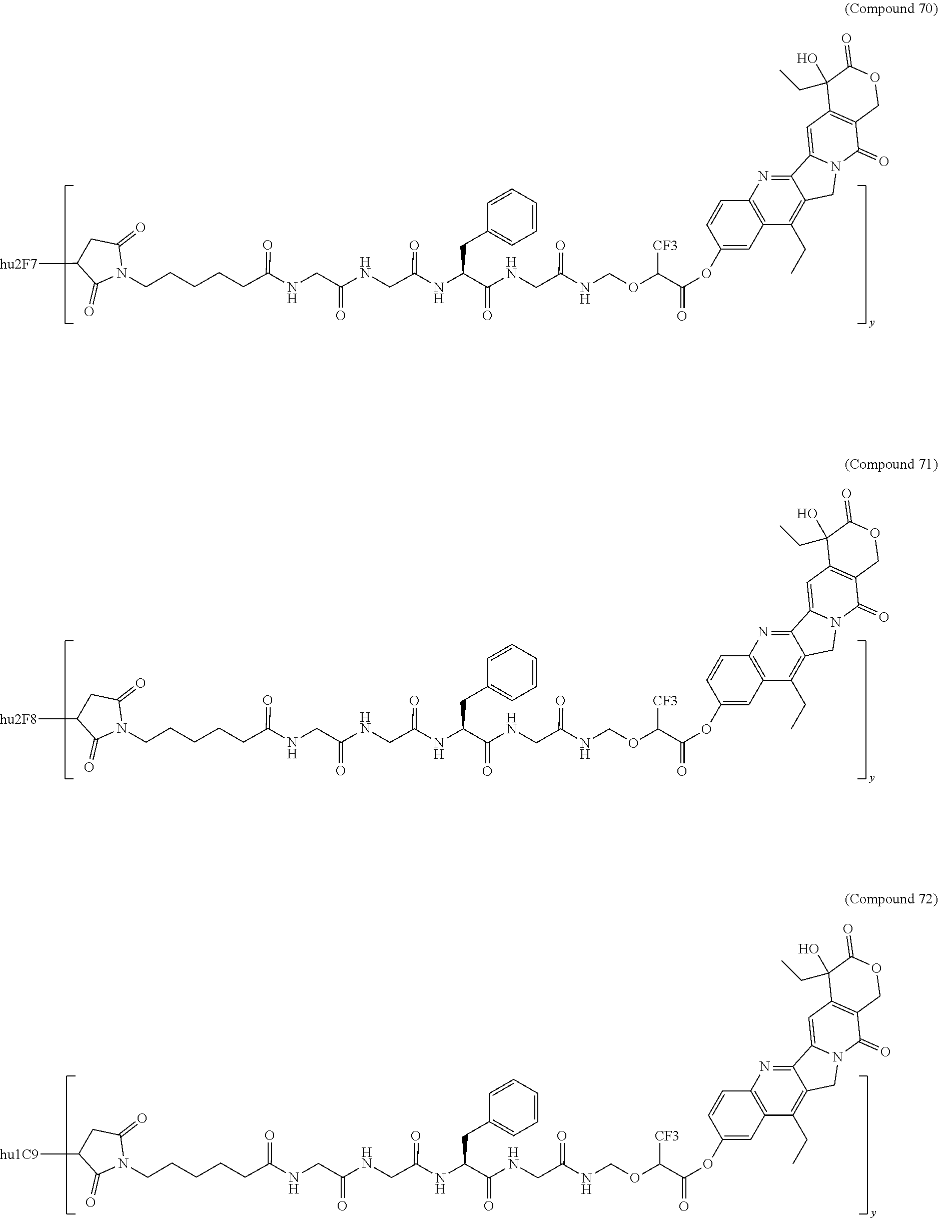 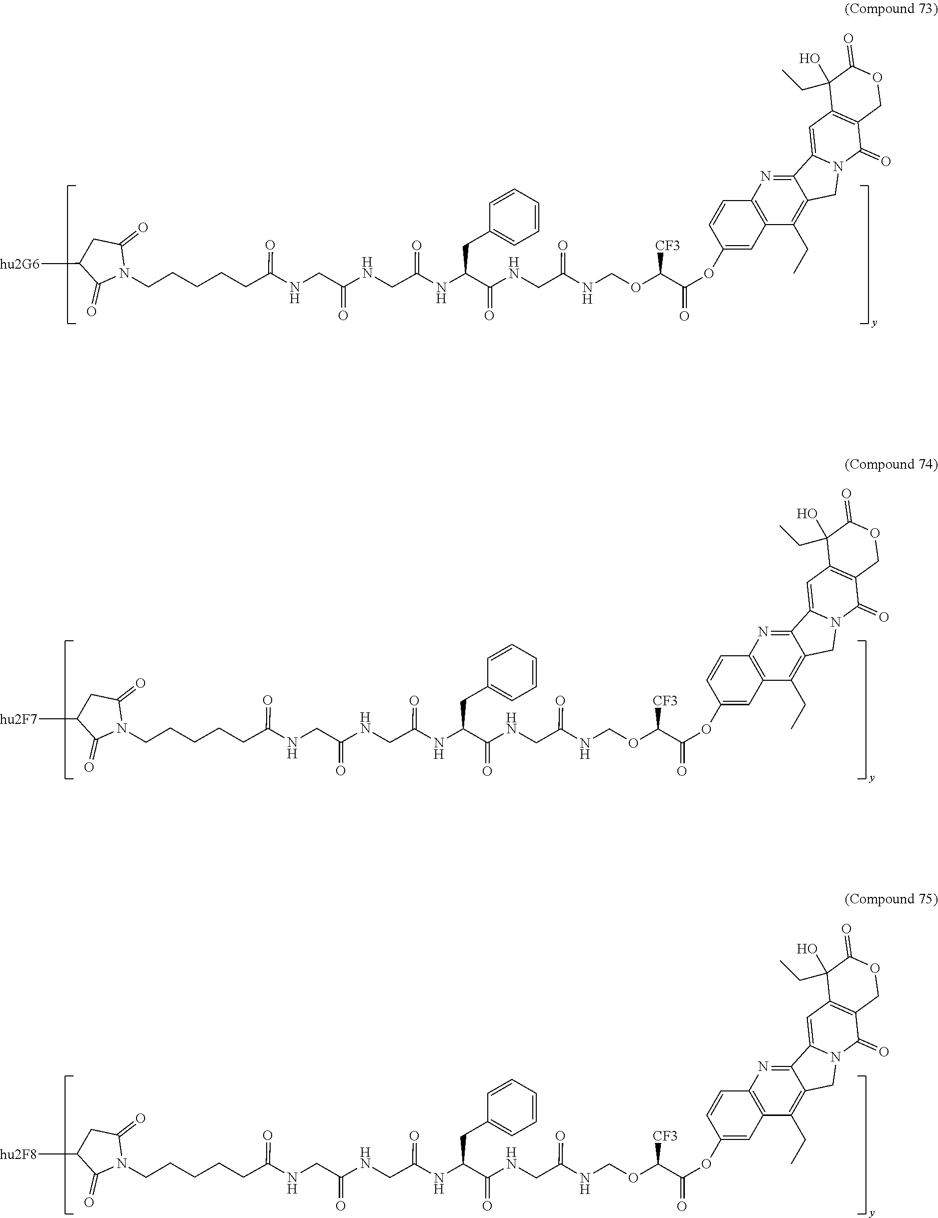 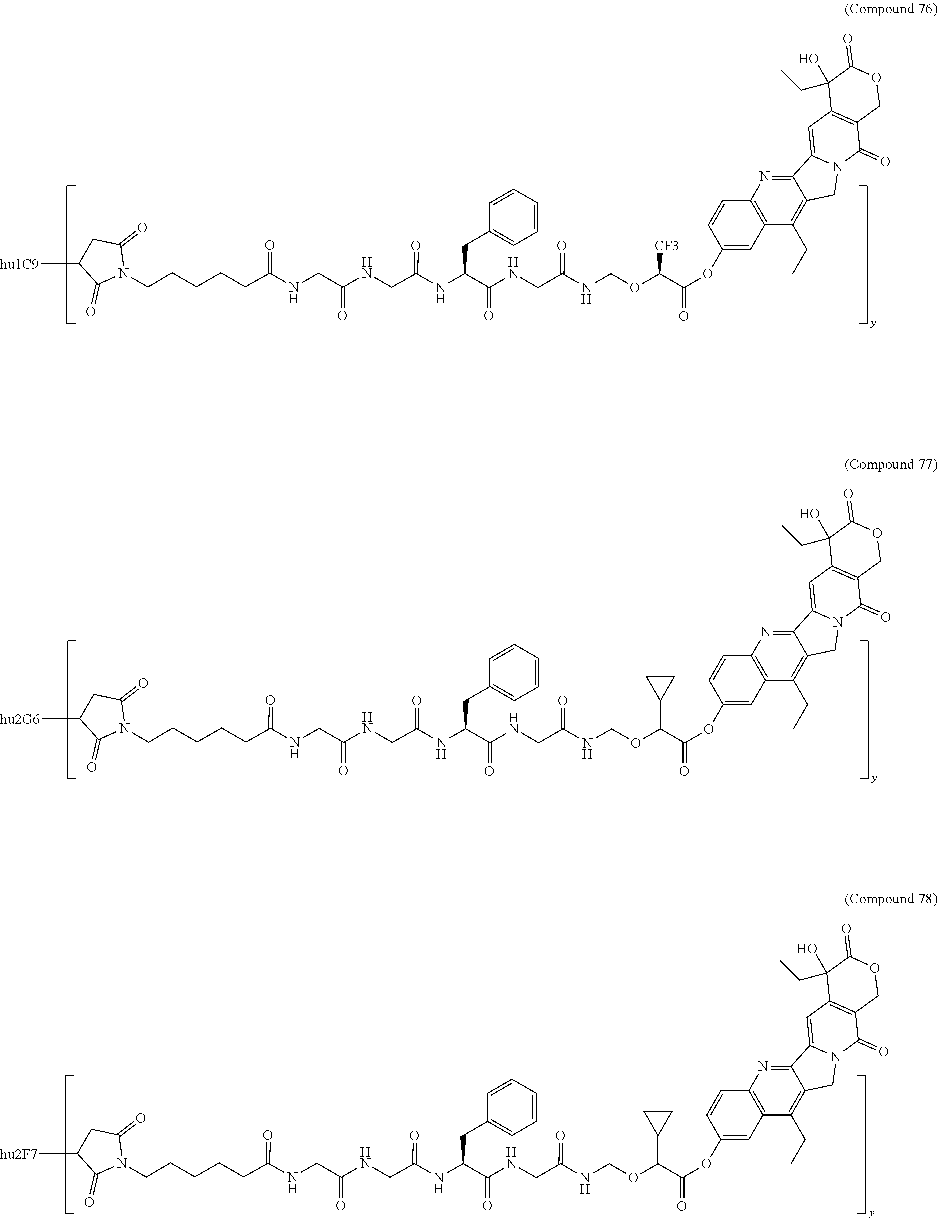 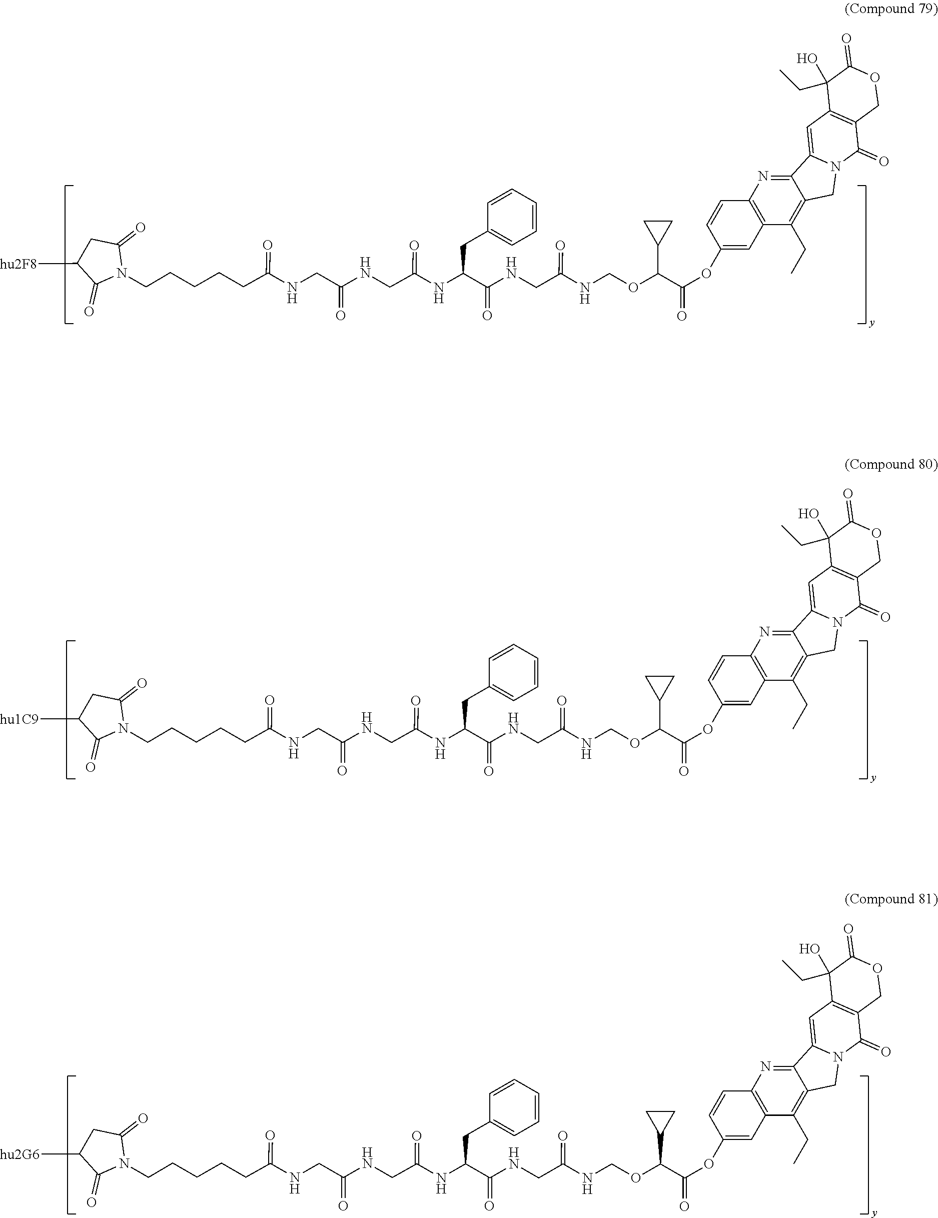 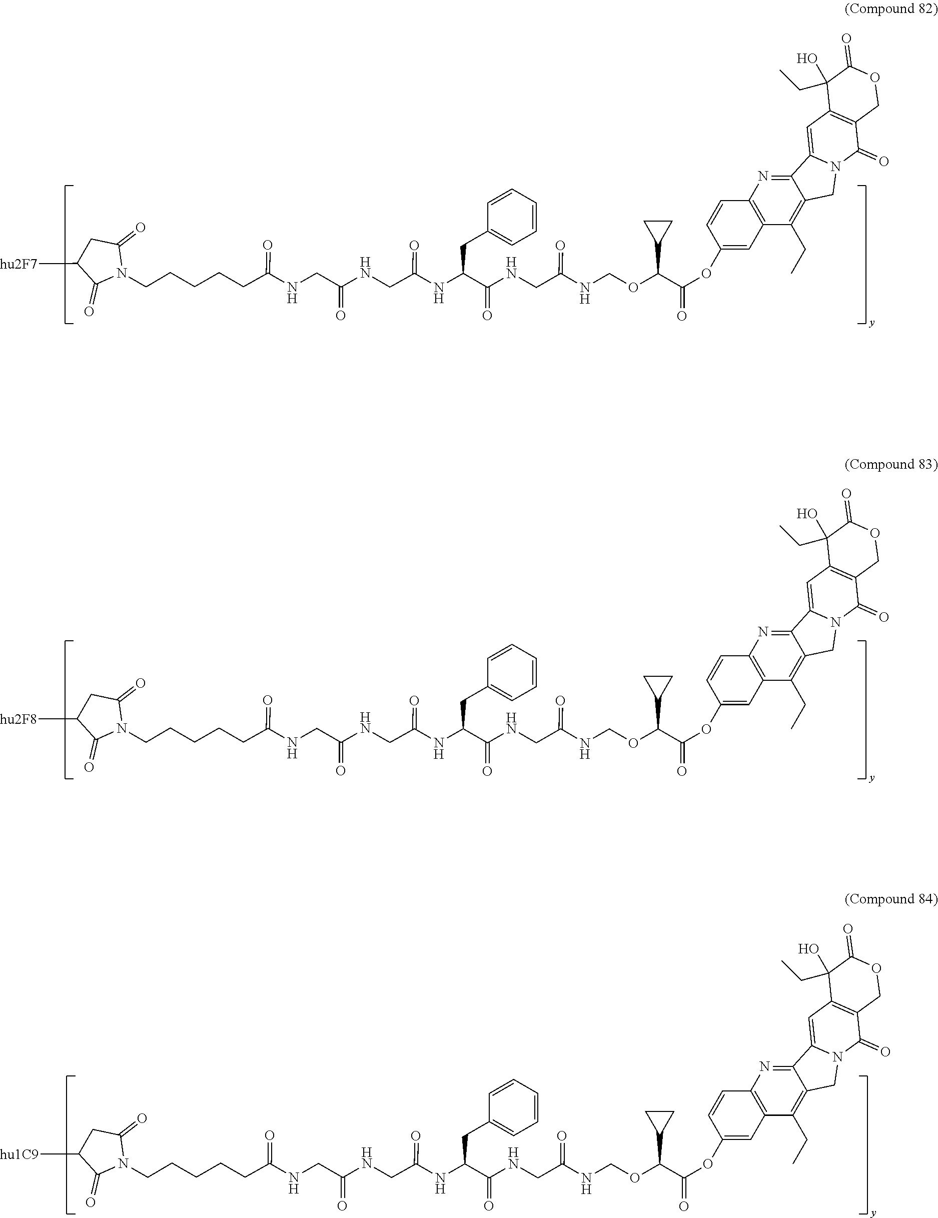 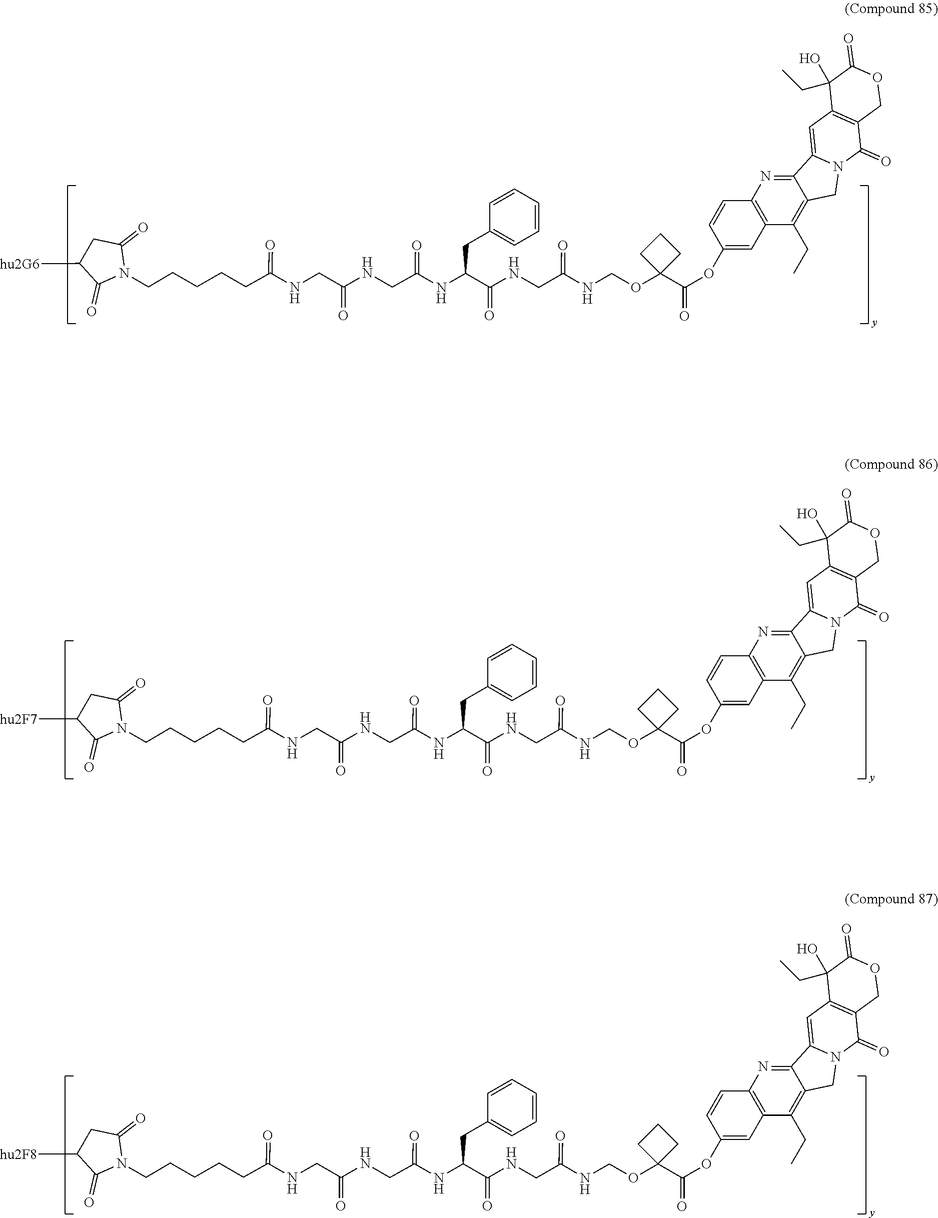 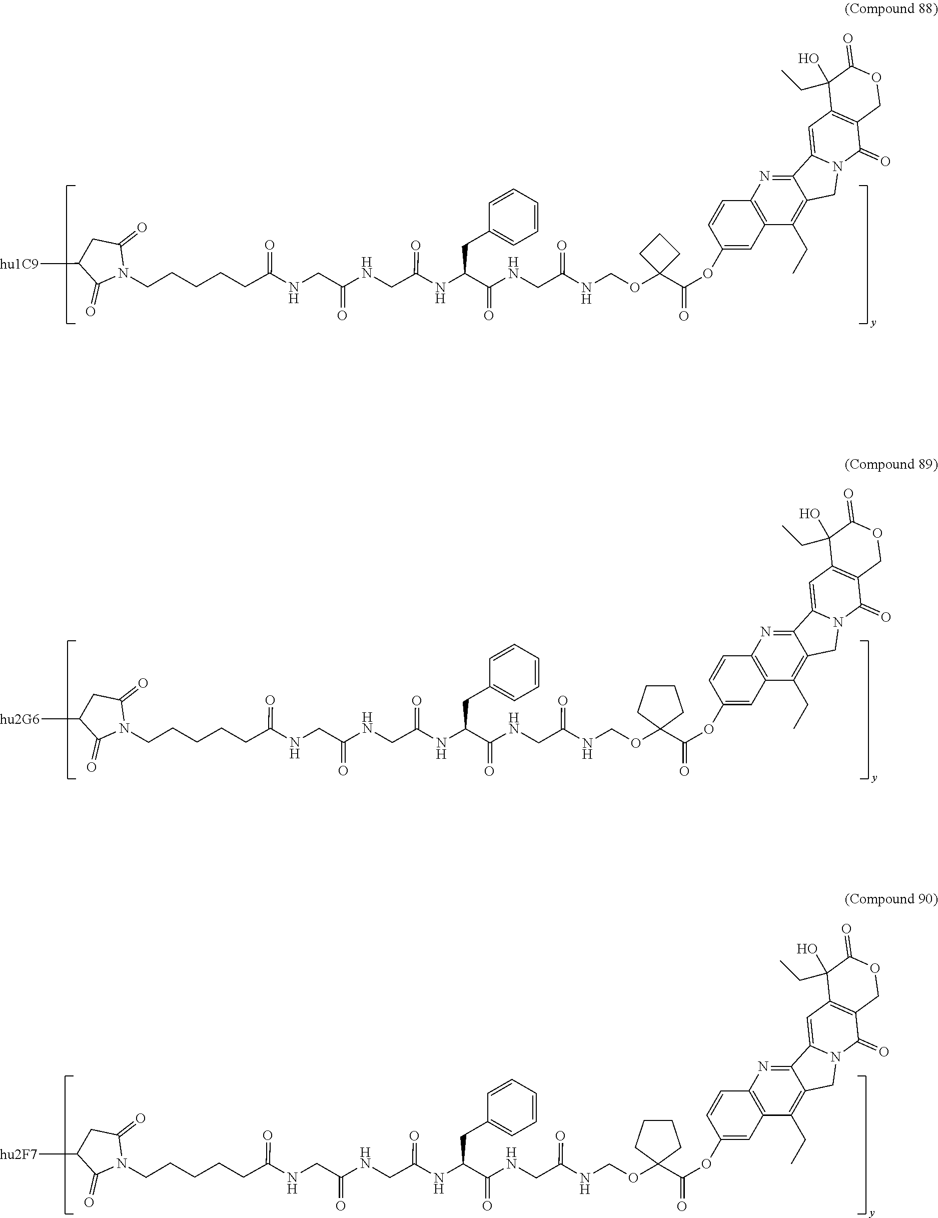 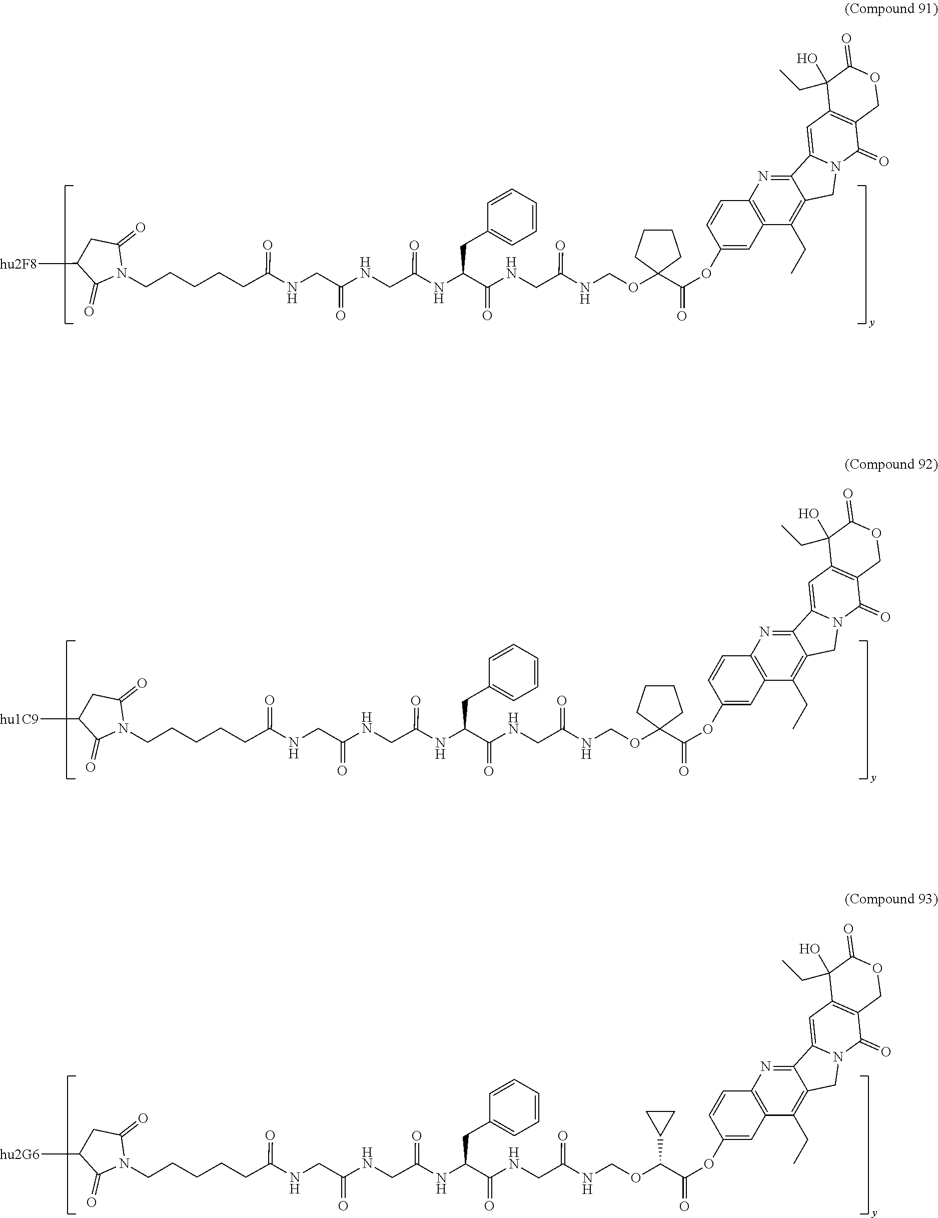 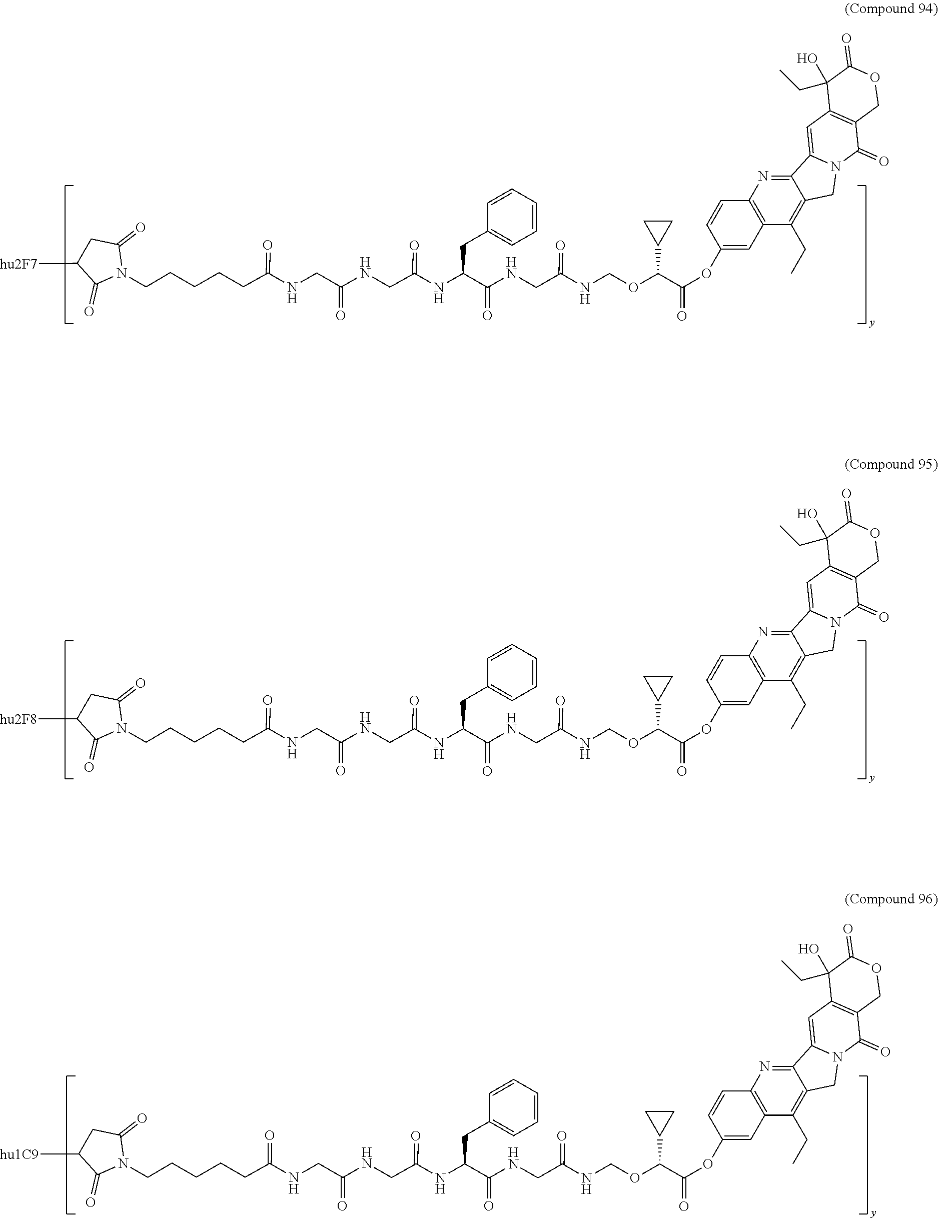 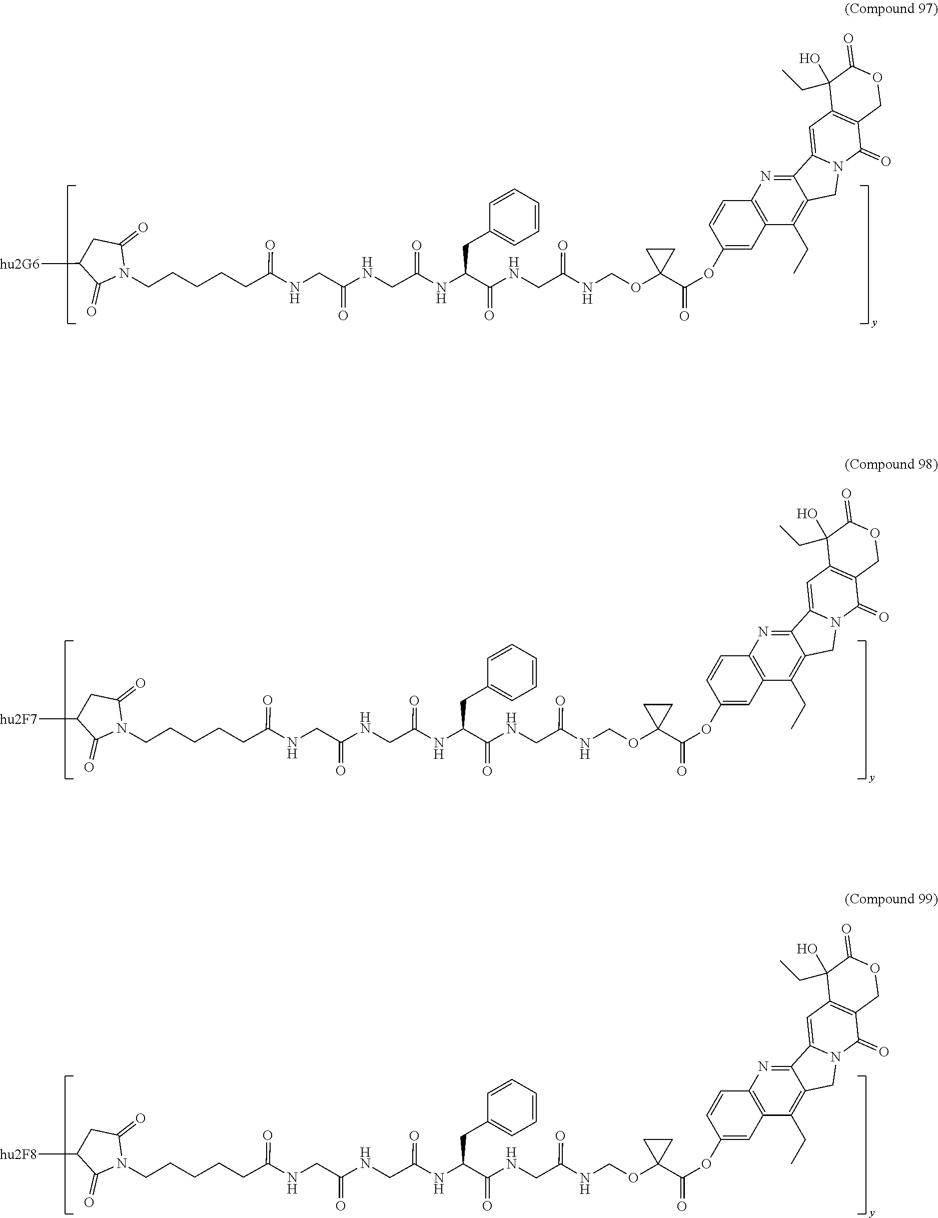 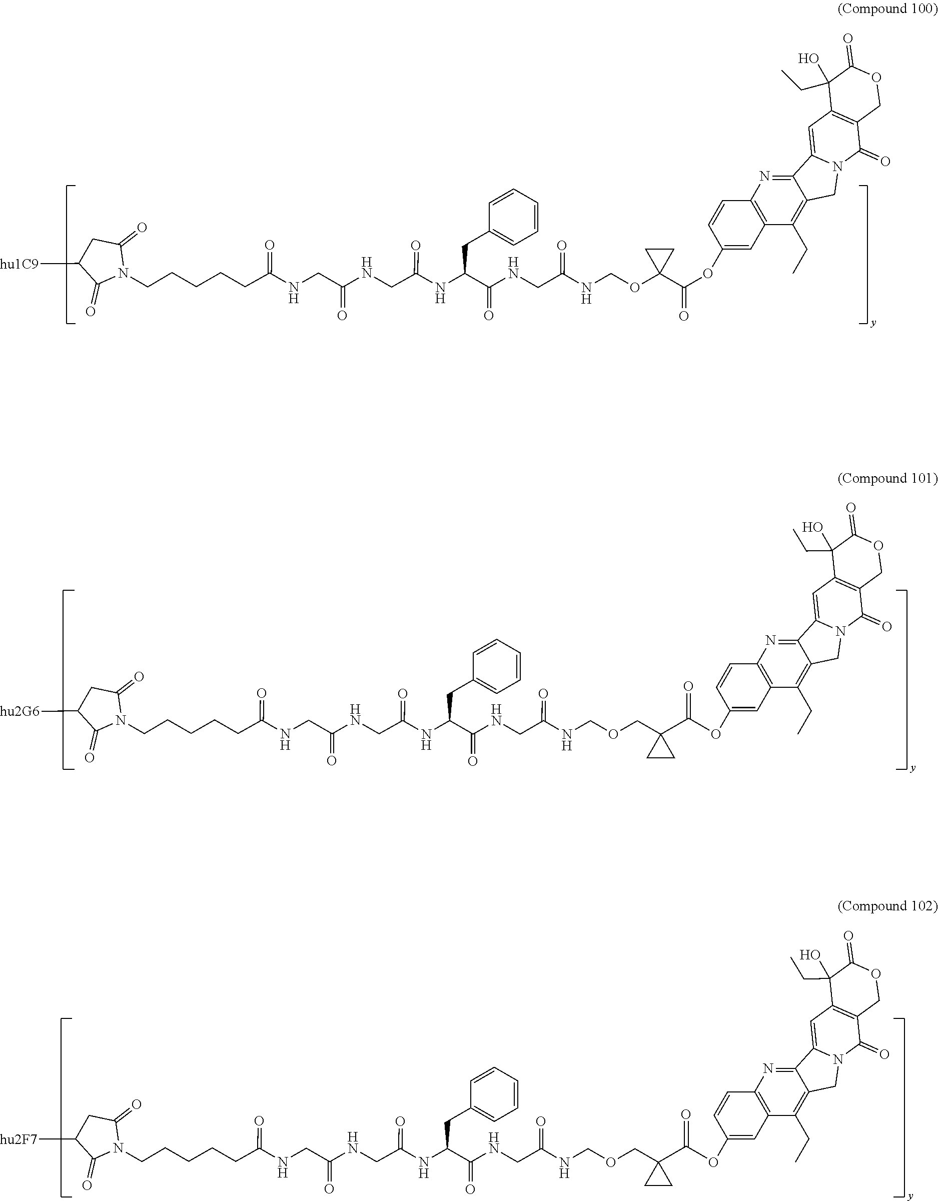 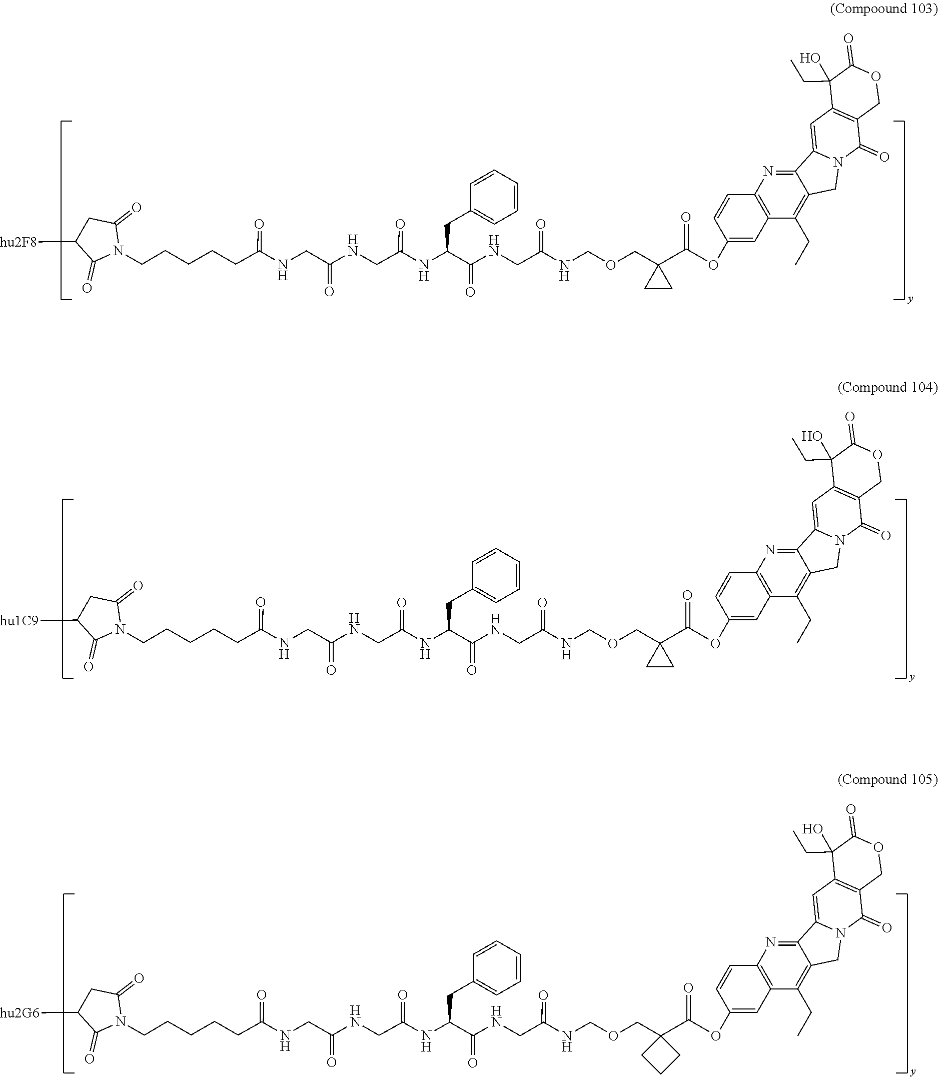 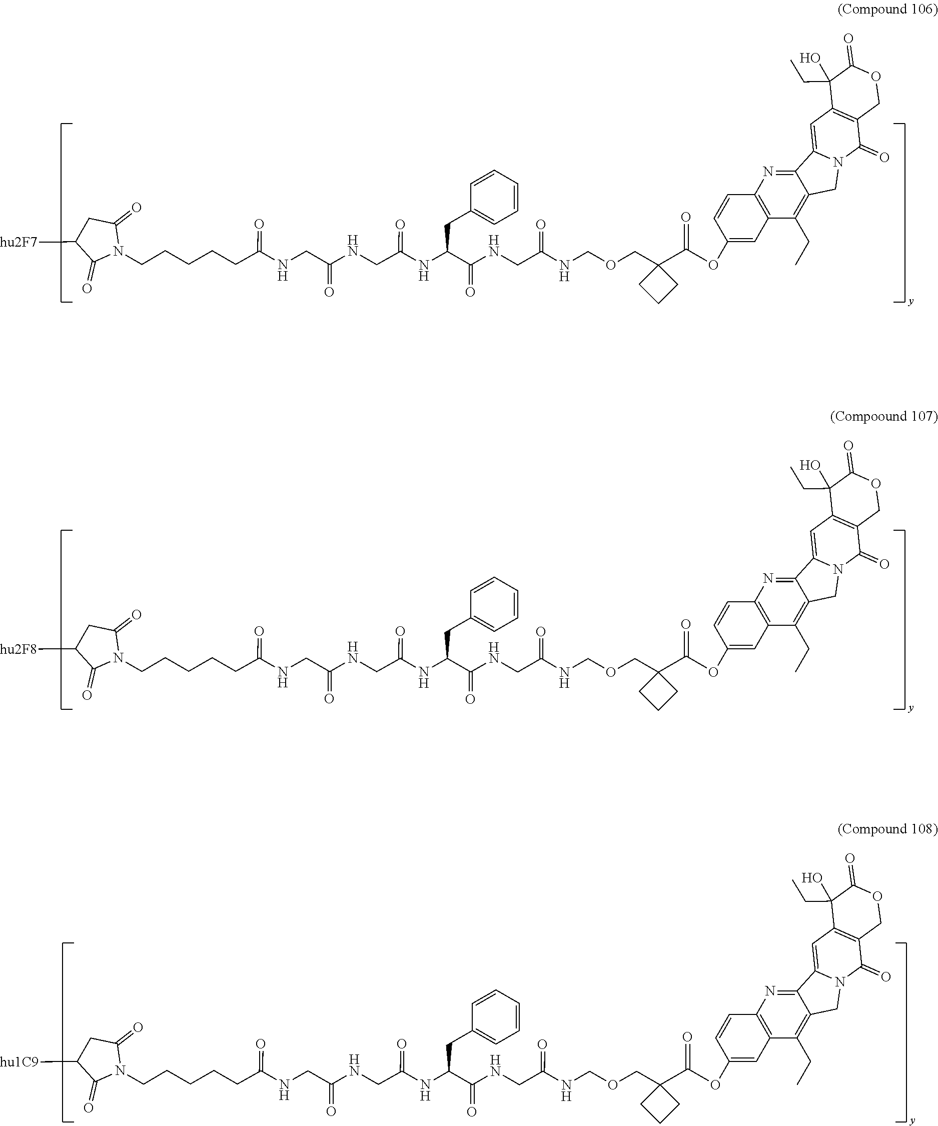 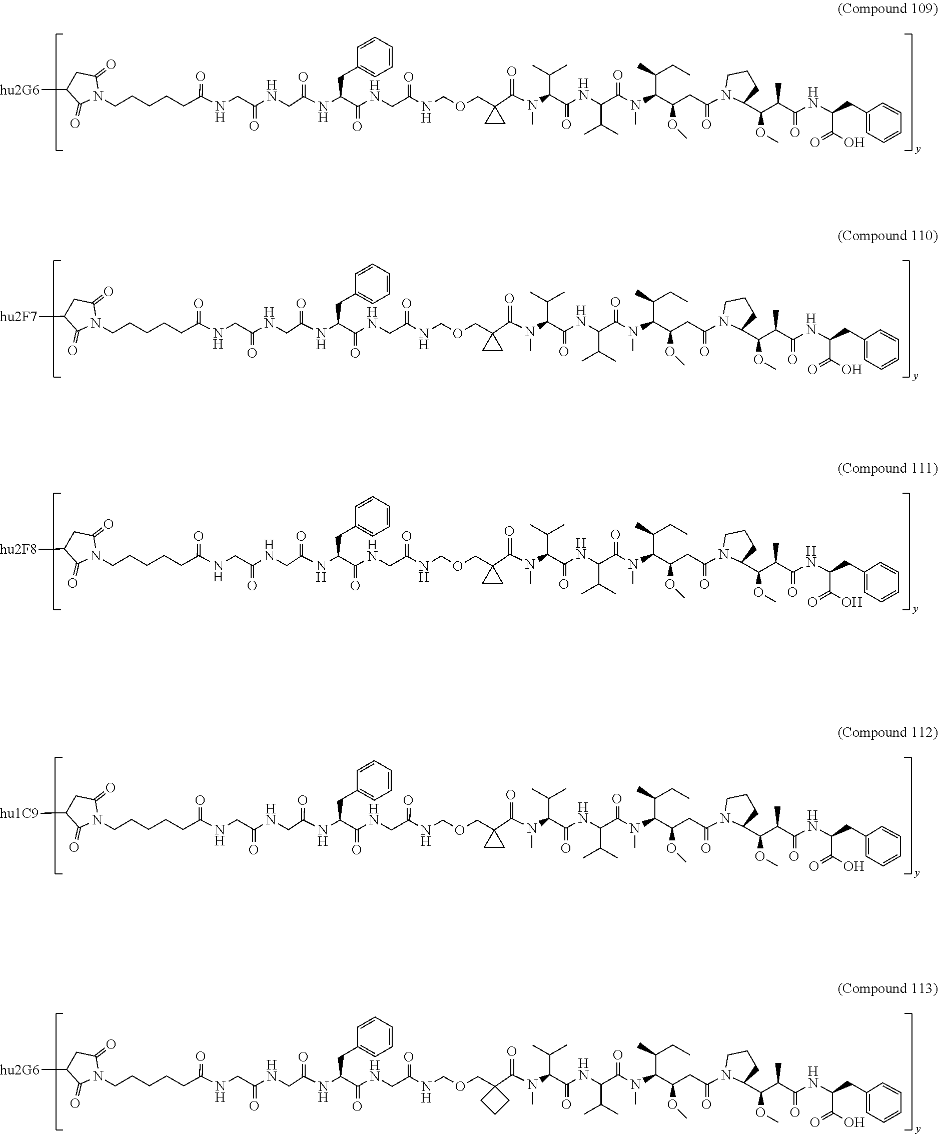 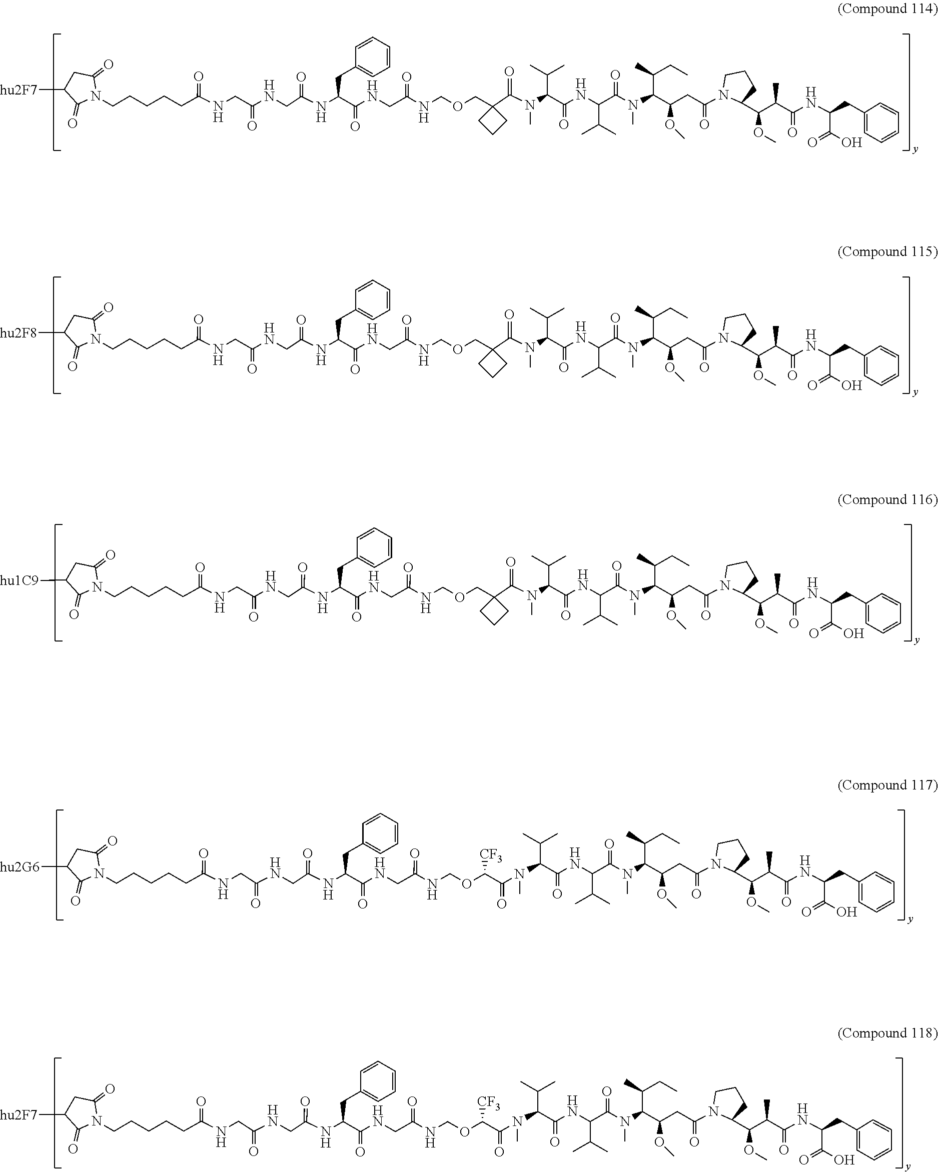 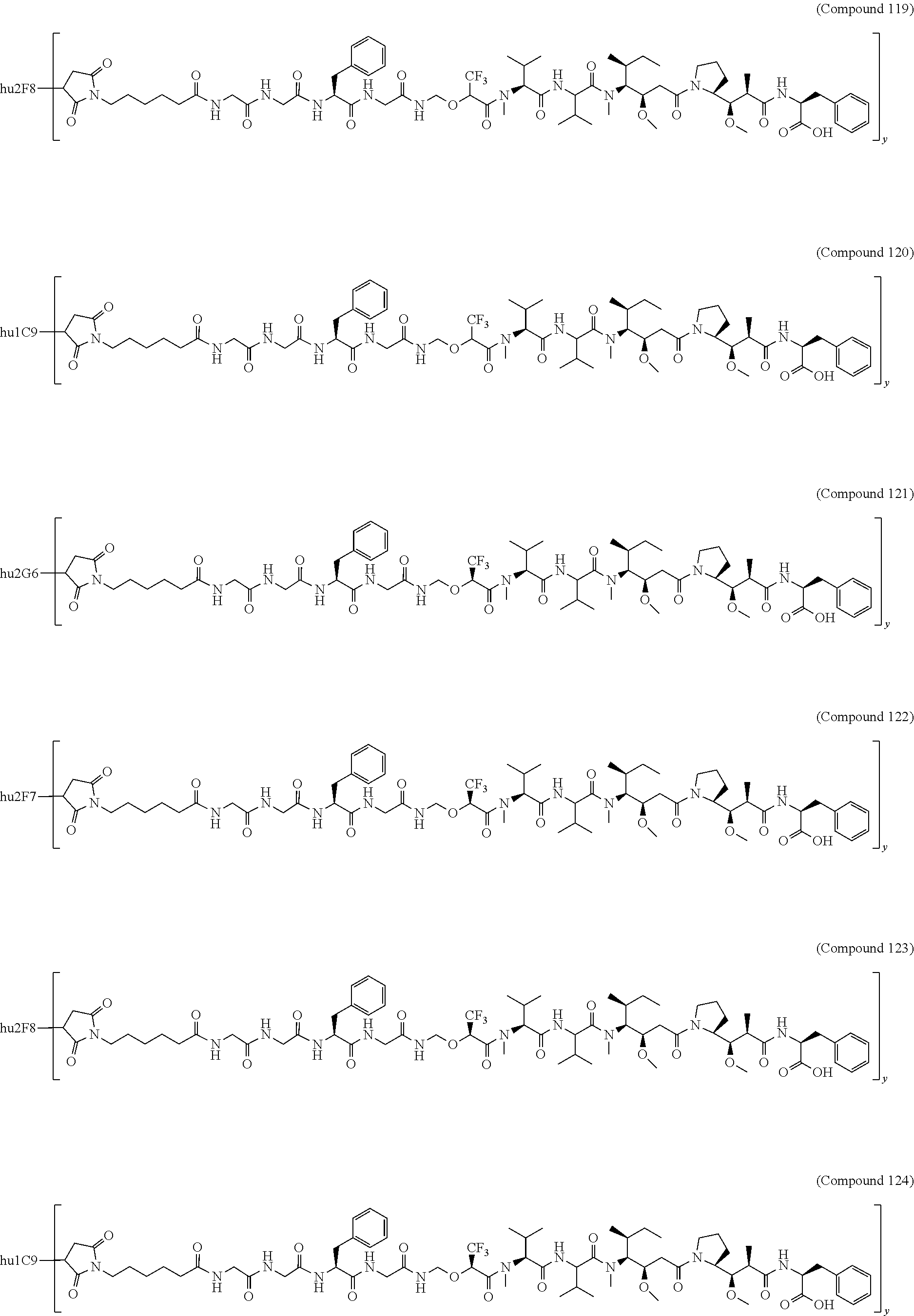 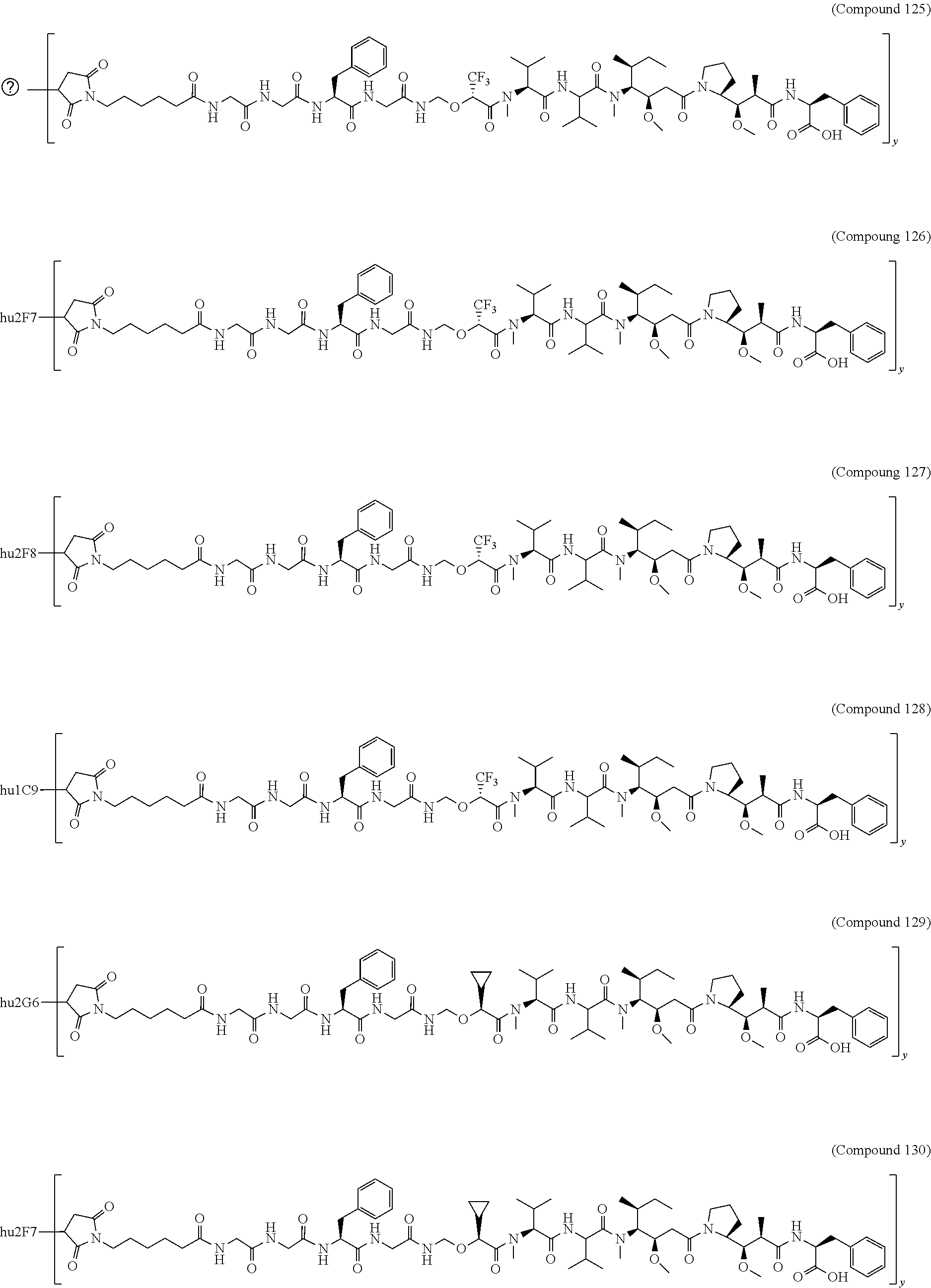 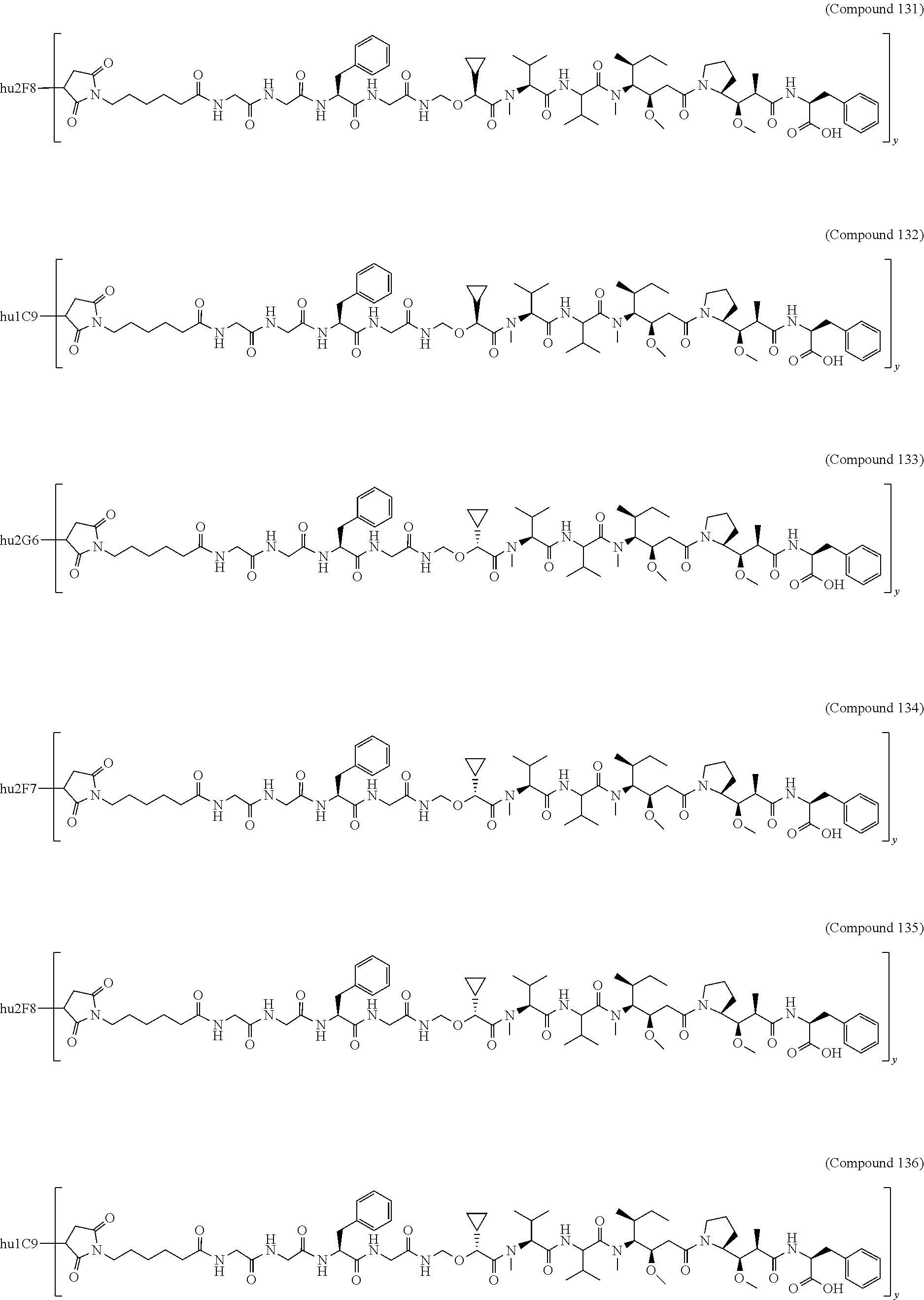 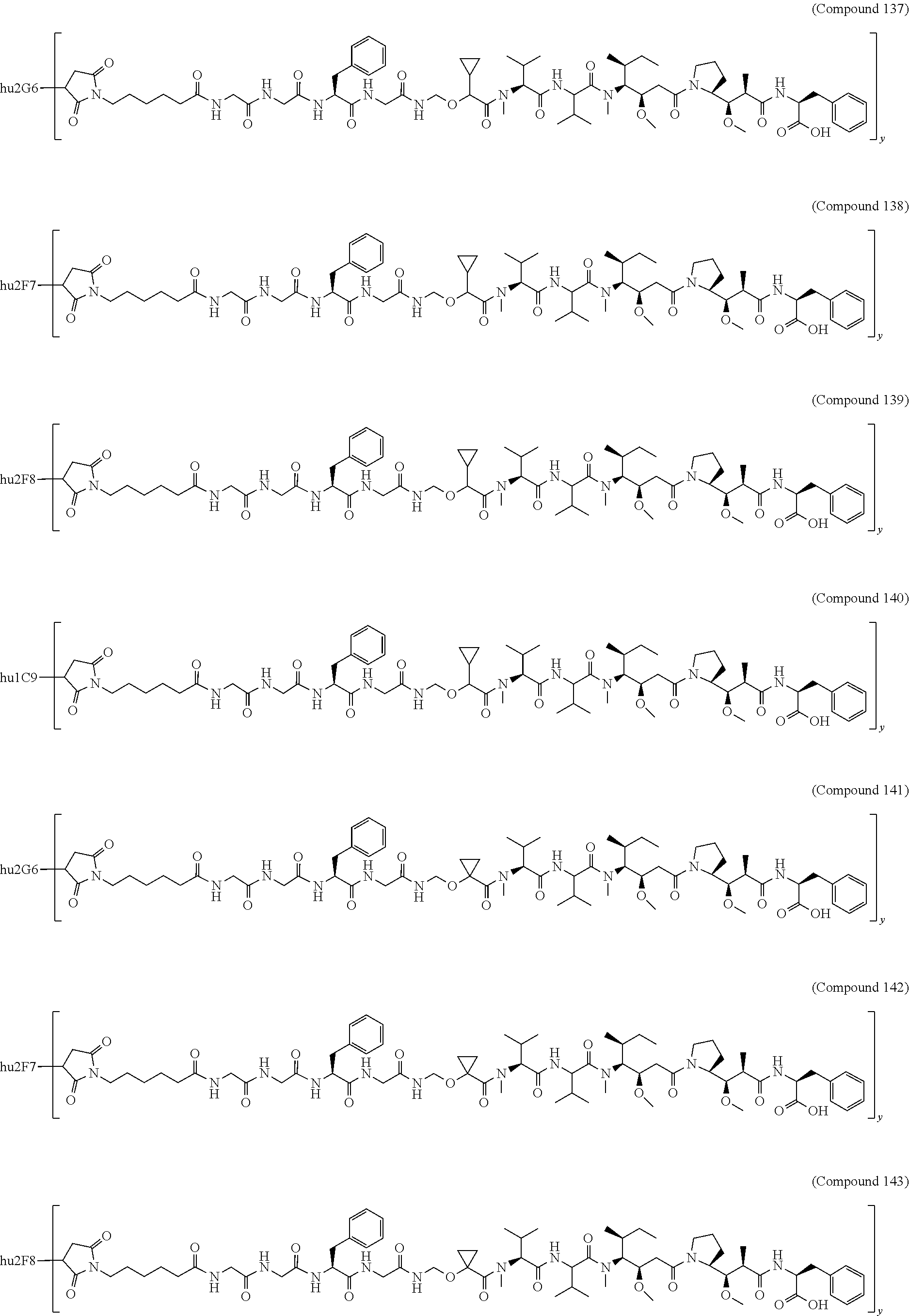 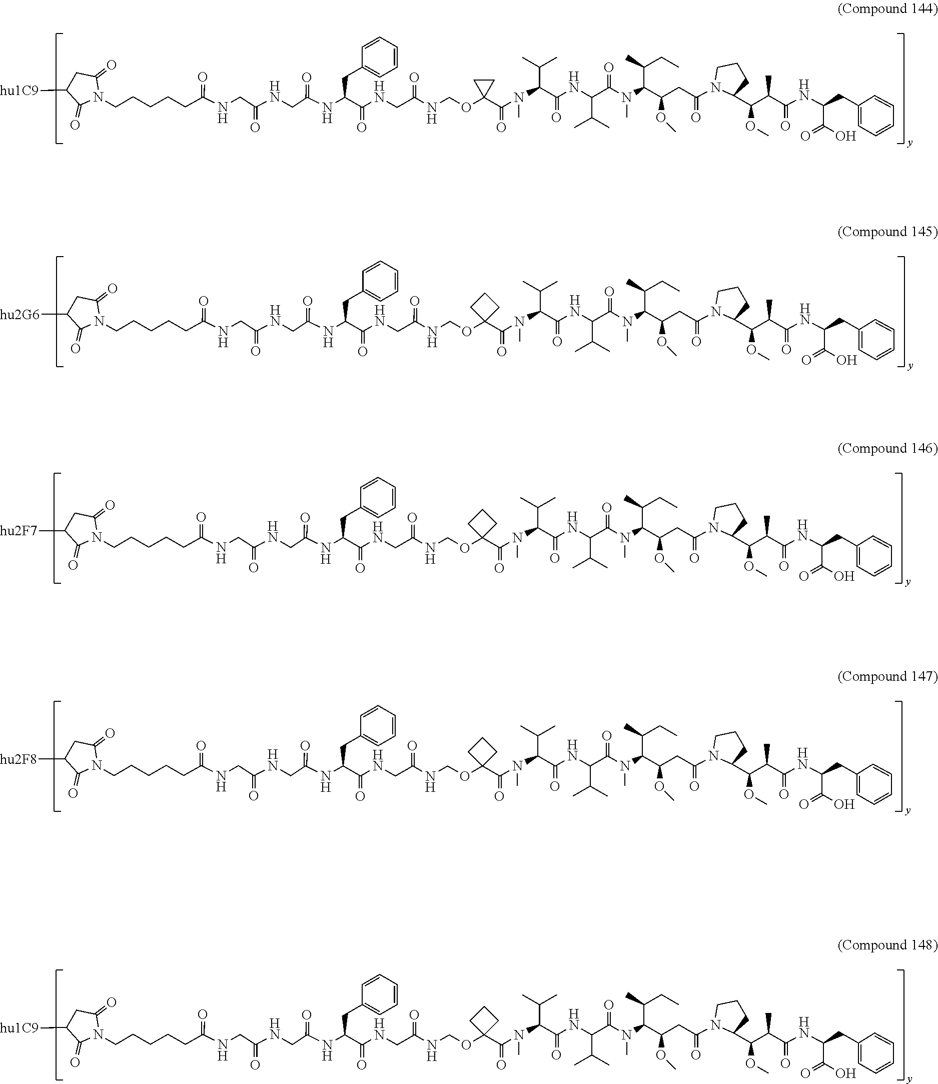 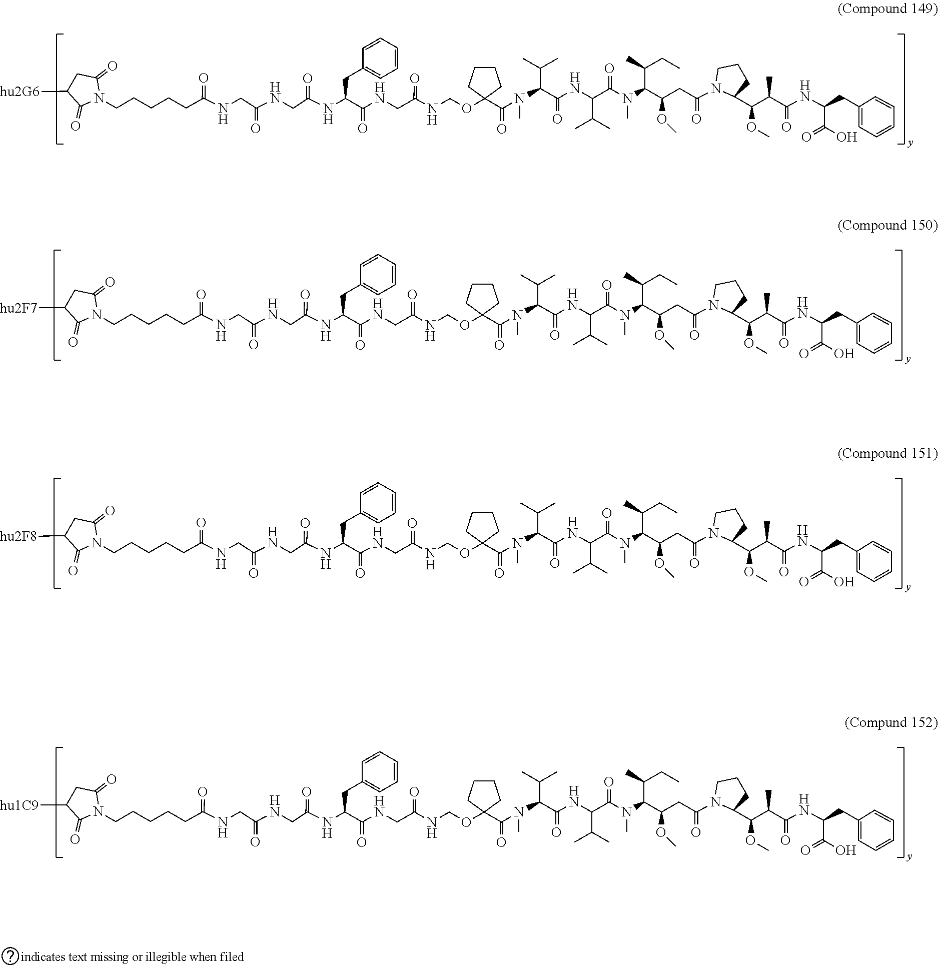
wherein, y is selected from 4 to 8.

57. The antibody-drug conjugate of general formula (A) or the pharmaceutically acceptable salt or solvate thereof according to claim 56, wherein, y is selected from 6 to 8.

58. A method for preparing the antibody-drug conjugate of general formula (IV) according to claim 52 or the pharmaceutically acceptable salt or solvate thereof, which comprises the following steps: 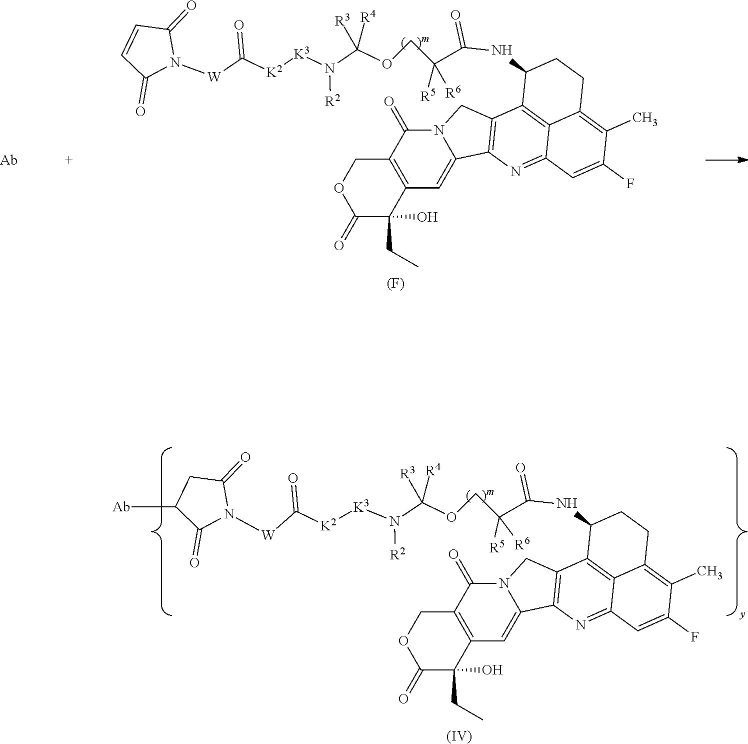
once Ab is reduced, it is subjected to coupling reaction with general formula (F) to obtain the compound of general formula (IV);
wherein:
Ab is an anti-B7-H4 antibody or antigen-binding fragment thereof;
wherein:
W is selected from the group consisting of C₁₋₈ alkyl, C₁₋₈ alkyl-cycloalkyl and linear heteroalkyl of 1 to 8 atoms, the heteroalkyl comprises 1 to 3 heteroatom(s) selected from the group consisting of N, O and S, wherein the C₁₋₈ alkyl, cycloalkyl and linear heteroalkyl are optionally further substituted with one or more substituents selected from the group consisting of halogen, hydroxyl, cyano, amino, alkyl, chloroalkyl, deuterated alkyl, alkoxyl and cycloalkyl;
K² is selected from the group consisting of —NR¹(CH₂CH₂O)ₚ₁CH₂CH₂C(O)—, —NR¹(CH₂CH₂O)ₚ₁CH₂C(O)—, —S(CH₂)ₚ₁C(O)— or bond, R¹ is selected from the group consisting of hydrogen atom, alkyl, haloalkyl, deuterated alkyl and hydroxyalkyl, and p₁ is an integer of 1 to 20;
K³ is a peptide residue consisting of 2 to 7 amino acids, the amino acid(s) can be substituted or unsubstituted; if being substituted, the substituent(s) can be substituted at any available attachment point, and the substituent(s) is/are one or more independently selected from the group consisting of halogen, hydroxyl, cyano, amino, alkyl, chloroalkyl, deuterated alkyl, alkoxyl and cycloalkyl;
R¹ is independently selected from the group consisting of hydrogen atom, alkyl, haloalkyl, deuterated alkyl and hydroxyalkyl;
R³ and R⁴ are each independently selected from the group consisting of hydrogen atom, halogen, alkyl, haloalkyl, deuterated alkyl and hydroxyalkyl;
R⁵ is selected from the group consisting of halogen, haloalkyl, deuterated alkyl, cycloalkyl, heterocyclyl, aryl and heteroaryl;
R⁶ is selected from the group consisting of hydrogen atom, halogen, haloalkyl, deuterated alkyl, cycloalkyl, heterocyclyl, aryl and heteroaryl;
or, R⁵ and R⁶ and the carbon atom to which they are linked form a cycloalkyl or heterocyclyl;
m is an integer of 0 to 4;
y is 1 to 10, y is a decimal or an integer.

59. The method according to claim 58, wherein the general formula (F) is a compound of general formula (F-1): 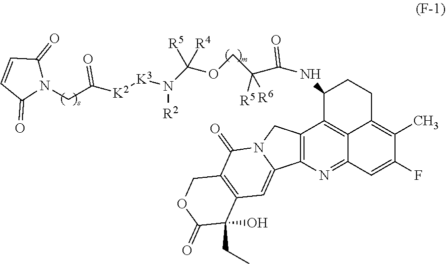
or a tautomer, mesomer, racemate, enantiomer, diastereomer or mixture form thereof, or the pharmaceutically acceptable salt thereof,
wherein
K² is a bond;
K³ is the tetrapeptide residue GGFG;
R⁵ is selected from the group consisting of haloalkyl and C₃₋₆ cycloalkyl;
R⁶ is selected from the group consisting of hydrogen, haloalkyl and C₃₋₆ cycloalkyl;
or, R⁵ and R⁶ and the carbon atom) to which they are linked form a C₃₋₆ cycloalkyl;
R², R³ or R⁴ are each independently hydrogen or alkyl;
s is an integer of 2 to 8;
m is an integer of 0 to 4.

60. The method according to claim 59, wherein the compound of general formula (F) or general formula (F-1) is selected from the group consisting of:

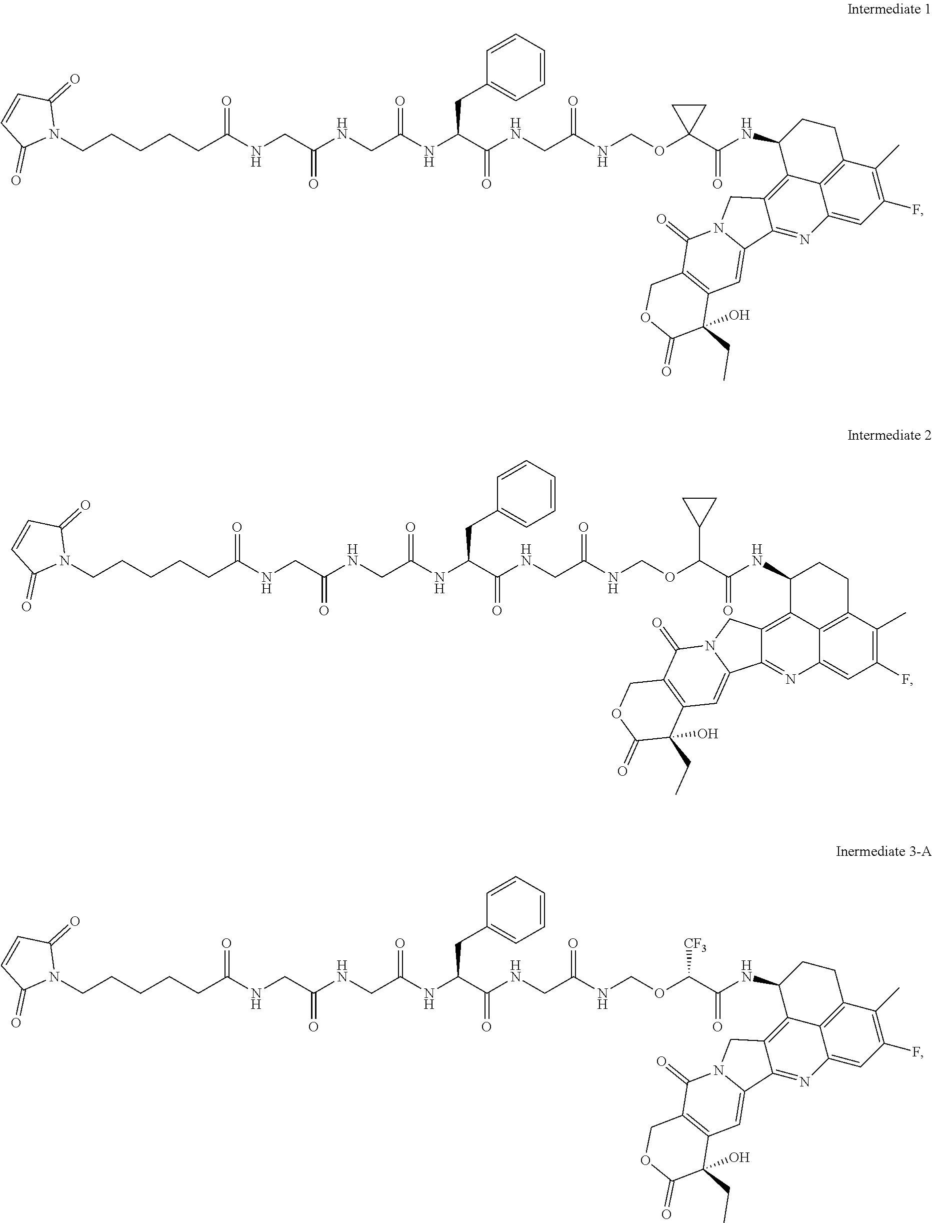

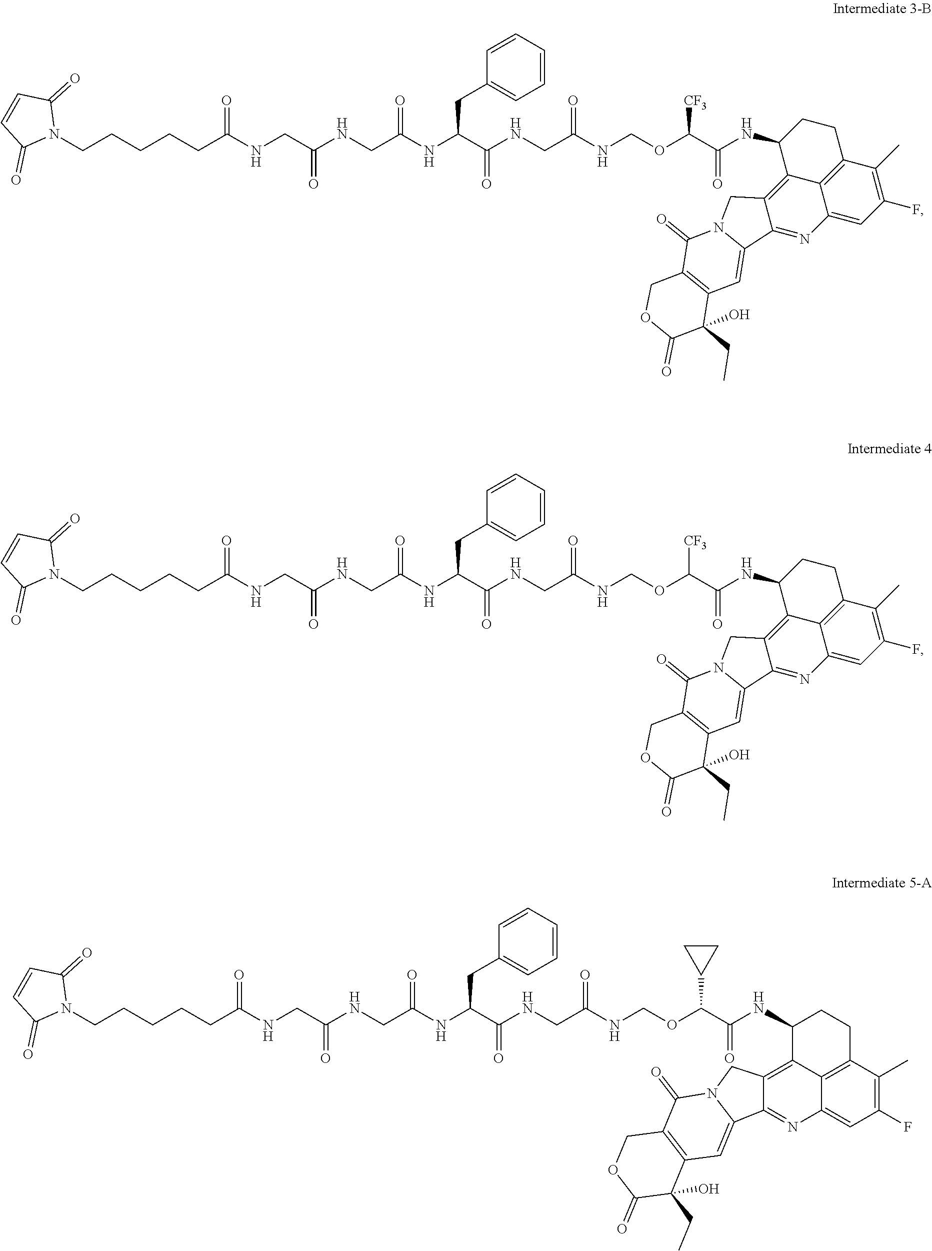

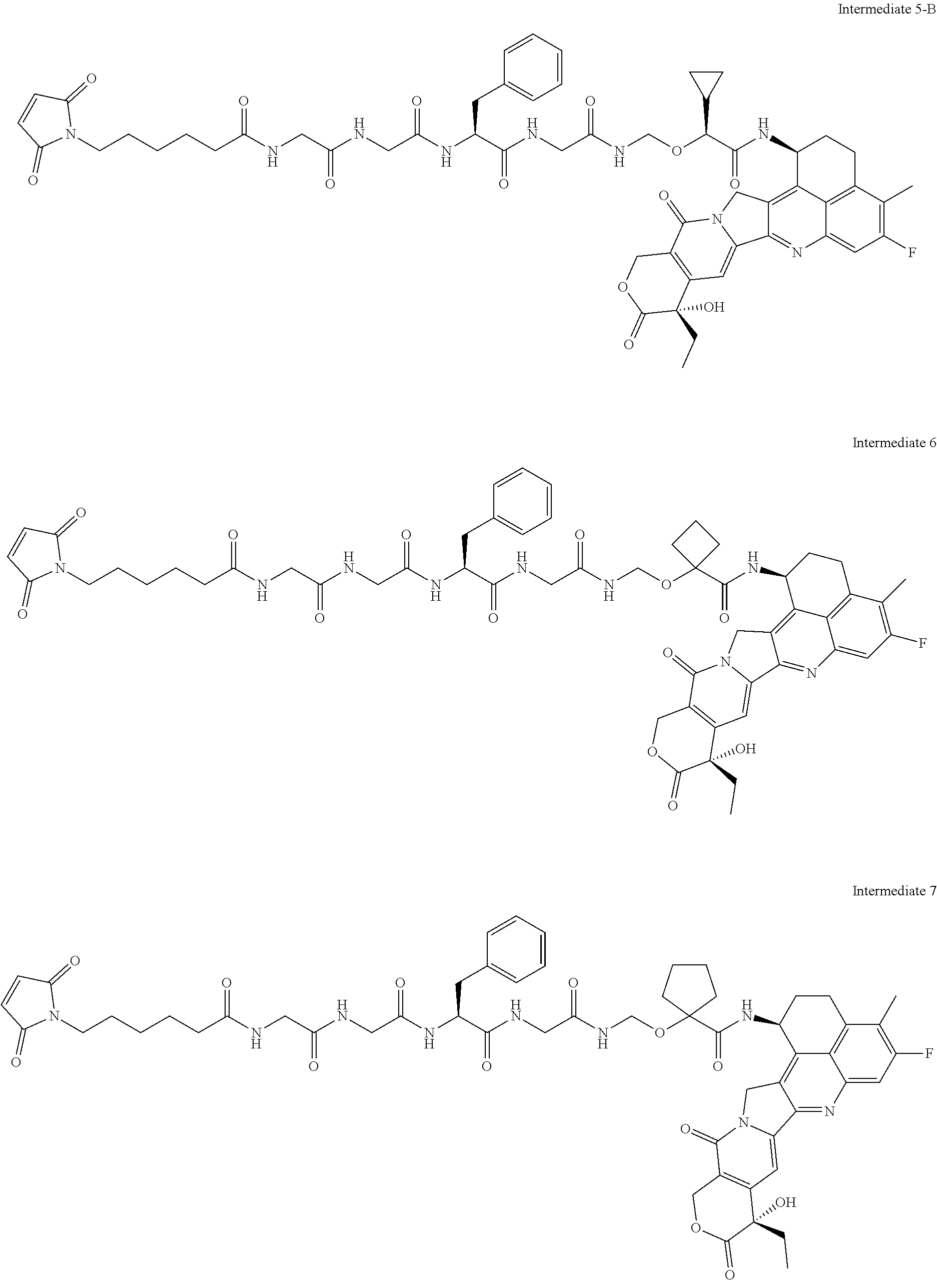

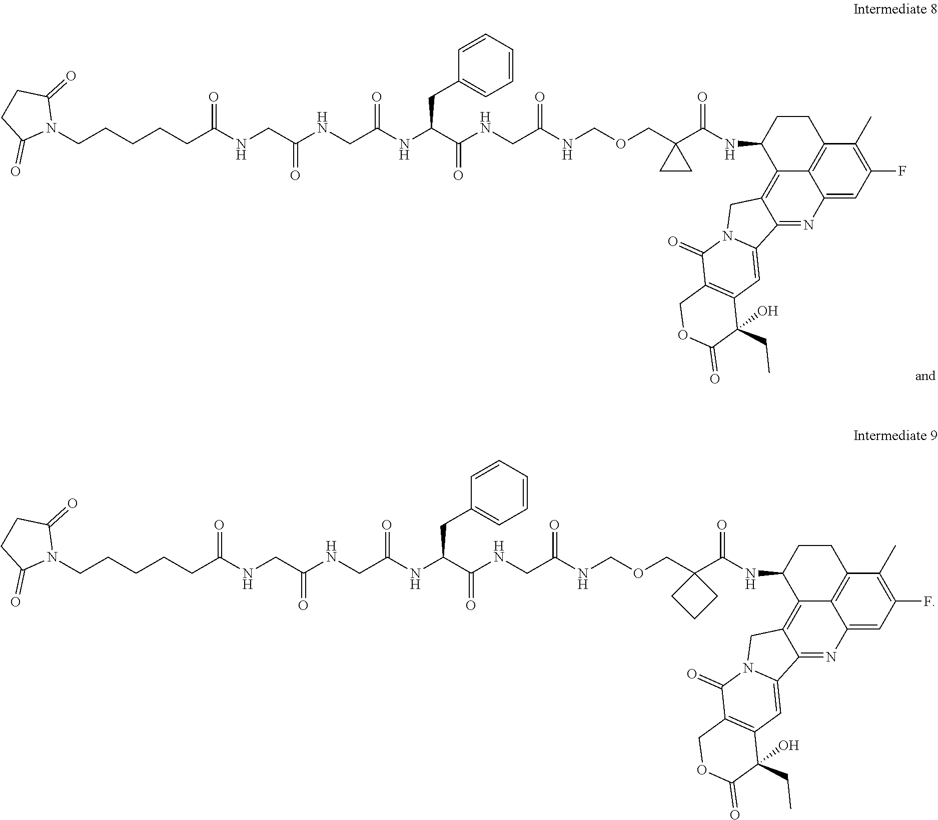

61. A pharmaceutical composition comprising the antibody-drug conjugate according to claim 18, or the pharmaceutically acceptable salt or solvate thereof, and one or more pharmaceutically acceptable carriers.

62. A method for treating a disease related to human B7-H4 in a subject comprising administering to the subject the antibody-drug conjugate or the pharmaceutically acceptable salt or solvate thereof according to claim 18.

63. The method according to claim 62, wherein the disease is a cancer with high B7-H4 expression, the cancer is selected from the group consisting of astroblastoma of human brain, human pharyngeal cancer, adrenal tumor, AIDS-related cancer, alveolar soft-part sarcoma, astrocytoma, bladder cancer, bone cancer, brain and spinal cord cancer, metastatic brain tumor, breast cancer, carotid body tumor, cervical cancer, chondrosarcoma, chordoma, chromophobe cell carcinoma of kidney, clear cell carcinoma, colon cancer, colorectal cancer, connective tissue proliferative small round cell tumor, ependymoma, Ewing's sarcoma, extraosseous mucoid chondrosarcoma, fibrogenesis imperfecta ossium of bone, fibrous dysplasia of bone, gallbladder or cholangiocarcinoma, gastric cancer, gestational trophoblastic disease, germ cell tumor, head and neck cancer, hepatocellular carcinoma, islet cell tumor, Kaposi's sarcoma, kidney cancer, leukemia, liposarcoma/malignant lipomatous tumor, liver cancer, lymphoma, lung cancer, medulloblastoma, melanoma, meningioma, multiple endocrine neoplasia, multiple myeloma, myelodysplastic syndrome, neuroblastoma, neuroendocrine tumor, ovarian cancer, pancreatic cancer, papillary thyroid cancer, parathyroid adenoma, pediatric cancer, peripheral schwannoma, pheocytoma, pituitary tumor, prostate cancer, posterior uveal melanoma, renal metastatic cancer, rhabdoid tumor, rhabdomyosarcoma, sarcoma, skin cancer, soft tissue sarcoma, squamous cell carcinoma, synovial sarcoma, testicular cancer, thymic cancer, thyroid metastatic cancer and uterine cancer.


SEQUENCE LISTING (55 sequences)

SEQ ID NO: 1 - protein, 276 aa, Artificial Sequence
  A synthetic huB7-H4-His
MASLGQILFWSIISIIIILAGAIALIIGFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKADYKDDDDKGSHHHHHHHH

SEQ ID NO: 2 - protein, 463 aa, Artificial Sequence
  A synthetic huB7-H4-Fc
FGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKAGSGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 10 aa, Artificial Sequence
  A synthetic HCDR1
GFTFSRYGMS

SEQ ID NO: 4 - protein, 17 aa, Artificial Sequence
  A synthetic HCDR2
GINGGGSYTYYLDTVKG

SEQ ID NO: 5 - protein, 9 aa, Artificial Sequence
  A synthetic HCDR3
QGSNYYFDY

SEQ ID NO: 6 - protein, 11 aa, Artificial Sequence
  A synthetic LCDR1
HASQGISSNIG

SEQ ID NO: 7 - protein, 7 aa, Artificial Sequence
  A synthetic LCDR2
HGTNLED

SEQ ID NO: 8 - protein, 9 aa, Artificial Sequence
  A synthetic LCDR3
VQYAQFPYT

SEQ ID NO: 9 - protein, 10 aa, Artificial Sequence
  A synthetic HCDR1
GFTFSNYYMS

SEQ ID NO: 10 - protein, 17 aa, Artificial Sequence
  A synthetic HCDR2
YVSSGGGSTYYSDSVKG

SEQ ID NO: 11 - protein, 11 aa, Artificial Sequence
  A synthetic HCDR3
ESYSQGNYFDY

SEQ ID NO: 12 - protein, 11 aa, Artificial Sequence
  A synthetic LCDR1
RASQSISDYLH

SEQ ID NO: 13 - protein, 7 aa, Artificial Sequence
  A synthetic LCDR2
FASQSIS

SEQ ID NO: 14 - protein, 9 aa, Artificial Sequence
  A synthetic LCDR3
QNGHSFSLT

SEQ ID NO: 15 - protein, 118 aa, Artificial Sequence
  A synthetic hu2G6 HCVR
EVQLLESGGGLVQPGGSLRLSCAASGFTFSRYGMSWVRQAPGKGLEWVSGINGGGSYTYYLDTVKGRFTISRDNARNTLYLQMSSLRAEDTAVYYCVSQGSNYYFDYWGQGTLVTVSS

SEQ ID NO: 16 - protein, 107 aa, Artificial Sequence
  A synthetic hu2G6 LCVR
DIRMTQSPSSLSASVGDRVTITCHASQGISSNIGWLQQKPGKAPKALIYHGTNLEDGVPSRFSGSGSGADYTLTISSLQPEDFATYYCVQYAQFPYTFGGGTKVEIK

SEQ ID NO: 17 - protein, 120 aa, Artificial Sequence
  A synthetic hu2F7 HCVR
EVQLVESGGGLVQPGGSLRLSCAASGFTFSNYYMSWVRQAPGKGLEWVAYVSSGGGSTYYSDSVKGRFTISRDNAKNTLYLQMSSLRAEDTAVYYCTRESYSQGNYFDYWGQGTTVTVSS

SEQ ID NO: 18 - protein, 107 aa, Artificial Sequence
  A synthetic hu2F7 LCVR
EIVMTQSPATLSLSPGERATLSCRASQSISDYLHWYQQKPGQSPRLLIKFASQSISGIPARFSGSGSGTDFTLTISSLEPEDFAVYYCQNGHSFSLTFGQGTKLEIK

SEQ ID NO: 19 - protein, 448 aa, Artificial Sequence
  A synthetic hu2G6 HC
EVQLLESGGGLVQPGGSLRLSCAASGFTFSRYGMSWVRQAPGKGLEWVSGINGGGSYTYYLDTVKGRFTISRDNARNTLYLQMSSLRAEDTAVYYCVSQGSNYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 214 aa, Artificial Sequence
  A synthetic hu2G6 LC
DIRMTQSPSSLSASVGDRVTITCHASQGISSNIGWLQQKPGKAPKALIYHGTNLEDGVPSRFSGSGSGADYTLTISSLQPEDFATYYCVQYAQFPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 450 aa, Artificial Sequence
  A synthetic hu2F7 HC
EVQLVESGGGLVQPGGSLRLSCAASGFTFSNYYMSWVRQAPGKGLEWVAYVSSGGGSTYYSDSVKGRFTISRDNAKNTLYLQMSSLRAEDTAVYYCTRESYSQGNYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 214 aa, Artificial Sequence
  A synthetic hu2F7 LC
EIVMTQSPATLSLSPGERATLSCRASQSISDYLHWYQQKPGQSPRLLIKFASQSISGIPARFSGSGSGTDFTLTISSLEPEDFAVYYCQNGHSFSLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 10 aa, Artificial Sequence
  A synthetic HCDR1
GYTFTNSWMN

SEQ ID NO: 24 - protein, 17 aa, Artificial Sequence
  A synthetic HCDR2
GIYPNSGNIEYNEKFKG

SEQ ID NO: 25 - protein, 6 aa, Artificial Sequence
  A synthetic HCDR3
DSRFSY

SEQ ID NO: 26 - protein, 11 aa, Artificial Sequence
  A synthetic LCDR1
KASQDVRTAVA

SEQ ID NO: 27 - protein, 7 aa, Artificial Sequence
  A synthetic LCDR2
STSYRYT

SEQ ID NO: 28 - protein, 9 aa, Artificial Sequence
  A synthetic LCDR3
QQHYSTPLT

SEQ ID NO: 29 - protein, 7 aa, Artificial Sequence
  A synthetic HCDR1
GDTFTTY

SEQ ID NO: 30 - protein, 6 aa, Artificial Sequence
  A synthetic HCDR2
YLNSGS

SEQ ID NO: 31 - protein, 6 aa, Artificial Sequence
  A synthetic HCDR3
DSRFSY

SEQ ID NO: 32 - protein, 11 aa, Artificial Sequence
  A synthetic LCDR1
KASQDVSTAVA

SEQ ID NO: 33 - protein, 7 aa, Artificial Sequence
  A synthetic LCDR2
SASYRYT

SEQ ID NO: 34 - protein, 9 aa, Artificial Sequence
  A synthetic LCDR3
QQHYNTPLT

SEQ ID NO: 35 - protein, 115 aa, Artificial Sequence
  A synthetic hu2F8 HCVR
EVQLVQSGAEVKKPGASVKVSCKASGYTFTSSWMNWVRQAPGQRLEWMGGIYPNRGNIEYNEKFKGRVTLTVDTSASTAYMELSSLRSEDTAVYYCARDSRFSYWGQGTLVTVSS

SEQ ID NO: 36 - protein, 107 aa, Artificial Sequence
  A synthetic hu2F8 LCVR
DIQMTQSPSSLSASVGDRVTITCKASQDVRTAVAWYQQKPGKAPKLLISSTSYRYTGVPSRFSGSGSGTDFTFTISSLQPEDIATYYCQQHYSTPLTFGGGTKVEIK

SEQ ID NO: 37 - protein, 115 aa, Artificial Sequence
  A synthetic hu1C9 HCVR
EVQLVQSGAEVKKPGASVKVSCKASGDTFTTYWMNWVRQAPGQRLEWMGGIYLNRGSSEYNEKFKGRVTLTVDTSASTAYMELSSLRSEDTAVYYCARDSRFSYWGQGTLVTVSS

SEQ ID NO: 38 - protein, 107 aa, Artificial Sequence
  A synthetic hu1C9 LCVR
DIQMTQSPSSLSASVGDRVTITCKASQDVSTAVAWYQQKPGKAPKLLISSASYRYTGVPSRFSGSGSGTDFTFTISSLQPEDIATYYCQQHYNTPLTFGGGTKVEIK

SEQ ID NO: 39 - protein, 445 aa, Artificial Sequence
  A synthetic hu2F8 HC
EVQLVQSGAEVKKPGASVKVSCKASGYTFTSSWMNWVRQAPGQRLEWMGGIYPNRGNIEYNEKFKGRVTLTVDTSASTAYMELSSLRSEDTAVYYCARDSRFSYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 214 aa, Artificial Sequence
  A synthetic hu2F8 LC
DIQMTQSPSSLSASVGDRVTITCKASQDVRTAVAWYQQKPGKAPKLLISSTSYRYTGVPSRFSGSGSGTDFTFTISSLQPEDIATYYCQQHYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 445 aa, Artificial Sequence
  A synthetic hu1C9HC
EVQLVQSGAEVKKPGASVKVSCKASGDTFTTYWMNWVRQAPGQRLEWMGGIYLNRGSSEYNEKFKGRVTLTVDTSASTAYMELSSLRSEDTAVYYCARDSRFSYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 214 aa, Artificial Sequence
  A synthetic hu1C9 LC
DIQMTQSPSSLSASVGDRVTITCKASQDVSTAVAWYQQKPGKAPKLLISSASYRYTGVPSRFSGSGSGTDFTFTISSLQPEDIATYYCQQHYNTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 10 aa, Artificial Sequence
  A synthetic optimized HCDR1
GYTFTSSWMN

SEQ ID NO: 44 - protein, 17 aa, Artificial Sequence
  A synthetic optimized HCDR2
GIYPNRGNIEYNEKFKG

SEQ ID NO: 45 - protein, 6 aa, Artificial Sequence
  A synthetic optimized HCDR2
YLNRGS

SEQ ID NO: 46 - protein, 118 aa, Artificial Sequence
  A synthetic 2G6 HCVR
EVQLVESGGGLVKPGGSLKLSCAASGFTFSRYGMSWVRQTPEKRLEWVAGINGGGSYTYYLDTVKGRFTISRDNSRNTLYLQMSSLRSEDTAMYYCVSQGSNYYFDYWGQGTTLTVSS

SEQ ID NO: 47 - protein, 107 aa, Artificial Sequence
  A synthetic 2G6 LCVR
DIRMTQSPSSMSVSLGDTVSITCHASQGISSNIGWLQQKPGKSFKALIYHGTNLEDGVPSRFSGSGSGADYSLTISSLESEDFADYYCVQYAQFPYTFGGGTKLEIK

SEQ ID NO: 48 - protein, 120 aa, Artificial Sequence
  A synthetic 2F7 HCVR
EVQLVESGGGLVQPGGSLKLSCAASGFTFSNYYMSWVRQTPEKRLEWVAYVSSGGGSTYYSDSVKGRFTISRDNAKNTLYLQMSSLKPEDTAMYYCTRESYSQGNYFDYWGQGTTLTVSS

SEQ ID NO: 49 - protein, 107 aa, Artificial Sequence
  A synthetic 2F7 LCVR
DIVMTQSPATLSVTPGDRVSLSCRASQSISDYLHWYQQKSHESPRLLIKFASQSISGIPSRFSGSGSGSDFTLSINSVEPEDVGVYYCQNGHSFSLTFGAGTKLELK

SEQ ID NO: 50 - protein, 115 aa, Artificial Sequence
  A synthetic 2F8 HCVR
QVQLQQPGSVLVRPGASVKLSCKASGYTFTNSWMNWAKLRPGQGLEWIGGIYPNSGNIEYNEKFKGKATLTVDTSSSTAYMDLTSLTSEDSAVYYCARDSRFSYWGQGTLVTVSA

SEQ ID NO: 51 - protein, 107 aa, Artificial Sequence
  A synthetic 2F8 LCVR
DIVMTQSHKFMSTSVGDRVSITCKASQDVRTAVAWYQQKPGQSPKLLISSTSYRYTGVPDRFTGSGSGTDFTFIISSVQAEDLAVYYCQQHYSTPLTFGAGTKLELK

SEQ ID NO: 52 - protein, 115 aa, Artificial Sequence
  A synthetic 1C9 HCVR
QVQLQQPGSVLVRPGASVKLSCKASGDTFTTYWMNWVKQRPGQGLEWIGGIYLNSGSSEYNEKFKGKATLSVDTSSSTAYMDLSSLTSEDSAVYYCARDSRFSYWGQGTLVTVSA

SEQ ID NO: 53 - protein, 107 aa, Artificial Sequence
  A synthetic 1C9 LCVR
DIVMTQSHKFLSTSVGDRVSITCKASQDVSTAVAWYQQKPGQSPELLISSASYRYTGVPDRFTGSGSGTDFTFTISSVQAEDLAVYYCQQHYNTPLTFGAGTQLELK

SEQ ID NO: 54 - protein, 330 aa, Artificial Sequence
  A synthetic IgG1 C
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 107 aa, Artificial Sequence [sequence omitted]